(19) United States
(12) Patent Application Publication
(10) Pub. No.: US 2023/0151102 A1
(43) Pub. Date: May 18, 2023

(54) METHODS AND COMPOSITIONS FOR PREVENTING ADSORPTION OF THERAPEUTIC PROTEINS TO DRUG DELIVERY SYSTEM COMPONENTS

(71) Applicant: Aptevo Research and Development LLC, Seattle, WA (US)

(72) Inventors: Jonathan CLAPPER, Seattle, WA (US); Siowfong WEE, Seattle, WA (US); Gang LI, Seattle, WA (US)

(21) Appl. No.: 17/802,927

(22) PCT Filed: Jan. 13, 2021
(86) PCT No.: PCT/US2021/013304

§ 371 Date: Aug. 26, 2022

Related U.S. Application Data
(60) Provisional application No. 62/960,602, filed on Jan. 13, 2020.

(51) Int. Cl. C07K 16/28; A61K 9/08; A61K 9/00; A61K 47/26; A61K 47/12

(52) U.S. Cl. CPC C07K 16/2866; C07K 16/2809; A61K 9/08; A61K 9/0019; A61K 47/26; A61K 47/12; A61K 2039/54

(57) ABSTRACT

The disclosure provides compositions and methods that reduce protein loss during drug delivery due to adsorption of the protein onto one or more components of a drug delivery system. In some embodiments, the disclosure provides a composition for preventing protein adsorption to one or more components of a drug delivery system, the composition comprising succinate and polysorbate 80. In some embodiments, the composition further comprises a therapeutic protein.

### CROSS REFERENCE TO RELATED APPLICATION

This application claims priority to U.S. Provisional Application No. 62/960,602, filed Jan. 13, 2020, which is incorporated by reference herein in its entirety for all purposes.

### FIELD OF THE DISCLOSURE

The disclosure relates to intravenous delivery of therapeutic proteins. More specifically, the disclosure relates to methods and compositions for preventing adsorption of therapeutic proteins to one or more components of an intravenous drug delivery system. The disclosure also relates to methods for intravenous treatment of a patient with a therapeutic protein.

### SEQUENCE LISTING

This application contains a Sequence Listing which has been submitted electronically and is hereby incorporated by reference in its entirety. The Sequence Listing was recorded Jan. 13, 2021, is named APVO_060_01WO_SeqList_ST25.txt, and is about 301 kilobytes in size.

### BACKGROUND

Protein-based therapeutics have been highly successful in the clinic. There are hundreds of therapeutic proteins approved for clinical use in the US and Europe. Approved therapeutic proteins include, for example, antibody-based drugs, Fc fusion proteins, anticoagulants, blood factors, bone morphogenetic proteins, engineered protein scaffolds, enzymes, growth factors, hormones, interferons, interleukins, and thrombolytics.

Many therapeutic proteins are administered via the intravenous route. When delivering a protein via the intravenous (i.v) route, contact surfaces are of particular concern because proteins tend to adsorb to such surfaces due to their amphipathic nature. With the widespread use of a variety of plastic polymers in syringes, i.v. containers (e.g., i.v. bags) and lines, the risk of protein loss by adsorption is substantial, especially at low concentrations. Protein adsorption phenomena may compromise the intended therapeutic benefit, drive up dosage levels, and increase treatment costs.

There remains a need in the art for improved compositions and methods for intravenous delivery of therapeutic proteins that reduce protein loss due to adsorption of the protein onto one or more components of a drug delivery system.

### SUMMARY

The instant disclosure provides compositions that can be used to reduce or eliminate protein adsorption to one or more components of a drug delivery system. The compositions may be contacted with a surface of one or more components of a drug delivery system, before the same surface is contacted with a therapeutic protein. The compositions described herein may be referred to as IVSS (Intravenous Solution Stabilizer) compositions.

Provided herein is a composition for reducing adsorption of a therapeutic protein to one or more components of an intravenous drug delivery system, the composition comprising succinate and polysorbate 80. In some embodiments, the composition comprises about 1 to about 10 mM succinate, and about 0.001% (w/v) to about 0.01% (w/v) polysorbate 80. In some embodiments, the composition comprises about 4 mM to about 6 mM succinate, such as about 5 mM succinate. In some embodiments, the composition comprises about 0.002% (w/v) to about 0.008% (w/v) polysorbate 80, such as about 0.004% (w/v) polysorbate 80. In some embodiments, the pH of the composition is about 5.0 to about 7.0, such as about 6.0. In some embodiments, the composition comprises about 5 mM succinate and about 0.0004% (w/v) polysorbate 80 in water, wherein the pH of the composition is about 6.0, and wherein the composition is formulated for injection.

The compositions disclosed herein can be utilized with any therapeutic protein that has the propensity, whether due to size, charge, and/or other characteristic, to adhere to the plastic tubing and bags used in the delivery of an intravenous drug. Accordingly, in some embodiments, the composition comprises a therapeutic protein. The therapeutic protein can be a monospecific or multispecific binding protein. In some embodiments, the therapeutic protein forms a homodimer. In some embodiments, the therapeutic protein forms a heterodimer. In some embodiments, the therapeutic protein is in a format selected from the group consisting of scFv-Fc-scFv (e.g., ADAPTIR®), quadromas, Kλ-bodies, dAbs, diabodies, TandAbs, nanobodies, DOCK-AND-LOCKs® (DNLs®), CrossMab Fabs, CrossMab VH-VLs, strand-exchange engineered domain bodies (SEEDbodies), Affibodies, Fynomers, Kunitz Domains, Albu-dabs, two engineered Fv fragments with exchanged VHs (e.g., a dual-affinity re-targeting molecules (D.A.R.T.s)), scFv x scFv (e.g., BiTE), DVD-IG, Covx-bodies, peptibodies, scFv-Igs, SVD-Igs, dAb-Igs, Knobs-in-Holes, IgG1 antibodies comprising matched mutations in the CH3 domain (e.g., DuoBody antibodies) and triomAbs.

In some embodiments, the therapeutic protein comprises at least a first binding domain. The first binding domain may be a single chain variable fragment (scFv). In some embodiments, the therapeutic protein comprises at least a first binding domain and a second binding domain, wherein the first binding domain may be a single chain variable fragment (scFv) and the second binding domain may be a scFv. In some embodiments, the first binding domain specifically binds to a tumor antigen and the second binding domain specifically binds to CD3 (for instance, CD3c). In some embodiments, the first binding domain specifically binds to CD3 and the second binding domain specifically binds to a tumor antigen.

In some embodiments, the first binding domain specifically binds to a tumor antigen and the second binding domain specifically binds to 4-1BB or OX40. In some embodiments, the first binding domain specifically binds to 4-1BB or OX40 and the second binding domain specifically binds to a tumor antigen. For instance, in some embodiments, the binding domain specifically binds to 4-1BB and the second binding domain specifically binds to a tumor antigen.

Also provided is a composition for reducing protein adsorption to one or more components of an intravenous drug delivery system, the composition comprising about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a 4-1BB binding domain, an immunoglobulin hinge domain, an immunoglobulin Fc domain, and a tumor antigen domain or, in order from amino terminus to carboxyl terminus, a tumor antigen binding domain, an immunoglobulin hinge domain, an immunoglobulin Fc domain, and a 4-1BB binding domain. In some embodiments, the binding domain specifically binds to OX40 and the second binding domain specifically binds to a tumor antigen.

Also provided is a composition for reducing protein adsorption to one or more components of an intravenous drug delivery system, the composition comprising about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus an OX40 binding domain, an immunoglobulin hinge domain, an immunoglobulin Fc domain, and a tumor antigen domain or, in order from amino terminus to carboxyl terminus, a tumor antigen binding domain, an immunoglobulin hinge domain, an immunoglobulin Fc domain, and an OX40 binding domain.

In some embodiments, the first binding domain specifically binds to 4-1BB and the second binding domain specifically binds to OX40, or the first binding domain specifically binds to OX40 and the second binding domain specifically binds to 4-1BB. Also provided is a composition for reducing protein adsorption to one or more components of an intravenous drug delivery system, the composition comprising about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus, a 4-1BB binding domain, an immunoglobulin hinge domain, an immunoglobulin Fc domain, and an OX40 binding domain or, in order from amino terminus to carboxyl terminus, an OX40 binding domain, an immunoglobulin hinge domain, an immunoglobulin Fc domain, and a 4-1BB binding domain.

In some embodiments, the first binding domain specifically binds to CD123 and/or the second binding domain specifically binds CD3c. In some embodiments, the therapeutic protein comprises, in order from amino terminus to carboxyl terminus the first binding domain, a hinge region, an immunoglobulin constant region, and the second binding domain. In some embodiments, the immunoglobulin constant region comprises immunoglobulin CH2 and CH3 domains of IgG1, IgG2, IgG3, IgG4, IgA1, IgA2 or IgD. In some embodiments, the first binding domain comprises an immunoglobulin heavy chain variable region (VH) comprising HCDR1, HCDR2, and HCDR3, and an immunoglobulin light chain variable region (VL) comprising LCDR1, LCDR2, and LCDR3. In some embodiments, the HCDR1 comprises SEQ ID NO: 10, the HCDR2 comprises SEQ ID NO: 11, and the HDCR3 comprises SEQ ID NO: 12. In some embodiments, the LCDR1 comprises SEQ ID NO: 13, the LCDR2 comprises SEQ ID NO: 14, and the LCDR3 comprises SEQ ID NO: 15. In some embodiments, the HCDR1 comprises SEQ ID NO: 10, the HCDR2 comprises SEQ ID NO: 11, and the HDCR3 comprises SEQ ID NO: 12; and the LCDR1 comprises SEQ ID NO: 13, the LCDR2 comprises SEQ ID NO: 14, and the LCDR3 comprises SEQ ID NO: 15. In some embodiments, the first binding domain comprises a sequence at least 95% identical to SEQ ID NO: 18. In some embodiments, the second binding domain comprises an immunoglobulin heavy chain variable region (VH) comprising HCDR1, HCDR2, and HCDR3, and an immunoglobulin light chain variable region (VL) comprising LCDR1, LCDR2, and LCDR3. In some embodiments, the HCDR1 comprises SEQ ID NO: 19, the HCDR2 comprises SEQ ID NO: 20, and the HDCR3 comprises SEQ ID NO: 21. In some embodiments, the LCDR1 comprises SEQ ID NO: 22, the LCDR2 comprises SEQ ID NO: 23, and the LCDR3 comprises SEQ ID NO: 24. In some embodiments, the HCDR1 comprises SEQ ID NO: 19, the HCDR2 comprises SEQ ID NO: 20, and the HDCR3 comprises SEQ ID NO: 21, the LCDR1 comprises SEQ ID NO: 22, the LCDR2 comprises SEQ ID NO: 23, and the LCDR3 comprises SEQ ID NO: 24. In some embodiments, the second binding domain comprises a sequence at least 95% or 100% identical to SEQ ID NO: 27. In some embodiments, the therapeutic protein comprises the sequence of SEQ ID NO: 31.

In some embodiments, the concentration of the therapeutic protein is about 0.01 μg/mL to about 2.0 μg/mL. In some embodiments, the concentration of the therapeutic protein is about 0.01, about 0.02, about 0.03, about 0.04, about 0.05, about 0.06, about 0.07, about 0.08, or about 0.09 μg/mL. In some embodiments, the concentration of the therapeutic protein is about 0.1, about 0.2, about 0.3, about 0.4, about 0.5, about 0.6, about 0.7, about 0.8, or about 0.9 μg/mL. In some embodiments, the concentration of the therapeutic protein is about 1.0, about 1.1, about 1.2, about 1.3, about 1.4, about 1.5, about 1.6, about 1.7, about 1.8, about 1.9, or about 2.0 μg/mL.

In some embodiments, the composition comprises about 25 to about 150 mM succinate, and about 0.01% to about 0.1% (w/v) polysorbate 80. The composition may be, for example, at a 10×-50× concentration. In some embodiments, the composition is at a 20× concentration. In some embodiments, the composition comprises about 75 mM to about 125 mM succinate, such as about 100 mM succinate. In some embodiments, the composition comprises about 0.05% (w/v) to about 0.1% (w/v) polysorbate 80, such as about 0.08% (w/v) polysorbate 80. In some embodiments, the pH of the composition is about 5.0 to about 7.0, such as about 6.0. In some embodiments, the composition comprises about 100 mM succinate and about 0.08% (w/v) polysorbate 80 in water, wherein the pH of the composition is about 6.0, and wherein the composition is formulated for injection.

Also provided is a composition for reducing protein adsorption to one or more components of an intravenous drug delivery system, the composition comprising about 100 mM succinate, about 0.08% (w/v) polysorbate 80, and a therapeutically effective amount of a therapeutic protein.

Also provided is a composition for reducing protein adsorption to one or more components of an intravenous drug delivery system, the composition comprising about 1 to about 10 mM succinate, and about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a first binding domain that specifically binds to a first target, a hinge region, an immunoglobulin constant region, a second binding domain that specifically binds to a second target. In some embodiments, the first target is CD86. In some embodiments, the first target is CD123. In some embodiments, the second target is a receptor of IL-10. In some embodiments, the second target is CD3c. In some embodiments, the first target is CD86 and the second target is a receptor of IL-10. In some embodiments, the first target is CD123 and the second target is CD3c.

Also provided is a composition for reducing protein adsorption to one or more components of an intravenous drug delivery system, the composition comprising about 1 to about 10 mM succinate, and about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a first binding domain, a hinge region, an immunoglobulin constant region, and a second binding domain, wherein the first binding domain comprises an immunoglobulin heavy chain variable region (VH) comprising HCDR1, HCDR2, and HCDR3; and an immunoglobulin light chain variable region (VL) comprising LCDR1, LCDR2, and LCDR3; wherein the HCDR1 comprises SEQ ID NO: 10, the HCDR2 comprises SEQ ID NO: 11, and the HDCR3 comprises SEQ ID NO: 12; and wherein the LCDR1 comprises SEQ ID NO: 13, the LCDR2 comprises SEQ ID NO: 14, and the LCDR3 comprises SEQ ID NO: 15; wherein the second binding domain comprises an immunoglobulin heavy chain variable region (VH) comprising HCDR1, HCDR2, and HCDR3; and an immunoglobulin light chain variable region (VL) comprising LCDR1, LCDR2, and LCDR3; wherein the HCDR1 comprises SEQ ID NO: 19, the HCDR2 comprises SEQ ID NO: 20, and the HDCR3 comprises SEQ ID NO: 21; and wherein the LCDR1 comprises SEQ ID NO: 22, the LCDR2 comprises SEQ ID NO: 23, and the LCDR3 comprises SEQ ID NO: 24.

Also provided is a composition for reducing protein adsorption to one or more components of an intravenous drug delivery system, the composition comprising about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises the sequence of SEQ ID NO: 31.

Also provided is a composition for reducing protein adsorption to one or more components of an intravenous drug delivery system, the composition comprising about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus: a CD86 binding domain, an immunoglobulin hinge domain, an immunoglobulin Fc domain, and a monomeric IL-10 domain, wherein the CD86 binding domain comprises a variable heavy chain and a variable light chain that specifically bind CD86, wherein the immunoglobulin Fc domain is an IgG1 Fc domain that comprises two or more mutations that prevent or significantly reduce binding to Fc receptors FcγR, FcγRIIa, FcγRIIb, and FcγRIIIb, wherein the monomeric IL-10 domain comprises two subunits of human IL-10 separated by a short linker, and wherein the therapeutic protein is a homodimer.

Also provided is a composition for reducing protein adsorption to one or more components of an intravenous drug delivery system, the composition comprising about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a CD86 binding domain, an immunoglobulin hinge domain, an immunoglobulin Fc domain, and a monomeric IL-10 domain, wherein the CD86 binding domain comprises an immunoglobulin heavy chain variable region (VH) comprising HCDR1, HCDR2, and HCDR3; and an immunoglobulin light chain variable region (VL) comprising LCDR1, LCDR2, and LCDR3, wherein the amino acid sequence of HCDR1 is SEQ ID: NO 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6, wherein the monomeric IL-10 domain has an amino acid sequence of SEQ ID NO:28.

Also provided is a composition for reducing protein adsorption to one or more components of an intravenous drug delivery system, the composition comprising about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a CD86 binding domain, an immunoglobulin hinge domain, an immunoglobulin Fc domain, and a monomeric IL-10 domain, wherein the CD86 binding domain comprises the amino acid sequence of SEQ ID NO: 9, and wherein the monomeric IL-10 domain comprises the amino acid sequence of SEQ ID NO: 28.

Also provided is a composition for reducing protein adsorption to one or more components of an intravenous drug delivery system, the composition comprising about 1 to about 10 mM succinate, about 0.001% (w/v) to about 0.01% (w/v) polysorbate 80; and about 0.01 μg/mL to about 2.0 μg/mL of a therapeutic protein, wherein the therapeutic protein comprises the amino acid sequence of SEQ ID NO: 30.

The disclosure also provides a container adapted for holding a therapeutic protein, wherein an interior surface of the container is first contacted with a composition of the disclosure before it is contacted with a composition comprising the therapeutic protein. In some embodiments, the container is substantially free of latex. In some embodiments, the container is substantially free of bis(2-ethylhexyl) phthalate (DEHP). In some embodiments, the container is selected from the group consisting of an IV bag, a syringe, and a tube.

The disclosure also provides a method of preparing an intravenous drug delivery system for delivery of a therapeutic protein, the method comprising providing at least one container adapted to hold the therapeutic protein, and before the therapeutic protein is added to the at least one container, contacting an interior surface of the at least one container with a composition comprising about 1 to about 10 mM succinate, and about 0.001% to 0.01% (w/v) polysorbate 80. In some embodiments, the composition coats the interior surface of the at least one container and prevents the therapeutic protein from binding to the interior surface of the container. In some embodiments, the at least one container is substantially free of latex. In some embodiments, the at least one container is substantially free of bis(2-ethylhexyl) phthalate (DEHP). In some embodiments, the at least one container is selected from the group consisting of an IV bag, a syringe, and a tube.

The disclosure also provides a method of treating a subject by intravenous administration of a therapeutic protein, the method comprising providing at least one container adapted to hold the therapeutic protein, contacting an interior surface of the container with a composition comprising about 1 to about 10 mM succinate and about 0.001% to about 0.01% (w/v) polysorbate 80, contacting the interior surface of the container with a composition comprising the therapeutic protein, and intravenously administering the therapeutic protein to the patient. In some embodiments, the therapeutic protein comprises at least a first binding domain. In some embodiments, the first binding domain is a single chain variable fragment (scFv). In some embodiments, the therapeutic protein comprises at least a first binding domain and a second binding domain. In some embodiments, the first binding domain is a single chain variable fragment (scFv) and the second binding domain is an scFv. In some embodiments, the first binding domain specifically binds to CD123. In some embodiments, the second binding domain specifically binds CD3c. In some embodiments, the therapeutic protein comprises, in order from amino terminus to carboxyl terminus the first binding domain, a hinge region, an immunoglobulin constant region, and the second binding domain. In some embodiments, the immunoglobulin constant region comprises immunoglobulin CH2 and CH3 domains of IgG1, IgG2, IgG3, IgG4, IgA1, IgA2 or IgD. In some embodiments, the first binding domain comprises an immunoglobulin heavy chain variable region (VH) comprising HCDR1, HCDR2, and HCDR3, and an immunoglobulin light chain variable region (VL) comprising LCDR1, LCDR2, and LCDR3. In some embodiments, the HCDR1 comprises SEQ ID NO: 10, the HCDR2 comprises SEQ ID NO: 11, and the HDCR3 comprises SEQ ID NO: 12. In some embodiments, the LCDR1 comprises SEQ ID NO: 13, the LCDR2 comprises SEQ ID NO: 14, and the LCDR3 comprises SEQ ID NO: 15. In some embodiments, the HCDR1 comprises SEQ ID NO: 10, the HCDR2 comprises SEQ ID NO: 11, and the HDCR3 comprises SEQ ID NO: 12, and the LCDR1 comprises SEQ ID NO: 13, the LCDR2 comprises SEQ ID NO: 14, and the LCDR3 comprises SEQ ID NO: 15.

In some embodiments, the first binding domain comprises a sequence at least 95% or 100% identical to SEQ ID NO: 18. In some embodiments, the second binding domain comprises an immunoglobulin heavy chain variable region (VH) comprising HCDR1, HCDR2, and HCDR3 and an immunoglobulin light chain variable region (VL) comprising LCDR1, LCDR2, and LCDR3. In some embodiments, the HCDR1 comprises SEQ ID NO: 19, the HCDR2 comprises SEQ ID NO: 20, and the HDCR3 comprises SEQ ID NO: 21. In some embodiments, the LCDR1 comprises SEQ ID NO: 22, the LCDR2 comprises SEQ ID NO: 23, and the LCDR3 comprises SEQ ID NO: 24. In some embodiments, the HCDR1 comprises SEQ ID NO: 19, the HCDR2 comprises SEQ ID NO: 20, and the HDCR3 comprises SEQ ID NO: 21; and the LCDR1 comprises SEQ ID NO: 22, the LCDR2 comprises SEQ ID NO: 23, and the LCDR3 comprises SEQ ID NO: 24. In some embodiments, the second binding domain comprises a sequence at least 95% identical to SEQ ID NO: 27. In some embodiments, wherein the therapeutic protein comprises the sequence of SEQ ID NO: 31. In some embodiments, the therapeutic protein comprises, in order from amino terminus to carboxyl terminus a CD86 binding domain, an immunoglobulin hinge domain, an immunoglobulin Fc domain, and a monomeric IL-10 domain, wherein the CD86 binding domain comprises a variable heavy chain and a variable light chain that specifically bind CD86, wherein the immunoglobulin Fc domain is an IgG1 Fc domain that comprises two or more mutations that prevent or significantly reduce binding to Fc receptors FcγR, FcγRIIa, FcγRIIb, and FcγRIIIb, wherein the monomeric IL-10 domain comprises two subunits of human IL-10 separated by a short linker, and wherein the therapeutic protein is a homodimer. In some embodiments, the CD86 binding domain comprises an immunoglobulin heavy chain variable region (VH) comprising HCDR1, HCDR2, and HCDR3, and an immunoglobulin light chain variable region (VL) comprising LCDR1, LCDR2, and LCDR3. In some embodiments, the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6. In some embodiments, the CD86 binding domain comprises a variable heavy chain with an amino acid sequence at least 95% or 100% identical to SEQ ID NO: 7 and a variable light chain with an amino acid sequence at least 95% or 100% identical to SEQ ID NO: 8. In some embodiments, the CD86 binding domain comprises an amino acid sequence with at least about 95% or 100% identical to SEQ ID NO: 9. In some embodiments, the monomeric IL-10 domain comprises an amino acid sequence at least 95% or 100% identical to SEQ ID NO: 28. In some embodiments, the therapeutic protein comprises SEQ ID NO: 30 or an amino acid sequence at least about 90%, at least about 95%, at least about 98%, or at least about 99% identical to SEQ ID NO: 30. In some embodiments, the therapeutic protein is administered by intravenous infusion. In some embodiments, the composition coats an interior surface of the at least one container and prevents the therapeutic protein from binding to the interior surface of the container. In some embodiments, the subject is a mammal such as a human.

Also provided is a drug delivery system for delivering a therapeutic protein to a patient, the system comprising at least one container adapted to hold the therapeutic protein, wherein an interior surface of the at least one container is contacted with a composition comprising about 1 to about 10 mM succinate, and about 0.001% to 0.01% (w/v) polysorbate 80 before it is contacted with a composition comprising the therapeutic protein.

These and other embodiments are addressed in more detail in the detailed description set forth below.

### BRIEF DESCRIPTION OF THE DRAWINGS

FIG. 1A-D shows spectral scan profiles of an IVSS solution from 200 nm to 600 nm wavelength. Spectral scan profiles were generated at 3 days (FIG. 1A), 41 days (FIG. 1B), 77 days (FIG. 1C), and 144 days (FIG. 1D) after the solution was prepared.

FIG. 2 is a schematic showing the structure of an exemplary therapeutic protein for use with the compositions and methods of the invention. The therapeutic protein, referred to herein as Q0128, is a homodimeric protein comprising two identical polypeptides that are associated by disulfide bonds. Each polypeptide comprises a CD86 binding domain, an Fc domain, and a monomeric IL-10.

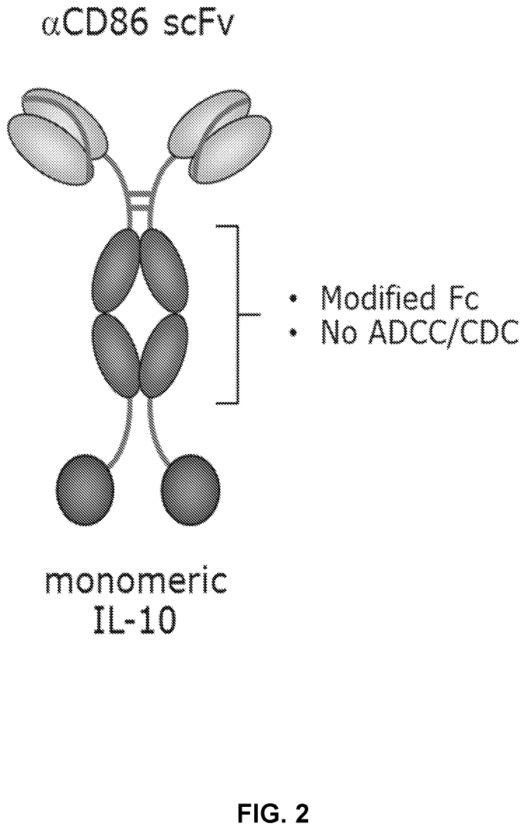

FIG. 3A and FIG. 3B are schematics showing the structures of exemplary therapeutic proteins for use with the compositions and methods of the invention. FIG. 3A shows a homodimeric protein comprising two identical polypeptides each comprising a CD3 binding domain and an Fc domain. FIG. 3B shows a homodimeric protein comprising two identical polypeptides each comprising a tumor binding domain (e.g., a CD123 binding domain), an Fc domain, and a CD3 binding domain. An exemplary CD123× CD3 bispecific therapeutic protein is referred to herein as TRI130.

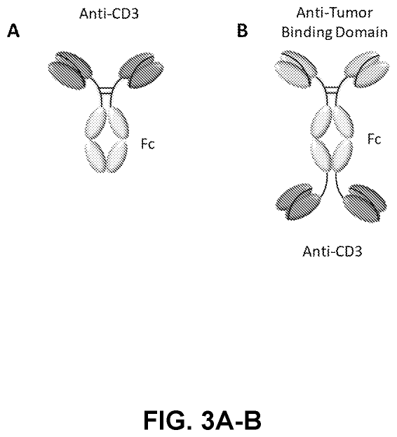

FIG. 4 is a schematic showing an exemplary protocol for using an IVSS solution to coat an interior surface of an IV bag, before a therapeutic protein is placed into the IV bag for administration to a subject in need thereof.

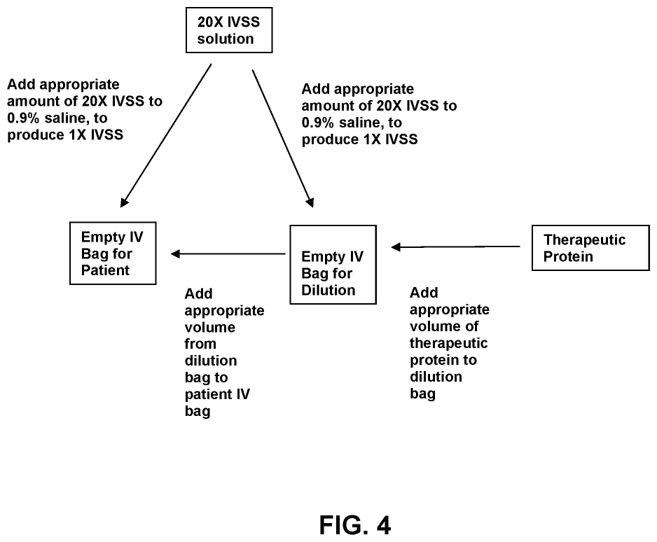

### DETAILED DESCRIPTION

The disclosure provides compositions and methods that reduce protein loss during drug delivery due to adsorption of the protein onto one or more components of a drug delivery system. The disclosure is based on the finding that protein adsorption to a surface (e.g., a surface of a drug delivery system component) can be reduced or eliminated by contacting the surface with a composition comprising succinate and polysorbate 80 before administration of the drug. Thus, in some embodiments, the disclosure provides a composition for preventing protein adsorption to one or more components of a drug delivery system, the composition comprising succinate and polysorbate 80.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. In addition, it should be understood that the polypeptides comprising the various combinations of the components (e.g., domains or regions) and substituents described herein, are disclosed by the present application to the same extent as if each polypeptide was set forth individually. Thus, selection of particular components of individual polypeptides is within the scope of the present disclosure.

### Definitions

The term “about” when immediately preceding a numerical value means±up to 10% of the numerical value. For example, “about 40” means±up to 10% of 40 (i.e., from 36 to 44), ±up to 10%, ±up to 9%, ±up to 8%, ±up to 7%, ±up to 6%, ±up to 5%, ±up to 4%, ±up to 3%, ±up to 2%, ±up to 1%, ±up to less than 1%, or any other value or range of values therein.

The terms “mcg” and “μg” are used interchangeably herein to refer to micrograms.

As used herein, “substantially” has its ordinary meaning as used in the art. For example, “substantially” may mean “significantly,” “considerably,” “largely,” “mostly,” or “essentially.” In some embodiments, “substantially” may refer to at least about 50%, at least about 60%, at least about 70%, at least about 80%, at least about 90%, at least about 95%, at least about 96%, at least about 97%, at least about 98%, or at least about 99%.

As used herein, the term “binding domain” or “binding region” refers to the domain, region, portion, or site of a protein, polypeptide, oligopeptide, or peptide or antibody or binding domain derived from an antibody that possesses the ability to specifically recognize and bind to a target molecule, such as an antigen, ligand, receptor, substrate, or inhibitor. Exemplary binding domains include single-chain antibody variable regions (e.g., domain antibodies, sFv, scFv, scFab), receptor ectodomains, and ligands (e.g., cytokines, chemokines). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains of the present disclosure that specifically bind a particular target, including Western blot, ELISA, phage display library screening, and BIACORE® interaction analysis.

A binding domain or protein “specifically binds” a target if it binds the target with an affinity or Ka (i.e., an equilibrium association constant of a particular binding interaction with units of 1/M) equal to or greater than 10⁵ M⁻¹, while not significantly binding other components present in a test sample. Binding domains can be classified as “high affinity” binding domains and “low affinity” binding domains. “High affinity” binding domains refer to those binding domains with a Ka of at least 10⁷ M⁻¹, at least 10⁸ M⁻¹, at least 10⁹ M⁻¹, at least 10¹⁰ at least 10¹¹ M⁻¹, at least 10¹² M⁻¹, or at least 10¹³ M⁻¹. “Low affinity” binding domains refer to those binding domains with a Ka of up to 10⁷ M⁻¹, up to 10⁶ M⁻¹, up to 10⁵ M⁻¹. Alternatively, affinity can be defined as an equilibrium dissociation constant (Kd) of a particular binding interaction with units of M (e.g., 10⁻⁵ M to 10⁻¹³ M). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

As used herein, a “conservative substitution” is recognized in the art as a substitution of one amino acid for another amino acid that has similar properties. Exemplary conservative substitutions are well-known in the art (see, e.g., WO 97/09433, page 10, published Mar. 13, 1997; Lehninger, Biochemistry, Second Edition; Worth Publishers, Inc. NY: NY (1975), pp. 71-77; Lewin, Genes IV, Oxford University Press, NY and Cell Press, Cambridge, Mass. (1990), p. 8). In certain embodiments, a conservative substitution includes a leucine to serine substitution.

As used herein, the term “derivative” refers to a modification of one or more amino acid residues of a peptide by chemical or biological means, either with or without an enzyme, e.g., by glycosylation, alkylation, acylation, ester formation, or amide formation.

As used herein, a polypeptide or amino acid sequence “derived from” a designated polypeptide or protein refers to the origin of the polypeptide. In certain embodiments, the polypeptide or amino acid sequence which is derived from a particular sequence (sometimes referred to as the “starting” or “parent” or “parental” sequence) has an amino acid sequence that is essentially identical to the starting sequence or a portion thereof, wherein the portion consists of at least 10-20 amino acids, at least 20-30 amino acids, or at least 30-50 amino acids, or at least 50-150 amino acids, or which is otherwise identifiable to one of ordinary skill in the art as having its origin in the starting sequence. For example, a binding domain can be derived from an antibody, e.g., a Fab, F(ab′)2, Fab′, scFv, single domain antibody (sdAb), etc.

Polypeptides derived from another polypeptide can have one or more mutations relative to the starting polypeptide, e.g., one or more amino acid residues which have been substituted with another amino acid residue or which has one or more amino acid residue insertions or deletions. The polypeptide can comprise an amino acid sequence which is not naturally occurring. Such variations necessarily have less than 100% sequence identity or similarity with the starting polypeptide. In one embodiment, the variant will have an amino acid sequence from about 60% to less than 100% amino acid sequence identity or similarity with the amino acid sequence of the starting polypeptide. In another embodiment, the variant will have an amino acid sequence from about 75% to less than 100%, from about 80% to less than 100%, from about 85% to less than 100%, from about 90% to less than 100%, from about 95% to less than 100% amino acid sequence identity or similarity with the amino acid sequence of the starting polypeptide.

As used herein, unless otherwise provided, a position of an amino acid residue in a variable region of an immunoglobulin molecule is numbered according to the IMGT numbering convention (Brochet, X, et al, Nucl. Acids Res. (2008) 36, W503-508) and a position of an amino acid residue in a constant region of an immunoglobulin molecule is numbered according to EU nomenclature (Ward et al., 1995 Therap. Immunol. 2:77-94). Other numbering conventions are known in the art (e.g., the Kabat numbering convention (Kabat, Sequences of Proteins of Immunological Interest, 5th ed. Bethesda, Md.: Public Health Service, National Institutes of Health (1991)).

As used herein, the term “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis). A “heterodimer” or “heterodimeric protein,” as used herein, refers to a dimer formed from two different polypeptides. A heterodimer does not include an antibody formed from four polypeptides (i.e., two light chains and two heavy chains). A “homodimer” or “homodimeric protein,” as used herein, refers to a dimer formed from two identical polypeptides. All disclosure of the polypeptide, including characteristics and activities (such as binding and RTCC) should be understood to include the polypeptide in its dimer form as well as other multimeric forms.

When a polypeptide of the invention is in dimeric form (i.e., a dimeric protein), it contains two binding sites at the amino-terminus and two binding sites at the carboxyl terminus. The binding domains are thus considered bivalent (i.e., two binding portions at each terminus) when the single chain polypeptides are dimerized.

A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and can comprise a human IgG hinge region.

An “altered wild-type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as disclosed in US 2013/0129723 and US 2013/0095097.

As used herein, the term “humanized” refers to a process of making an antibody or immunoglobulin binding proteins and polypeptides derived from a non-human species (e.g., mouse or rat) less immunogenic to humans, while still retaining antigen-binding properties of the original antibody, using genetic engineering techniques. In some embodiments, the binding domain(s) of an antibody or immunoglobulin binding proteins and polypeptides (e.g., light and heavy chain variable regions, Fab, scFv) are humanized. Non-human binding domains can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf BW, Dalton BJ, eds. Cellular adhesion: molecular definition to therapeutic potential. New York: Plenum Press, 1994: 291-312). If derived from a non-human source, other regions of the antibody or immunoglobulin binding proteins and polypeptides, such as the hinge region and constant region domains, can also be humanized.

An “immunoglobulin dimerization domain” or “immunoglobulin heterodimerization domain”, as used herein, refers to an immunoglobulin domain of a polypeptide chain that preferentially interacts or associates with a different immunoglobulin domain of a second polypeptide chain, wherein the interaction of the different immunoglobulin heterodimerization domains substantially contributes to or efficiently promotes heterodimerization of the first and second polypeptide chains (i.e., the formation of a dimer between two different polypeptide chains, which is also referred to as a “heterodimer”). The interactions between immunoglobulin heterodimerization domains “substantially contributes to or efficiently promotes” the heterodimerization of first and second polypeptide chains if there is a statistically significant reduction in the dimerization between the first and second polypeptide chains in the absence of the immunoglobulin heterodimerization domain of the first polypeptide chain and/or the immunoglobulin heterodimerization domain of the second polypeptide chain. In certain embodiments, when the first and second polypeptide chains are co-expressed, at least 60%, at least about 60% to about 70%, at least about 70% to about 80%, at least 80% to about 90%, about 91%, about 92%, about 93%, about 94%, about 95%, about 96%, about 97%, about 98%, or about 99% of the first and second polypeptide chains form heterodimers with each other. Representative immunoglobulin heterodimerization domains include an immunoglobulin CH1 domain, an immunoglobulin CL domain (e.g., Cκ or Cλ isotypes), or derivatives thereof, including wild type immunoglobulin CH1 and CL domains and altered (or mutated) immunoglobulin CH1 and CL domains, as provided therein.

An “immunoglobulin constant region” or “constant region” is a term defined herein to refer to a peptide or polypeptide sequence that corresponds to or is derived from part or all of one or more constant region domains. In certain embodiments, the immunoglobulin constant region corresponds to or is derived from part or all of one or more constant region domains, but not all constant region domains of a source antibody. In certain embodiments, the constant region comprises IgG CH2 and CH3 domains, e.g., IgG1 CH2 and CH3 domains. In certain embodiments, the constant region does not comprise a CH1 domain. In certain embodiments, the constant region domains making up the constant region are human. In some embodiments (for example, in certain variations of a CD123-binding polypeptide or protein comprising a second binding domain that specifically binds CD3 or another T-cell surface antigen), the constant region domains of a fusion protein of this disclosure lack or have minimal effector functions of antibody-dependent cell-mediated cytotoxicity (ADCC) and complement activation and complement-dependent cytotoxicity (CDC), while retaining the ability to bind some Fc receptors (such as FcRn, the neonatal Fc receptor) and retaining a relatively long half-life in vivo. In other variations, a fusion protein of this disclosure includes constant domains that retain such effector function of one or both of ADCC and CDC. In certain embodiments, a binding domain of this disclosure is fused to a human IgG1 constant region, wherein the IgG1 constant region has one or more of the following amino acids mutated: leucine at position 234 (L234), leucine at position 235 (L235), glycine at position 237 (G237), glutamate at position 318 (E318), lysine at position 320 (K320), lysine at position 322 (K322), or any combination thereof (numbering according to EU). For example, any one or more of these amino acids can be changed to alanine. In a further embodiment, an IgG1 Fc domain has each of L234, L235, G237, E318, K320, and K322 (according to EU numbering) mutated to an alanine (i.e., L234A, L235A, G237A, E318A, K320A, and K322A, respectively), and optionally an N297A mutation as well (i.e., essentially eliminating glycosylation of the CH2 domain). In another embodiment, the IgG1 Fc domain has each of L234A, L235A, G237A and K322A mutations.

“Fc region” or “Fc domain” refers to a polypeptide sequence corresponding to or derived from the portion of a source antibody that is responsible for binding to antibody receptors on cells and the C1q component of complement. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, CH2, and CH3 domains. However, more recently the term has been applied to a single chain consisting of CH3, CH2, and at least a portion of the hinge sufficient to form a disulfide-linked dimer with a second such chain. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

The terms patient and subject are used interchangeably herein. As used herein, the term “patient in need” or “subject in need” refers to a subject at risk of, or suffering from, a disease, disorder or condition that is amenable to treatment or amelioration with a therapeutic protein or a composition thereof provided herein. A subject in need may, for instance, be a patient diagnosed with a disease associated with the expression of CD123 such as acute myeloid leukemia (AML), B-lymphoid leukemia, blastic plasmocytoid dendritic neoplasms (BPDCN), hairy cell leukemia (HCL), myelodysplastic syndrome (MDS), acute lymphoblastic leukemia (ALL), refractory anemia with excess blasts (RAEB), chronic myeloid leukemia and Hodgkin's lymphoma.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

The term “expression” refers to the biosynthesis of a product encoded by a nucleic acid. For example, in the case of nucleic acid segment encoding a polypeptide of interest, expression involves transcription of the nucleic acid segment into mRNA and the translation of mRNA into one or more polypeptides.

The terms “expression unit” and “expression cassette” are used interchangeably herein and denote a nucleic acid segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. In addition to a transcriptional promoter and terminator, an expression unit can further include other nucleic acid segments such as, e.g., an enhancer or a polyadenylation signal.

The term “expression vector,” as used herein, refers to a nucleic acid molecule, linear or circular, comprising one or more expression units. In addition to one or more expression units, an expression vector can also include additional nucleic acid segments such as, for example, one or more origins of replication or one or more selectable markers. Expression vectors are generally derived from plasmid or viral DNA, or can contain elements of both.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. The comparison window for nucleic acid sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least 80%, at least 85%, at least 90%, at least 95%, at least 96%, at least 97%, at least 98%, or at least 99% sequence identity relative to each other.

“C D3” is known in the art as a multi-protein complex of six chains (see, e.g., Abbas and Lichtman, 2003; Janeway et al., p. 172 and 178, 1999), which are subunits of the T-cell receptor complex. In mammals, the CD3 subunits of the T-cell receptor complex are a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T-cell receptor chains. The intracellular tails of the CD3γ, CD3δ, and CD3ε chains each contain a single conserved motif known as an immunoreceptor tyrosine-based activation motif or ITAM, whereas each CD3ζ chain has three. It is believed the ITAMs are important for the signaling capacity of a TCR complex. CD3 as used in the present disclosure can be from various animal species, including human, monkey, mouse, rat, or other mammals.

The term “CD123” may refer to any isoform of CD123, also known as Cluster of Differentiation 123, Interleukin-3 receptor alpha chain, and IL3RA. CD123 associates with the beta chain of the interleukin-3 receptor to form the receptor. CD123 is a type I transmembrane glycoprotein, with an extracellular domain comprising a predicted Ig-like domain and two FnIII domains. The CD123-binding domains of the disclosure bind to the extracellular domain of CD123.

CD123 is also known as the alpha chain of the human interleukin-3 (IL-3) receptor. CD123 is a type I transmembrane glycoprotein and is a member of the cytokine receptor superfamily. The interleukin-3 receptor is a heterodimer formed by CD123 and the beta chain (CD131). IL-3 binds to CD123, and signal transduction is provided by CD131. IL-3 regulates the function and production of hematopoietic and immune cells and stimulates endothelial cell proliferation (Testa et al., Biomark Res. 2:4 (2014)).

CD123 is overexpressed in many hematologic malignancies, including a subset of acute myeloid leukemia (AML), B-lymphoid leukemia, blastic plasmocytoid dendritic neoplasms (BPDCN) and hairy cell leukemia. While most AML patients respond well to initial therapies, the majority of AML patients are ultimately diagnosed with relapsed or refractory disease (Ramos et al., J. Clin. Med. 4:665-695 (2015)). There is a need for molecules targeting CD123 with increased efficiency and potency and reduced adverse effects and that may be used to treat disorders associated with dysregulation of CD123.

“CD86” is known in the art as a surface molecule that belongs to the B7 receptor subfamily and functions as a T-cell costimulatory molecule (Lu et al. 1997; Vicenti et al. 2008). It is normally expressed on cells with Antigen Presenting Cell (APC) function such as dendritic cells, monocytes and activated but not resting B cells (Lu et al. 1997; Vicenti et al. 2008). It is expressed at high levels by naïve human monocytes and DC and it is further upregulated under some activation conditions (Hathcock et al. 1994); Sansom et al. 2003). Expression of CD86 on naive monocytes is estimated to be in the range of 2,000 to 5,000 copies per cell (Wolk et al. 2007). High levels of CD86 expression are associated with inflamed tissues in specific pathological conditions (Vuckovic et al. 2001; Nakazawa et al. 1999) CD86 and CD80, the latter a second member of the B7 family, facilitate T-cell activation by interacting with the T-cell co-receptor CD28.

A “CD86 binding domain” specifically binds to CD86. In some embodiments, the CD86-binding domain binds to an epitope located on the extracellular domain of CD86 (e.g., human CD86). In certain aspects, this epitope is a discontinuous and/or conformational epitope. In some embodiments, the CD86 binding domain binds CD86 but does not bind CD80. In some embodiments, the CD86 binding domain binds human CD86. In some embodiments, the CD86 binding domain binds to non-human primate CD86. In some embodiments, the CD86 binding domain binds human CD86 and also cross-reacts with cynomolgus CD86. In some embodiments, the CD86 binding domain binds to cynomolgus macaque monocytes and lineage negative populations (DC). In some embodiments, the CD86 binding domain is humanized.

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Proteins are defined herein in terms of their amino acid backbone structures; substituents such as carbohydrate groups are generally not specified, but may be present nonetheless. The terms “protein,” “polypeptide,” “therapeutic protein,” and “therapeutic polypeptide” are used interchangeably herein.

A therapeutic protein may be an antibody or an antigen-binding fragment of an antibody. In some embodiments, a therapeutic protein may also be an scFv-Fc-scFv molecule, bispecific T-cell engager (scFv-scFv) molecule, or dual affinity re-targeting molecule. In some embodiments, a therapeutic protein may be a recombinant multispecific protein. In other embodiments, a multispecific protein may be produced by chemically linking two different monoclonal antibodies or by fusing two hybridoma cell lines to produce a hybrid-hybridoma. Other multivalent formats that can be used for therapeutic proteins include, for example, scFv-Fc-scFv (e.g., ADAPTIR™), quadromas, Kλ-bodies, dAbs, diabodies, TandAbs, nanobodies, Small Modular ImmunoPharmaceutials (SMIPs™), DOCK-AND-LOCKs® (DNLs®), CrossMab Fabs, CrossMab VH-VLs, strand-exchange engineered domain bodies (SEEDbodies), Affibodies, Fynomers, Kunitz Domains, Albu-dabs, two engineered Fv fragments with exchanged VHs (e.g., a dual-affinity re-targeting molecules (D.A.R.T.s)), scFv x scFv (e.g., BiTE), DVD-IG, Covx-bodies, peptibodies, scFv-Igs, SVD-Igs, dAb-Igs, Knobs-in-Holes, IgG1 antibodies comprising matched mutations in the CH3 domain (e.g., DuoBody antibodies) and triomAbs. Exemplary bispecific formats are discussed in Garber et al., Nature Reviews Drug Discovery 13:799-801 (2014), which is herein incorporated by reference in its entirety. Additional exemplary bispecific formats are discussed in Liu et al. Front. Immunol. 8:38 doi: 10.2289/fimmu.2017.00038, and Brinkmann and Kontermann, MABS 9: 2, 182-212 (2017), each of which is herein incorporated by reference in its entirety. In certain embodiments, a bispecific antibody can be a F(ab′)2 fragment. A F(ab′)2 fragment contains the two antigen-binding arms of a tetrameric antibody molecule linked by disulfide bonds in the hinge region.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence, but is not necessarily at the carboxyl-terminus of the complete polypeptide.

As used herein, the term “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual, or prevent onset of additional associated diseases.

As used herein, the term “therapeutically effective amount (or dose)” or “effective amount (or dose)” of a specific binding molecule or compound refers to that amount of the compound sufficient to result in amelioration of one or more symptoms of the disease being treated in a statistically significant manner or a statistically significant improvement in organ function. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or concurrently in separate formulations).

The terms “light chain variable region” (also referred to as “light chain variable domain” or “VL” or V_{L}) and “heavy chain variable region” (also referred to as “heavy chain variable domain” or “VH” or V_{H}) refer to the variable binding region from an antibody light and heavy chain, respectively. The variable binding regions are made up of discrete, well-defined sub-regions known as “complementarity determining regions” (CDRs) and “framework regions” (FRs), generally comprising in order FR1-CDR1-FR2-CDR2-FR3-CDR3-FR4 from amino-terminus to carboxyl-terminus. In one embodiment, the FRs are humanized. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 domain of the heavy chain linked to the light chain via an inter-chain disulfide bond.

As used herein, a “container adapted to hold the therapeutic protein” refers to any clinically acceptable container suitable for holding and/or conveying a therapeutic protein. Non-limiting examples of such containers include, for example, IV bags, syringes, tubes/tubing, etc. In some embodiments, the container is substantially free of latex and/or bis(2-ethylhexyl) phthalate (DEHP).

An “intravenous drug delivery system” may refer to any clinically acceptable system used to prepare (e.g., dilute, mix, etc.) and/or deliver a drug to a subject or patient intravenously. Such systems may comprise, for example, an IV bag, a syringe, tubes/tubing, a pump, a needle, etc.

### Compositions for Preventing Protein Adsorption

Therapeutic proteins are often administered intravenously using a drug delivery system. For example, a sterile solution containing a protein therapeutic may be provided in an IV bag or other container, and injected/infused into the body of a patient through a tube attached to a needle, which is inserted into a vein of the patient. Thus, during administration of a therapeutic protein, the protein comes into contact with one or more surfaces of a drug delivery system, for example an interior surface of an IV bag or tube. Therapeutic proteins are known to be adsorbed to such surfaces, for example when charged amino acids on the surface of the protein interact with the surface. The tendency for proteins to remain attached to a surface depends largely on the material properties, such as surface energy, texture, and relative charge distribution. Larger proteins are more likely to adsorb and remain attached to a surface due to the higher number of contact sites between amino acids and the surface.

Protein adsorption can be a significant concern during administration of a therapeutic protein to a patient. For example, adsorption of a therapeutic protein to a surface of a drug delivery system may reduce the dose of the protein that is delivered to the patient. Protein adsorption may be particularly problematic during administration of protein therapeutics at low-dose and/or low concentration (i.e., 10 mcg/m L).

The instant disclosure provides compositions that can be used to reduce or eliminate protein adsorption to one or more components of a drug delivery system. The compositions may be contacted with a surface of one or more components of a drug delivery system, before administration of a therapeutic protein. In some embodiments, the composition coats an interior surface of at least one component of the drug delivery system and prevents the therapeutic protein from binding to the interior surface of that component.

The compositions for preventing protein adsorption may comprise a buffer and a surfactant. In some embodiments, the compositions may further comprise a therapeutic protein. The pH of the composition may be in the range of about 5.0 to about 7.0, for example about 5.0, about 5.25, about 5.5, about 5.75, about 6.0, about 6.25, about 6.5, about 6.75, or about 7.0.

In some embodiments, the composition comprises about 1 to about 10 mM of the buffer, and about 0.001% (w/v) to about 0.01% (w/v) of the surfactant. In embodiments, the composition comprises about 4 mM to about 6 mM of the buffer, for example about 5 mM of the buffer.

In further embodiments, the composition comprises about 25 to about 150 mM of the buffer. In embodiments, the composition comprises about 75 to about 125 mM of the buffer, for example about 100 mM of the buffer.

In some embodiments, the composition comprises about 0.002% (w/v) to about 0.008% (w/v) of the surfactant. In embodiments, the composition comprises about 0.004% (w/v) of the surfactant.

In some embodiments, the composition comprises about 0.05% (w/v) to about 0.1% (w/v) of the surfactant. For example, the composition may comprise about 0.05% (w/v) to about 0.1% (w/v) of the surfactant. In specific embodiments, the composition comprises about 0.08% (w/v) of the surfactant.

In some embodiments, the buffer may be a succinate buffer. In some embodiments, the surfactant may be polysorbate-80. In further embodiments, the buffer may be succinate and the surfactant may be polysorbate-80. Succinate is a salt or ester of succinic acid. Polysorbate 80 is a nonionic surfactant and emulsifier.

In some embodiments, a composition for reducing adsorption of a therapeutic protein to one or more components of an intravenous drug delivery system comprises succinate and polysorbate 80. In some embodiments, the composition comprises about 25 to about 150 mM succinate. In some embodiments, the composition comprises about 75 to about 125 mM succinate, for example about 100 mM succinate. In some embodiments, the composition comprises about 0.002% (w/v) to about 0.008% (w/v) polysorbate 80. In embodiments, the composition comprises about 0.004% (w/v) polysorbate 80. In embodiments, the composition comprises about 0.05% (w/v) to about 0.1% (w/v) polysorbate 80. For example, the composition may comprise about 0.05% (w/v) to about 0.1% (w/v) polysorbate 80. In some embodiments, the composition comprises about 0.08% (w/v) polysorbate 80.

In embodiments, the composition comprises about 4 mM to about 6 mM succinate, for example about 5 mM succinate. In some embodiments, the composition comprises about 0.002% (w/v) to about 0.008% (w/v) polysorbate 80, such as about 0.004% (w/v) polysorbate 80. In some embodiments, the composition comprises about 1 to about 10 mM succinate, and about 0.001% (w/v) to about 0.01% (w/v) polysorbate 80. In some embodiments, the composition comprises about 5 mM succinate and about 0.0004% (w/v) polysorbate 80 in water, wherein the pH of the composition is about 6.0, and wherein the composition is formulated for injection.

In some embodiments, the composition further comprises a therapeutic protein. The concentration of the therapeutic protein may be about 0.01 μg/mL to about 2.0 μg/mL. In some embodiments, the concentration of the therapeutic protein is about 0.01, about 0.02, about 0.03, about 0.04, about 0.05, about 0.06, about 0.07, about 0.08, or about 0.09 μg/mL. In some embodiments, the concentration of the therapeutic protein is about 0.1, about 0.2, about 0.3, about 0.4, about 0.5, about 0.6, about 0.7, about 0.8, or about 0.9 μg/mL. In some embodiments, the concentration of the therapeutic protein is about 1.0, about 1.1, about 1.2, about 1.3, about 1.4, about 1.5, about 1.6, about 1.7, about 1.8, about 1.9, or about 2.0 μg/mL.

In some embodiments, a composition for reducing protein adsorption to one or more components of an intravenous drug delivery system comprises about 100 mM succinate, about 0.08% (w/v) polysorbate 80, and a therapeutically effective amount of a therapeutic protein.

In some embodiments, a composition for reducing protein adsorption to one or more components of an intravenous drug delivery system comprises about 1 to about 10 mM succinate, and about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a first binding domain that specifically binds to a first target, a hinge region, an immunoglobulin constant region, a second binding domain that specifically binds to a second target. In some embodiments, the first target is CD86. In some embodiments, the first target is CD123. In some embodiments, the second target is a receptor of IL-10. In some embodiments, the second target is CD3c. In some embodiments, the first target is CD86 and the second target is a receptor of IL-10. In some embodiments, the first target is CD123 and the second target is CD3c.

In some embodiments, a composition for reducing protein adsorption to one or more components of an intravenous drug delivery system comprises about 1 to about 10 mM succinate, and about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a first binding domain, a hinge region, an immunoglobulin constant region, and a second binding domain, wherein the first binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3; wherein the HCDR1 comprises SEQ ID NO: 10, the HCDR2 comprises SEQ ID NO: 11, and the HDCR3 comprises SEQ ID NO: 12; and wherein the LCDR1 comprises SEQ ID NO: 13, the LCDR2 comprises SEQ ID NO: 14, and the LCDR3 comprises SEQ ID NO: 15, wherein the second binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3; wherein the HCDR1 comprises SEQ ID NO: 19, the HCDR2 comprises SEQ ID NO: 20, and the HDCR3 comprises SEQ ID NO: 21; and wherein the LCDR1 comprises SEQ ID NO: 22, the LCDR2 comprises SEQ ID NO: 23, and the LCDR3 comprises SEQ ID NO: 24.

In some embodiments, a composition for reducing protein adsorption to one or more components of an intravenous drug delivery system comprises about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises the sequence of SEQ ID NO: 31.

In some embodiments, a composition for reducing protein adsorption to one or more components of an intravenous drug delivery system comprises about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a CD86 binding domain, an immunoglobulin hinge domain, an immunoglobulin Fc domain, and a monomeric IL-10 domain, wherein the CD86 binding domain comprises a variable heavy chain and a variable light chain that specifically bind CD86, wherein the immunoglobulin Fc domain is an IgG1 Fc domain that comprises two or more mutations that prevent or significantly reduce binding to Fc receptors FcγR, FcγRIIa, FcγRIIb, and FcγRIIIb, wherein the monomeric IL-10 domain comprises two subunits of human IL-10 separated by a short linker, and wherein the therapeutic protein is a homodimer.

In some embodiments, a composition for reducing protein adsorption to one or more components of an intravenous drug delivery system comprises about 1 to about 10 mM succinate, about 0.001% (w/v) to about 0.01% (w/v) polysorbate 80, and about 0.01 μg/mL to about 2.0 μg/mL of a therapeutic protein, wherein the therapeutic protein comprises, in order from amino terminus to carboxyl terminus a CD86 binding domain, an immunoglobulin hinge domain, an immunoglobulin Fc domain, and a monomeric IL-10 domain, wherein the CD86 binding domain comprises an immunoglobulin heavy chain variable region (VH) comprising HCDR1, HCDR2, and HCDR3; and an immunoglobulin light chain variable region (VL) comprising LCDR1, LCDR2, and LCDR3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6, wherein the monomeric IL-10 domain has an amino acid sequence of SEQ ID NO:28.

In some embodiments, a composition for reducing protein adsorption to one or more components of an intravenous drug delivery system comprises about 1 to about 10 mM succinate, about 0.001% (w/v) to about 0.01% (w/v) polysorbate 80; and about 0.01 μg/mL to about 2.0 μg/mL of a therapeutic protein, wherein the therapeutic protein comprises, in order from amino terminus to carboxyl terminus: a CD86 binding domain, an immunoglobulin hinge domain, an immunoglobulin Fc domain, and a monomeric IL-10 domain, wherein the CD86 binding domain comprises the amino acid sequence of SEQ ID NO: 9, and wherein the monomeric IL-10 domain comprises the amino acid sequence of SEQ ID NO: 28.

In some embodiments, a composition for reducing protein adsorption to one or more components of an intravenous drug delivery system comprises about 1 to about 10 mM succinate, about 0.001% (w/v) to about 0.01% (w/v) polysorbate 80; and about 0.01 μg/mL to about 2.0 μg/mL of a therapeutic protein, wherein the therapeutic protein comprises the amino acid sequence of SEQ ID NO: 30.

In some embodiments, the composition may be provided at a concentration that is greater than 1×. For example, the composition may be at a 10× to a 50× concentration. In some embodiments, the composition may be at a 2×, 5×, 10×, 15×, 20×, 25×, 30×, 35×, 40×, 45×, or 50× concentration. In some embodiments, the composition is at a 20× concentration. As used in this context, “X” indicates that the solution is in a concentrated form that must usually be diluted to a 1× concentration for use. For example, a 5× concentrated solution must be diluted 5-fold, while a 100× concentrated solution must be diluted 100-fold. The dilution may be performed using, for example, water or saline.

In some embodiments, the composition comprises about 25 to about 150 mM succinate, and about 0.01% to about 0.1% (w/v) polysorbate 80. In some embodiments, the composition comprises about 75 mM to about 125 mM succinate, such as about 100 mM succinate. In some embodiments, the composition comprises about 0.05% (w/v) to about 0.1% (w/v) polysorbate 80, such as about 0.08% (w/v) polysorbate 80. In some embodiments, the pH of the composition is about 5.0 to about 7.0, such as about 6.0. In some embodiments, the composition comprises about 100 mM succinate and about 0.08% (w/v) polysorbate 80 in water, wherein the pH of the composition is about 6.0, and wherein the composition is formulated for injection.

In some embodiments, a 20×IVSS solution is provided, wherein the 20× solution comprises about 25 to about 150 mM succinate, and about 0.01% to about 0.1% (w/v) polysorbate 80. In some embodiments, the 20×IVSS solution comprises about 100 mM succinate and about 0.08% (w/v) polysorbate 80. In some embodiments, the 20×IVSS solution comprises about 100 mM succinate and about 0.08% (w/v) polysorbate 80 in water, wherein the pH of the composition is about 6.0, and wherein the composition is formulated for injection.

In some embodiments, the 20×IVSS solution is diluted to a 1× concentration. In some embodiments, a 1×IVSS solution comprises about 1 to about 10 mM succinate, and about 0.001% (w/v) to about 0.01% (w/v) polysorbate 80. In some embodiments, a 1×IVSS solution comprises about 5 mM succinate and about 0.004% (w/v) polysorbate 80. In some embodiments, the 1×IVSS solution comprises about 5 mM succinate and about 0.004% (w/v) polysorbate 80 in water, wherein the pH of the composition is about 6.0, and wherein the composition is formulated for injection. In some embodiments, the 1×IVSS solution further comprises a therapeutic protein, such as an anti-CD123× anti-CD3 bispecific binding protein, or an anti-CD86×monomeric IL-10 binding protein.

In some embodiments, the 1×IVSS solution (with or without the therapeutic protein) is used to coat at least one component of a drug delivery system adapted for delivery of the therapeutic protein, before delivery of the therapeutic protein.

In some embodiments, the composition may further comprise one or more additional components, such as a pharmaceutically acceptable carrier or excipient.

### Therapeutic Proteins

The compositions and methods described herein may be used in connection with the preparation, storage, and/or administration of many different types of therapeutic proteins, to prevent adsorption thereof to one or more surfaces. The therapeutic proteins may be, for example, antibody-based drugs, Fc fusion proteins, anticoagulants, blood factors, bone morphogenetic proteins, engineered protein scaffolds, enzymes, growth factors, hormones, cytokines, interferons, interleukins, or thrombolytics. In some embodiments the therapeutic protein is a ligand for a target receptor.

### Binding Domain

In some embodiments, the therapeutic proteins comprise at least one binding domain. The binding domain may provide for specific binding to at least one cell-surface molecule (e.g., a cell-surface receptor). The binding domain can be in the form of an antibody, or fragment thereof, or a fusion protein of any of a variety of different formats (e.g., the fusion protein can be in the form of a bispecific or multispecific molecule). In other embodiments, the binding domain can comprise, for example, a particular cytokine or a molecule that targets the binding domain polypeptide to, for example, a particular cell type, a toxin, an additional cell receptor, or an antibody.

In some embodiments, a binding domain described herein is derived from an antibody and comprises a variable heavy chain (V_{H}) and a variable light chain (V_{L}). For example, a single chain variable fragment (scFv) comprising a V_{H} and V_{L} chain. These binding domains and variable chains may be arranged in any order that still retains some binding to the target(s). In some embodiments, a binding domain comprises (i) an immunoglobulin heavy chain variable region (V_{H}) comprising HCDR1, HCDR2, and HCDR3; and (ii) an immunoglobulin light chain variable region (V_{L}) comprising LCDR1, LCDR2, and LCDR3.

In some embodiments, the polypeptides and proteins described herein comprise binding domains that are scFvs. In such embodiments, the binding domains may be referred to as scFv domains. In some embodiments, a binding domain is a single-chain Fv fragment (scFv) that comprises V_{H} and V_{L} regions specific for a target of interest. In certain embodiments, the V_{H} and V_{L} regions are human or humanized. In some variations, a binding domain is a single-chain Fv (scFv) comprising V_{L} and V_{H} regions joined by a peptide linker.

In certain embodiments, the binding domains of the polypeptides described herein comprise (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some embodiments, amino acid sequences provided for polypeptide constructs do not include the human immunoglobulin leader sequences. CDR sequences and amino acid substitution positions shown are those defined using the IMGT criteria (Brochet et al., Nucl. Acids Res. (2008) 36, W503-508).

In certain embodiments, a binding domain V_{L} and/or V_{H} region of the present disclosure is derived from a V_{L} and/or V_{H} of a parent V_{L} and/or V_{H} region (e.g., 1618/1619 as described in PCT Application Publication No. WO 2016/185016) and optionally contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the V_{L} and/or V_{H} sequence of a known monoclonal antibody. The insertion(s), deletion(s) or substitution(s) can be anywhere in the V_{L} and/or V_{H} region, including at the amino- or carboxyl-terminus or both ends of this region, provided that each CDR comprises zero changes or at most one, two, or three changes. In some embodiments, the binding domain containing the modified V_{L} and/or V_{H} region can still specifically bind its target with an affinity similar to or greater than the parent binding domain.

The use of peptide linkers for joining V_{L} and V_{H} regions is well-known in the art, and a large number of publications exist within this particular field. In some embodiments, a peptide linker is a 15mer consisting of three repeats of a Gly-Gly-Gly-Gly-Ser (SEQ ID NO: 128) amino acid sequence ((Gly₄Ser)₃) (SEQ ID NO: 59). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). In certain embodiments, the V_{L} and V_{H} regions are joined by a peptide linker having an amino acid sequence comprising the formula (Gly₄Ser)ₙ, wherein n=1-5 (SEQ ID NO: 129). For instance, in one embodiment of the invention, the linker comprises (Gly₄Ser)₄ (SEQ ID NO:61). Other suitable linkers can be obtained by optimizing a simple linker through random mutagenesis. In some embodiments, the V_{H} region of the scFv described herein may be positioned N-terminally to a linker sequence. In some embodiments, the V_{L} region of the scFvs described herein may be positioned C-terminally to the linker sequence.

### Hinge

In addition to a binding domain, the therapeutic polypeptides may further comprise a hinge region. In some embodiments, the hinge is an altered immunoglobulin hinge in which one or more cysteine residues in a wild type immunoglobulin hinge region are substituted with one or more other amino acid residues (e.g., serine or alanine). Exemplary altered immunoglobulin hinges, carboxyl-terminus linkers, and amino-terminus linkers include an immunoglobulin human IgG1 hinge region having one, two or three cysteine residues found in a wild type human IgG1 hinge substituted by one, two or three different amino acid residues (e.g., serine or alanine). An altered immunoglobulin hinge can additionally have a proline substituted with another amino acid (e.g., serine or alanine). For example, the above-described altered human IgG1 hinge can additionally have a proline located carboxyl-terminal to the three cysteines of wild type human IgG1 hinge region substituted by another amino acid residue (e.g., serine, alanine). In one embodiment, the prolines of the core hinge region are not substituted. In certain embodiments, a hinge, a carboxyl-terminus linker, or an amino-terminus linker polypeptide comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a wild type immunoglobulin hinge region, such as a wild type human IgG1 hinge, a wild type human IgG2 hinge, or a wild type human IgG4 hinge.

### Immunoglobulin Constant Domain

The therapeutic proteins may also comprise an immunoglobulin constant (Fc) domain (also referred to herein as a constant region, Fc domain, Fc region, and the like). In certain embodiments, the constant region comprises IgG CH2 and CH3 domains, e.g., IgG1 CH2 and CH3 domains. In certain embodiments, the constant region does not comprise a CH1 domain. In certain embodiments, the constant domains making up the constant region are human or derived from human sequences. In some embodiments, the Fc domain comprises mutations at positions 234, 235, 237 and 322. In some embodiments, the Fc domain comprises mutations at positions 234, 235, 237, 318, 320 and 322. In some embodiments, the Fc domain comprises mutations L234A, L235A, G237A and K322A. In some embodiments, the Fc domain comprises mutations L234A, L235A, G237A, E318A, K320A, and K322A. In some embodiments, the Fc domain is derived from IgG1. In some embodiments, the Fc domain that is derived from IgG1 comprises two or more mutations that prevent the polypeptide from depleting CD86 and/or IL-10R expressing cells when administered to a patient. In some embodiments, the two or more mutations in the IgG1 Fc domain prevent or substantially reduce signaling through Fc-mediated cross-linking.

In some embodiments, the immunoglobulin constant region comprises an amino acid sequence of any one of SEQ ID NO: 32-35, or a variant thereof. The inclusion of an immunoglobulin constant region slows clearance of the polypeptides and proteins of the present invention from circulation after administration to a subject. By mutations or other alterations, an immunoglobulin constant region further enables relatively easy modulation of polypeptide effector functions (e.g., ADCC, ADCP, CDC, complement fixation, and binding to Fc receptors), which can either be increased or decreased depending on the disease being treated, as known in the art and described herein. In certain embodiments, the polypeptides and proteins described herein comprise an immunoglobulin constant region capable of mediating one or more of these effector functions. In other embodiments, one or more of these effector functions are reduced or absent in an immunoglobulin constant region of a polypeptide or protein described in the present disclosure, as compared to a corresponding wild-type immunoglobulin constant region.

An immunoglobulin constant region present in the polypeptides and proteins of the present disclosure can comprise or can be derived from part or all of: a CH2 domain, a CH3 domain, a CH4 domain, or any combination thereof. For example, an immunoglobulin constant region can comprise a CH2 domain, a CH3 domain, both CH2 and CH3 domains, both CH3 and CH4 domains, two CH3 domains, a CH4 domain, two CH4 domains, and a CH2 domain and part of a CH3 domain. In certain embodiments, the polypeptides or proteins described herein do not comprise a CH1 domain.

A polypeptide or protein described herein may comprise a wild type immunoglobulin CH2 domain or an altered immunoglobulin CH2 domain from certain immunoglobulin classes or subclasses (e.g., IgG1, IgG2, IgG3, IgG4, IgA1, IgA2, or IgD) and from various species (including human, mouse, rat, and other mammals). In certain embodiments, a CH2 domain of a polypeptide or a protein described herein is a wild type human immunoglobulin CH2 domain, such as wild type CH2 domains of human IgG1, IgG2, IgG3, IgG4, IgA1, IgA2, or IgD, as set forth in SEQ ID NOs: 115, 199-201 and 195-197, respectively, of U.S. Patent Application Publication No. 2013/0129723 (said sequences incorporated by reference herein). In certain embodiments, the CH2 domain is a wild type human IgG1 CH2 domain as set forth in SEQ ID NO: 115 of U.S. Patent Application Publication No. US 2013/0129723 (said sequence incorporated by reference herein).

In certain embodiments, an altered CH2 region in a polypeptide or a protein of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin CH2 region, such as the CH2 region of wild type human IgG1, IgG2, or IgG4, or mouse IgG2a (e.g., IGHG2c).

An altered immunoglobulin CH2 region in a polypeptide or protein of the present disclosure can be derived from a CH2 region of various immunoglobulin isotypes, such as IgG1, IgG2, IgG3, IgG4, IgA1, IgA2, and IgD, from various species (including human, mouse, rat, and other mammals). In certain embodiments, an altered immunoglobulin CH2 region in a fusion protein of the present disclosure can be derived from a CH2 region of human IgG1, IgG2 or IgG4, or mouse IgG2a (e.g., IGHG2c), whose sequences are set forth in SEQ ID NOs: 115, 199, 201, and 320 of U.S. Patent Application Publication No. 2013/0129723 (said sequences incorporated by reference herein). In certain embodiments, an altered CH2 domain of a polypeptide or a protein described herein is an altered human IgG1 CH2 domain with mutations known in the art that enhance or reduce immunological activities (i.e., effector functions) such as ADCC, ADCP, CDC, complement fixation, Fc receptor binding, or any combination thereof.

In certain embodiments, a CH2 domain of a polypeptide or a protein described herein is an altered immunoglobulin CH2 region (e.g., an altered human IgG1 CH2 domain) that comprises one or more amino acid deletions or substitutions. In some embodiments, the CH2 domain comprises an amino acid substitution at the asparagine of position 297 (e.g., asparagine to alanine). Such an amino acid substitution reduces or eliminates glycosylation at this site and abrogates efficient Fc binding to FcγR and C1q. The sequence of an altered human IgG1 CH2 domain with an Asn to Ala substitution at position 297 is set forth in SEQ ID NO: 324 of U.S. Patent Application Publication No. 2013/0129723 (said sequence incorporated by reference herein). In some embodiments, the altered CH2 domain comprises at least one substitution or deletion at positions 234 to 238. For example, an immunoglobulin CH2 region can comprise a substitution at position 234, 235, 236, 237 or 238; positions 234 and 235; positions 234 and 236; positions 234 and 237; positions 234 and 238; positions 234-236; positions 234, 235 and 237; positions 234, 236 and 238; positions 234, 235, 237, and 238; positions 236-238; or any other combination of two, three, four, or five amino acids at positions 234-238. In some embodiments, an altered CH2 region comprises one or more (e.g., two, three, four or five) amino acid deletions at positions 234-238, for instance, at one of position 236 or position 237 while the other position is substituted. In certain embodiments, the amino acid residues at one or more of positions 234-238 has been replaced with one or more alanine residues. In further embodiments, only one of the amino acid residues at positions 234-238 have been deleted while one or more of the remaining amino acids at positions 234-238 can be substituted with another amino acid (e.g., alanine or serine).

In some embodiments, the above-noted mutation(s) decrease or eliminate the ADCC activity or Fc receptor-binding capability of a polypeptide that comprises the altered CH2 domain.

In certain other embodiments, a CH2 domain of a polypeptide or a protein described herein is an altered immunoglobulin CH2 region (e.g., an altered human IgG1 CH2 domain) that comprises one or more amino acid substitutions at positions 253, 310, 318, 320, 322, and 331. For example, an immunoglobulin CH2 region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. In such embodiments, the above-noted mutation(s) decrease or eliminate the CDC activity of a polypeptide comprising the altered CH2 domain.

In certain other embodiments, in addition to the amino acid substitution at position 297, an altered CH2 region of a polypeptide or a protein described herein (e.g., an altered human IgG1 CH2 domain) can further comprise one or more (e.g., two, three, four, or five) additional substitutions at positions 234-238. For example, an immunoglobulin CH2 region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, an altered CH2 region can comprise one or more (e.g., two, three, four or five) amino acid deletions at positions 234-238, such as at position 236 or position 237. The additional mutation(s) decreases or eliminates the ADCC activity or Fc receptor-binding capability of a polypeptide comprising the altered CH2 domain. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., alanine or serine).

In certain embodiments, in addition to one or more (e.g., 2, 3, 4, or 5) amino acid substitutions at positions 234-238, a mutated CH2 region of a polypeptide or a protein described herein (e.g., an altered human IgG1 CH2 domain) in a fusion protein of the present disclosure can contain one or more (e.g., 2, 3, 4, 5, or 6) additional amino acid substitutions (e.g., substituted with alanine) at one or more positions involved in complement fixation (e.g., at positions 1253, H310, E318, K320, K322, or P331). Examples of mutated immunoglobulin CH2 regions include human IgG1, IgG2, IgG4 and mouse IgG2a CH2 regions with alanine substitutions at positions 234, 235, 237 (if present), 318, 320 and 322. An exemplary mutated immunoglobulin CH2 region is mouse IGHG2c CH2 region with alanine substitutions at L234, L235, G237, E318, K320, and K322.

In still further embodiments, in addition to the amino acid substitution at position 297 and the additional deletion(s) or substitution(s) at positions 234-238, an altered CH2 region of a polypeptide or a protein described herein (e.g., an altered human IgG1 CH2 domain) can further comprise one or more (e.g., two, three, four, five, or six) additional substitutions at positions 253, 310, 318, 320, 322, and 331. For example, an immunoglobulin CH2 region can comprise a (1) substitution at position 297, (2) one or more substitutions or deletions or a combination thereof at positions 234-238, and one or more (e.g., 2, 3, 4, 5, or 6) amino acid substitutions at positions 1253, H310, E318, K320, K322, and P331, such as one, two, three substitutions at positions E318, K320 and K322. The amino acids at the above-noted positions can be substituted by alanine or serine.

In certain embodiments, an immunoglobulin CH2 region of a polypeptide or a protein described herein comprises: (i) an amino acid substitution at the asparagines of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322.

Exemplary altered immunoglobulin CH2 regions with amino acid substitutions at the asparagine of position 297 include: human IgG1 CH2 region with alanine substitutions at L234, L235, G237 and N297 and a deletion at G236 (SEQ ID NO: 325 of U.S. Patent Application Publication No. 2013/0129723, said sequence incorporated by reference herein), human IgG2 CH2 region with alanine substitutions at V234, G236, and N297 (SEQ ID NO: 326 of U.S. Patent Application Publication No. 2013/0129723, said sequence incorporated by reference herein), human IgG4 CH2 region with alanine substitutions at F234, L235, G237 and N297 and a deletion of G236 (SEQ ID NO: 322 of U.S. Patent Application Publication No. 2013/0129723, said sequence incorporated by reference herein), human IgG4 CH2 region with alanine substitutions at F234 and N297 (SEQ ID NO: 343 of U.S. Patent Application Publication No. US 2013/0129723, said sequence incorporated by reference herein), human IgG4 CH2 region with alanine substitutions at L235 and N297 (SEQ ID NO: 344 of U.S. Patent Application Publication No. 2013/0129723, said sequence incorporated by reference herein), human IgG4 CH2 region with alanine substitutions at G236 and N297 (SEQ ID NO: 345 of U.S. Patent Application Publication No. 2013/0129723, said sequence incorporated by reference herein), and human IgG4 CH2 region with alanine substitutions at G237 and N297 (SEQ ID NO: 346 of U.S. Patent Application Publication No. 2013/0129723, said sequence incorporated by reference herein). These CH2 regions can be used in a polypeptide of the present disclosure.

In certain embodiments, in addition to the amino acid substitutions described above, an altered CH2 region of a polypeptide or a protein described herein (e.g., an altered human IgG1 CH2 domain) can contain one or more additional amino acid substitutions at one or more positions other than the above-noted positions. Such amino acid substitutions can be conservative or non-conservative amino acid substitutions. For example, in certain embodiments, P233 can be changed to E233 in an altered IgG2 CH2 region (see, e.g., SEQ ID NO: 326 of U.S. Patent Application Publication No. 2013/0129723, said sequence incorporated by reference herein). In addition or alternatively, in certain embodiments, the altered CH2 region can contain one or more amino acid insertions, deletions, or both. The insertion(s), deletion(s) or substitution(s) can be anywhere in an immunoglobulin CH2 region, such as at the N- or C-terminus of a wild type immunoglobulin CH2 region resulting from linking the CH2 region with another region (e.g., a binding domain or an immunoglobulin heterodimerization domain) via a hinge.

In certain embodiments, an altered CH2 domain of a polypeptide or protein described herein is a human IgG1 CH2 domain with alanine substitutions at positions 235, 318, 320, and 322 (i.e., a human IgG1 CH2 domain with L235A, E318A, K320A and K322A substitutions) (SEQ ID NO: 595 of U.S. Patent Application Publication No. 2013/0129723, said sequence incorporated by reference herein), and optionally an N297 mutation (e.g., to alanine). In certain other embodiments, an altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO: 596 of U.S. Patent Application Publication No. 2013/0129723, said sequence incorporated by reference herein), and optionally an N297 mutation (e.g., to alanine).

In some embodiments, an immunoglobulin constant region of a polypeptide or a protein described herein comprises a human IgG1 CH2 domain comprising the substitutions L234A, L235A, G237A, and K322A, according to the EU numbering system.

The CH3 domain that can form an immunoglobulin constant region of a polypeptide or a protein described herein can be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain of a polypeptide described herein is a wild type human immunoglobulin CH3 domain, such as wild type CH3 domains of human IgG1, IgG2, IgG3, IgG4, IgA1, IgA2, IgD, IgE, or IgM as set forth in SEQ ID NOs: 116, 208-210, 204-207, and 212, respectively of U.S. Patent Application Publication No. 2013/0129723 (said sequences incorporated by reference herein). In certain embodiments, the CH3 domain is a wild type human IgG1 CH3 domain as set forth in SEQ ID NO: 116 of U.S. Patent Application Publication No. 2013/0129723 (said sequence incorporated by reference herein).

In certain embodiments, a CH3 domain of a polypeptide described herein is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain can be a human IgG1 CH3 domain with one or two mutations at positions H433 and N434 (positions are numbered according to EU numbering). The mutations in such positions can be involved in complement fixation. In certain other embodiments, an altered CH3 domain of a polypeptide described herein can be a human IgG1 CH3 domain but with one or two amino acid substitutions at position F405 or Y407. The amino acids at such positions are involved in interacting with another CH3 domain. In certain embodiments, an altered CH3 domain of polypeptide described herein can be an altered human IgG1 CH3 domain with its last lysine deleted. The sequence of this altered CH3 domain is set forth in SEQ ID NO: 761 of U.S. Patent Application Publication No. 2013/0129723 (said sequence incorporated by reference herein).

In certain embodiments, a polypeptide or a protein described herein comprises a CH3 domain that comprises so called “knobs-into-holes” mutations (see, Marvin and Zhu, Acta Pharmacologica Sinica 26:649-58, 2005; Ridgway et al., Protein Engineering 9:617-21, 1966). More specifically, mutations can be introduced into each of the CH3 domains of each polypeptide chain so that the steric complementarity required for CH3/CH3 association obligates these two CH3 domains to pair with each other. For example, a CH3 domain in one single chain polypeptide of a polypeptide heterodimer can contain a T366W mutation (a “knob” mutation, which substitutes a small amino acid with a larger one), and a CH3 domain in the other single chain polypeptide of the polypeptide heterodimer can contain a Y407A mutation (a “hole” mutation, which substitutes a large amino acid with a smaller one). Other exemplary knobs-into-holes mutations include (1) a T366Y mutation in one CH3 domain and a Y407T in the other CH3 domain, and (2) a T366W mutation in one CH3 domain and T366S, L368A and Y407V mutations in the other CH3 domain.

The CH4 domain that can form an immunoglobulin constant region a polypeptide or a protein described herein can be a wild type immunoglobulin CH4 domain or an altered immunoglobulin CH4 domain thereof from IgE or IgM molecules. In certain embodiments, the CH4 domain of a polypeptide described herein is a wild type human immunoglobulin CH4 domain, such as wild type CH4 domain of human IgE and IgM molecules as set forth in SEQ ID NOs: 213 and 214, respectively, of U.S. Patent Application Publication No. 2013/0129723 (said sequences incorporated by reference herein). In certain embodiments, a CH4 domain of a polypeptide described herein is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, an immunoglobulin constant region of a polypeptide or a protein described herein comprises a combination of CH2, CH3 or CH4 domains (i.e., more than one constant region domain selected from CH2, CH3 and CH4). For example, the immunoglobulin constant region can comprise CH2 and CH3 domains or CH3 and CH4 domains. In certain other embodiments, the immunoglobulin constant region can comprise two CH3 domains and no CH2 or CH4 domains (i.e., only two or more CH3). The multiple constant region domains that form an immunoglobulin constant region of the polypeptides described herein can be based on or derived from the same immunoglobulin molecule, or the same class or subclass immunoglobulin molecules. In certain embodiments, the immunoglobulin constant region is an IgG CH2-CH3 (e.g., IgG1 CH2-CH3, IgG2 CH2-CH3, and IgG4 CH2-CH3) and can be a human (e.g., human IgG1, IgG2, and IgG4) CH2CH3. For example, in certain embodiments, the immunoglobulin constant region of a polypeptide described herein comprises (1) wild type human IgG1 CH2 and CH3 domains, (2) human IgG1 CH2 with N297A substitution (i.e., CH2(N297A)) and wild type human IgG1 CH3, or (3) human IgG1 CH2(N297A) and an altered human IgG1 CH3 with the last lysine deleted. Alternatively, the multiple constant region domains of a polypeptide or a protein described herein can be based on or derived from different immunoglobulin molecules, or different classes or subclasses immunoglobulin molecules. For example, in certain embodiments, an immunoglobulin constant region comprises both human IgM CH3 domain and human IgG1 CH3 domain. The multiple constant region domains that form an immunoglobulin constant region of a polypeptide described herein can be directly linked together or can be linked to each other via one or more (e.g., about 2-10) amino acids.

Exemplary immunoglobulin constant regions that can be used in a polypeptide or a protein described herein are set forth in SEQ ID NOs: 305-309, 321, 323, 341, 342, and 762 of U.S. Patent Application Publication No. 2013/0129723 (said sequences incorporated by reference herein). Further exemplary immunoglobulin constant regions that can be used in a polypeptide or a protein described herein are provided in the table below.

TABLE 1
Exemplary immunoglobulin constant regions

| | | DNA | | AA |
|---|---|---|---|---|
| | | SEQ ID | | SEQ ID |
| Name | DNA Sequence | NO: | AA Sequence | NO: |
| SS-Fc | TCGAGTGAGCCCAAATCTTCT | 32 | SSEPKSSDKTH | 33 |
| domain | GACAAAACTCACACATGCCCA | | TCPPCPAPEAA | |
| | CCGTGCCCAGCACCTGAAGCC | | GAPSVFLFPPK | |
| | GCGGGTGCACCGTCAGTCTTC | | PKDTLMISRTP | |
| | CTCTTCCCCCCAAAACCCAAG | | EVTCVVVDVSH | |
| | GACACCCTCATGATCTCCCGG | | EDPEVKFNWYV | |
| | ACCCCTGAGGTCACATGCGTG | | DGVEVHNAKTK | |
| | GTGGTGGACGTGAGCCACGAA | | PREEQYNSTYR | |
| | GACCCTGAGGTCAAGTTCAAC | | WSVLTVLHQDW | |
| | TGGTACGTGGACGGCGTGGAG | | LNGKEYKCAVS | |
| | GTGCATAATGCCAAGACAAAG | | NKALPAPIEKT | |
| | CCGCGGGAGGAGCAGTACAAC | | ISKAKGQPREP | |
| | AGCACGTACCGTGTGGTCAGC | | QVYTLPPSRDE | |
| | GTCCTCACCGTCCTGCACCAG | | LTKNQVSLTCL | |
| | GACTGGCTGAATGGCAAGGAA | | VKGFYPSDIAV | |
| | TACAAGTGCGCGGTCTCCAAC | | EWESNGQPENN | |
| | AAAGCCCTCCCAGCCCCCATC | | YKTTPPVLDSD | |
| | GAGAAAACCATCTCCAAAGCC | | GSFFLYSKLTV | |
| | AAAGGGCAGCCCCGAGAACCA | | DKSRWQQGNVF | |
| | CAGGTGTACACCCTGCCCCCA | | SCSVMHEALHN | |
| | TCCCGGGATGAGCTGACCAAG | | HYTQKSLSLSP | |
| | AACCAGGTCAGCCTGACCTGC | | G | |
| | CTGGTCAAAGGCTTCTATCCA | | | |
| | AGCGACATCGCCGTGGAGTGG | | | |
| | GAGAGCAATGGGCAGCCGGAG | | | |
| | AACAACTACAAGACCACGCCT | | | |
| | CCCGTGCTGGACTCCGACGGC | | | |
| | TCCTTCTTCCTCTACAGCAAG | | | |
| | CTCACCGTGGACAAGAGCAGG | | | |
| | TGGCAGCAGGGGAACGTCTTC | | | |
| | TCATGCTCCGTGATGCATGAG | | | |
| | GCTCTGCACAACCACTACACG | | | |
| | CAGAAGAGCCTCTCCCTGTCT | | | |
| | CCGGGT | | | |
| Delta | GAGCCCAAATCTTCTGACAAA | 34 | EPKSSDKTHTC | 35 |
| SS-Fc | ACTCACACATGCCCACCGTGC | | PPCPAPEAAGA | |
| domain | CCAGCACCTGAAGCCGCGGGT | | PSVFLFPPKPK | |
| | GCACCGTCAGTCTTCCTCTTC | | DTLMISRTPEV | |
| | CCCCCAAAACCCAAGGACACC | | TCVWDVSHEDP | |
| | CTCATGATCTCCCGGACCCCT | | EVKFNWYVDGV | |
| | GAGGTCACATGCGTGGTGGTG | | EVHNAKTKPRE | |
| | GACGTGAGCCACGAAGACCCT | | EQYNSTYRVVS | |
| | GAGGTCAAGTTCAACTGGTAC | | VLTVLHQDWLN | |
| | GTGGACGGCGTGGAGGTGCAT | | GKEYKCAVSNK | |
| | AATGCCAAGACAAAGCCGCGG | | ALPAPIEKTIS | |
| | GAGGAGCAGTACAACAGCACG | | KAKGQPREPQV | |
| | TACCGTGTGGTCAGCGTCCTC | | YTLPPSRDELT | |
| | ACCGTCCTGCACCAGGACTGG | | KNQVSLTCLVK | |
| | CTGAATGGCAAGGAATACAAG | | GFYPSDIAVEW | |
| | TGCGCGGTCTCCAACAAAGCC | | ESNGQPENNYK | |
| | CTCCCAGCCCCCATCGAGAAA | | TTPPVLDSDGS | |
| | ACCATCTCCAAAGCCAAAGGG | | FFLYSKLTVDK | |
| | CAGCCCCGAGAACCACAGGTG | | SRWQQGNVFSC | |
| | TACACCCTGCCCCCATCCCGG | | SVMHEALHNHY | |
| | GATGAGCTGACCAAGAACCAG | | TQKSLSLSPG | |
| | GTCAGCCTGACCTGCCTGGTC | | | |
| | AAAGGCTTCTATCCAAGCGAC | | | |
| | ATCGCCGTGGAGTGGGAGAGC | | | |
| | AATGGGCAGCCGGAGAACAAC | | | |
| | TACAAGACCACGCCTCCCGTG | | | |
| | CTGGACTCCGACGGCTCCTTC | | | |
| | TTCCTCTACAGCAAGCTCACC | | | |
| | GTGGACAAGAGCAGGTGGCAG | | | |
| | CAGGGGAACGTCTTCTCATGC | | | |
| | TCCGTGATGCATGAGGCTCTG | | | |
| | CACAACCACTACACGCAGAAG | | | |
| | AGCCTCTCCCTGTCTCCGGGT | | | |

In certain embodiments, the immunoglobulin constant regions of each polypeptide chain of a homodimeric or heterodimeric protein described herein are identical to each other. In certain other embodiments, the immunoglobulin constant region of one polypeptide chain of a heterodimeric protein is different from the immunoglobulin constant region of the other polypeptide chain of the heterodimer. For example, one immunoglobulin constant region of a heterodimeric protein can contain a CH3 domain with a “knob” mutation, whereas the other immunoglobulin constant region of the heterodimeric protein can contain a CH3 domain with a “hole” mutation.

### Fc-Binding Domain Linker

In some embodiments, the polypeptide may further comprise a Fc-binding domain linker linking the binding domains (e.g., linking the scFv domains). In some embodiments, the Fc-binding domain linker is a Gly₄Ser linker (SEQ ID NO: 128). In some embodiments, the Fc-binding domain linker is a 20mer consisting of four repeats of a Gly-Gly-Gly-Gly-Ser (SEQ ID NO: 128) amino acid sequence ((Gly₄Ser)₄) (SEQ ID NO:61). In some embodiments, the Fc-binding domain linker comprises an amino acid sequence selected from any one of SEQ ID NOs 50-70. Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). In certain embodiments, the V_{L} and V_{H} regions are joined by a peptide linker having an amino acid sequence comprising the formula (Gly₄Ser)ₙ, wherein n=1-5 (SEQ ID NO: 129). Other suitable linkers can be obtained by optimizing a simple linker through random mutagenesis. In some embodiments, bispecific molecules do not comprise a hinge region or a constant region.

In certain embodiments, a Fc-binding domain linker is a flexible linker sequence comprising glycine-serine (e.g., Gly₄Ser, SEQ ID NO: 128) repeats. In certain embodiments, the linker comprises three Gly₄Ser repeats (SEQ ID NO: 61) followed by a proline residue. In certain embodiments the proline residue is followed by an amino acid selected from the group consisting of glycine, arginine and serine. In some embodiments, a Fc-binding domain linker comprises or consists of a sequence selected from SEQ ID NO: 50-70.

Some exemplary hinge and Fc-binding domain linker sequences suitable for use in accordance with the present disclosure are shown in the Tables 2 and 3 below. Additional exemplary hinge and linker regions are set forth in SEQ ID NOs: 241-244, 601, 78, 763-791, 228, 379-434, 618-749 of US 2013/0129723 (said sequences incorporated by reference herein).

TABLE 2
Exemplary hinges and linkers

| | Amino Acid | |
|---|---|---|
| Name | Sequence | SEQ ID NO |
| sss(s)-hlgG1 | EPKSSDKTHTSPPSS | SEQ ID NO: 36 |
| hinge | | |
| csc(s)-hlgG1 | EPKSCDKTHTSPPCS | SEQ ID NO: 37 |
| hinge | | |
| ssc(s)-hlgG1 | EPKSSDKTHTSPPCS | SEQ ID NO: 38 |
| hinge | | |
| scc(s)-hlgG1 | EPKSSDKTHTCPPCS | SEQ ID NO: 39 |
| hinge | | |
| css(s)-hlgG1 | EPKSCDKTHTSPPSS | SEQ ID NO: 40 |
| hinge | | |
| scs(s)-hlgG1 | EPKSSDKTHTCPPSS | SEQ ID NO: 41 |
| hinge | | |
| ccc(s)-hlgG1 | EPKSCDKTHTSPPCS | SEQ ID NO: 42 |
| hinge | | |
| ccc(p)-hlgG1 | EPKSCDKTHTSPPCP | SEQ ID NO: 43 |
| hinge | | |
| sss(p)-hlgG1 | EPKSSDKTHTSPPSP | SEQ ID NO: 44 |
| hinge | | |
| csc(p)-hlgG1 | EPKSCDKTHTSPPCP | SEQ ID NO: 45 |
| hinge | | |
| ssc(p)-hlgG1 | EPKSSDKTHTSPPCP | SEQ ID NO: 46 |
| hinge | | |
| scc(p)-hlgG1 | EPKSSDKTHTCPPCP | SEQ ID NO: 47 |
| hinge | | |
| css(p)-hlgG1 | EPKSCDKTHTSPPSP | SEQ ID NO: 48 |
| hinge | | |
| scs(p)-hlgG1 | EPKSSDKTHTCPPSP | SEQ ID NO: 49 |
| hinge | | |
| Scppcp | SCPPCP | SEQ ID NO: 50 |
| STD1 | NYGGGGSGGGGSGGG | SEQ ID NO: 51 |
| | GSGNS | |
| STD2 | NYGGGGSGGGGSGGG | SEQ ID NO: 52 |
| | GSGNYGGGGSGGGGS | |
| | GGGGSGNS | |
| H1 | NS | — |
| H2 | GGGGSGNS | SEQ ID NO: 53 |
| H3 | NYGGGGSGNS | SEQ ID NO: 54 |
| H4 | GGGGSGGGGSGNS | SEQ ID NO: 55 |
| H5 | NYGGGGSGGGGSGNS | SEQ ID NO: 56 |
| H6 | GGGGSGGGGSGGGGS | SEQ ID NO: 57 |
| | GNS | |
| H7 | GCPPCPNS | SEQ ID NO: 58 |
| (G₄S)₃ | GGGGSGGGGSGGGGS | SEQ ID NO: 59 |
| H105 | SGGGGSGGGGSGGGG | SEQ ID NO: 60 |
| | S | |
| (G₄S)₄ | GGGGSGGGGSGGGGS | SEQ ID NO: 61 |
| | GGGGS | |
| (G₄S)₅ | GGGGSGGGGSGGGGS | SEQ ID NO: 62 |
| | GGGGSGGGGS | |
| H75 (NKG2A | QRHNNSSLNTGTQMA | SEQ ID NO: 63 |
| quadruple | GHSPNS | |
| mutant) | | |
| H83 (NKG2A | SSLNTGTQMAGHSPN | SEQ ID NO: 64 |
| derived) | S | |
| H106(NKG2A | QRHNNSSLNTGTQMA | SEQ ID NO: 65 |
| derived) | GHS | |
| H81 (NKG2D | EVQIPLTESYSPNS | SEQ ID NO: 66 |
| derived) | | |
| H91 (NKG2D | NSLANQEVQIPLTES | SEQ ID NO: 67 |
| derived) | YSPNS | |
| H94 | SGGGGSGGGGSGGGG | SEQ ID NO: 68 |
| | SPNS | |
| H111 | SGGGGSGGGGSGGGG | SEQ ID NO: 69 |
| | SPGS | |
| H114 | GGGGSGGGGSGGGGS | SEQ ID NO: 70 |
| | PS | |

TABLE 3
Exemplary hinges and linkers (derived
from H7 hinge, stalk region of a type II
C-lectin, or interdomain region of a
type I transmembrane protein)

| | | Molecule | |
|---|---|---|---|
| | | and/or hinge | |
| | | from which | SEQ ID |
| Name | Amino Acid Sequence | derived | NO |
| H16 | LSVKADFLTPSIGNS | CD80 | SEQ ID |
| | | | NO: 71 |
| H17 | LSVKADFLTPSISCPPCPNS | CD80 + H7 | SEQ ID |
| | | | NO: 72 |
| H18 | LSVLANFSQPEIGNS | CD86 | SEQ ID |
| | | | NO: 73 |
| H19 | LSVLANFSQPEISCPPCPNS | CD86 + H7 | SEQ ID |
| | | | NO: 74 |
| H20 | LKIQERVSKPKISNS | CD2 | SEQ ID |
| | | | NO: 75 |
| H21 | LKIQERVSKPKISCPPCPNS | CD2 + H7 | SEQ ID |
| | | | NO: 76 |
| H22 | LNVSERPFPPHIQNS | CD22 | SEQ ID |
| | | | NO: 77 |
| H23 | LDVSERPFPPHIQSCPPCPNS | CD22 + H7 | SEQ ID |
| | | | NO: 78 |
| H24 | REQLAEVTLSLKANS | CD80 | SEQ ID |
| | | | NO: 79 |
| H25 | REQLAEVTLSLKACPPCPNS | CD80 + H7 | SEQ ID |
| | | | NO: 80 |
| H26 | RIHQMNSELSVLANS | CD86 | SEQ ID |
| | | | NO: 81 |
| H27 | RIHQMNSELSVLACPPCPNS | CD86 + H7 | SEQ ID |
| | | | NO: 82 |
| H28 | DTKGKNVLEKIFSNS | CD2 | SEQ ID |
| | | | NO: 83 |
| H30 | LPPETQESQEVTLNS | CD22 | SEQ ID |
| | | | NO: 84 |
| H32 | RIHLNVSERPFPPNS | CD22 | SEQ ID |
| | | | NO: 85 |
| H33 | RIHLNVSERPFPPCPPCPNS | CD22 + H7 | SEQ ID |
| | | | NO: 86 |
| H36 | GCPPCPGGGGSNS | H7 | SEQ ID |
| | | | NO: 87 |
| H40 | GCPPCPANS | H7 | SEQ ID |
| | | | NO: 88 |
| H41 | GCPPCPANS | H7 | SEQ ID |
| | | | NO: 89 |
| H42 | GCPPCPNS | H7 | SEQ ID |
| | | | NO: 90 |
| H44 | GGGASCPPCPGNS | H7 | SEQ ID |
| | | | NO: 91 |
| H45 | GGGASCPPCAGNS | H7 | SEQ ID |
| | | | NO: 92 |
| H46 | GGGASCPPCANS | H7 | SEQ ID |
| | | | NO: 93 |
| H47 | LSVKADFLTPSIGNS | CD80 | SEQ ID |
| | | | NO: 94 |
| H48 | ADFLTPSIGNS | CD80 | SEQ ID |
| | | | NO: 95 |
| H50 | LSVLANFSQPEIGNS | CD86 | SEQ ID |
| | | | NO: 96 |
| H51 | LSVLANFSQPEIGNS | CD86 | SEQ ID |
| | | | NO: 97 |
| H52 | SQPEIVPISNS | CD86 | SEQ ID |
| | | | NO: 98 |
| H53 | SQPEIVPISCPPCPNS | CD86 + H7 | SEQ ID |
| | | | NO: 99 |
| H54 | SVLANFSQPEISCPPCPNS | CD86 + H7 | SEQ ID |
| | | | NO: 100 |
| H55 | RIHQMNSELSVLANS | CD86 | SEQ ID |
| | | | NO: 101 |
| H56 | QMNSELSVLANS | CD86 | SEQ ID |
| | | | NO: 102 |
| H57 | VSERPFPPNS | CD22 | SEQ ID |
| | | | NO: 103 |
| H58 | KPFFTCGSADTCPNS | CD72 | SEQ ID |
| | | | NO: 104 |
| H59 | KPFFTCGSADTCPNS | CD72 | SEQ ID |
| | | | NO: 105 |
| H60 | QYNCPGQYTFSMPNS | CD69 | SEQ ID |
| | | | NO: 106 |
| H61 | EPAFTPGPNIELQKDSDCPNS | CD94 | SEQ ID |
| | | | NO: 107 |
| H62 | QRHNNSSLNTRTQKARHCPNS | NKG2A | SEQ ID |
| | | | NO: 108 |
| H63 | NSLFNQEVQIPLTESYCPNS | NKG2D | SEQ ID |
| | | | NO: 109 |

In addition to the aforementioned domains, the therapeutic polypeptides can further comprise immunoglobulin dimerization/heterodimerization domains, junctional amino acids, tags, additional binding domains, etc. In some embodiments, the polypeptides and proteins described herein are conjugated to a drug or a toxic moiety.

### Bispecific/Multispecific Proteins

In some embodiments, a therapeutic protein may be a bispecific or multispecific protein. Non-limiting examples of bispecific molecules include an scFv-Fc-scFv molecule, an scFv-Ig molecule and an scFv-scFv molecule. In some embodiments, the bispecific molecules described herein comprise or consist of a first binding domain scFv linked to a second binding domain scFv and do not include other sequences such as an immunoglobulin constant region. In some embodiments, a therapeutic protein may be a bispecific or multispecific protein that comprises, from amino-terminus to carboxyl-terminus, or in order from carboxyl-terminus to amino-terminus, (i) a first binding domain, (ii) a hinge region, (iii) an immunoglobulin constant region, (iv) (optionally) a Fc-binding domain linker, and (v) a second binding domain.

### Homodimers/Heterodimers

In some embodiments, a therapeutic protein may be a homodimer or a heterodimer. In some embodiments, a therapeutic protein is a dimer of two identical polypeptides, wherein each polypeptide comprises, in order from amino-terminus to carboxyl-terminus, or in order from carboxyl-terminus to amino-terminus (i) a first binding domain, (ii) a hinge region, and (iii) an immunoglobulin constant region, (iv) (optionally) a Fc-binding domain linker, and (v) a second binding domain. In some embodiments, the bispecific or multispecific protein is a dimer of two identical polypeptides, wherein each polypeptide comprises, in order from amino-terminus to carboxyl-terminus, or in order from carboxyl-terminus to amino-terminus: (i) a first binding domain, (ii) a hinge region, (iii) an immunoglobulin constant region, (iv) (optionally) a Fc-binding domain linker, and (v) a second binding domain. In other embodiments, the bispecific proteins described herein are diabodies.

In certain embodiments, a hinge present in a polypeptide that forms a heterodimer with another polypeptide chain can be an immunoglobulin hinge, such as a wild-type immunoglobulin hinge region or an altered immunoglobulin hinge region thereof. In certain embodiments, a hinge of one polypeptide chain of a heterodimeric protein is identical to a corresponding hinge of the other polypeptide chain of the heterodimer. In certain other embodiments, a hinge of one chain is different from that of the other chain (in their length or sequence). The different hinges in the different chains allow different manipulation of the binding affinities of the binding domains to which the hinges are connected, so that the heterodimer is able to preferentially bind to the target of one binding domain over the target of the other binding domain.

In other embodiments, the polypeptides and proteins described herein include a heterodimerization domain that is capable of heterodimerization with a different heterodimerization domain in a second, non-identical polypeptide chain. In certain variations, the second polypeptide chain for heterodimerization includes a second binding domain. Accordingly, in certain embodiments of the present disclosure, two non-identical polypeptide chains, one comprising the polypeptide comprising a first binding domain and the second optionally comprising a second binding domain, dimerize to form a heterodimeric binding protein.

Dimerization/heterodimerization domains can be used where it is desired to form heterodimers from two non-identical polypeptide chains, where one or both polypeptide chains comprise a binding domain. In certain embodiments, one polypeptide chain member of certain heterodimers described herein does not contain a binding domain. Examples of types of heterodimers include those described in U.S. Patent Application Publication Nos. 2013/0095097 and 2013/0129723, and International PCT Publication No. WO 2016/094873.

In certain embodiments, the first and second polypeptide chains dimerize via the inclusion of an “immunoglobulin dimerization domain” or “immunoglobulin heterodimerization domain.” An “immunoglobulin dimerization domain” or “immunoglobulin heterodimerization domain” refers herein to an immunoglobulin domain of a first polypeptide chain that preferentially interacts or associates with a different immunoglobulin domain of a second polypeptide chain, wherein the interaction of the different immunoglobulin domains substantially contributes to or efficiently promotes heterodimerization of the first and second polypeptide chains (i.e., the formation of a dimer between two different polypeptide chains, which is also referred to as a “heterodimer”). The immunoglobulin heterodimerization domains in the polypeptide chains of a heterodimer are different from each other and thus can be differentially modified to facilitate heterodimerization of both chains and to minimize homodimerization of either chain. Immunoglobulin heterodimerization domains provided herein allow for efficient heterodimerization between different polypeptides and facilitate purification of the resulting heterodimeric protein.

As provided herein, immunoglobulin heterodimerization domains useful for promoting heterodimerization of two different polypeptide chains according to the present disclosure include wild-type and altered immunoglobulin CH1 and CL domains, for instance, human CH1 and CL domains. In certain embodiments, an immunoglobulin heterodimerization domain is a wild-type CH1 domain, such as a wild type IgG1, IgG2, IgG3, IgG4, IgA1, IgA2, IgD, IgE, or IgM CH1 domain, for example, as set forth in SEQ ID NOs: 114, 186-192 and 194, respectively, of U.S. Patent Application Publication No. 2013/0129723 or SEQ ID NO: 114 of U.S. Patent Application Publication No. 2013/0129723 (said sequence incorporated by reference herein). In further embodiments, a cysteine residue of a wild-type CH1 domain (e.g., a human CH1) involved in forming a disulfide bond with a wild type immunoglobulin CL domain (e.g., a human CL) is deleted or substituted in the altered immunoglobulin CH1 domain such that a disulfide bond is not formed between the altered CH1 domain and the wild-type CL domain.

In certain embodiments, an immunoglobulin heterodimerization domain is a wild-type CL domain, such as a wild type Cκ domain or a wild type Cλ domain, for example, as set forth in SEQ ID NOs: 112 and 113, respectively, of U.S. Patent Application Publication No. 2013/0129723 (said sequences incorporated by reference herein). In further embodiments, an immunoglobulin heterodimerization domain is an altered immunoglobulin CL domain, such as an altered Cκ or Cλ domain, for instance, an altered human Cκ or human Cλ domain. In certain embodiments, a cysteine residue of a wild-type CL domain involved in forming a disulfide bond with a wild type immunoglobulin CH1 domain is deleted or substituted in the altered immunoglobulin CL domain, for example a Cκ domain as set forth in SEQ ID NO: 141 of U.S. Patent Application Publication No. 2013/0129723 or a Cλ domain as set forth in SEQ ID NO: 140 of U.S. Patent Application Publication No. 2013/0129723 (said sequences incorporated by reference herein). In certain embodiments, only the last cysteine of the wild type human Cκ domain is deleted in the altered Cκ domain because the first arginine deleted from the wild type human Cκ domain can be provided by a linker that has an arginine at its carboxyl-terminus and links the amino-terminus of the altered Cκ domain with another domain (e.g., an immunoglobulin sub-region, such as a sub-region comprising immunoglobulin CH2 and CH3 domains).

In further embodiments, an immunoglobulin heterodimerization domain is an altered Cκ domain that contains one or more amino acid substitutions, as compared to a wild type Cκ domain, at positions that may be involved in forming the interchain-hydrogen bond network at a Cκ-Cκ interface. For example, in certain embodiments, an immunoglobulin heterodimerization domain is an altered human Cκ domain having one or more amino acids at positions N29, N30, Q52, V55, T56, S68 or T70 that are substituted with a different amino acid. The numbering of the amino acids is based on their positions in the altered human Cκ sequence as set forth in SEQ ID NO: 141 of U.S. Patent Application Publication No. 2013/0129723 (said sequence incorporated by reference herein). In certain embodiments, an immunoglobulin heterodimerization domain is an altered human Cκ domain having one, two, three or four amino acid substitutions at positions N29, N30, V55, or T70. The amino acid used as a substitute at the above-noted positions can be an alanine, or an amino acid residue with a bulk side chain moiety such as arginine, tryptophan, tyrosine, glutamate, glutamine, lysine aspartate, methionine, serine or phenylalanine. Altered human Cκ domains are those that facilitate heterodimerization with a CH1 domain, but minimize homodimerization with another Cκ domain. Representative altered human Cκ domains are set forth in SEQ ID NOs: 142-178 of U.S. Patent Application Publication No. 2013/0129723; SEQ ID NOs: 160 (N29W V55A T70A), 161 (N29Y V55A T70A), 202 (T70E N29A N30A V55A), 167 (N30R V55A T70A), 168 (N30K V55A T70A), 170 (N30E V55A T70A), 172 (V55R N29A N30A), 175 (N29W N30Y V55A T70E), 176 (N29Y N30Y V55A T70E), 177 (N30E V55A T70E), 178 (N30Y V55A T70E), 838 (N30D V55A T70E), 839 (N30M V55A T70E), 840 (N30S V55A T70E), and 841 (N30F V55A T70E) of U.S. Patent Application Publication No. 2013/0129723 (said sequences incorporated by reference herein).

In certain embodiments, in addition to or alternative to the mutations in Cκ domains described herein, both the immunoglobulin heterodimerization domains (i.e., immunoglobulin CH1 and CL domains) of a polypeptide heterodimer have mutations so that the resulting immunoglobulin heterodimerization domains form salt bridges (i.e., ionic interactions) between the amino acid residues at the mutated sites. For example, the immunoglobulin heterodimerization domains of a polypeptide heterodimer can be a mutated CH1 domain in combination with a mutated Cκ domain. In the mutated CH1 domain, valine at position 68 (V68) of the wild type human CH1 domain is substituted by an amino acid residue having a negative charge (e.g., aspartate or glutamate), whereas leucine at position 29 (L29) of a mutated human Cκ domain in which the first arginine and the last cysteine have been deleted is substituted by an amino acid residue having a positive charge (e.g., lysine, arginine or histidine). The charge-charge interaction between the amino acid residue having a negative charge of the resulting mutated CH1 domain and the amino acid residue having a positive charge of the resulting mutated Cκ domain forms a salt bridge, which stabilizes the heterodimeric interface between the mutated CH1 and Cκ domains. Alternatively, V68 of the wild type CH1 can be substituted by an amino acid residue having a positive charge, whereas L29 of a mutated human Cκ domain in which the first arginine and the last cysteine have been deleted can be substituted by an amino acid residue having a negative charge. Exemplary mutated CH1 sequences in which V68 is substituted by an amino acid with either a negative or positive charge are set forth in SEQ ID NOs: 844 and 845 of U.S. Patent Application Publication No. 2013/0129723 (said sequences incorporated by reference herein). Exemplary mutated Cκ sequences in which L29 is substituted by an amino acid with either a negative or positive charge are set forth in SEQ ID NOs: 842 and 843 of U.S. Patent Application Publication No. 2013/0129723 (said sequences incorporated by reference herein).

Positions other than V68 of human CH1 domain and L29 of human Cκ domain can be substituted with amino acids having opposite charges to produce ionic interactions between the amino acids in addition or alternative to the mutations in V68 of CH1 domain and L29 of Cκ domain. Such positions can be identified by any suitable method, including random mutagenesis, analysis of the crystal structure of the CH1-Cκ pair to identify amino acid residues at the CH1-Cκ interface, and further identifying suitable positions among the amino acid residues at the CH1-Cκ interface using a set of criteria (e.g., propensity to engage in ionic interactions, proximity to a potential partner residue, etc.).

In certain embodiments, polypeptide heterodimers of the present disclosure contain only one pair of immunoglobulin heterodimerization domains. For example, a first chain of a polypeptide heterodimer can comprise a CH1 domain as an immunoglobulin heterodimerization domain, while a second chain can comprise a CL domain (e.g., a Cκ or Cλ) as an immunoglobulin heterodimerization domain. Alternatively, a first chain can comprise a CL domain (e.g., a Cκ or Cλ) as an immunoglobulin heterodimerization domain, while a second chain can comprise a CH1 domain as an immunoglobulin heterodimerization domain. As set forth herein, the immunoglobulin heterodimerization domains of the first and second chains are capable of associating to form a heterodimeric protein of this disclosure.

In certain other embodiments, heterodimeric proteins of the present disclosure can have two pairs of immunoglobulin heterodimerization domains. For example, a first chain of a heterodimer can comprise two CH1 domains, while a second chain can have two CL domains that associate with the two CH1 domains in the first chain. Alternatively, a first chain can comprise two CL domains, while a second chain can have two CH1 domains that associate with the two CL domains in the first chain. In certain embodiments, a first polypeptide chain comprises a CH1 domain and a CL domain, while a second polypeptide chain comprises a CL domain and a CH1 domain that associate with the CH1 domain and the CL domain, respectively, of the first polypeptide chain.

In the embodiments where a heterodimeric protein comprises only one heterodimerization pair (i.e., one immunoglobulin heterodimerization domain in each chain), the immunoglobulin heterodimerization domain of each chain can be located amino-terminal to the immunoglobulin constant region of that chain. Alternatively, the immunoglobulin heterodimerization domain in each chain can be located carboxyl-terminal to the immunoglobulin constant region of that chain.

In the embodiments where a heterodimeric protein comprises two heterodimerization pairs (i.e., two immunoglobulin heterodimerization domains in each chain), both immunoglobulin heterodimerization domains in each chain can be located amino-terminal to the immunoglobulin constant region of that chain. Alternatively, both immunoglobulin heterodimerization domains in each chain can be located carboxyl-terminal to the immunoglobulin constant region of that chain. In further embodiments, one immunoglobulin heterodimerization domain in each chain can be located amino-terminal to the immunoglobulin constant region of that chain, while the other immunoglobulin heterodimerization domain of each chain can be located carboxyl-terminal to the immunoglobulin constant region of that chain. In other words, in those embodiments, the immunoglobulin constant region is interposed between the two immunoglobulin heterodimerization domains of each chain.

Polypeptides and proteins described herein may be made using scaffolding as generally disclosed in U.S. Patent Application Publication Nos. 2013/0129723 and 2013/0095097, which are each incorporated herein by reference in their entirety. The polypeptides described herein may comprise two non-identical polypeptide chains, each polypeptide chain comprising an immunoglobulin heterodimerization domain. The interfacing immunoglobulin heterodimerization domains are different. In one embodiment, the immunoglobulin heterodimerization domain comprises a CH1 domain or a derivative thereof. In another embodiment, the immunoglobulin heterodimerization domain comprises a CL domain or a derivative thereof. In one embodiment, the CL domain is a Cκ or Cλ isotype or a derivative thereof. Exemplary therapeutic proteins: Anti-CD86× Mono IL-10 Polypeptides and Dimers thereof

In some embodiments, the therapeutic protein included in the compositions described herein may be an IL-10 delivery polypeptide comprising a CD86 binding domain and a monomeric IL-10 domain. In some embodiments, the therapeutic protein may be an IL-10 delivery polypeptide comprising a CD86 binding domain, an immunoglobulin Fc domain, and a monomeric IL-10 domain. In some embodiments, the protein therapeutic may be an IL-10 delivery polypeptide comprising a CD86 binding domain, an immunoglobulin hinge domain, an immunoglobulin Fc domain, (optionally) a Fc-binding domain linker and a monomeric IL-10 domain.

Thus, in some embodiments, the IL-10 delivery polypeptides may comprise or consist of a CD86 binding domain and a monomeric IL-10 domain. IL-10 delivery polypeptides of the disclosure may be described as fusion proteins. Also provided are dimers of such IL-10 delivery polypeptides, e.g., homodimers and heterodimers.

The CD86 surface molecule belongs to the B7 receptor subfamily and functions as a T-cell costimulatory molecule (Lu et al. 1997; Vicenti et al. 2008). It is normally expressed on cells with Antigen Presenting cell (APC) function such as Dendritic cells, monocytes and activated but not resting B cells (Lu et al. 1997; Vicenti et al. 2008). It is expressed at high levels by naïve human monocytes and DC and it is further upregulated under some activation conditions (Hathcock et al. 1994; Sansom et al. 2003). Expression of CD86 on naïve monocytes is estimated to be in the range of 2,000 to 5,000 copies per cell (Wolk et al. 2007). High levels of CD86 expression are associated with inflamed tissues in specific pathological conditions (Vuckovic et al. 2001; Nakazawa et al. 1999) CD86 and CD80, the latter a second member of the B7 family, facilitate T-cell activation by interacting with the T-cell co-receptor CD28.

A CD86 binding domain specifically binds to CD86. In some embodiments, the CD86-binding domain binds to an epitope located on the extracellular domain of CD86 (e.g., human CD86). In certain aspects, this epitope is a discontinuous and/or conformational epitope. In some embodiments, the CD86 binding domain binds CD86 but does not bind CD80. In some embodiments, the CD86 binding domain binds human CD86. In some embodiments, the CD86 binding domain binds to non-human primate CD86. In some embodiments, the CD86 binding domain binds human CD86 and also cross-reacts with cynomolgus CD86. In some embodiments, the CD86 binding domain binds to cynomolgus macaque monocytes and lineage negative populations (DC). In some embodiments, the CD86 binding domain is humanized.

In some cases, a CD86 binding domain of an IL-10 delivery polypeptide may be a humanized CD86 binding domain derived from the FUN-1 antibody (see, e.g., Nozawa et al., J. Pathol. 1993; 169(3):309-315). For example, a CD86-binding domain polypeptide may comprise (i) an immunoglobulin heavy chain variable region (V_{H}) comprising HCDR1, HCDR2, and HCDR3; and (2) an immunoglobulin light chain variable region (V_{L}) comprising LCDR1, LCDR2, and LCDR3. In some embodiments, at least one of the HCDR1, HCDR2, HDCR3, LCDR1, LCDR2, and LCDR3 are derived from the FUN1 antibody. In some embodiments, the HCDR1 comprises the amino acid sequence of SEQ ID NO: 1. In some embodiments, the HCDR2 comprises the amino acid sequence of SEQ ID NO: 2. In some embodiments, the HCDR3 comprises the amino acid sequence of SEQ ID NO: 3. In some embodiments, the LCDR1 comprises the amino acid sequence of SEQ ID NO: 4. In some embodiments, the LCDR2 comprises the amino acid sequence of SEQ ID NO: 5. In some embodiments, the LCDR3 comprises the amino acid sequence of SEQ ID NO: 6. In some embodiments, the HCDR1, HCDR2, and HCDR3 comprise SEQ ID NO: 1, 2, and 3, respectively. In some embodiments, the LCDR1, LCDR2, and LCDR3 comprise SEQ ID NO: 4, 5, and 6, respectively. In some embodiments, the amino acid sequence of HCDR1 is SEQ ID NO:1, the amino acid sequence of HCDR2 is SEQ ID NO:2, the amino acid sequence of HCDR3 is SEQ ID NO:3, the amino acid sequence of LCDR1 is SEQ ID NO:4, the amino acid sequence of LCDR2 is SEQ ID NO:5, and the amino acid sequence of LCDR3 is SEQ ID NO:6.

In certain embodiments, a CD86 binding domain comprises a sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) of SEQ ID NO:8. In some embodiments, a CD86-binding domain polypeptide comprises an amino acid sequence of a heavy chain variable region (V_{H}) of SEQ ID NO:7. In certain embodiments, the CD86 binding domain comprises a variable heavy chain with the amino acid sequence of SEQ ID NO:7 and a variable light chain with the amino acid sequence of SEQ ID NO:8.

CD86-binding domains suitable for use in the polypeptides of the instant disclosure may comprise or consist of an scFv. In some embodiments, the scFv may be in the V_{H}-V_{L} orientation or the V_{L}-V_{H} orientation. In some embodiments, the scFV may comprise a linker between the V_{H} and V_{L} regions. In some reasons, the linker may comprise a (Gly-Ser₄)ₙ, wherein n=an integer from 1 to 5 (SEQ ID NO: 129). In particular embodiments, n=4 (SEQ ID NO: 61).

In some embodiments, a CD86-binding domain comprises an anti-CD86 scFv that is at least about 82%, at least about 85%, at least about 87%, at least about 90%, at least about 91%, at least about 92%, at least about 93%, at least about 94%, at least about 95%, at least about 96%, at least about 97%, at least about 98%, at least about 99%, or 100% identical to an amino acid sequence of SEQ ID NO: 9. In some embodiments, the CD86 binding domain comprises an amino acid sequence with at least about 95% or 100% identity to SEQ ID NO:9.

The cytokine IL-10 is a key player in the suppression of inflammation. The critical role of IL-10 to limit inflammatory processes in preclinical and human studies has been extensively documented since its discovery over 20 years ago (Moore et al., 2001). However, multiple attempts at developing IL-10 as a therapy for a variety of inflammatory diseases have shown limited success in the clinic. There is increasing clinical evidence that while IL-10 suppresses antigen presentation and promotes antigen-specific tolerance, it also stimulates the effector function of various lymphocyte populations. This is best illustrated by the recent clinical success of IL-10 at enhancing anti-tumor responses in cancer patients, through the stimulation of cytotoxic T cells (Chan et al. 2015). Therefore, it is likely that the pleiotropic effects of IL-10, combined with its short half-life and the widespread expression of the IL-10R, have thwarted its ability to inhibit local inflammation in clinical studies.

IL-10 is a cytokine that exerts both suppressive and stimulatory functions. IL-10 is normally expressed by T cells and monocytes, macrophages, and dendritic cells. One of the main functions of IL-10 is to prevent T-cell activation through the suppression of antigen presentation by dendritic cells (DC) and macrophages (Moore et al. 2001). In addition to inducing antigen presenting function, IL-10 also induces differentiation of regulatory DC (Amodio et al. 2012). Unlike regular DC, regulatory DC induce the differentiation of antigen-dependent regulatory T cells (Tr1) (Gregori et al. 2010, Pacciani et al. 2010, Gregori et al. 2011). The critical role of IL-10 in suppressing inflammatory processes in multiple animal models and human diseases has been documented extensively (Kuhn et al. 1993; Steidler et al. 2000; Lindsay et al. 2003). In juxtaposition to its well characterized immunosuppressive function, IL-10 also stimulates the function of other cell types. Among its stimulatory functions are the enhancement of immunoglobulin secretion by B cells (Rousset et al. 1992; Fluckiger et al. 1993; Bachereau et al. 1994) and of cytotoxic effector function by T cells (Mumm et al. 2011; Chan et al 2015). There have been multiple attempts at developing IL-10 as a therapy for the treatment of autoimmune conditions in patients (Colombel et al. 2001; Fedorak et al. 2000; Schreiber et al. 2000; Kimball et al. 2002). However, the pleiotropic effects of IL-10, its short half-life and the widespread expression of the IL-10R are very likely causes for the lack of efficacy using IL-10 as a drug to inhibit inflammation (Herfarth et al. 2002).

IL-10 binds to the IL-10 receptor (IL-10R). The IL-10R is expressed on the surface of most hematopoietic cells at very low copy numbers, estimated to be around a few hundred receptors per cell (Carson et al. 1995; Jurlander et al. 1997). The IL-10R is composed of two chains: the IL-10R1 chain which associates with affinity to IL-10, and the IL-10R2 chain which has a low affinity interaction with IL-10 and participates in receptor complexes with other class 2 cytokine family members (Walter 2014). Both chains contribute to signal transduction but all IL-10-specific functions appear to reside in the IL-10R1 chain. IL-10 is a non-covalent homodimer of two intertwined polypeptide chains, expressed by T cells and monocytes/macrophages. IL-10 induces dimerization of two IL-10R complexes triggering signal transduction through the phosphorylation and activation of the transcription factor of STAT3, predominantly, although STAT1 can also be activated (Walter 2014; Donnelly et al. 1999). As described earlier, IL-10 can mediate suppressive or stimulatory functions depending on the cells type. It suppresses activation and secretion of inflammatory cytokines by myeloid cells, such as DC and monocytes, and macrophages (Sabat et al. 2010; Mosser et al. 2008; Ouyang et al. 2011). It induces differentiation of regulatory DC which induce the differentiation of regulatory T cells (Tr1 (Roncarolo). But it also promotes growth and differentiation of B cells (Rousset et al. 1992; Fluckiger et al. 1993; Banchereau et al. 1994) and the effector function of cyototoxic CD8+ T cells (Mumm et al. 2011; Chan et al. 2015). The critical role of the IL-10 pathway as a key negative regulator of inflammation is highlighted by the consequences of IL-10 deficiency in various animal models (Kuhn et al. 1993; Steidler et al. 2000; Lindsay et al. 2003).

Described herein is a modified version of IL-10 (monomeric IL-10, monoIL10 or mono-IL10) that maintains its suppressive function while reducing its stimulatory properties. The monomeric form of IL-10 can still interact with the IL-10R, but can no longer trigger downstream events on human lymphocytes while showing mildly attenuated function on myeloid cells. More specifically, monomeric IL-10 interacts with and signals through the IL-10R but shows lower affinity for the IL-10R (Josephson et al. 2000), and it interacts with the receptor in a different configuration than wt IL-10: wt IL-10 dimer/soluble IL-10R1 at 1:2 versus monoIL10/soluble IL-10R at 1:1. In spite of the reduced affinity, monomeric IL-10 retains biological activity on cells but with reduced potency. From a manufacturing perspective, it is notable that monomeric IL-10 displays greater thermal stability than wt IL-10 (Josephson et al, 2000; Westerhof et al. 2012).

In some aspects, an IL-10 delivery polypeptide comprises a monomeric IL-10 domain that comprises an amino acid insertion in the DE loop between IL-10 subdomains that allows intramolecular folding of the subdomains. In some embodiments, the amino acid insertion is 4-8 amino acids in length. In some embodiments, the amino acid insertion is 5-10 amino acids in length. In some embodiments, the amino acid insertion is 6 amino acids in length. An example of a monomeric IL-10 described herein was engineered by introducing 6 amino acids (GGGSGG, SEQ ID NO:130) in the DE loop of wildtype IL-10 that leads to the intramolecular folding of a monomer (Josephson et al. 2000). Thus, in some embodiments, the monomeric IL-10 comprises a 6 amino acid insertion in the DE loop between IL-10 subdomains that allows intramolecular folding of the subdomains. In certain embodiments, the monomeric IL-10 comprises an amino acid sequence that is at least about 85%, at least about 90%, at least about 91%, at least about 92%, at least about 93%, at least about 94%, at least about 95%, at least about 96%, at least about 97%, at least about 98%, at least about 99%, at least about 99.5%, or 100% identical to the sequence of SEQ ID NO: 28.

In some embodiments, the IL-10 delivery polypeptides comprising or consisting of a CD86 binding domain and a monomeric IL-10 domain may further comprise an immunoglobulin Fc domain. In certain embodiments, the constant region comprises IgG CH2 and CH3 domains, e.g., IgG1 CH2 and CH3 domains. In certain embodiments, the constant region does not comprise a CH1 domain. In certain embodiments, the constant domains making up the constant region are human or derived from human sequences. In some embodiments, the Fc domain comprises mutations at positions 234, 235, 237 and 322. In some embodiments, the Fc domain comprises mutations at positions 234, 235, 237, 318, 320 and 322. In some embodiments, the Fc domain comprises mutations L234A, L235A, G237A and K322A. In some embodiments, the Fc domain comprises mutations L234A, L235A, G237A, E318A, K320A, and K322A. In some embodiments, the Fc domain is derived from IgG1. In some embodiments, the Fc domain that is derived from IgG1 comprises two or more mutations that prevent the polypeptide from depleting CD86 and/or IL-10R expressing cells when administered to a patient. In some embodiments, the two or more mutations in the IgG1 Fc domain prevent or substantially reduce signaling through Fc-mediated cross-linking.

In some embodiments, the IL-10 delivery peptide may further comprise a Fc-binding domain linker. The Fc-binding domain linker may comprise 1-100 amino acids, for example 8-15 amino acids. In some embodiments, the Fc-binding domain linker comprises an amino acid sequence derived from a type II C-lectin protein, wherein the type II C-lectin protein may be NKG2A. In some embodiments, the Fc-binding domain linker comprises any one of SEQ ID NO: 50-70. In some embodiments, the Fc-binding domain linker comprises an amino acid sequence containing (Gly₄Ser)ₙ, wherein n=1-5 (SEQ ID NO: 129). In particular embodiments n=4 (SEQ ID NO: 61). In some embodiments, the Fc-binding domain linker does not contain a protease cleavage site.

The IL-10 delivery peptide may further comprise a hinge region, such as a hinge region derived from an IgG. In some embodiments, the hinge region has one or more mutated cysteine residues. In some embodiments, the hinge region comprises any one of SEQ ID NO: 71-109.

In some embodiments, an IL-10 delivery polypeptide comprises, from amino to carboxy terminus, a CD86 binding domain, an immunoglobulin hinge domain, an immunoglobulin Fc domain, a Fc-binding domain linker and a monomeric IL-10 domain. In some embodiments, the CD86 binding domain comprises SEQ ID NO:9 or an amino acid sequence with at least about 95% to 100% identity to SEQ ID NO:9, and wherein the monomeric IL-10 domain comprises SEQ ID NO:28 or an amino acid sequence with at least about 95% to 100% identity to SEQ ID NO:28. In some embodiments, and IL10 delivery peptide comprises, from amino terminus to carboxy terminus, a CD86 binding domain of SEQ ID NO:9 and a monomeric IL-10 of SEQ ID NO:28.

In some embodiments, an IL-10 delivery polypeptide comprises, from amino terminus to carboxyl terminus, a CD86 binding domain, an immunoglobulin hinge domain, an immunoglobulin Fc domain, (optionally) a Fc-binding domain linker, and a monomeric IL-10 domain, wherein the CD86 binding domain comprises a variable heavy chain and a variable light chain that specifically bind CD86, wherein the immunoglobulin Fc domain is an IgG1 Fc domain that comprises two or more mutations that prevent or significantly reduce binding to Fc receptors FcγR, FcγRIIa, FcγRIIb, and/or FcγRIIIb, wherein the Fc-binding domain linker comprises a flexible linker between 8-20 amino acids in length and free of glycosylation sites, wherein the monomeric IL-10 domain comprises two subunits of human IL-10 separated by a short linker, and wherein the IL-10 delivery polypeptide forms a dimeric protein with an identical IL-10 delivery polypeptide.

In some embodiments, the IL-10 delivery polypeptide comprises SEQ ID NO: 30 or an amino acid sequence at least about 90%, at least about 95%, at least about 98%, or at least about 99% identical to SEQ ID NO: 30. In some embodiments, the IL-10 delivery polypeptide consists essentially of SEQ ID NO: 30 or consists of SEQ ID NO: 30. In some embodiments, the IL-10 delivery polypeptide is encoded by a nucleic acid having the sequence of SEQ ID NO: 29, or a sequence at least about 90%, at least about 95%, at least about 98%, or at least about 99% identical thereto. Q0128 is an example of an IL-10 delivery polypeptide (or fusion protein) having an amino acid sequence of SEQ ID N0:30.

In some embodiments, the IL-10 delivery polypeptide binds specifically to cells expressing IL-10R and CD86. In some embodiments, the IL-10 delivery polypeptide is a dimer, such as a homodimer or a heterodimer. In some embodiments, the IL-10 delivery polypeptide is a monomer.

In some embodiments, the polypeptide, when dimerized to an identical IL-10 delivery polypeptide, induces STAT3 phosphorylation in monocytes and dendritic cells. The dendritic cells may be tolerogenic dendritic cells.

In some embodiments, the IL-10 delivery polypeptide, when dimerized to an identical IL-10 delivery polypeptide, does not induce phosphorylation on B, T and NK lymphocytes or induces minimal phosphorylation on B, T and NK lymphocytes as compared to IL-10. In some embodiments, the anti-CD86 domain enhances the signal of the monomeric IL-10 domain in vivo as compared to an Fc-monomeric IL-10 or Fc-IL-10 molecule that does not comprise a CD86 binding domain.

In some embodiments, the IL-10 delivery polypeptide, when dimerized to an identical IL-10 delivery polypeptide, exhibits increased potency as compared to IL-10.

In some embodiments, the IL-10 delivery polypeptide, when dimerized to an identical IL-10 delivery polypeptide, does not stimulate activated T cells.

In some embodiments, the IL-10 delivery polypeptide, when dimerized to an identical IL-10 delivery polypeptide, does not stimulate B cells or minimally stimulates activated B cells as compared to IL-10.

In some embodiments, the IL-10 delivery polypeptide, when dimerized to an identical IL-10 delivery polypeptide, does not induce IgM secretion or minimally induces IgM secretion as compared to IL-10.

In some embodiments, the IL-10 delivery polypeptide, when dimerized to an identical IL-10 delivery polypeptide, inhibits T cell proliferation.

In some embodiments, the IL-10 delivery polypeptide, when dimerized to an identical IL-10 delivery polypeptide, inhibits antigen presenting cell function.

In some embodiments, less than 20% CD86 receptor occupancy on monocytes is required to achieve maximum inhibition of antigen presentation when the IL-10 delivery polypeptide is dimerized to an identical IL-10 delivery polypeptide and administered to a human or non-human primate.

The anti-CD86× mono-IL10 molecules described herein are designed to treat inflammatory conditions, such as psoriasis, by delivering a modified version of IL-10 (monomeric IL-10) to antigen presenting cells. These molecules function as an improved version of IL-10 that maintains its suppressive function while reducing its stimulatory properties. They achieve this dual goal via the combination of two mechanisms. First, the monomeric form of IL-10 present in these molecules can still interact with the IL-10R, but can no longer trigger downstream events on human lymphocytes while showing mildly attenuated function on myeloid cells. Second, coupling the monomeric IL-10 to an anti-CD86 targeting arm, enhances the signal of monomeric IL-10 specifically on CD86 expressing cells. The inclusion of an Fc portion in the molecule increases its half-life compared to that of wt IL-10, which is less than 4 hours (Huhn et al. 1996). The resulting molecules suppress antigen presenting function and T-cell activation, induces regulatory DC, but does not stimulate the function of naïve or activated B or T cells. The minimal concentration at which these molecules elicit optimal function in vitro and in vivo is below the levels required for CD86 receptor saturation. Therefore, these molecules function through delivery of monoIL10 and not through CD86 blockade.

The therapeutic proteins for use in the compositions of the invention may be selected from any of the therapeutic proteins described above. For example, the therapeutic binding proteins may comprise a first binding domain and a second binding domain, optionally separated by at least an immunoglobulin constant region. In some embodiments, the first binding domain and/or the second binding domain is conjugated to a drug or a toxin.

In some embodiments, the first or second binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3. In some embodiments, the VH, the VL, or both the VH and the VL are humanized. The amino acid sequence of HCDR1 may be SEQ ID NO:1, the amino acid sequence of HCDR2 may be SEQ ID NO:2, the amino acid sequence of HCDR3 may be SEQ ID NO:3, the amino acid sequence of LCDR1 may be SEQ ID NO:4, the amino acid sequence of LCDR2 may be SEQ ID NO:5 and the amino acid sequence of LCDR3 may be SEQ ID NO:6. VH comprises SEQ ID NO:7 or an amino acid sequence at least about 90%, at least about 92%, at least about 95%, at least about 96%, at least about 97%, at least about 98% or at least about 99% identical to SEQ ID NO:7. In some embodiments, the VL comprises SEQ ID NO:8 or an amino acid sequence at least about 90%, at least about 92%, at least about 95%, at least about 96%, at least about 97%, at least about 98% or at least about 99% identical to SEQ ID NO:8. In some embodiments, the VH comprises SEQ ID NO:7 and the VL comprises SEQ ID NO:8.

In some embodiments, the first binding domain or the second binding domain is a single chain variable fragment (scFv). The light chain variable region of the scFv may be carboxy-terminal or amino-terminal to the heavy chain variable region of said scFv. In some embodiments, the scFv comprises a linker polypeptide, which may be located between the light chain variable region and the heavy chain variable region of the scFv. The linker polypeptide may comprise the formula (Gly₄Ser)ₙ, wherein n=1-5 (SEQ ID NO: 129).

In some embodiments, the first binding domain or the second binding domain specifically binds to an antigen-presenting cell. In some embodiments, the first binding domain or the second binding domain binds to a receptor of IL-10.

In some embodiments, the first binding domain or the second binding domain specifically binds to CD86.

In some embodiments, the binding domain comprises an immunoglobulin heavy chain variable region (VH) comprising HCDR1 of SEQ ID NO:1, HCDR2 of SEQ ID NO:2, and HCDR3 of SEQ ID NO:3; and an immunoglobulin light chain variable region (VL) comprising LCDR1 of SEQ ID NO:4, LCDR2 of SEQ ID NO:5, and LCDR3 of SEQ ID NO:6.

In some embodiments, the binding domain comprises an immunoglobulin heavy chain variable region (VH) comprising SEQ ID NO:7, or a sequence at least 95% identical thereto; and an immunoglobulin light chain variable region (VL) comprising SEQ ID NO:8, or a sequence at least 95% identical thereto.

In some embodiments, the binding domain comprises SEQ ID NO:9, or a sequence at least 95%, at least 96%, at least 97%, at least 98%, or at least 99% identical thereto.

In some embodiments, the first or second binding domain binds specifically to a cytokine receptor. The cytokine receptor may be, for example, the IL-10 receptor (IL-10R).

In some embodiments, the first or second binding domain comprises a cytokine or a recombinant variant of the cytokine. The cytokine or recombinant variant may be a monomeric IL-10. In some embodiments, the monomeric IL-10 binds specifically to IL-10 receptor (IL-10R). In embodiments, the monomeric IL-10 comprises an amino acid insertion in the DE loop between IL-10 subdomains that allows intramolecular folding of the subdomains. The amino acid insertion may be 4-8 amino acids or 5-10 amino acids. In embodiments, the monomeric IL-10 comprises SEQ ID NO: 28.

In some embodiments, the therapeutic proteins comprise an immunoglobulin constant region. In some embodiments, the immunoglobulin constant region is a human Fc domain. In some embodiments, the immunoglobulin constant region comprises immunoglobulin CH2 and CH3 domains of IgG1, IgG2, IgG3, IgG4, IgA1, IgA2 or IgD. In some embodiments, the immunoglobulin constant region comprises a human IgG1 CH2 domain comprising the substitutions L234A, L235A, G237A, and K322A, according to the EU numbering system. In some embodiments, the immunoglobulin constant region comprises a human IgG1 CH2 domain comprising the substitutions L234A, L235A, G237A, E318A, K320A and K322A, according to the EU numbering system. In some embodiments, the immunoglobulin constant region comprises SEQ ID NO: 131.

In some embodiments, the therapeutic proteins comprise a hinge region, for example, a hinge region derived from an immunoglobulin hinge region. In embodiments, the hinge region comprises SEQ ID NO: 47.

In some embodiments, the therapeutic proteins comprise a Fc-binding domain linker. In some embodiments, the Fc-binding domain linker comprises a Gly₄Ser (SEQ ID NO: 128) sequence, such as a (Gly₄Ser)ₙ, wherein n=1-5 (SEQ ID NO: 129).

In some embodiments, the Fc-binding domain linker comprises a sequence derived from a stalk region of a type II C-lectin protein. The type II C-lectin protein may be CD69, CD72, CD94, NKG2A or NKG2D. In some embodiments, the Fc-binding domain linker comprises SEQ ID NO:132.

In some embodiments, a therapeutic protein comprises, in order from amino terminus to carboxyl terminus a CD86 binding domain, an immunoglobulin hinge domain, an immunoglobulin Fc domain, and a monomeric IL-10 domain, wherein the CD86 binding domain comprises a variable heavy chain and a variable light chain that specifically bind CD86, wherein the immunoglobulin Fc domain is an IgG1 Fc domain that comprises two or more mutations that prevent or significantly reduce binding to Fc receptors FcγR, FcγRIIa, FcγRIIb, and FcγRIIIb, wherein the monomeric IL-10 domain comprises two subunits of human IL-10 separated by a short linker, and wherein the therapeutic protein is a homodimer. In some embodiments, the CD86 binding domain comprises an immunoglobulin heavy chain variable region (VH) comprising HCDR1, HCDR2, and HCDR3; and an immunoglobulin light chain variable region (VL) comprising LCDR1, LCDR2, and LCDR3. In some embodiments, the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6. In some embodiments, the CD86 binding domain comprises a variable heavy chain with an amino acid sequence at least 95% identical to SEQ ID NO: 7 and a variable light chain with an amino acid sequence at least 95% identical to SEQ ID NO: 8. In some embodiments, the CD86 binding domain comprises an amino acid sequence that is at least about 95% or 100% identical to SEQ ID NO: 9. In some embodiments, the monomeric IL-10 domain comprises an amino acid sequence at least 95% or 100% identical to SEQ ID NO: 28. In some embodiments, the therapeutic protein comprises SEQ ID NO: 30 or an amino acid sequence at least about 90%, at least about 95%, at least about 98%, or at least about 99% identical to SEQ ID NO: 30.

In some embodiments, the first or second binding domain binds specifically to an antigen-presenting cell, e.g., a monocyte or a dendritic cell. The antigen-presenting cell may be a monocyte or a dendritic cell, such as a CD86-expressing monocyte or a CD86-expressing dendritic cell. In some embodiments, a first or second binding domain of the therapeutic protein binds specifically to CD86.

In some embodiments, the therapeutic protein does not exhibit or exhibits minimal antibody-dependent cell-mediated cytotoxicity (ADCC) activity and/or complement-dependent cytotoxicity (CDC) activity.

In some embodiments, about 80% or more, about 85% or more, about 90% or more or about 95% or more of the weight of the therapeutic protein in the composition is not present as an aggregate. The aggregate percentage may be measured by size exclusion high performance liquid chromatography.

In some embodiments, the therapeutic protein does not aggregate or minimally aggregates after at least one freezing event and subsequent thawing event. In some embodiments, a composition of the disclosure comprising glutamate buffer has a lower relative amount of the multispecific protein present as a high molecular weight species after at least one freezing event and subsequent thawing event than the relative amount in a composition comprising a non-glutamate buffer and the same multispecific protein as measured by size exclusion high performance liquid chromatography. The freezing event may be, for example, at −80° C. or at −20° C.

In some embodiments, the compositions described herein comprise about 1-20 mg/m, about 1-12 mg/ml, or about 5-10 mg/ml of a therapeutic protein. In embodiments, the compositions comprise from about 1 mg/ml to about 12 mg/ml, or from about 5 mg/ml to about 10 mg/ml of a therapeutic protein. In further embodiments, the compositions comprise about 1, about 2, about 3, about 4, about 5, about 6, about 7, about 8, about 9, about 10, about 11, or about 12 mg/ml of a therapeutic protein. In particular embodiments, the compositions comprise about 2 mg/ml of a therapeutic protein.

### Exemplary Protein Therapeutics: Anti-CD123× Anti-CD3 Polypeptides and Dimers Thereof

An exemplary protein therapeutic may bind both CD123-expressing cells and the T-cell receptor complex on T-cells to induce target-dependent T-cell cytotoxicity, activation and proliferation.

Thus, in certain embodiments, the therapeutic protein used in connection with the methods and compositions described herein is a bispecific single chain molecule comprising a CD123 binding domain and a CD3 binding domain. In some embodiments, a CD123 and/or a CD3 binding domain is derived from an antibody and comprises a variable heavy chain (VH) and a variable light chain (VL). For example, the CD123 and/or CD3 binding domains may be a scFv that comprises a VH and a VL. These binding domains and variable chains may be arranged in any order that still retains some binding to the target(s). For example, the variable domains may be arranged in the order such as (VH CD123)-(VL CD123)-(VH CD3)-(VL CD3); (VL CD123)-(VH CD123)-(VH CD3)-(VL CD3); (VH CD123)-(VL CD123)-(VL CD3)-(VH CD3); (VL CD123)-(VH CD123)-(VL CD3)-(VH CD3); (VH CD3)-(VL CD3)-(VH CD123)-(VL CD123); (VL CD3)-(VH CD3)-(VL CD123)-(VH CD123); (VH CD3)-(VL CD3)-(VL CD123)-(VH CD123); or (VL CD3)-(VH CD3)-(VH CD123)-(VL CD123). The pairs of VH regions and VL regions in the binding domain binding to CD3 may be in the format of a single chain antibody (scFv). The VH and VL regions may be arranged in the order VH-VL or VL-VH. In some embodiments, the scFv may bind to CD123 more effectively than the antibody comprising the same VH and VL region sequences in the same orientation. In certain embodiments, the scFv may bind more effectively to CD123 in the VL-VH orientation than in the VH-VL orientation, or vice versa. The VH-region may be positioned N-terminally to a linker sequence. The VL region may be positioned C-terminally to the linker sequence. The domain arrangement in the CD3 binding domain of the bispecific single chain molecule may be VH-VL, with the CD3 binding domain located C-terminally to the CD123-binding domain. A bispecific molecule may comprise a scFv binding to CD123 linked to a scFv binding to CD3. These scFvs may be linked with a short peptide. In some embodiments, bispecific single chain molecules do not comprise a hinge region or a constant region (see, for example, US 2013/0295121, WO 2010/037836, WO 2004/106381 and WO 2011/121110; each incorporated herein by reference in its entirety).

The CD123-bispecific binding construct may comprise one or more sequences shown in Table 4, Table 5, and/or Table 6.

TABLE 4
Binding Polypeptide Sequences and Components

| | | | SEQ ID NOs: |
|---|---|---|---|
| | | Amino Acid | nucleotide |
| Name | Nucleotide Sequence | Sequence | (amino acid) |
| OMT1 | gacatcgtgatgacccagtctccagactccctggctgtgtctctgggcgag | divmtqspdslavslger | SEQ ID NO: 133 |
| variable | agggccaccatcaactgcaagtccagccacagtgttttatacagctccaac | atincksshsvlyssnnk | (SEQ ID |
| light chain | aataagaactacttagcttggtaccagcagaaaccaggacagcctcctaag | nylawyqqkpgqppklli | NO: 134) |
| domain | ctgctcatttactgggcatctacccgggaatccggggtccctgaccgattc | ywastresgvpdrfsgsg | |
| | agtggcagcgggtctgggacagatttcactctcaccatcagcagcctgcag | sgtdftltisslqaedva | |
| | gctgaagatgtggcagtttattactgtcagcaatattatagtactcctccg | vyycqqyystppttfggg | |
| | accactttcggcggagggaccaaggtggagatcaaa | tkveik | |
| OMT1 | gaggtgcagctgttggagtctgggggaggcttggtacagcctggggggtcc | evqllesggglvqpggsl | SEQ ID NO: 135 |
| variable | ctgagactctcctgtgcagcctctggattcacctttagcagctatggcatg | rlscaasgftfssygmsw | (SEQ ID |
| heavy chain | agctgggtccgccaggctccagggaaggggctggagggggtctcagctatt | vrqapgkglegvsaisgs | NO: 136) |
| domain | agtggtagtggtggtagcacatactacgcagactccgtgaagggccggttc | ggstyyadsvkgrftisr | |
| | accatctccagagacaattccaagaacacgctgtatctgcaaatgaacagc | dnskntlylqmnslraed | |
| | ctgagagccgaggacacggccgtatattactgtgcgaaagaaaagttacga | tavyycakeklryfdwls | |
| | tattttgactggttatccgatgcttttgatatctggggccaagggacaatg | dafdiwgqgtmvtvss | |
| | gtcaccgtctcttca | | |
| OMT1 | cacagtgttttatacagctccaacaataagaactac | HSVLYSSNNKNY | SEQ ID NO: 137 |
| CDR L1 | | | (SEQ ID |
| | | | NO: 138) |
| OMT1 | tgggcatct | WAS | SEQ ID NO: 139 |
| CDR L2 | | | (SEQ ID |
| | | | NO: 140) |
| OMT1 | cagcaatattatagtactcctccgaccact | QQYYSTPPTT | SEQ ID NO: 141 |
| CDR L3 | | | (SEQ ID |
| | | | NO: 142) |
| OMT1 | ggattcacctttagcagctatggc | GFTFSSYG | SEQ ID NO: 143 |
| CDR H1 | | | (SEQ ID |
| | | | NO: 144) |
| OMT1 | attagtggtagtggtggtagcaca | ISGSGGST | SEQ ID NO: 145 |
| CDR H2 | | | (SEQ ID |
| | | | NO: 146) |
| OMT1 | gcgaaagaaaagttacgatattttgactggttatccgatgcttttgatatc | AKEKLRYFDWLSDAFDI | SEQ ID NO: 147 |
| CDR H3 | | | (SEQ ID |
| | | | NO: 148) |
| DB8 | gacatccagatgacccagtctccatcctccctgtctgcatctgtaggagac | diqmtqspsslsasvgdr | SEQ ID NO: 149 |
| variable | agagtcaccatcacttgccgggcaagtcagagcattagcagctatctgaat | vtitcrasqsissylnwy | (SEQ ID |
| light chain | tggtatcagcagaaaccagggaaagcccctaagctcctgatctatgctgca | qqkpgkapklliyaassl | NO: 150) |
| domain | tccagtttgcaaagtggggtcccatcaaggttcagtggcagtggatctggg | qsgvpsrfsgsgsgtdft | |
| | acagatttcactctcaccatcagcagtctgcaacctgaagattttgcaact | ltisslqpedfatyycqq | |
| | tactactgtcaacagagttacagtacccctctcactttcggcggaggtacc | systpltfgggtkveik | |
| | aaggtggagatcaaa | | |
| DB8 | caggtgcagctggtgcagtctggggctgaggtgaagaagcctggggcctca | qvqlvqsgaevkkpgasv | SEQ ID NO: 151 |
| variable | gtgaaggtttcctgcaaggcatctggatacatcttcaccgactactatatg | kvsckasgyiftdyymhw | (SEQ ID |
| heavy chain | cactgggtgcgtcaggcccctggacaagggcttgagtggatgggatggatg | vrqapgqglewmgwmspn | NO: 152) |
| domain | agccctaacagtggtaacacaggctatgcacagaagttccagggccgtgtc | sgntgyaqkfqgrvtmtr | |
| | accatgacccgcgacacgtccacgagcacagtctacatggagctgagcagc | dtststvymelssirsed | |
| | ctgcgttctgaggacacggccgtgtattactgtgcgagagatgcggcggat | tavyycardaadygdyva | |
| | tacggtgactacgttgcttttgatatctggggccaagggacaatggtcacc | fdiwgqgtmvtvss | |
| | gtctcttca | | |
| DB8 | cagagcattagcagctat | QSISSY | SEQ ID NO: 153 |
| CDR L1 | | | (SEQ ID |
| | | | NO: 154) |
| DB8 | gctgcatcc | AAS | SEQ ID NO: 155 |
| CDR L2 | | | (SEQ ID |
| | | | NO: 156) |
| DB8 | caacagagttacagtacccctctcact | QQSYSTPLT | SEQ ID NO: 157 |
| CDR L3 | | | (SEQ ID |
| | | | NO: 158) |
| DB8 | ggatacatcttcaccgactactat | GYIFTDYY | SEQ ID NO: 159 |
| CDR H1 | | | (SEQ ID |
| | | | NO: 160) |
| DB8 | atgagccctaacagtggtaacaca | MSPNSGNT | SEQ ID NO: 161 |
| CDR H2 | | | (SEQ ID |
| | | | NO: 162) |
| DB8 | gcgagagatgcggcggattacggtgactacgttgcttttgatatc | ARDAADYGDYVAFDI | SEQ ID NO: 163 |
| CDR H3 | | | (SEQ ID |
| | | | NO: 164) |
| DB60 | gacatccagatgacccagtctccatcctccctgtctgcatctgtaggagac | diqmtqspsslsasvgdr | SEQ ID NO: 165 |
| variable | agagtcaccatcacttgccgggcaagtcagagcattagcagctatctgaat | vtitcrasqsissylnwy | (SEQ ID |
| light chain | tggtatcagcagaaaccagggaaagcccctaagctcctgatctatgctgca | qqkpgkapklliyaassl | NO: 166) |
| domain | tccagtttgcaaagtggggtcccatcaaggttcagtggcagtggatctggg | qsgvpsrfsgsgsgtdft | |
| | acagatttcactctcaccatcagcagtctgcaacctgaagattttgcaact | ltisslqpedfatyycqq | |
| | tactactgtcaacagagttacagtacccctctcactttcggcggaggtacc | systpltfgggtkveik | |
| | aaggtggagatcaaa | | |
| DB60 | caggtgcagctggtgcagtctggggctgaggtgaagaagcctggggcctca | qvqlvqsgaevkkpgasv | SEQ ID NO: 167 |
| variable | gtgaaggtttcctgcaaggcatctggatacaccttcaccagctactatatg | kvsckasgytftsyymhw | (SEQ ID |
| heavy chain | cactgggtgcgtcaggcccctggacaagggcttgagtggatggggtggatc | vrqapgqglewmgwinpn | NO: 168) |
| domain | aaccctaacagtggtgacacaagctatgcacagaagttccagggccgtgtc | sgdtsyaqkfqgrvtmtr | |
| | accatgacccgcgacacgtccacgagcacagtctacatggagctgagcagc | dtststvymelsslrsed | |
| | ctgcgttctgaggacacggccgtgtattactgtgcgcaggatagtagtggt | tavyycaqdssgsgafdi | |
| | tccggggcttttgatatctggggccaagggacaatggtcaccgtctcttca | wgqgtmvtvss | |
| DB60 | cagagcattagcagctat | QSISSY | SEQ ID NO: 169 |
| CDR L1 | | | (SEQ ID |
| | | | NO: 170) |
| DB60 | gctgcatcc | AAS | SEQ ID NO: 171 |
| CDR L2 | | | (SEQ ID |
| | | | NO: 172) |
| DB60 | caacagagttacagtacccctctcact | QQSYSTPLT | SEQ ID NO: 173 |
| CDR L3 | | | (SEQ ID |
| | | | NO: 174) |
| DB60 | ggatacaccttcaccagctactat | GYTFTSYY | SEQ ID NO: 175 |
| CDR H1 | | | (SEQ ID |
| | | | NO: 176) |
| DB60 | atcaaccctaacagtggtgacaca | INPNSGDT | SEQ ID NO: 177 |
| CDR H2 | | | (SEQ ID |
| | | | NO: 178) |
| DB60 | gcgcaggatagtagtggttccggggcttttgatatc | AQDSSGSGAFDI | SEQ ID NO: 179 |
| CDR H3 | | | (SEQ ID |
| | | | NO: 180) |
| DB65 | gacatccagatgacccagtctccatcctccctgtctgcatctgtaggagac | diqmtqspsslsasvgdr | SEQ ID NO: 181 |
| variable | agagtcaccatcacttgccgggcaagtcagagcattagcagctatctgaat | vtitcrasqsissylnwy | (SEQ ID |
| light chain | tggtatcagcagaaaccagggaaagcccctaagctcctgatctatgctgca | qqkpgkapklliyaassl | NO: 182) |
| domain | tccagtttgcaaagtggggtcccatcaaggttcagtggcagtggatctggg | qsgvpsrfsgsgsgtdft | |
| | acagatttcactctcaccatcagcagtctgcaacctgaagattttgcaact | ltisslqpedfatyycqq | |
| | tactactgtcaacagagttacagtacccctctcactttcggcggaggtacc | systpltfgggtkveik | |
| | aaggtggagatcaaa | | |
| DB65 | caggtgcagctggtgcagtctggggctgaggtgaagaagcctggggcctca | qvqlvqsgaevkkpgasv | SEQ ID NO: 183 |
| variable | gtgaaggtttcctgcaaggcatctggatacaccttcaccggctactatatg | kvsckasgytftgyymhw | (SEQ ID |
| heavy chain | cactgggtgcgtcaggcccctggacaagggcttgagtggatgggatggatg | vrqapgqglewmgwmnpn | NO: 184) |
| domain | aaccctaacagtggtaacacaggctatgcacagaagttccagggccgtgtc | sgntgyaqkfqgrvtmtr | |
| | accatgacccgcgacacgtccacgagcacagtctacatggagctgagcagc | dtststvymelsslrsed | |
| | ctgcgttctgaggacacggccgtgtattactgtgcgaaagaggaaccgatt | tavyycakeepifgvvmd | |
| | tttggagtggttatggatgcttttgatatctggggccaagggacaatggtc | afdiwgqgtmvtvss | |
| | accgtctcctca | | |
| DB65 | cagagcattagcagctat | QSISSY | SEQ ID NO: 185 |
| CDR L1 | | | (SEQ ID |
| | | | NO: 186) |
| DB65 | gctgcatcc | AAS | SEQ ID NO: 187 |
| CDR L2 | | | (SEQ ID |
| | | | NO: 188) |
| DB65 | caacagagttacagtacccctctcact | QQSYSTPLT | SEQ ID NO: 189 |
| CDR L3 | | | (SEQ ID |
| | | | NO: 190) |
| DB65 | ggatacaccttcaccggctactat | GYTFTGYY | SEQ ID NO: 191 |
| CDR H1 | | | (SEQ ID |
| | | | NO: 192) |
| DB65 | atgaaccctaacagtggtaacaca | MNPNSGNT | SEQ ID NO: 193 |
| CDR H2 | | | (SEQ ID |
| | | | NO: 194) |
| DB65 | gcgaaagaggaaccgatttttggagtggttatggatgcttttgatatc | AKEEPIFGVVMDAFDI | SEQ ID NO: 195 |
| CDR H3 | | | (SEQ ID |
| | | | NO: 196) |
| DB82 | gacatccagatgacccagtctccatcctccctgtctgcatctgtaggagac | diqmtqspsslsasvgdr | SEQ ID NO: 197 |
| variable | cgcgtcaccatcacttgccgggcaagtcagaccataaacaactatttgaac | vtitcrasqtinnylnwy | (SEQ ID |
| light chain | tggtatcagcagaaaccagggaaagcccctaagctcctgatctattctgca | qqkpgkapklliysastl | NO: 198) |
| domain | tctactttgcaaagtggggtcccatcacgtttcagtggcagtggatctggg | qsgvpsrfsgsgsgtdft | |
| | acagatttcactctcaccatcagcagtctgcaacctgaagattttgcaact | ltisslqpedfatyychq | |
| | tactactgtcaccagagttacacttcacctctcactttcggcggaggtacc | sytspltfgggtkveik | |
| | aaggtggagatcaaa | | |
| DB82 | gaggtgcagctggtggagtctgggggaggcttggtacagcctggggggtcc | evqlvesggglvqpggsl | SEQ ID NO: 199 |
| variable | ctgcgcctctcctgtgcagcctctggattcacctttagcagctatgccatg | riscaasgftfssyamsw | (SEQ ID |
| heavy chain | agctgggtccgccaggctccagggaaggggctggagtgggtctcagttatt | vrqapgkglewvsvisan | NO: 200) |
| domain | agtgccaatagtgctggtctaggccatgcggactctgtgaagggccggttc | saglghadsvkgrftisr | |
| | accatctcccgcgacaattccaagaacacgctgtatctgcaaatgaacagc | dnskntlylqmnslraed | |
| | ctgcgcgccgaggacacggccgtatattactgtgcgagagtgggctatagc | tavyycarvgysssadaf | |
| | agctcggctgatgcttttgatatctggggccaagggacaatggtcaccgtc | diwgqgtmvtvss | |
| | tcctcg | | |
| DB82 | cagaccataaacaactat | QTINNY | SEQ ID NO: 201 |
| CDR L1 | | | (SEQ ID |
| | | | NO: 202) |
| DB82 | tctgcatct | SAS | SEQ ID NO: 203 |
| CDR L2 | | | (SEQ ID |
| | | | NO: 204) |
| DB82 | caccagagttacacttcacctctcact | HQSYTSPLT | SEQ ID NO: 205 |
| CDR L3 | | | (SEQ ID |
| | | | NO: 206) |
| DB82 | ggattcacctttagcagctatgcc | GFTFSSYA | SEQ ID NO: 207 |
| CDR H1 | | | (SEQ ID |
| | | | NO: 208) |
| DB82 | attagtgccaatagtgctggtcta | ISANSAGL | SEQ ID NO: 209 |
| CDR H2 | | | (SEQ ID |
| | | | NO: 210) |
| DB82 | gcgagagtgggctatagcagctcggctgatgcttttgatatc | ARVGYSSSADAFDI | SEQ ID NO: 211 |
| CDR H3 | | | (SEQ ID |
| | | | NO: 212) |
| DB83 | gatgttgtgatgactcagtctccactctccctgcccgtcacccctggagag | dvvmtqsplslpvtpgep | SEQ ID NO: 213 |
| variable | ccggcctccatctcctgcaggtctagtcagagcctcctgcatagtaatgga | asiscrssqsllhsngdn | (SEQ ID |
| light chain | gacaactatttggattggtacctgcagaagccagggcagtctccacagctc | yldwylqkpgqspqlliy | NO: 214) |
| domain | ctgatctatttgggttctaatcgggcctccggggtccctgaccgtttcagt | lgsnrasgvpdrfsgsgs | |
| | ggcagtggatcaggcacagattttacactgaaaatcagccgtgtggaggct | gtdftlkisrveaedvgv | |
| | gaggatgttggggtttattactgcatgcaagctacacactggccactcact | yycmqathwpltfgpgtk | |
| | ttcggccctggtaccaaagtggatatcaaa | vdik | |
| DB83 | caggtgcagctggtgcagtctggggctgaggtgaagaagcctggggcctca | qvqlvqsgaevkkpgasv | SEQ ID NO: 215 |
| variable | gtgaaggtttcctgcaaggcatctggatacaccttcactagctatgctatg | kvsckasgytftsyamhw | (SEQ ID |
| heavy chain | cattgggtgcgtcaggcccctggacaagggcttgagtggatgggacttgtt | vrqapgqglewmglvdpe | NO: 216) |
| domain | gatcctgaagatggtgaaacaatatatgcagagaagttccagggccgtgtc | dgetiyaekfqgrvtmtr | |
| | accatgacccgcgacacgtccacgagcacagtctacatggagctgagcagc | dtststvymelsslrsed | |
| | ctgcgttctgaggacacggccgtgtattactgtgcgagacgaacgtattac | tavyycarrtyyydssgs | |
| | tatgatagtagtggttcccgttatgcttttgatatctggggccaagggacc | ryafdiwgqgttvtvss | |
| | acggtcaccgtctcttca | | |
| DB83 | cagagcctcctgcatagtaatggagacaactat | QSLLHSNGDNY | SEQ ID NO: 217 |
| CDR L1 | | | (SEQ ID |
| | | | NO: 218) |
| DB83 | ttgggttct | LGS | SEQ ID NO: 219 |
| CDR L2 | | | (SEQ ID |
| | | | NO: 220) |
| DB83 | atgcaagctacacactggccactcact | MQATHWPLT | SEQ ID NO: 221 |
| CDR L3 | | | (SEQ ID |
| | | | NO: 222) |
| DB83 | ggatacaccttcactagctatgct | GYTFTSYA | SEQ ID NO: 223 |
| CDR H1 | | | (SEQ ID |
| | | | NO: 224) |
| DB83 | gttgatcctgaagatggtgaaaca | VDPEDGET | SEQ ID NO: 225 |
| CDR H2 | | | (SEQ ID |
| | | | NO: 226) |
| DB83 | gcgagacgaacgtattactatgatagtagtggttcccgttatgcttttgat | ARRTYYYDSSGSRYAFDI | SEQ ID NO: 227 |
| CDR H3 | atc | | (SEQ ID |
| | | | NO: 228) |
| DB86 | gacatccagatgacccagtctccatcctccctgtctgcatctgtaggagac | diqmtqspsslsasvgdr | SEQ ID NO: 229 |
| variable | cgcgtcaccatcacttgccgggcaagtcagggcatcagaaatgatttaggt | vtitcrasqgirndlgwy | (SEQ ID |
| light chain | tggtatcagcagaaaccagggaaagcccctaagctcctgatctatgctgca | qqkpgkapklliyaastl | NO: 230) |
| domain | tccactttgcaatcaggggtcccatcacgtttcagtggcagtggatctggg | qsgvpsrfsgsgsgtdft | |
| | acagatttcactctcaccatcagcagtctgcaacctgaagattttgcaact | ltisslqpedfatyycqq | |
| | tactactgtcaacagagttacggtgcccccctcactttcggcggaggtacc | sygapltfgggtkveik | |
| | aaggtggagatcaaa | | |
| DB86 | caggtgcagctggtgcagtctggggctgaggtgaagaagcctggggcctca | qvqlvqsgaevkkpgasv | SEQ ID NO: 231 |
| variable | gtgaaggtttcctgcaaggcatctggatatatgttcagtggccattctgca | kvsckasgymfsghsahw | (SEQ ID |
| heavy chain | cactgggtgcgtcaggcccctggacaagggcttgagtggatgggatggatg | vrqapgqglewmgwmnpn | NO: 232) |
| domain | aaccctaacagtggtaacacaggctatgcacagaagttccagggccgtgtc | sgntgyaqkfqgrvtmtr | |
| | accatgacccgcgacacgtccacgagcacagtctacatggagctgagcagc | dtststvymelsslrsed | |
| | ctgcgttctgaggacacggccgtgtattactgtgcgagagatagcagtggc | tavyycardssgwydvfd | |
| | tggtacgatgtctttgactactggggccaggggaccctggtcaccgtctcc | ywgqgtlvtvss | |
| | tca | | |
| DB86 | cagggcatcagaaatgat | QGIRND | SEQ ID NO: 233 |
| CDR L1 | | | (SEQ ID |
| | | | NO: 234) |
| DB86 | gctgcatcc | AAS | SEQ ID NO: 235 |
| CDR L2 | | | (SEQ ID |
| | | | NO: 236) |
| DB86 | caacagagttacggtgcccccctc | QQSYGAPLT | SEQ ID NO: 237 |
| CDR L3 | | | (SEQ ID |
| | | | NO: 238) |
| DB86 | ggatatatgttcagtggccattct | GYMFSGHS | SEQ ID NO: 239 |
| CDR H1 | | | (SEQ ID |
| | | | NO: 240) |
| DB86 | atgaaccctaacagtggtaacaca | MNPNSGNT | SEQ ID NO: 241 |
| CDR H2 | | | (SEQ ID |
| | | | NO: 242) |
| DB86 | gcgagagatagcagtggctggtacgatgtctttgactac | ARDSSGWYDVFDY | SEQ ID NO: 243 |
| CDR H3 | | | (SEQ ID |
| | | | NO: 244) |
| DB280 | gacatccagatgacccagtctccatcctccctgtctgcatctgtaggagac | diqmtqspsslsasvgdr | SEQ ID NO: 245 |
| variable | agagtcaccatcacttgccgggcaagtcagagcattagcagctatctgaat | vtitcrasqsissylnwy | (SEQ ID |
| light chain | tggtatcagcagaaaccagggaaagcccctaagctcctgatctatgctgca | qqkpgkapklliyaassl | NO: 246) |
| domain | tccagtttgcaaagtggggtcccatcaaggttcagtggcagtggatctggg | qsgvpsrfsgsgsgtdft | |
| | acagatttcactctcaccatcagcagtctgcaacctgaagattttgcaact | ltisslqpedfatyycqq | |
| | tactactgtcaacagagttacagtacccctctcactttcggcggaggtacc | systpltfgggtkveik | |
| | aaggtggagatcaaa | | |
| DB280 | caggtgcagctggtgcagtctggggctgaggtgaagaagcctggggcctca | qvqlvqsgaevkkpgasv | SEQ ID NO: 247 |
| variable | gtgaaggtttcctgcaaggcatctggatacagcctcaacttatactatatg | kvsckasgyslnlyymhw | (SEQ ID |
| heavy chain | cactgggtgcgtcaggcccctggacaagggcttgagtggatgggatggatg | vrqapgqglewmgwmnpn | NO: 248) |
| domain | aaccctaacagtggtaacacaggctatgcacagaagttccagggccgtgtc | sgntgyaqkfqgrvtmtr | |
| | accatgacccgcgacacgtccacgagcacagtctacatggagctgagcagc | dtststvymelsslrsed | |
| | ctgcgttctgaggacacggccgtgtattactgtgcgagcctcgattgtagt | tavyycasldcsggscys | |
| | ggtggtagctgctactccgaatatgatgcttttgatatctggggccaaggg | eydafdiwgqgttvtvss | |
| | accacggtcaccgtctcctca | | |
| DB280 | cagagcattagcagctat | QSISSY | SEQ ID NO: 249 |
| CDR L1 | | | (SEQ ID |
| | | | NO: 250) |
| DB280 | gctgcatcc | AAS | SEQ ID NO: 251 |
| CDR L2 | | | (SEQ ID |
| | | | NO: 252) |
| DB280 | caacagagttacagtacccctctcact | QQSYSTPLT | SEQ ID NO: 253 |
| CDR L3 | | | (SEQ ID |
| | | | NO: 254) |
| DB280 | ggatacagcctcaacttatactat | GYSLNLYY | SEQ ID NO: 255 |
| CDR H1 | | | (SEQ ID |
| | | | NO: 256) |
| DB280 | atgaaccctaacagtggtaacaca | MNPNSGNT | SEQ ID NO: 257 |
| CDR H2 | | | (SEQ ID |
| | | | NO: 258) |
| DB280 | gcgagcctcgattgtagtggtggtagctgctactccgaatatgatgctttt | ASLDCSGGSCYSEYDAFD | SEQ ID NO: 259 |
| CDR H3 | gatatc | I | (SEQ ID |
| | | | NO: 260) |
| DB331 | gacatccagatgacccagtctccatcctccctgtctgcatctgtaggagac | diqmtqspsslsasvgdr | SEQ ID NO: 261 |
| variable | agagtcaccatcacttgccgggcaagtcagagcattagcagctatctgaat | vtitcrasqsissylnwy | (SEQ ID NO: |
| light chain | tggtatcagcagaaaccagggaaagcccctaagctcctgatctatgctgca | qqkpgkapklliyaassl | 262) |
| domain | tccagtttgcaaagtggggtcccatcaaggttcagtggcagtggatctggg | qsgvpsrfsgsgsgtdft | |
| | acagatttcactctcaccatcagcagtctgcaacctgaagattttgcaact | ltisslqpedfatyycqq | |
| | tactactgtcaacagagttacagtacccctctcactttcggcggaggtacc | systpltfgggtkveik | |
| | aaggtggagatcaaa | | |
| DB331 | caggtgcagctggtgcagtctggggctgaggtgaagaagcctggggcctca | qvqlvqsgaevkkpgasv | SEQ ID NO: 263 |
| variable | gtgaaggtttcctgcaaggcatctggatacaccttcaccagctactatatg | kvsckasgytftsyymhw | (SEQ ID |
| heavy chain | cactgggtgcgtcaggcccctggacaagggcttgagtggatgggatggatg | vrqapgqglewmgwmnpn | NO: 264) |
| domain | aaccctaacagtggtaacacaggctatgcacagaagttccagggccgtgtc | sgntgyaqkfqgrvtmtr | |
| | accatgacccgcgacacgtccacgagcacagtctacatggagctgagcagc | dtststvymelssirsed | |
| | ctgcgttctgaggacacggccgtgtattactgtgcaacagatctcgcgggg | tavyycatdlageaifdp | |
| | gaagccttgttcgacccctggggccagggcaccctggtcaccgtctcctca | wgqgtlvtvss | |
| DB331 | cagagcattagcagctat | QSISSY | SEQ ID NO: 265 |
| CDR L1 | | | (SEQ ID |
| | | | NO: 266) |
| DB331 | gctgcatcc | AAS | SEQ ID NO: 267 |
| CDR L2 | | | (SEQ ID |
| | | | NO: 268) |
| DB331 | caacagagttacagtacccctctcact | QQSYSTPLT | SEQ ID NO: 269 |
| CDR L3 | | | (SEQ ID |
| | | | NO: 270) |
| DB331 | ggatacaccttcaccagctactat | GYTFTSYY | SEQ ID NO: 271 |
| CDR H1 | | | (SEQ ID |
| | | | NO: 272) |
| DB331 | atgaaccctaacagtggtaacaca | MNPNSGNT | SEQ ID NO: 273 |
| CDR H2 | | | (SEQ ID |
| | | | NO: 274) |

| | | | |
|---|---|---|---|
| DB331 | gcaacagatctcgcgggggaagccttgttcgacccc | ATDLAGEALFDP | SEQ ID NO: 275 |
| CDR H3 | | | (SEQ ID |
| | | | NO: 276) |
| DB415 | gacatccagatgacccagtctccatcctccctgtctgcatctgtaggagac | diqmtqspsslsasvgdr | SEQ ID NO: 277 |
| variable | agagtcaccatcacttgccgggcaagtcagagcattagcagctatctgaat | vtitcrasqsissylnwy | (SEQ ID |
| light chain | tggtatcagcagaaaccagggaaagcccctaagctcctgatctatgctgca | qqkpgkapklliyaassl | NO: 278) |
| domain | tccagtttgcaaagtggggtcccatcaaggttcagtggcagtggatctggg | qsgvpsrfsgsgsgtdft | |
| | acagatttcactctcaccatcagcagtctgcaacctgaagattttgcaact | ltisslqpedfatyycqq | |
| | tactactgtcaacagagttacagtacccctctcactttcggcggaggtacc | systpltfgggtkveik | |
| | aaggtggagatcaaa | | |
| DB415 | gaggtgcagctggtggagtctgggggaggcttggtacagcctggggggtcc | evqlvesggglvqpggsl | SEQ ID NO: 279 |
| variable | ctgcgcctctcctgtgcagcctctggaatcaccttcagtagttatggcatg | riscaasgitfssygmhw | (SEQ ID |
| heavy chain | cattgggtccgccaggctccagggaaggggctggagtgggtctcaggtatt | vrqapgkglewvsgiswn | NO: 280) |
| domain | agttggaatagtggtaacagagtctatgtggactctgtgaagggccggttc | sgnrvyvdsvkgrftisr | |
| | accatctcccgcgacaattccaagaacacgctgtatctgcaaatgaacagc | dnskntlylqmnslraed | |
| | ctgcgcgccgaggacacggccgtatattactgtgcgagagatactaatgat | tavyycardtndafdiwg | |
| | gcttttgatatctggggccaagggaccacggtcaccgtctcctca | qgttvtvss | |
| DB415 | cagagcattagcagctat | QSISSY | SEQ ID NO: 281 |
| CDR L1 | | | (SEQ ID |
| | | | NO: 282) |
| DB415 | gctgcatcc | AAS | SEQ ID NO: 283 |
| CDR L2 | | | (SEQ ID |
| | | | NO: 284) |
| DB415 | caacagagttacagtacccctctcact | QQSYSTPLT | SEQ ID NO: 285 |
| CDR L3 | | | (SEQ ID |
| | | | NO: 286) |
| DB415 | ggaatcaccttcagtagttatggc | GITFSSYG | SEQ ID NO: 287 |
| CDR H1 | | | (SEQ ID |
| | | | NO: 288) |
| DB415 | attagttggaatagtggtaacaga | ISWNSGNR | SEQ ID NO: 289 |
| CDR H2 | | | (SEQ ID |
| | | | NO: 290) |
| DB415 | gcgagagatactaatgatgcttttgatatc | ARDTNDAFDI | SEQ ID NO: 291 |
| CDR H3 | | | (SEQ ID |
| | | | NO: 292) |
| DB435 | gacatccagatgacccagtctccatcctccctgtctgcatctgtaggagac | diqmtqspsslsasvgdr | SEQ ID NO: 293 |
| variable | agagtcaccatcacttgccgggcaagtcagagcattagcagctatctgaat | vtitcrasqsissylnwy | (SEQ ID |
| light chain | tggtatcagcagaaaccagggaaagcccctaagctcctgatctatgctgca | qqkpgkapklliyaassl | NO: 294) |
| domain | tccagtttgcaaagtggggtcccatcaaggttcagtggcagtggatctggg | qsgvpsrfsgsgsgtdft | |
| | acagatttcactctcaccatcagcagtctgcaacctgaagattttgcaact | ltisslqpedfatyycqq | |
| | tactactgtcaacagagttacagtacccctctcactttcggcggaggtacc | systpltfgggtkveik | |
| | aaggtggagatcaaa | | |
| DB435 | caggtgcagctggtgcagtctggggctgaggtgaagaagcctggggcctca | qvqlvqsgaevkkpgasv | SEQ ID NO: 295 |
| variable | gtgaaggtttcctgcaaggcatctggaggcaccttcagcagctatgctatc | kvsckasggtfssyaisw | (SEQ ID |
| heavy chain | agctgggtgcgtcaggcccctggacaagggcttgagtggatgggctggatc | vrqapgqglewmgwitph | NO: 296) |
| domain | acccctcacaatggtaacataaagtatgcacgggagttccagggccgtgtc | ngnikyarefqgrvtmtr | |
| | accatgacccgcgacacgtccacgagcacagtctacatggagctgagcagc | dtststvymelsslrsed | |
| | ctgcgttctgaggacacggccgtgtattactgtgcgaaagatctgaactgg | tavyycakdlnwnaafdy | |
| | aacgcagcctttgactactggggccaggggaccctggtcaccgtctcctca | wgqgtlvtvss | |
| DB435 | cagagcattagcagctat | QSISSY | SEQ ID NO: 297 |
| CDR L1 | | | (SEQ ID |
| | | | NO: 298) |
| DB435 | gctgcatcc | AAS | SEQ ID NO: 299 |
| CDR L2 | | | (SEQ ID |
| | | | NO: 300) |
| DB435 | caacagagttacagtacccctctcact | QQSYSTPLT | SEQ ID NO: 301 |
| CDR L3 | | | (SEQ ID |
| | | | NO: 302) |
| DB435 | ggaggcaccttcagcagctatgct | GGTFSSYA | SEQ ID NO: 303 |
| CDR H1 | | | (SEQ ID |
| | | | NO: 304) |
| DB435 | atcacccctcacaatggtaacata | ITPHNGNI | SEQ ID NO: 305 |
| CDR H2 | | | (SEQ ID |
| | | | NO: 306) |
| DB435 | gcgaaagatctgaactggaacgcagcctttgactac | AKDLNWNAAFDY | SEQ ID NO: 307 |
| CDR H3 | | | (SEQ ID |
| | | | NO: 308) |
| MT1 VHVL x | atggaagcaccagcgcagcttctcttcctcctgctactctggctcccagat | evqllesggglvqpggsl | SEQ ID NO: 309 |
| TSC456 | accaccggtgaggtgcagctgttggagtctgggggaggcttggtacagcct | rlscaasgftfssygmsw | (SEQ ID |
| scFv-Fc- | ggggggtccctgagactctcctgtgcagcctctggattcacctttagcagc | vrqapgkglegvsaisgs | NO: 310) |
| scFv | tatggcatgagctgggtccgccaggctccagggaaggggctggagggggtc | ggstyyadsvkgrftisr | |
| TRI129 | tcagctattagtggtagtggtggtagcacatactacgcagactccgtgaag | dnskntlylqmnslraed | |
| | ggccggttcaccatctccagagacaattccaagaacacgctgtatctgcaa | tavyycakeklryfdwls | |
| | atgaacagcctgagagccgaggacacggccgtatattactgtgcgaaagaa | dafdiwgqgtmvtvssgg | |
| | aagttacgatattttgactggttatccgatgcttttgatatctggggccaa | ggsggggsggggsggggs | |
| | gggacaatggtcaccgtctcttcaggtggaggcggttcaggcggaggtgga | divmtqspdslavslger | |
| | tccggcggtggcggctccggtggcggcggatctgacatcgtgatgacccag | atincksshsvlyssnnk | |
| | tctccagactccctggctgtgtctctgggcgagagggccaccatcaactgc | nylawyqqkpgqppklli | |
| | aagtccagccacagtgttttatacagctccaacaataagaactacttagct | ywastresgvpdrfsgsg | |
| | tggtaccagcagaaaccaggacagcctcctaagctgctcatttactgggca | sgtdftltisslqaedva | |
| | tctacccgggaatccggggtccctgaccgattcagtggcagcgggtctggg | vyycqqyystppttfggg | |
| | acagatttcactctcaccatcagcagcctgcaggctgaagatgtggcagtt | tkveiksssepkssdkth | |
| | tattactgtcagcaatattatagtactcctccgaccactttcggcggaggg | tcppcpapeaagapsvfl | |
| | accaaggtggagatcaaatcctcgagtgagcccaaatcttctgacaaaact | fppkpkdtlmisrtpevt | |
| | cacacatgcccaccgtgcccagcacctgaagccgcgggtgcaccgtcagtc | cvvvdvshedpevkfnwy | |
| | ttcctcttccccccaaaacccaaggacaccctcatgatctcccggacccct | vdgvevhnaktkpreeqy | |
| | gaggtcacatgcgtggtggtggacgtgagccacgaagaccctgaggtcaag | nstyrvvsvltvlhqdwi | |
| | ttcaactggtacgtggacggcgtggaggtgcataatgccaagacaaagccg | ngkeykcavsnkalpapi | |
| | cgggaggagcagtacaacagcacgtaccgtgtggtcagcgtcctcaccgtc | ektiskakgqprepqvyt | |
| | ctgcaccaggactggctgaatggcaaggaatacaagtgcgcggtctccaac | lppsrdeltknqvsltcl | |
| | aaagccctcccagcccccatcgagaaaaccatctccaaagccaaagggcag | vkgfypsdiavewesngq | |
| | ccccgagaaccacaggtgtacaccctgcccccatcccgggatgagctgacc | pennykttppvldsdgsf | |
| | aagaaccaggtcagcctgacctgcctggtcaaaggcttctatccaagcgac | flyskltvdksrwqqgnv | |
| | atcgccgtggagtgggagagcaatgggcagccggagaacaactacaagacc | fscsvmhealhnhytqks | |
| | acgcctcccgtgctggactccgacggctccttcttcctctacagcaagctc | lslspgsggggsggggsg | |
| | accgtggacaagagcaggtggcagcaggggaacgtcttctcatgctccgtg | gggspsqvqlvqsgpevk | |
| | atgcatgaggctctgcacaaccactacacgcagaagagcctctccctgtct | kpgssvkvsckasgytfs | |
| | ccgggttccggaggagggggttcaggtgggggaggttctggcggcggggga | rstmhwvrqapgqglewi | |
| | agcccttcacaggtgcaactggtgcagagtggacccgaggttaaaaaacca | gyinpssaytnynqkfkd | |
| | gggtcctccgttaaggttagctgcaaagcctctggctacacattttccagg | rvtitadkststaymels | |
| | agtacaatgcactgggtgaggcaggctcctggacagggactcgagtggatc | slrsedtavyycarpqvh | |
| | gggtatatcaacccatctagcgcctataccaattacaaccaaaagtttaag | ydyngfpywgqgtlvtvs | |
| | gaccgagttaccattaccgctgacaaatccaccagtacagcttatatggag | sggggsggggsggggsgg | |
| | ctgtcatctcttaggtccgaggacactgctgtttattactgcgctcgtcct | ggsdiqmtqspstlsasv | |
| | caggttcactatgactataatggttttccctactggggtcagggaaccctg | gdrvtmtcsasssvsymn | |
| | gtgactgtctcttctggcggtggaggcagcggtgggggtgggtctggaggc | wyqqkpgkapkrwiydss | |
| | ggtggcagtggcggcggaggctctgatattcagatgactcagtctcctagc | klasgvpsrfsgsgsgtd | |
| | actctcagcgccagcgtgggggatcgtgtgacaatgacttgctccgctagc | ytltisslqpddfatyyc | |
| | agtagtgtgtcttacatgaattggtatcagcagaagcccgggaaagcacct | qqwsrnpptfgggtkvei | |
| | aagcgctggatctatgactcttccaagctggcaagtggtgtcccctcacgg | krs | |
| | ttctctggctcaggttctggtactgactatactttgactatctcctccctc | | |
| | cagcccgatgatttcgctacctattattgtcagcagtggagccgtaaccca | | |
| | cccactttcggaggcggtaccaaagtggagatcaagaggtcataa | | |
| OMT1 | atggaagcaccagcgcagcttctcttcctcctgctactctggctcccagat | divmtqspdslavslger | SEQ ID NO: 311 |
| VLVH x | accaccggtgacatcgtgatgacccagtctccagactccctggctgtgtct | atincksshsvlyssnnk | (SEQ ID |
| TSC456 | ctgggcgagagggccaccatcaactgcaagtccagccacagtgttttatac | nylawyqqkpgqppklli | NO: 312) |
| scFv-Fc- | agctccaacaataagaactacttagcttggtaccagcagaaaccaggacag | ywastresgvpdrfsgsg | |
| scFv | cctcctaagctgctcatttactgggcatctacccgggaatccggggtccct | sgtdftltisslqaedva | |
| TRI130 | gaccgattcagtggcagcgggtctgggacagatttcactctcaccatcagc | vyycqqyystppttfggg | |
| | agcctgcaggctgaagatgtggcagtttattactgtcagcaatattatagt | tkveikggggsggggsgg | |
| | actcctccgaccactttcggcggagggaccaaggtggagatcaaaggtgga | ggsggggsevqllesggg | |
| | ggcggttcaggcggaggtggatccggcggtggcggctccggtggcggcgga | Ivqpggslrlscaasgft | |
| | tctgaggtgcagctgttggagtctgggggaggcttggtacagcctgggggg | fssygmswvrqapgkgle | |
| | tccctgagactctcctgtgcagcctctggattcacctttagcagctatggc | gvsaisgsggstyyadsv | |
| | atgagctgggtccgccaggctccagggaaggggctggagggggtctcagct | kgrftisrdnskntlylq | |
| | attagtggtagtggtggtagcacatactacgcagactccgtgaagggccgg | mnslraedtavyycakek | |
| | ttcaccatctccagagacaattccaagaacacgctgtatctgcaaatgaac | lryfdwisdafdiwgqgt | |
| | agcctgagagccgaggacacggccgtatattactgtgcgaaagaaaagtta | mvtvsssepkssdkthtc | |
| | cgatattttgactggttatccgatgcttttgatatctggggccaagggaca | ppcpapeaagapsvflfp | |
| | atggtcaccgtctcctcgagtgagcccaaatcttctgacaaaactcacaca | pkpkdtlmisrtpevtcv | |
| | tgcccaccgtgcccagcacctgaagccgcgggtgcaccgtcagtcttcctc | vvdvshedpevkfnwyvd | |
| | ttccccccaaaacccaaggacaccctcatgatctcccggacccctgaggtc | gvevhnaktkpreeqyns | |
| | acatgcgtggtggtggacgtgagccacgaagaccctgaggtcaagttcaac | tyrvvsvltvlhqdwing | |
| | tggtacgtggacggcgtggaggtgcataatgccaagacaaagccgcgggag | keykcavsnkalpapiek | |
| | gagcagtacaacagcacgtaccgtgtggtcagcgtcctcaccgtcctgcac | tiskakgqprepqvytip | |
| | caggactggctgaatggcaaggaatacaagtgcgcggtctccaacaaagcc | psrdeltknqvsltclvk | |
| | ctcccagcccccatcgagaaaaccatctccaaagccaaagggcagccccga | gfypsdiavewesngqpe | |
| | gaaccacaggtgtacaccctgcccccatcccgggatgagctgaccaagaac | nnykttppvldsdgsffl | |
| | caggtcagcctgacctgcctggtcaaaggcttctatccaagcgacatcgcc | yskltvdksrwqqgnvfs | |
| | gtggagtgggagagcaatgggcagccggagaacaactacaagaccacgcct | csvmhealhnhytqksls | |
| | cccgtgctggactccgacggctccttcttcctctacagcaagctcaccgtg | lspgsggggsggggsggg | |
| | gacaagagcaggtggcagcaggggaacgtcttctcatgctccgtgatgcat | gspsqvqIvqsgpevkkp | |
| | gaggctctgcacaaccactacacgcagaagagcctctccctgtctccgggt | gssvkvsckasgytfsrs | |
| | tccggaggagggggttcaggtgggggaggttctggcggcgggggaagccct | tmhwvrqapgqglewigy | |
| | tcacaggtgcaactggtgcagagtggacccgaggttaaaaaaccagggtcc | inpssaytnynqkfkdrv | |
| | tccgttaaggttagctgcaaagcctctggctacacattttccaggagtaca | titadkststaymelssl | |
| | atgcactgggtgaggcaggctcctggacagggactcgagtggatcgggtat | rsedtavyycarpqvhyd | |
| | atcaacccatctagcgcctataccaattacaaccaaaagtttaaggaccga | yngfpywgqgtlvtvssg | |
| | gttaccattaccgctgacaaatccaccagtacagcttatatggagctgtca | gggsggggsggggsgggg | |
| | tctcttaggtccgaggacactgctgtttattactgcgctcgtcctcaggtt | sdiqmtqspstlsasvgd | |
| | cactatgactataatggttttccctactggggtcagggaaccctggtgact | rvtmtcsasssvsymnwy | |
| | gtctcttctggcggtggaggcagcggtgggggtgggtctggaggcggtggc | qqkpgkapkrwiydsskl | |
| | agtggcggcggaggctctgatattcagatgactcagtctcctagcactctc | asgvpsrfsgsgsgtdyt | |
| | agcgccagcgtgggggatcgtgtgacaatgacttgctccgctagcagtagt | ltisslqpddfatyycqq | |
| | gtgtcttacatgaattggtatcagcagaagcccgggaaagcacctaagcgc | wsrnpptfgggtkveikr | |
| | tggatctatgactcttccaagctggcaagtggtgtcccctcacggttctct | s | |
| | ggctcaggttctggtactgactatactttgactatctcctccctccagccc | | |
| | gatgatttcgctacctattattgtcagcagtggagccgtaacccacccact | | |
| | ttcggaggcggtaccaaagtggagatcaagaggtcataa | | |
| DB8 VHVL x | atggaagcaccagcgcagcttctcttcctcctgctactctggctcccagat | qvqlvqsgaevkkpgasv | SEQ ID NO: 313 |
| TSC456 | accaccggtcaggtgcagctggtgcagtctggggctgaggtgaagaagcct | kvsckasgyiftdyymhw | (SEQ ID |
| scFv-Fc- | ggggcctcagtgaaggtttcctgcaaggcatctggatacatcttcaccgac | vrqapgqglewmgwmspn | NO: 314) |
| scFv | tactatatgcactgggtgcgtcaggcccctggacaagggcttgagtggatg | sgntgyaqkfqgrvtmtr | |
| TRI123 | ggatggatgagccctaacagtggtaacacaggctatgcacagaagttccag | dtststvymelssirsed | |
| | ggccgtgtcaccatgacccgcgacacgtccacgagcacagtctacatggag | tavyycardaadygdyva | |
| | ctgagcagcctgcgttctgaggacacggccgtgtattactgtgcgagagat | fdiwgqgtmvtvssgggg | |
| | gcggcggattacggtgactacgttgcttttgatatctggggccaagggaca | sggggsggggsggggsdi | |
| | atggtcaccgtctcttcaggcggcggcggcagcggcggcggcggcagcggc | qmtqspsslsasvgdrvt | |
| | ggcggaggctccggcggcggcggcagcgacatccagatgacccagtctcca | itcrasqsissylnwyqq | |
| | tcctccctgtctgcatctgtaggagacagagtcaccatcacttgccgggca | kpgkapklliyaasslqs | |
| | agtcagagcattagcagctatctgaattggtatcagcagaaaccagggaaa | gvpsrfsgsgsgtdftlt | |
| | gcccctaagctcctgatctatgctgcatccagtttgcaaagtggggtccca | isslqpedfatyycqqsy | |
| | tcaaggttcagtggcagtggatctgggacagatttcactctcaccatcagc | stpltfgggtkveiksss | |
| | agtctgcaacctgaagattttgcaacttactactgtcaacagagttacagt | epkssdkthtcppcpape | |
| | acccctctcactttcggcggaggtaccaaggtggagatcaaatcctcgagt | aagapsvflfppkpkdtl | |
| | gagcccaaatcttctgacaaaactcacacatgcccaccgtgcccagcacct | misrtpevtcvvvdvshe | |
| | gaagccgcgggtgcaccgtcagtcttcctcttccccccaaaacccaaggac | dpevkfnwyvdgvevhna | |
| | accctcatgatctcccggacccctgaggtcacatgcgtggtggtggacgtg | ktkpreeqynstyrvvsv | |
| | agccacgaagaccctgaggtcaagttcaactggtacgtggacggcgtggag | ltvlhqdwingkeykcav | |
| | gtgcataatgccaagacaaagccgcgggaggagcagtacaacagcacgtac | snkalpapiektiskakg | |
| | cgtgtggtcagcgtcctcaccgtcctgcaccaggactggctgaatggcaag | qprepqvytippsrdelt | |
| | gaatacaagtgcgcggtctccaacaaagccctcccagcccccatcgagaaa | knqvsltclvkgfypsdi | |
| | accatctccaaagccaaagggcagccccgagaaccacaggtgtacaccctg | avewesngqpennykttp | |
| | cccccatcccgggatgagctgaccaagaaccaggtcagcctgacctgcctg | pvldsdgsfflyskltvd | |
| | gtcaaaggcttctatccaagcgacatcgccgtggagtgggagagcaatggg | ksrwqqgnvfscsvmhea | |
| | cagccggagaacaactacaagaccacgcctcccgtgctggactccgacggc | lhnhytqkslslspgsgg | |
| | tccttcttcctctacagcaagctcaccgtggacaagagcaggtggcagcag | ggsggggsggggspsqvq | |
| | gggaacgtcttctcatgctccgtgatgcatgaggctctgcacaaccactac | lvqsgpevkkpgssvkvs | |
| | acgcagaagagcctctccctgtctccgggttccggaggagggggttcaggt | ckasgytfsrstmhwvrq | |
| | gggggaggttctggcggcgggggaagcccttcacaggtgcaactggtgcag | apgqglewigyinpssay | |
| | agtggacccgaggttaaaaaaccagggtcctccgttaaggttagctgcaaa | tnynqkfkdrvtitadks | |
| | gcctctggctacacattttccaggagtacaatgcactgggtgaggcaggct | tstaymelsslrsedtav | |
| | cctggacagggactcgagtggatcgggtatatcaacccatctagcgcctat | yycarpqvhydyngfpyw | |
| | accaattacaaccaaaagtttaaggaccgagttaccattaccgctgacaaa | gqgtlvtvssggggsggg | |
| | tccaccagtacagcttatatggagctgtcatctcttaggtccgaggacact | gsggggsggggsdiqmtq | |
| | gctgtttattactgcgctcgtcctcaggttcactatgactataatggtttt | spstlsasvgdrvtmtcs | |
| | ccctactggggtcagggaaccctggtgactgtctcttctggcggtggaggc | asssvsymnwyqqkpgka | |
| | agcggtgggggtgggtctggaggcggtggcagtggcggcggaggctctgat | pkrwiydssklasgvpsr | |
| | attcagatgactcagtctcctagcactctcagcgccagcgtgggggatcgt | fsgsgsgtdytltisslq | |
| | gtgacaatgacttgctccgctagcagtagtgtgtcttacatgaattggtat | pddfatyycqqwsrnppt | |
| | cagcagaagcccgggaaagcacctaagcgctggatctatgactcttccaag | fgggtkveikrs | |
| | ctggcaagtggtgtcccctcacggttctctggctcaggttctggtactgac | | |
| | tatactttgactatctcctccctccagcccgatgatttcgctacctattat | | |
| | tgtcagcagtggagccgtaacccacccactttcggaggcggtaccaaagtg | | |
| | gagatcaagaggtcataa | | |
| DB8 VLVH x | atggaagcaccagcgcagcttctcttcctcctgctactctggctcccagat | diqmtqspsslsasvgdr | SEQ ID NO: 315 |
| TSC456 | accaccggtgacatccagatgacccagtctccatcctccctgtctgcatct | vtitcrasqsissylnwy | (SEQ ID |
| scFv-Fc- | gtaggagacagagtcaccatcacttgccgggcaagtcagagcattagcagc | qqkpgkapklliyaassl | NO: 316) |
| scFv | tatctgaattggtatcagcagaaaccagggaaagcccctaagctcctgatc | qsgvpsrfsgsgsgtdft | |
| TRI124 | tatgctgcatccagtttgcaaagtggggtcccatcaaggttcagtggcagt | ltisslqpedfatyycqq | |
| | ggatctgggacagatttcactctcaccatcagcagtctgcaacctgaagat | systpltfgggtkveikg | |
| | tttgcaacttactactgtcaacagagttacagtacccctctcactttcggc | gggsggggsggggsgggg | |
| | ggaggtaccaaggtggagatcaaaggcggcggcggcagcggcggcggcggc | sqvqlvqsgaevkkpgas | |
| | agcggcggcggaggctccggcggcggcggcagccaggtgcagctggtgcag | vkvsckasgyiftdyymh | |
| | tctggggctgaggtgaagaagcctggggcctcagtgaaggtttcctgcaag | wvrqapgqglewmgwmsp | |
| | gcatctggatacatcttcaccgactactatatgcactgggtgcgtcaggcc | nsgntgyaqkfqgrvtmt | |
| | cctggacaagggcttgagtggatgggatggatgagccctaacagtggtaac | rdtststvymelssirse | |
| | acaggctatgcacagaagttccagggccgtgtcaccatgacccgcgacacg | dtavyycardaadygdyv | |
| | tccacgagcacagtctacatggagctgagcagcctgcgttctgaggacacg | afdiwgqgtmvtvsssep | |
| | gccgtgtattactgtgcgagagatgcggcggattacggtgactacgttgct | kssdkthtcppcpapeaa | |
| | tttgatatctggggccaagggacaatggtcaccgtctcctcgagtgagccc | gapsvflfppkpkdtlmi | |
| | aaatcttctgacaaaactcacacatgcccaccgtgcccagcacctgaagcc | srtpevtcvwdvshedpe | |
| | gcgggtgcaccgtcagtcttcctcttccccccaaaacccaaggacaccctc | vkfnwyvdgvevhnaktk | |
| | atgatctcccggacccctgaggtcacatgcgtggtggtggacgtgagccac | preeqynstyrvvsvltv | |
| | gaagaccctgaggtcaagttcaactggtacgtggacggcgtggaggtgcat | lhqdwingkeykcavsnk | |
| | aatgccaagacaaagccgcgggaggagcagtacaacagcacgtaccgtgtg | alpapiektiskakgqpr | |
| | gtcagcgtcctcaccgtcctgcaccaggactggctgaatggcaaggaatac | epqvytippsrdeltknq | |
| | aagtgcgcggtctccaacaaagccctcccagcccccatcgagaaaaccatc | vsltclvkgfypsdiave | |
| | tccaaagccaaagggcagccccgagaaccacaggtgtacaccctgccccca | wesngqpennykttppvl | |
| | tcccgggatgagctgaccaagaaccaggtcagcctgacctgcctggtcaaa | dsdgsfflyskltvdksr | |
| | ggcttctatccaagcgacatcgccgtggagtgggagagcaatgggcagccg | wqqgnvfscsvmhealhn | |
| | gagaacaactacaagaccacgcctcccgtgctggactccgacggctccttc | hytqkslslspgsggggs | |
| | ttcctctacagcaagctcaccgtggacaagagcaggtggcagcaggggaac | ggggsggggspsqvqlvq | |
| | gtcttctcatgctccgtgatgcatgaggctctgcacaaccactacacgcag | sgpevkkpgssvkvscka | |
| | aagagcctctccctgtctccgggttccggaggagggggttcaggtggggga | sgytfsrstmhwvrqapg | |
| | ggttctggcggcgggggaagcccttcacaggtgcaactggtgcagagtgga | qglewigyinpssaytny | |
| | cccgaggttaaaaaaccagggtcctccgttaaggttagctgcaaagcctct | nqkfkdrvtitadkstst | |
| | ggctacacattttccaggagtacaatgcactgggtgaggcaggctcctgga | aymelsslrsedtavyyc | |
| | cagggactcgagtggatcgggtatatcaacccatctagcgcctataccaat | arpqvhydyngfpywgqg | |
| | tacaaccaaaagtttaaggaccgagttaccattaccgctgacaaatccacc | tlvtvssggggsggggsg | |
| | agtacagcttatatggagctgtcatctcttaggtccgaggacactgctgtt | gggsggggsdiqmtqsps | |
| | tattactgcgctcgtcctcaggttcactatgactataatggttttccctac | tlsasvgdrvtmtcsass | |
| | tggggtcagggaaccctggtgactgtctcttctggcggtggaggcagcggt | svsymnwyqqkpgkapkr | |
| | gggggtgggtctggaggcggtggcagtggcggcggaggctctgatattcag | wiydssklasgvpsrfsg | |
| | atgactcagtctcctagcactctcagcgccagcgtgggggatcgtgtgaca | sgsgtdytltisslqpdd | |
| | atgacttgctccgctagcagtagtgtgtcttacatgaattggtatcagcag | fatyycqqwsrnpptfgg | |
| | aagcccgggaaagcacctaagcgctggatctatgactcttccaagctggca | gtkveikrs | |
| | agtggtgtcccctcacggttctctggctcaggttctggtactgactatact | | |
| | ttgactatctcctccctccagcccgatgatttcgctacctattattgtcag | | |
| | cagtggagccgtaacccacccactttcggaggcggtaccaaagtggagatc | | |
| | aagaggtcataa | | |
| DB8 VHVL x | atggaagcaccagcgcagcttctcttcctcctgctactctggctcccagat | qvqlvqsgaevkkpgasv | SEQ ID NO: 319 |
| TSC456 | accaccggtcaggtgcagctggtgcagtctggggctgaggtgaagaagcct | kvsckasgyiftdyymhw | (SEQ ID |
| scFv-Fc- | ggggcctcagtgaaggtttcctgcaaggcatctggatacatcttcaccgac | vrqapgqglewmgwmspn | NO: 320) |
| scFv | tactatatgcactgggtgcgtcaggcccctggacaagggcttgagtggatg | sgntgyaqkfqgrvtmtr | |
| TRI137 | ggatggatgagccctaacagtggtaacacaggctatgcacagaagttccag | dtststvymelssirsed | |
| | ggccgtgtcaccatgacccgcgacacgtccacgagcacagtctacatggag | tavyycardaadygdyva | |
| | ctgagcagcctgcgttctgaggacacggccgtgtattactgtgcgagagat | fdiwgqgtmvtvssgggg | |
| | gcggcggattacggtgactacgttgcttttgatatctggggccaagggaca | sggggsggggsggggsdi | |
| | atggtcaccgtctcttcaggtggaggcggttcaggcggaggtggatccggc | qmtqspsslsasvgdrvt | |
| | ggtggcggctccggtggcggcggatctgacatccagatgacccagtctcca | itcrasqsissylnwyqq | |
| | tcctccctgtctgcatctgtaggagacagagtcaccatcacttgccgggca | kpgkapklliyaasslqs | |
| | agtcagagcattagcagctatctgaattggtatcagcagaaaccagggaaa | gvpsrfsgsgsgtdftlt | |
| | gcccctaagctcctgatctatgctgcatccagtttgcaaagtggggtccca | isslqpedfatyycqqsy | |
| | tcaaggttcagtggcagtggatctgggacagatttcactctcaccatcagc | stpltfgggtkveiksss | |
| | agtctgcaacctgaagattttgcaacttactactgtcaacagagttacagt | epkssdkthtcppcpape | |
| | acccctctcactttcggcggaggtaccaaggtggagatcaaatcctcgagt | aagapsvflfppkpkdtl | |
| | gagcccaaatcttctgacaaaactcacacatgcccaccgtgcccagcacct | misrtpevtcvvvdvshe | |
| | gaagccgcgggtgcaccgtcagtcttcctcttccccccaaaacccaaggac | dpevkfnwyvdgvevhna | |
| | accctcatgatctcccggacccctgaggtcacatgcgtggtggtggacgtg | ktkpreeqynstyrvvsv | |
| | agccacgaagaccctgaggtcaagttcaactggtacgtggacggcgtggag | ltvlhqdwingkeykcav | |
| | gtgcataatgccaagacaaagccgcgggaggagcagtacaacagcacgtac | snkalpapiektiskakg | |
| | cgtgtggtcagcgtcctcaccgtcctgcaccaggactggctgaatggcaag | qprepqvytippsrdelt | |
| | gaatacaagtgcgcggtctccaacaaagccctcccagcccccatcgagaaa | knqvsltclvkgfypsdi | |
| | accatctccaaagccaaagggcagccccgagaaccacaggtgtacaccctg | avewesngqpennykttp | |
| | cccccatcccgggatgagctgaccaagaaccaggtcagcctgacctgcctg | pvldsdgsfflyskltvd | |
| | gtcaaaggcttctatccaagcgacatcgccgtggagtgggagagcaatggg | ksrwqqgnvfscsvmhea | |
| | cagccggagaacaactacaagaccacgcctcccgtgctggactccgacggc | lhnhytqkslslspgsgg | |
| | tccttcttcctctacagcaagctcaccgtggacaagagcaggtggcagcag | ggsggggsggggspsqvq | |
| | gggaacgtcttctcatgctccgtgatgcatgaggctctgcacaaccactac | lvqsgpevkkpgssvkvs | |
| | acgcagaagagcctctccctgtctccgggttccggaggagggggttcaggt | ckasgytfsrstmhwvrq | |
| | gggggaggttctggcggcgggggaagcccttcacaggtgcaactggtgcag | apgqglewigyinpssay | |
| | agtggacccgaggttaaaaaaccagggtcctccgttaaggttagctgcaaa | tnynqkfkdrvtitadks | |
| | gcctctggctacacattttccaggagtacaatgcactgggtgaggcaggct | tstaymelsslrsedtav | |
| | cctggacagggactcgagtggatcgggtatatcaacccatctagcgcctat | yycarpqvhydyngfpyw | |
| | accaattacaaccaaaagtttaaggaccgagttaccattaccgctgacaaa | gqgtlvtvssggggsggg | |
| | tccaccagtacagcttatatggagctgtcatctcttaggtccgaggacact | gsggggsggggsdiqmtq | |
| | gctgtttattactgcgctcgtcctcaggttcactatgactataatggtttt | spstlsasvgdrvtmtcs | |
| | ccctactggggtcagggaaccctggtgactgtctcttctggcggtggaggc | asssvsymnwyqqkpgka | |
| | agcggtgggggtgggtctggaggcggtggcagtggcggcggaggctctgat | pkrwiydssklasgvpsr | |
| | attcagatgactcagtctcctagcactctcagcgccagcgtgggggatcgt | fsgsgsgtdytltisslq | |
| | gtgacaatgacttgctccgctagcagtagtgtgtcttacatgaattggtat | pddfatyycqqwsrnppt | |
| | cagcagaagcccgggaaagcacctaagcgctggatctatgactcttccaag | fgggtkveikrs | |
| | ctggcaagtggtgtcccctcacggttctctggctcaggttctggtactgac | | |
| | tatactttgactatctcctccctccagcccgatgatttcgctacctattat | | |
| | tgtcagcagtggagccgtaacccacccactttcggaggcggtaccaaagtg | | |
| | gagatcaagaggtcatga | | |
| DB60 VHVL x | atggaagcaccagcgcagcttctcttcctcctgctactctggctcccagat | qvqlvqsgaevkkpgasv | SEQ ID NO: 321 |
| TSC456 | accaccggtcaggtgcagctggtgcagtctggggctgaggtgaagaagcct | kvsckasgytftsyymhw | (SEQ ID |
| ScFv-Fc- | ggggcctcagtgaaggtttcctgcaaggcatctggatacaccttcaccagc | vrqapgqglewmgwinpn | NO: 322) |
| scFv | tactatatgcactgggtgcgtcaggcccctggacaagggcttgagtggatg | sgdtsyaqkfqgrvtmtr | |
| TRI125 | gggtggatcaaccctaacagtggtgacacaagctatgcacagaagttccag | dtststvymelsslrsed | |
| | ggccgtgtcaccatgacccgcgacacgtccacgagcacagtctacatggag | tavyycaqdssgsgafdi | |
| | ctgagcagcctgcgttctgaggacacggccgtgtattactgtgcgcaggat | wgqgtmvtvssggggsgg | |
| | agtagtggttccggggcttttgatatctggggccaagggacaatggtcacc | ggsggggsggggsdiqmt | |
| | gtctcttcaggcggcggcggcagcggcggcggcggcagcggcggcggaggc | qspsslsasvgdrvtitc | |
| | tccggcggcggcggcagcgacatccagatgacccagtctccatcctccctg | rasqsissylnwyqqkpg | |
| | tctgcatctgtaggagacagagtcaccatcacttgccgggcaagtcagagc | kapklliyaasslqsgvp | |
| | attagcagctatctgaattggtatcagcagaaaccagggaaagcccctaag | srfsgsgsgtdftltiss | |
| | ctcctgatctatgctgcatccagtttgcaaagtggggtcccatcaaggttc | lqpedfatyycqqsystp | |
| | agtggcagtggatctgggacagatttcactctcaccatcagcagtctgcaa | ltfgggtkveiksssepk | |
| | cctgaagattttgcaacttactactgtcaacagagttacagtacccctctc | ssdkthtcppcpapeaag | |
| | actttcggcggaggtaccaaggtggagatcaaatcctcgagtgagcccaaa | apsvfifppkpkdtlmis | |
| | tcttctgacaaaactcacacatgcccaccgtgcccagcacctgaagccgcg | rtpevtcvvvdvshedpe | |
| | ggtgcaccgtcagtcttcctcttccccccaaaacccaaggacaccctcatg | vkfnwyvdgvevhnaktk | |
| | atctcccggacccctgaggtcacatgcgtggtggtggacgtgagccacgaa | preeqynstyrvvsvltv | |
| | gaccctgaggtcaagttcaactggtacgtggacggcgtggaggtgcataat | lhqdwlngkeykcavsnk | |
| | gccaagacaaagccgcgggaggagcagtacaacagcacgtaccgtgtggtc | alpapiektiskakgqpr | |
| | agcgtcctcaccgtcctgcaccaggactggctgaatggcaaggaatacaag | epqvytlppsrdeltknq | |
| | tgcgcggtctccaacaaagccctcccagcccccatcgagaaaaccatctcc | vsltclvkgfypsdiave | |
| | aaagccaaagggcagccccgagaaccacaggtgtacaccctgcccccatcc | wesngqpennykttppvl | |
| | cgggatgagctgaccaagaaccaggtcagcctgacctgcctggtcaaaggc | dsdgsfflyskltvdksr | |
| | ttctatccaagcgacatcgccgtggagtgggagagcaatgggcagccggag | wqqgnvfscsvmhealhn | |
| | aacaactacaagaccacgcctcccgtgctggactccgacggctccttcttc | hytqkslslspgsggggs | |
| | ctctacagcaagctcaccgtggacaagagcaggtggcagcaggggaacgtc | ggggsggggspsqvqlvq | |
| | ttctcatgctccgtgatgcatgaggctctgcacaaccactacacgcagaag | sgpevkkpgssvkvscka | |
| | agcctctccctgtctccgggttccggaggagggggttcaggtgggggaggt | sgytfsrstmhwvrqapg | |
| | tctggcggcgggggaagcccttcacaggtgcaactggtgcagagtggaccc | qglewigyinpssaytny | |
| | gaggttaaaaaaccagggtcctccgttaaggttagctgcaaagcctctggc | nqkfkdrvtitadkstst | |
| | tacacattttccaggagtacaatgcactgggtgaggcaggctcctggacag | aymelsslrsedtavyyc | |
| | ggactcgagtggatcgggtatatcaacccatctagcgcctataccaattac | arpqvhydyngfpywgqg | |
| | aaccaaaagtttaaggaccgagttaccattaccgctgacaaatccaccagt | tlvtvssggggsggggsg | |
| | acagcttatatggagctgtcatctcttaggtccgaggacactgctgtttat | gggsggggsdiqmtqsps | |
| | tactgcgctcgtcctcaggttcactatgactataatggttttccctactgg | tlsasvgdrvtmtcsass | |
| | ggtcagggaaccctggtgactgtctcttctggcggtggaggcagcggtggg | svsymnwyqqkpgkapkr | |
| | ggtgggtctggaggcggtggcagtggcggcggaggctctgatattcagatg | wiydssklasgvpsrfsg | |
| | actcagtctcctagcactctcagcgccagcgtgggggatcgtgtgacaatg | sgsgtdytltisslqpdd | |
| | acttgctccgctagcagtagtgtgtcttacatgaattggtatcagcagaag | fatyycqqwsrnpptfgg | |
| | cccgggaaagcacctaagcgctggatctatgactcttccaagctggcaagt | gtkveikrs | |
| | ggtgtcccctcacggttctctggctcaggttctggtactgactatactttg | | |
| | actatctcctccctccagcccgatgatttcgctacctattattgtcagcag | | |
| | tggagccgtaacccacccactttcggaggcggtaccaaagtggagatcaag | | |
| | aggtcataa | | |
| DB65 VHVL x | atggaagcaccagcgcagcttctcttcctcctgctactctggctcccagat | qvqlvqsgaevkkpgasv | SEQ ID NO: 323 |
| TSC456 | accaccggtcaggtgcagctggtgcagtctggggctgaggtgaagaagcct | kvsckasgytftgyymhw | (SEQ ID |
| ScFv-Fc- | ggggcctcagtgaaggtttcctgcaaggcatctggatacaccttcaccggc | vrqapgqglewmgwmnpn | NO: 324) |
| scFv | tactatatgcactgggtgcgtcaggcccctggacaagggcttgagtggatg | sgntgyaqkfqgrvtmtr | |
| TRI126 | ggatggatgaaccctaacagtggtaacacaggctatgcacagaagttccag | dtststvymelsslrsed | |
| | ggccgtgtcaccatgacccgcgacacgtccacgagcacagtctacatggag | tavyycakeepifgvvmd | |
| | ctgagcagcctgcgttctgaggacacggccgtgtattactgtgcgaaagag | afdiwgqgtmvtvssggg | |
| | gaaccgatttttggagtggttatggatgcttttgatatctggggccaaggg | gsggggsggggsggggsd | |
| | acaatggtcaccgtctcctcaggcggcggcggcagcggcggcggcggcagc | iqmtqspsslsasvgdrv | |
| | ggcggcggaggctccggcggcggcggcagcgacatccagatgacccagtct | titcrasqsissylnwyq | |
| | ccatcctccctgtctgcatctgtaggagacagagtcaccatcacttgccgg | qkpgkapklliyaasslq | |
| | gcaagtcagagcattagcagctatctgaattggtatcagcagaaaccaggg | sgvpsrfsgsgsgtdftl | |
| | aaagcccctaagctcctgatctatgctgcatccagtttgcaaagtggggtc | tisslqpedfatyycqqs | |
| | ccatcaaggttcagtggcagtggatctgggacagatttcactctcaccatc | ystpltfgggtkveikss | |
| | agcagtctgcaacctgaagattttgcaacttactactgtcaacagagttac | sepkssdkthtcppcpap | |
| | agtacccctctcactttcggcggaggtaccaaggtggagatcaaatcctcg | eaagapsvflfppkpkdt | |
| | agtgagcccaaatcttctgacaaaactcacacatgcccaccgtgcccagca | lmisrtpevtcvvvdvsh | |
| | cctgaagccgcgggtgcaccgtcagtcttcctcttccccccaaaacccaag | edpevkfnwyvdgvevhn | |
| | gacaccctcatgatctcccggacccctgaggtcacatgcgtggtggtggac | aktkpreeqynstyrvvs | |
| | gtgagccacgaagaccctgaggtcaagttcaactggtacgtggacggcgtg | vltvlhqdwingkeykca | |
| | gaggtgcataatgccaagacaaagccgcgggaggagcagtacaacagcacg | vsnkalpapiektiskak | |
| | taccgtgtggtcagcgtcctcaccgtcctgcaccaggactggctgaatggc | gqprepqvytlppsrdel | |
| | aaggaatacaagtgcgcggtctccaacaaagccctcccagcccccatcgag | tknqvsltclvkgfypsd | |
| | aaaaccatctccaaagccaaagggcagccccgagaaccacaggtgtacacc | iavewesngqpennyktt | |
| | ctgcccccatcccgggatgagctgaccaagaaccaggtcagcctgacctgc | ppvldsdgsfflyskltv | |
| | ctggtcaaaggcttctatccaagcgacatcgccgtggagtgggagagcaat | dksrwqqgnvfscsvmhe | |
| | gggcagccggagaacaactacaagaccacgcctcccgtgctggactccgac | alhnhytqkslslspgsg | |
| | ggctccttcttcctctacagcaagctcaccgtggacaagagcaggtggcag | gggsggggsggggspsqv | |
| | caggggaacgtcttctcatgctccgtgatgcatgaggctctgcacaaccac | qIvqsgpevkkpgssvkv | |
| | tacacgcagaagagcctctccctgtctccgggttccggaggagggggttca | sckasgytfsrstmhwvr | |
| | ggtgggggaggttctggcggcgggggaagcccttcacaggtgcaactggtg | qapgqglewigyinpssa | |
| | cagagtggacccgaggttaaaaaaccagggtcctccgttaaggttagctgc | ytnynqkfkdrvtitadk | |
| | aaagcctctggctacacattttccaggagtacaatgcactgggtgaggcag | ststaymelsslrsedta | |
| | gctcctggacagggactcgagtggatcgggtatatcaacccatctagcgcc | vyycarpqvhydyngfpy | |
| | tataccaattacaaccaaaagtttaaggaccgagttaccattaccgctgac | wgqgtlvtvssggggsgg | |
| | aaatccaccagtacagcttatatggagctgtcatctcttaggtccgaggac | ggsggggsggggsdiqmt | |
| | actgctgtttattactgcgctcgtcctcaggttcactatgactataatggt | qspstlsasvgdrvtmtc | |
| | tttccctactggggtcagggaaccctggtgactgtctcttctggcggtgga | sasssvsymnwyqqkpgk | |
| | ggcagcggtgggggtgggtctggaggcggtggcagtggcggcggaggctct | apkrwiydssklasgvps | |
| | gatattcagatgactcagtctcctagcactctcagcgccagcgtgggggat | rfsgsgsgtdytltissl | |
| | cgtgtgacaatgacttgctccgctagcagtagtgtgtcttacatgaattgg | qpddfatyycqqwsrnpp | |
| | tatcagcagaagcccgggaaagcacctaagcgctggatctatgactcttcc | tfgggtkveikrs | |
| | aagctggcaagtggtgtcccctcacggttctctggctcaggttctggtact | | |
| | gactatactttgactatctcctccctccagcccgatgatttcgctacctat | | |
| | tattgtcagcagtggagccgtaacccacccactttcggaggcggtaccaaa | | |
| | gtggagatcaagaggtcataa | | |
| DB82 VLVH x | atggaagcaccagcgcagcttctcttcctcctgctactctggctcccagat | diqmtqspsslsasvgdr | SEQ ID NO: 325 |
| TSC456 | accaccggtgacatccagatgacccagtctccatcctccctgtctgcatct | vtitcrasqtinnylnwy | (SEQ ID |
| ScFv-Fc- | gtaggagaccgcgtcaccatcacttgccgggcaagtcagaccataaacaac | qqkpgkapklliysastl | NO: 326) |
| scFv | tatttgaactggtatcagcagaaaccagggaaagcccctaagctcctgatc | qsgvpsrfsgsgsgtdft | |
| TRI127 | tattctgcatctactttgcaaagtggggtcccatcacgtttcagtggcagt | ltisslqpedfatyychq | |
| | ggatctgggacagatttcactctcaccatcagcagtctgcaacctgaagat | sytspltfgggtkveikg | |
| | tttgcaacttactactgtcaccagagttacacttcacctctcactttcggc | gggsggggsggggsgggg | |
| | ggaggtaccaaggtggagatcaaaggcggcggcggcagcggcggcggcggc | sevqlvesggglvqpggs | |
| | agcggcggcggaggctccggcggcggcggcagcgaggtgcagctggtggag | lrlscaasgftfssyams | |
| | tctgggggaggcttggtacagcctggggggtccctgcgcctctcctgtgca | wvrqapgkglewvsvisa | |
| | gcctctggattcacctttagcagctatgccatgagctgggtccgccaggct | nsaglghadsvkgrftis | |
| | ccagggaaggggctggagtgggtctcagttattagtgccaatagtgctggt | rdnskntlylqmnslrae | |
| | ctaggccatgcggactctgtgaagggccggttcaccatctcccgcgacaat | dtavyycarvgysssada | |
| | tccaagaacacgctgtatctgcaaatgaacagcctgcgcgccgaggacacg | fdiwgqgtmvtvsssepk | |
| | gccgtatattactgtgcgagagtgggctatagcagctcggctgatgctttt | ssdkthtcppcpapeaag | |
| | gatatctggggccaagggacaatggtcaccgtctcctcgagtgagcccaaa | apsvfifppkpkdtlmis | |
| | tcttctgacaaaactcacacatgcccaccgtgcccagcacctgaagccgcg | rtpevtcvvvdvshedpe | |
| | ggtgcaccgtcagtcttcctcttccccccaaaacccaaggacaccctcatg | vkfnwyvdgvevhnaktk | |
| | atctcccggacccctgaggtcacatgcgtggtggtggacgtgagccacgaa | preeqynstyrvvsvltv | |
| | gaccctgaggtcaagttcaactggtacgtggacggcgtggaggtgcataat | lhqdwlngkeykcavsnk | |
| | gccaagacaaagccgcgggaggagcagtacaacagcacgtaccgtgtggtc | alpapiektiskakgqpr | |
| | agcgtcctcaccgtcctgcaccaggactggctgaatggcaaggaatacaag | epqvytlppsrdeltknq | |
| | tgcgcggtctccaacaaagccctcccagcccccatcgagaaaaccatctcc | vsltclvkgfypsdiave | |
| | aaagccaaagggcagccccgagaaccacaggtgtacaccctgcccccatcc | wesngqpennykttppvl | |
| | cgggatgagctgaccaagaaccaggtcagcctgacctgcctggtcaaaggc | dsdgsfflyskltvdksr | |
| | ttctatccaagcgacatcgccgtggagtgggagagcaatgggcagccggag | wqqgnvfscsvmhealhn | |
| | aacaactacaagaccacgcctcccgtgctggactccgacggctccttcttc | hytqkslslspgsggggs | |
| | ctctacagcaagctcaccgtggacaagagcaggtggcagcaggggaacgtc | ggggsggggspsqvqlvq | |
| | ttctcatgctccgtgatgcatgaggctctgcacaaccactacacgcagaag | sgpevkkpgssvkvscka | |
| | agcctctccctgtctccgggttccggaggagggggttcaggtgggggaggt | sgytfsrstmhwvrqapg | |
| | tctggcggcgggggaagcccttcacaggtgcaactggtgcagagtggaccc | qglewigyinpssaytny | |
| | gaggttaaaaaaccagggtcctccgttaaggttagctgcaaagcctctggc | nqkfkdrvtitadkstst | |
| | tacacattttccaggagtacaatgcactgggtgaggcaggctcctggacag | aymelsslrsedtavyyc | |
| | ggactcgagtggatcgggtatatcaacccatctagcgcctataccaattac | arpqvhydyngfpywgqg | |
| | aaccaaaagtttaaggaccgagttaccattaccgctgacaaatccaccagt | tIvtvssggggsggggsg | |
| | acagcttatatggagctgtcatctcttaggtccgaggacactgctgtttat | gggsggggsdiqmtqsps | |
| | tactgcgctcgtcctcaggttcactatgactataatggttttccctactgg | tlsasvgdrvtmtcsass | |
| | ggtcagggaaccctggtgactgtctcttctggcggtggaggcagcggtggg | svsymnwyqqkpgkapkr | |
| | ggtgggtctggaggcggtggcagtggcggcggaggctctgatattcagatg | wiydssklasgvpsrfsg | |
| | actcagtctcctagcactctcagcgccagcgtgggggatcgtgtgacaatg | sgsgtdytltisslqpdd | |
| | acttgctccgctagcagtagtgtgtcttacatgaattggtatcagcagaag | fatyycqqwsrnpptfgg | |
| | cccgggaaagcacctaagcgctggatctatgactcttccaagctggcaagt | gtkveikrs | |
| | ggtgtcccctcacggttctctggctcaggttctggtactgactatactttg | | |
| | actatctcctccctccagcccgatgatttcgctacctattattgtcagcag | | |
| | tggagccgtaacccacccactttcggaggcggtaccaaagtggagatcaag | | |
| | aggtcataa | | |
| DB83 VHVL x | atggaagcaccagcgcagcttctcttcctcctgctactctggctcccagat | qvqlvqsgaevkkpgasv | SEQ ID NO: 331 |
| TSC456 | accaccggtcaggtgcagctggtgcagtctggggctgaggtgaagaagcct | kvsckasgytftsyamhw | (SEQ ID |
| ScFv-Fc- | ggggcctcagtgaaggtttcctgcaaggcatctggatacaccttcactagc | vrqapgqglewmglvdpe | NO: 332) |
| scFv | tatgctatgcattgggtgcgtcaggcccctggacaagggcttgagtggatg | dgetiyaekfqgrvtmtr | |
| TRI134 | ggacttgttgatcctgaagatggtgaaacaatatatgcagagaagttccag | dtststvymelsslrsed | |
| | ggccgtgtcaccatgacccgcgacacgtccacgagcacagtctacatggag | tavyycarrtyyydssgs | |
| | ctgagcagcctgcgttctgaggacacggccgtgtattactgtgcgagacga | ryafdiwgqgttvtvssg | |
| | acgtattactatgatagtagtggttcccgttatgcttttgatatctggggc | gggsggggsggggsgggg | |
| | caagggaccacggtcaccgtctcttcaggcggcggcggcagcggcggcggc | sdvvmtqsplslpvtpge | |
| | ggcagcggcggcggaggctccggcggcggcggcagcgatgttgtgatgact | pasiscrssqsllhsngd | |
| | cagtctccactctccctgcccgtcacccctggagagccggcctccatctcc | nyldwylqkpgqspqlli | |
| | tgcaggtctagtcagagcctcctgcatagtaatggagacaactatttggat | ylgsnrasgvpdrfsgsg | |
| | tggtacctgcagaagccagggcagtctccacagctcctgatctatttgggt | sgtdftlkisrveaedvg | |
| | tctaatcgggcctccggggtccctgaccgtttcagtggcagtggatcaggc | vyycmqathwpltfgpgt | |
| | acagattttacactgaaaatcagccgtgtggaggctgaggatgttggggtt | kvdiksssepkssdktht | |
| | tattactgcatgcaagctacacactggccactcactttcggccctggtacc | cppcpapeaagapsvflf | |
| | aaagtggatatcaaatcctcgagtgagcccaaatcttctgacaaaactcac | ppkpkdtlmisrtpevtc | |
| | acatgcccaccgtgcccagcacctgaagccgcgggtgcaccgtcagtcttc | vvvdvshedpevkfnwyv | |
| | ctcttccccccaaaacccaaggacaccctcatgatctcccggacccctgag | dgvevhnaktkpreeqyn | |
| | gtcacatgcgtggtggtggacgtgagccacgaagaccctgaggtcaagttc | styrvvsvltvlhqdwln | |
| | aactggtacgtggacggcgtggaggtgcataatgccaagacaaagccgcgg | gkeykcavsnkalpapie | |
| | gaggagcagtacaacagcacgtaccgtgtggtcagcgtcctcaccgtcctg | ktiskakgqprepqvytl | |
| | caccaggactggctgaatggcaaggaatacaagtgcgcggtctccaacaaa | ppsrdeltknqvsltclv | |
| | gccctcccagcccccatcgagaaaaccatctccaaagccaaagggcagccc | kgfypsdiavewesngqp | |
| | cgagaaccacaggtgtacaccctgcccccatcccgggatgagctgaccaag | ennykttppvldsdgsff | |
| | aaccaggtcagcctgacctgcctggtcaaaggcttctatccaagcgacatc | lyskltvdksrwqqgnvf | |
| | gccgtggagtgggagagcaatgggcagccggagaacaactacaagaccacg | scsvmhealhnhytqksl | |
| | cctcccgtgctggactccgacggctccttcttcctctacagcaagctcacc | slspgsggggsggggsgg | |
| | gtggacaagagcaggtggcagcaggggaacgtcttctcatgctccgtgatg | ggspsqvqlvqsgpevkk | |
| | catgaggctctgcacaaccactacacgcagaagagcctctccctgtctccg | pgssvkvsckasgytfsr | |
| | ggttccggaggagggggttcaggtgggggaggttctggcggcgggggaagc | stmhwvrqapgqglewig | |
| | ccttcacaggtgcaactggtgcagagtggacccgaggttaaaaaaccaggg | yinpssaytnynqkfkdr | |
| | tcctccgttaaggttagctgcaaagcctctggctacacattttccaggagt | vtitadkststaymelss | |
| | acaatgcactgggtgaggcaggctcctggacagggactcgagtggatcggg | lrsedtavyycarpqvhy | |
| | tatatcaacccatctagcgcctataccaattacaaccaaaagtttaaggac | dyngfpywgqgtlvtvss | |
| | cgagttaccattaccgctgacaaatccaccagtacagcttatatggagctg | ggggsggggsggggsggg | |
| | tcatctcttaggtccgaggacactgctgtttattactgcgctcgtcctcag | gsdiqmtqspstlsasvg | |
| | gttcactatgactataatggttttccctactggggtcagggaaccctggtg | drvtmtcsasssvsymnw | |
| | actgtctcttctggcggtggaggcagcggtgggggtgggtctggaggcggt | yqqkpgkapkrwiydssk | |
| | ggcagtggcggcggaggctctgatattcagatgactcagtctcctagcact | lasgvpsrfsgsgsgtdy | |
| | ctcagcgccagcgtgggggatcgtgtgacaatgacttgctccgctagcagt | tltisslqpddfatyycq | |
| | agtgtgtcttacatgaattggtatcagcagaagcccgggaaagcacctaag | qwsrnpptfgggtkveik | |
| | cgctggatctatgactcttccaagctggcaagtggtgtcccctcacggttc | rs | |
| | tctggctcaggttctggtactgactatactttgactatctcctccctccag | | |
| | cccgatgatttcgctacctattattgtcagcagtggagccgtaacccaccc | | |
| | actttcggaggcggtaccaaagtggagatcaagaggtcataa | | |
| DB86 VHVL x | atggaagcaccagcgcagcttctcttcctcctgctactctggctcccagat | qvqlvqsgaevkkpgasv | SEQ ID NO: 333 |
| TSC456 | accaccggtcaggtgcagctggtgcagtctggggctgaggtgaagaagcct | kvsckasgymfsghsahw | (SEQ ID |
| ScFv-Fc- | ggggcctcagtgaaggtttcctgcaaggcatctggatatatgttcagtggc | vrqapgqglewmgwmnpn | NO: 334) |
| scFv | cattctgcacactgggtgcgtcaggcccctggacaagggcttgagtggatg | sgntgyaqkfqgrvtmtr | |
| TRI128 | ggatggatgaaccctaacagtggtaacacaggctatgcacagaagttccag | dtststvymelsslrsed | |
| | ggccgtgtcaccatgacccgcgacacgtccacgagcacagtctacatggag | tavyycardssgwydvfd | |
| | ctgagcagcctgcgttctgaggacacggccgtgtattactgtgcgagagat | ywgqgtlvtvssggggsg | |
| | agcagtggctggtacgatgtctttgactactggggccaggggaccctggtc | gggsggggsggggsdiqm | |
| | accgtctcctcaggtggaggcggttcaggcggaggtggatccggcggtggc | tqspsslsasvgdrvtit | |
| | ggctccggtggcggcggatctgacatccagatgacccagtctccatcctcc | crasqgirndlgwyqqkp | |
| | ctgtctgcatctgtaggagaccgcgtcaccatcacttgccgggcaagtcag | gkapklliyaastlqsgv | |
| | ggcatcagaaatgatttaggttggtatcagcagaaaccagggaaagcccct | psrfsgsgsgtdftltis | |
| | aagctcctgatctatgctgcatccactttgcaatcaggggtcccatcacgt | slqpedfatyycqqsyga | |
| | ttcagtggcagtggatctgggacagatttcactctcaccatcagcagtctg | pltfgggtkveiksssep | |
| | caacctgaagattttgcaacttactactgtcaacagagttacggtgccccc | kssdkthtcppcpapeaa | |
| | ctcactttcggcggaggtaccaaggtggagatcaaatcctcgagtgagccc | gapsvflfppkpkdtlmi | |
| | aaatcttctgacaaaactcacacatgcccaccgtgcccagcacctgaagcc | srtpevtcvwdvshedpe | |
| | gcgggtgcaccgtcagtcttcctcttccccccaaaacccaaggacaccctc | vkfnwyvdgvevhnaktk | |
| | atgatctcccggacccctgaggtcacatgcgtggtggtggacgtgagccac | preeqynstyrvvsvltv | |
| | gaagaccctgaggtcaagttcaactggtacgtggacggcgtggaggtgcat | lhqdwlngkeykcavsnk | |
| | aatgccaagacaaagccgcgggaggagcagtacaacagcacgtaccgtgtg | alpapiektiskakgqpr | |
| | gtcagcgtcctcaccgtcctgcaccaggactggctgaatggcaaggaatac | epqvytippsrdeltknq | |
| | aagtgcgcggtctccaacaaagccctcccagcccccatcgagaaaaccatc | vsItclvkgfypsdiave | |
| | tccaaagccaaagggcagccccgagaaccacaggtgtacaccctgccccca | wesngqpennykttppvl | |
| | tcccgggatgagctgaccaagaaccaggtcagcctgacctgcctggtcaaa | dsdgsfflyskltvdksr | |
| | ggcttctatccaagcgacatcgccgtggagtgggagagcaatgggcagccg | wqqgnvfscsvmhealhn | |
| | gagaacaactacaagaccacgcctcccgtgctggactccgacggctccttc | hytqkslslspgsggggs | |
| | ttcctctacagcaagctcaccgtggacaagagcaggtggcagcaggggaac | ggggsggggspsqvqlvq | |
| | gtcttctcatgctccgtgatgcatgaggctctgcacaaccactacacgcag | sgpevkkpgssvkvscka | |
| | aagagcctctccctgtctccgggttccggaggagggggttcaggtggggga | sgytfsrstmhwvrqapg | |
| | ggttctggcggcgggggaagcccttcacaggtgcaactggtgcagagtgga | qglewigyinpssaytny | |
| | cccgaggttaaaaaaccagggtcctccgttaaggttagctgcaaagcctct | nqkfkdrvtitadkstst | |
| | ggctacacattttccaggagtacaatgcactgggtgaggcaggctcctgga | aymelsslrsedtavyyc | |
| | cagggactcgagtggatcgggtatatcaacccatctagcgcctataccaat | arpqvhydyngfpywgqg | |
| | tacaaccaaaagtttaaggaccgagttaccattaccgctgacaaatccacc | tlvtvssggggsggggsg | |
| | agtacagcttatatggagctgtcatctcttaggtccgaggacactgctgtt | gggsggggsdiqmtqsps | |
| | tattactgcgctcgtcctcaggttcactatgactataatggttttccctac | tlsasvgdrvtmtcsass | |
| | tggggtcagggaaccctggtgactgtctcttctggcggtggaggcagcggt | svsymnwyqqkpgkapkr | |
| | gggggtgggtctggaggcggtggcagtggcggcggaggctctgatattcag | wiydssklasgvpsrfsg | |
| | atgactcagtctcctagcactctcagcgccagcgtgggggatcgtgtgaca | sgsgtdytltisslqpdd | |
| | atgacttgctccgctagcagtagtgtgtcttacatgaattggtatcagcag | fatyycqqwsrnpptfgg | |
| | aagcccgggaaagcacctaagcgctggatctatgactcttccaagctggca | gtkveikrs | |
| | agtggtgtcccctcacggttctctggctcaggttctggtactgactatact | | |
| | ttgactatctcctccctccagcccgatgatttcgctacctattattgtcag | | |
| | cagtggagccgtaacccacccactttcggaggcggtaccaaagtggagatc | | |
| | aagaggtcataa | | |
| DB280 | atggaagcaccagcgcagcttctcttcctcctgctactctggctcccagat | qvqlvqsgaevkkpgasv | SEQ ID NO: 327 |
| VHVL x | accaccggtcaggtgcagctggtgcagtctggggctgaggtgaagaagcct | kvsckasgyslnlyymhw | (SEQ ID |
| TSC456 | ggggcctcagtgaaggtttcctgcaaggcatctggatacagcctcaactta | vrqapgqglewmgwmnpn | NO: 328) |
| scFv-Fc- | tactatatgcactgggtgcgtcaggcccctggacaagggcttgagtggatg | sgntgyaqkfqgrvtmtr | |
| scFv | ggatggatgaaccctaacagtggtaacacaggctatgcacagaagttccag | dtststvymelsslrsed | |
| TRI131 | ggccgtgtcaccatgacccgcgacacgtccacgagcacagtctacatggag | tavyycasldcsggscys | |
| | ctgagcagcctgcgttctgaggacacggccgtgtattactgtgcgagcctc | eydafdiwgqgttvtvss | |
| | gattgtagtggtggtagctgctactccgaatatgatgcttttgatatctgg | ggggsggggsggggsggg | |
| | ggccaagggaccacggtcaccgtctcctcaggcggcggcggcagcggcggc | gsdiqmtqspsslsasvg | |
| | ggcggcagcggcggcggaggctccggcggcggcggcagcgacatccagatg | drvtitcrasqsissyln | |
| | acccagtctccatcctccctgtctgcatctgtaggagacagagtcaccatc | wyqqkpgkapklliyaas | |
| | acttgccgggcaagtcagagcattagcagctatctgaattggtatcagcag | slqsgvpsrfsgsgsgtd | |
| | aaaccagggaaagcccctaagctcctgatctatgctgcatccagtttgcaa | ftltisslqpedfatyyc | |
| | agtggggtcccatcaaggttcagtggcagtggatctgggacagatttcact | qqsystpltfgggtkvei | |
| | ctcaccatcagcagtctgcaacctgaagattttgcaacttactactgtcaa | ksssepkssdkthtcppc | |
| | cagagttacagtacccctctcactttcggcggaggtaccaaggtggagatc | papeaagapsvflfppkp | |
| | aaatcctcgagtgagcccaaatcttctgacaaaactcacacatgcccaccg | kdtlmisrtpevtcvvvd | |
| | tgcccagcacctgaagccgcgggtgcaccgtcagtcttcctcttcccccca | vshedpevkfnwyvdgve | |
| | aaacccaaggacaccctcatgatctcccggacccctgaggtcacatgcgtg | vhnaktkpreeqynstyr | |
| | gtggtggacgtgagccacgaagaccctgaggtcaagttcaactggtacgtg | vvsvltvlhqdwingkey | |
| | gacggcgtggaggtgcataatgccaagacaaagccgcgggaggagcagtac | kcavsnkalpapiektis | |
| | aacagcacgtaccgtgtggtcagcgtcctcaccgtcctgcaccaggactgg | kakgqprepqvytlppsr | |
| | ctgaatggcaaggaatacaagtgcgcggtctccaacaaagccctcccagcc | deltknqvsltclvkgfy | |
| | cccatcgagaaaaccatctccaaagccaaagggcagccccgagaaccacag | psdiavewesngqpenny | |
| | gtgtacaccctgcccccatcccgggatgagctgaccaagaaccaggtcagc | kttppvldsdgsfflysk | |
| | ctgacctgcctggtcaaaggcttctatccaagcgacatcgccgtggagtgg | ltvdksrwqqgnvfscsv | |
| | gagagcaatgggcagccggagaacaactacaagaccacgcctcccgtgctg | mhealhnhytqkslslsp | |
| | gactccgacggctccttcttcctctacagcaagctcaccgtggacaagagc | gsggggsggggsggggsp | |
| | aggtggcagcaggggaacgtcttctcatgctccgtgatgcatgaggctctg | sqvqlvqsgpevkkpgss | |
| | cacaaccactacacgcagaagagcctctccctgtctccgggttccggagga | vkvsckasgytfsrstmh | |
| | gggggttcaggtgggggaggttctggcggcgggggaagcccttcacaggtg | wvrqapgqglewigyinp | |
| | caactggtgcagagtggacccgaggttaaaaaaccagggtcctccgttaag | ssaytnynqkfkdrvtit | |
| | gttagctgcaaagcctctggctacacattttccaggagtacaatgcactgg | adkststaymelsslrse | |
| | gtgaggcaggctcctggacagggactcgagtggatcgggtatatcaaccca | dtavyycarpqvhydyng | |
| | tctagcgcctataccaattacaaccaaaagtttaaggaccgagttaccatt | fpywgqgtlvtvssgggg | |
| | accgctgacaaatccaccagtacagcttatatggagctgtcatctcttagg | sggggsggggsggggsdi | |
| | tccgaggacactgctgtttattactgcgctcgtcctcaggttcactatgac | qmtqspstlsasvgdrvt | |
| | tataatggttttccctactggggtcagggaaccctggtgactgtctcttct | mtcsasssvsymnwyqqk | |
| | ggcggtggaggcagcggtgggggtgggtctggaggcggtggcagtggcggc | pgkapkrwiydssklasg | |
| | ggaggctctgatattcagatgactcagtctcctagcactctcagcgccagc | vpsrfsgsgsgtdytlti | |
| | gtgggggatcgtgtgacaatgacttgctccgctagcagtagtgtgtcttac | sslqpddfatyycqqwsr | |
| | atgaattggtatcagcagaagcccgggaaagcacctaagcgctggatctat | npptfgggtkveikrs | |
| | gactcttccaagctggcaagtggtgtcccctcacggttctctggctcaggt | | |
| | tctggtactgactatactttgactatctcctccctccagcccgatgatttc | | |
| | gctacctattattgtcagcagtggagccgtaacccacccactttcggaggc | | |
| | ggtaccaaagtggagatcaagaggtcataa | | |
| DB331 | atggaagcaccagcgcagcttctcttcctcctgctactctggctcccagat | qvqlvqsgaevkkpgasv | SEQ ID NO: 329 |
| VHVL x | accaccggtcaggtgcagctggtgcagtctggggctgaggtgaagaagcct | kvsckasgytftsyymhw | (SEQ ID |
| TSC456 | ggggcctcagtgaaggtttcctgcaaggcatctggatacaccttcaccagc | vrqapgqglewmgwmnpn | NO: 330) |
| scFv-Fc- | tactatatgcactgggtgcgtcaggcccctggacaagggcttgagtggatg | sgntgyaqkfqgrvtmtr | |
| scFv | ggatggatgaaccctaacagtggtaacacaggctatgcacagaagttccag | dtststvymelsslrsed | |
| TRI132 | ggccgtgtcaccatgacccgcgacacgtccacgagcacagtctacatggag | tavyycatdlagealfdp | |
| | ctgagcagcctgcgttctgaggacacggccgtgtattactgtgcaacagat | wgqgtlvtvssggggsgg | |
| | ctcgcgggggaagccttgttcgacccctggggccagggcaccctggtcacc | ggsggggsggggsdiqmt | |
| | gtctcctcaggcggcggcggcagcggcggcggcggcagcggcggcggaggc | qspsslsasvgdrvtitc | |
| | tccggcggcggcggcagcgacatccagatgacccagtctccatcctccctg | rasqsissylnwyqqkpg | |
| | tctgcatctgtaggagacagagtcaccatcacttgccgggcaagtcagagc | kapklliyaasslqsgvp | |
| | attagcagctatctgaattggtatcagcagaaaccagggaaagcccctaag | srfsgsgsgtdftltiss | |
| | ctcctgatctatgctgcatccagtttgcaaagtggggtcccatcaaggttc | lqpedfatyycqqsystp | |
| | agtggcagtggatctgggacagatttcactctcaccatcagcagtctgcaa | ltfgggtkveiksssepk | |
| | cctgaagattttgcaacttactactgtcaacagagttacagtacccctctc | ssdkthtcppcpapeaag | |
| | actttcggcggaggtaccaaggtggagatcaaatcctcgagtgagcccaaa | apsvflfppkpkdtlmis | |
| | tcttctgacaaaactcacacatgcccaccgtgcccagcacctgaagccgcg | rtpevtcvvvdvshedpe | |
| | ggtgcaccgtcagtcttcctcttccccccaaaacccaaggacaccctcatg | vkfnwyvdgvevhnaktk | |
| | atctcccggacccctgaggtcacatgcgtggtggtggacgtgagccacgaa | preeqynstyrvvsvltv | |
| | gaccctgaggtcaagttcaactggtacgtggacggcgtggaggtgcataat | lhqdwlngkeykcavsnk | |
| | gccaagacaaagccgcgggaggagcagtacaacagcacgtaccgtgtggtc | alpapiektiskakgqpr | |
| | agcgtcctcaccgtcctgcaccaggactggctgaatggcaaggaatacaag | epqvytlppsrdeltknq | |
| | tgcgcggtctccaacaaagccctcccagcccccatcgagaaaaccatctcc | vsltclvkgfypsdiave | |
| | aaagccaaagggcagccccgagaaccacaggtgtacaccctgcccccatcc | wesngqpennykttppvl | |
| | cgggatgagctgaccaagaaccaggtcagcctgacctgcctggtcaaaggc | dsdgsfflyskltvdksr | |
| | ttctatccaagcgacatcgccgtggagtgggagagcaatgggcagccggag | wqqgnvfscsvmhealhn | |
| | aacaactacaagaccacgcctcccgtgctggactccgacggctccttcttc | hytqkslslspgsggggs | |
| | ctctacagcaagctcaccgtggacaagagcaggtggcagcaggggaacgtc | ggggsggggspsqvqlvq | |
| | ttctcatgctccgtgatgcatgaggctctgcacaaccactacacgcagaag | sgpevkkpgssvkvscka | |
| | agcctctccctgtctccgggttccggaggagggggttcaggtgggggaggt | sgytfsrstmhwvrqapg | |
| | tctggcggcgggggaagcccttcacaggtgcaactggtgcagagtggaccc | qglewigyinpssaytny | |
| | gaggttaaaaaaccagggtcctccgttaaggttagctgcaaagcctctggc | nqkfkdrvtitadkstst | |
| | tacacattttccaggagtacaatgcactgggtgaggcaggctcctggacag | aymelsslrsedtavyyc | |
| | ggactcgagtggatcgggtatatcaacccatctagcgcctataccaattac | arpqvhydyngfpywgqg | |
| | aaccaaaagtttaaggaccgagttaccattaccgctgacaaatccaccagt | tlvtvssggggsggggsg | |
| | acagcttatatggagctgtcatctcttaggtccgaggacactgctgtttat | gggsggggsdiqmtqsps | |
| | tactgcgctcgtcctcaggttcactatgactataatggttttccctactgg | tlsasvgdrvtmtcsass | |
| | ggtcagggaaccctggtgactgtctcttctggcggtggaggcagcggtggg | svsymnwyqqkpgkapkr | |
| | ggtgggtctggaggcggtggcagtggcggcggaggctctgatattcagatg | wiydssklasgvpsrfsg | |
| | actcagtctcctagcactctcagcgccagcgtgggggatcgtgtgacaatg | sgsgtdytltisslqpdd | |
| | acttgctccgctagcagtagtgtgtcttacatgaattggtatcagcagaag | fatyycqqwsrnpptfgg | |
| | cccgggaaagcacctaagcgctggatctatgactcttccaagctggcaagt | gtkveikrs | |
| | ggtgtcccctcacggttctctggctcaggttctggtactgactatactttg | | |
| | actatctcctccctccagcccgatgatttcgctacctattattgtcagcag | | |
| | tggagccgtaacccacccactttcggaggcggtaccaaagtggagatcaag | | |
| | aggtcataa | | |
| DB415 | atggaagcaccagcgcagcttctcttcctcctgctactctggctcccagat | evqlvesggglvqpggsl | SEQ ID NO: 335 |
| VHVL x | accaccggtgaggtgcagctggtggagtctgggggaggcttggtacagcct | riscaasgitfssygmhw | (SEQ ID |
| TSC456 | ggggggtccctgcgcctctcctgtgcagcctctggaatcaccttcagtagt | vrqapgkglewvsgiswn | NO: 336) |
| scFv-Fc- | tatggcatgcattgggtccgccaggctccagggaaggggctggagtgggtc | sgnrvyvdsvkgrftisr | |
| scFv | tcaggtattagttggaatagtggtaacagagtctatgtggactctgtgaag | dnskntlylqmnslraed | |
| TRI138 | ggccggttcaccatctcccgcgacaattccaagaacacgctgtatctgcaa | tavyycardtndafdiwg | |
| | atgaacagcctgcgcgccgaggacacggccgtatattactgtgcgagagat | qgttvtvssggggsgggg | |
| | actaatgatgcttttgatatctggggccaagggaccacggtcaccgtctcc | sggggsggggsdiqmtqs | |
| | tcaggtggaggcggttcaggcggaggtggatccggcggtggcggctccggt | psslsasvgdrvtitcra | |
| | ggcggcggatctgacatccagatgacccagtctccatcctccctgtctgca | sqsissylnwyqqkpgka | |
| | tctgtaggagacagagtcaccatcacttgccgggcaagtcagagcattagc | pklliyaasslqsgvpsr | |
| | agctatctgaattggtatcagcagaaaccagggaaagcccctaagctcctg | fsgsgsgtdftltisslq | |
| | atctatgctgcatccagtttgcaaagtggggtcccatcaaggttcagtggc | pedfatyycqqsystplt | |
| | agtggatctgggacagatttcactctcaccatcagcagtctgcaacctgaa | fgggtkveiksssepkss | |
| | gattttgcaacttactactgtcaacagagttacagtacccctctcactttc | dkthtcppcpapeaagap | |
| | ggcggaggtaccaaggtggagatcaaatcctcgagtgagcccaaatcttct | svflfppkpkdtlmisrt | |
| | gacaaaactcacacatgcccaccgtgcccagcacctgaagccgcgggtgca | pevtcvvvdvshedpevk | |
| | ccgtcagtcttcctcttccccccaaaacccaaggacaccctcatgatctcc | fnwyvdgvevhnaktkpr | |
| | cggacccctgaggtcacatgcgtggtggtggacgtgagccacgaagaccct | eeqynstyrvvsvltvlh | |
| | gaggtcaagttcaactggtacgtggacggcgtggaggtgcataatgccaag | qdwlngkeykcavsnkal | |
| | acaaagccgcgggaggagcagtacaacagcacgtaccgtgtggtcagcgtc | papiektiskakgqprep | |
| | ctcaccgtcctgcaccaggactggctgaatggcaaggaatacaagtgcgcg | qvytlppsrdeltknqvs | |
| | gtctccaacaaagccctcccagcccccatcgagaaaaccatctccaaagcc | ltclvkgfypsdiavewe | |
| | aaagggcagccccgagaaccacaggtgtacaccctgcccccatcccgggat | sngqpennykttppvlds | |
| | gagctgaccaagaaccaggtcagcctgacctgcctggtcaaaggcttctat | dgsfflyskltvdksrwq | |
| | ccaagcgacatcgccgtggagtgggagagcaatgggcagccggagaacaac | qgnvfscsvmhealhnhy | |
| | tacaagaccacgcctcccgtgctggactccgacggctccttcttcctctac | tqkslslspgsggggsgg | |
| | agcaagctcaccgtggacaagagcaggtggcagcaggggaacgtcttctca | ggsggggspsqvqlvqsg | |
| | tgctccgtgatgcatgaggctctgcacaaccactacacgcagaagagcctc | pevkkpgssvkvsckasg | |
| | tccctgtctccgggttccggaggagggggttcaggtgggggaggttctggc | ytfsrstmhwvrqapgqg | |
| | ggcgggggaagcccttcacaggtgcaactggtgcagagtggacccgaggtt | lewigyinpssaytnynq | |
| | aaaaaaccagggtcctccgttaaggttagctgcaaagcctctggctacaca | kfkdrvtitadkststay | |
| | ttttccaggagtacaatgcactgggtgaggcaggctcctggacagggactg | melsslrsedtavyycar | |
| | gagtggatcgggtatatcaacccatctagcgcctataccaattacaaccaa | pqvhydyngfpywgqgtl | |
| | aagtttaaggaccgagttaccattaccgctgacaaatccaccagtacagct | vtvssggggsggggsggg | |
| | tatatggagctgtcatctcttaggtccgaggacactgctgtttattactgc | gsggggsdiqmtqspstl | |
| | gctcgtcctcaggttcactatgactataatggttttccctactggggtcag | sasvgdrvtmtcsasssv | |
| | ggaaccctggtgactgtctcttctggcggtggaggcagcggtgggggtggg | symnwyqqkpgkapkrwi | |
| | tctggaggcggtggcagtggcggcggaggctctgatattcagatgactcag | ydssklasgvpsrfsgsg | |
| | tctcctagcactctcagcgccagcgtgggggatcgtgtgacaatgacttgc | sgtdytltisslqpddfa | |
| | tccgctagcagtagtgtgtcttacatgaattggtatcagcagaagcccggg | tyycqqwsrnpptfgggt | |
| | aaagcacctaagcgctggatctatgactcttccaagctggcaagtggtgtc | kveikrs | |
| | ccctcacggttctctggctcaggttctggtactgactatactttgactatc | | |
| | tcctccctccagcccgatgatttcgctacctattattgtcagcagtggagc | | |
| | cgtaacccacccactttcggaggcggtaccaaagtggagatcaagaggtca | | |
| | tga | | |
| DB435 | atggaagcaccagcgcagcttctcttcctcctgctactctggctcccagat | qvqlvqsgaevkkpgasv | SEQ ID NO: 317 |
| VHVL x | accaccggtcaggtgcagctggtgcagtctggggctgaggtgaagaagcct | kvsckasggtfssyaisw | (SEQ ID |
| TSC456 | ggggcctcagtgaaggtttcctgcaaggcatctggaggcaccttcagcagc | vrqapgqglewmgwitph | NO: 318) |
| scFv-Fc- | tatgctatcagctgggtgcgtcaggcccctggacaagggcttgagtggatg | ngnikyarefqgrvtmtr | |
| scFv | ggctggatcacccctcacaatggtaacataaagtatgcacgggagttccag | dtststvymelsslrsed | |
| TRI139 | ggccgtgtcaccatgacccgcgacacgtccacgagcacagtctacatggag | tavyycakdlnwnaafdy | |
| | ctgagcagcctgcgttctgaggacacggccgtgtattactgtgcgaaagat | wgqgtlvtvssggggsgg | |
| | ctgaactggaacgcagcctttgactactggggccaggggaccctggtcacc | ggsggggsggggsdiqmt | |
| | gtctcctcaggtggaggcggttcaggcggaggtggatccggcggtggcggc | qspsslsasvgdrvtitc | |
| | tccggtggcggcggatctgacatccagatgacccagtctccatcctccctg | rasqsissylnwyqqkpg | |
| | tctgcatctgtaggagacagagtcaccatcacttgccgggcaagtcagagc | kapklliyaasslqsgvp | |
| | attagcagctatctgaattggtatcagcagaaaccagggaaagcccctaag | srfsgsgsgtdftltiss | |
| | ctcctgatctatgctgcatccagtttgcaaagtggggtcccatcaaggttc | lqpedfatyycqqsystp | |
| | agtggcagtggatctgggacagatttcactctcaccatcagcagtctgcaa | ltfgggtkveiksssepk | |
| | cctgaagattttgcaacttactactgtcaacagagttacagtacccctctc | ssdkthtcppcpapeaag | |
| | actttcggcggaggtaccaaggtggagatcaaatcctcgagtgagcccaaa | apsvfifppkpkdtlmis | |
| | tcttctgacaaaactcacacatgcccaccgtgcccagcacctgaagccgcg | rtpevtcvvvdvshedpe | |
| | ggtgcaccgtcagtcttcctcttccccccaaaacccaaggacaccctcatg | vkfnwyvdgvevhnaktk | |
| | atctcccggacccctgaggtcacatgcgtggtggtggacgtgagccacgaa | preeqynstyrvvsvltv | |
| | gaccctgaggtcaagttcaactggtacgtggacggcgtggaggtgcataat | lhqdwlngkeykcavsnk | |
| | gccaagacaaagccgcgggaggagcagtacaacagcacgtaccgtgtggtc | alpapiektiskakgqpr | |
| | agcgtcctcaccgtcctgcaccaggactggctgaatggcaaggaatacaag | epqvytlppsrdeltknq | |
| | tgcgcggtctccaacaaagccctcccagcccccatcgagaaaaccatctcc | vsltclvkgfypsdiave | |
| | aaagccaaagggcagccccgagaaccacaggtgtacaccctgcccccatcc | wesngqpennykttppvl | |
| | cgggatgagctgaccaagaaccaggtcagcctgacctgcctggtcaaaggc | dsdgsfflyskltvdksr | |
| | ttctatccaagcgacatcgccgtggagtgggagagcaatgggcagccggag | wqqgnvfscsvmhealhn | |
| | aacaactacaagaccacgcctcccgtgctggactccgacggctccttcttc | hytqkslslspgsggggs | |
| | ctctacagcaagctcaccgtggacaagagcaggtggcagcaggggaacgtc | ggggsggggspsqvqlvq | |
| | ttctcatgctccgtgatgcatgaggctctgcacaaccactacacgcagaag | sgpevkkpgssvkvscka | |
| | agcctctccctgtctccgggttccggaggagggggttcaggtgggggaggt | sgytfsrstmhwvrqapg | |
| | tctggcggcgggggaagcccttcacaggtgcaactggtgcagagtggaccc | qglewigyinpssaytny | |
| | gaggttaaaaaaccagggtcctccgttaaggttagctgcaaagcctctggc | nqkfkdrvtitadkstst | |
| | tacacattttccaggagtacaatgcactgggtgaggcaggctcctggacag | aymelssirsedtavyyc | |
| | ggactggagtggatcgggtatatcaacccatctagcgcctataccaattac | arpqvhydyngfpywgqg | |
| | aaccaaaagtttaaggaccgagttaccattaccgctgacaaatccaccagt | tlvtvssggggsggggsg | |
| | acagcttatatggagctgtcatctcttaggtccgaggacactgctgtttat | gggsggggsdiqmtqsps | |
| | tactgcgctcgtcctcaggttcactatgactataatggttttccctactgg | tlsasvgdrvtmtcsass | |
| | ggtcagggaaccctggtgactgtctcttctggcggtggaggcagcggtggg | svsymnwyqqkpgkapkr | |
| | ggtgggtctggaggcggtggcagtggcggcggaggctctgatattcagatg | wiydssklasgvpsrfsg | |
| | actcagtctcctagcactctcagcgccagcgtgggggatcgtgtgacaatg | sgsgtdytltisslqpdd | |
| | acttgctccgctagcagtagtgtgtcttacatgaattggtatcagcagaag | fatyycqqwsrnpptfgg | |
| | cccgggaaagcacctaagcgctggatctatgactcttccaagctggcaagt | gtkveikrs | |
| | ggtgtcccctcacggttctctggctcaggttctggtactgactatactttg | | |
| | actatctcctccctccagcccgatgatttcgctacctattattgtcagcag | | |
| | tggagccgtaacccacccactttcggaggcggtaccaaagtggagatcaag | | |
| | aggtcatga | | |
| Cris7 and | | RSTMH | (SEQ ID |
| DRA222 VH | | | NO: 345) |
| CDR1 | | | |
| (Kabat) | | | |
| Cris7 and | | YINPSSAYTNYNQKFK | (SEQ ID |
| DRA222 VH | | | NO: 346) |
| CDR2 | | | |
| (Kabat) | | | |
| Cris7 and | | QVHYDYNGFPY | (SEQ ID |
| DRA222 VH | | | NO: 347) |
| CDR3 | | | |
| (Kabat) | | | |
| Cris7 and | | SASSSVSYMN | (SEQ ID |
| DRA222 VL | | | NO: 348) |
| CDR1 | | | |
| (Kabat) | | | |
| Cris7 and | | DSSKLAS | (SEQ ID |
| DRA222 VL | | | NO: 349) |
| CDR2 | | | |
| (Kabat) | | | |
| Cris7 and | | QQWSRNPPT | (SEQ ID |
| DRA222 VL | | | NO: 350) |
| CDR3 | | | |
| (Kabat) | | | |
| Cris7 and | | GYTFTRST | (SEQ ID |
| DRA222 VH | | | NO: 351) |
| CDR1 | | | |
| (IMGT) | | | |
| Cris7 and | | INPSSAYT | (SEQ ID |
| DRA222 VH | | | NO: 352) |
| CDR2 | | | |
| (IMGT) | | | |
| Cris7 and | | QQWSRNPPT | (SEQ ID |
| DRA222 VH | | | NO: 353) |
| CDR3 | | | |
| (IMGT) | | | |
| Cris7 and | | ASSSVSY | (SEQ ID |
| DRA222 VL | | | NO: 354) |
| CDR1 | | | |
| (IMGT) | | | |
| Cris7 and | | DSS | (SEQ ID |
| DRA222 VL | | | NO: 355) |
| CDR2 | | | |
| (IMGT) | | | |
| Cris7 and | | QQWSRNPPT | (SEQ ID |
| DRA222 VL | | | NO: 356) |
| CDR3 | | | |
| (IMGT) | | | |
| 12C VH | | KYAMN | (SEQ ID |
| CDR1 | | | NO: 357) |
| (Kabat) | | | |
| 12C VH | | RIRSKYNNYATYYADSVK | (SEQ ID |
| CDR2 | | D | NO: 358) |
| (Kabat) | | | |
| 12C VH | | HGNFGNSYISYWAY | (SEQ ID |
| CDR3 | | | NO: 359) |
| (Kabat) | | | |
| 12C VL | | GSSTGAVTSGNYPN | (SEQ ID |
| CDR1 | | | NO: 360) |
| (Kabat) | | | |
| 12C VL | | GTKFLAP | (SEQ ID |
| CDR2 | | | NO: 361) |
| (Kabat) | | | |
| 12C VL | | VLWYSNRWV | (SEQ ID |
| CDR3 | | | NO: 362) |
| (Kabat) | | | |
| 12C VH | | GFTFNKYA | (SEQ ID |
| CDR1 | | | NO: 363) |
| (IMGT) | | | |
| 12C VH | | IRSKYNNYAT | (SEQ ID |
| CDR2 | | | NO: 364) |
| (IMGT) | | | |
| 12C VH | | VRHGNFGNSYISYWAY | (SEQ ID |
| CDR3 | | | NO: 365) |
| (IMGT) | | | |
| 12C VL | | TGAVTSGNY | (SEQ ID |
| CDR1 | | | NO: 366) |
| (IMGT) | | | |
| 12C VL | | GTK | (SEQ ID |
| CDR2 | | | NO: 367) |
| (IMGT) | | | |
| 12C VL | | VLWYSNRWV | (SEQ ID |
| CDR3 | | | NO: 368) |
| (IMGT) | | | |
| HuM291 | | SYTMH | (SEQ ID |
| VH CDR1 | | | NO: 369) |
| (Kabat) | | | |
| HuM291 | | YINPRSGYTHYNQKLKD | (SEQ ID |
| VH CDR2 | | | NO: 370) |
| (Kabat) | | | |
| HuM291 | | SAYYDYDGFAY | (SEQ ID |
| VH CDR3 | | | NO: 371) |
| (Kabat) | | | |
| HuM291 VL | | SASSSVSYMN | (SEQ ID |
| CDR1 | | | NO: 372) |
| (Kabat) | | | |
| HuM291 VL | | DTSKLAS | (SEQ ID |
| CDR2 | | | NO: 373) |
| (Kabat) | | | |
| HuM291 VL | | QQWSSNPPT | (SEQ ID |
| CDR3 | | | NO: 374) |
| (Kabat) | | | |
| HuM291 | | GYTFISYT | (SEQ ID |
| VH CDR1 | | | NO: 375) |
| (IMGT) | | | |
| HuM291 | | INPRSGYT | (SEQ ID |
| VH CDR2 | | | NO: 376) |
| (IMGT) | | | |
| HuM291 | | ARSAYYDYDGFAY | (SEQ ID |
| VH CDR3 | | | NO: 377) |
| (IMGT) | | | |
| HuM291 VL | | ASSSVSY | (SEQ ID |
| CDR1 | | | NO: 378) |
| (IMGT) | | | |
| HuM291 VL | | DTS | (SEQ ID |
| CDR2 | | | NO: 379) |
| (IMGT) | | | |
| HuM291 VL | | QQWSSNPPT | (SEQ ID |
| CDR3 | | | NO: 380) |
| (IMGT) | | | |
| TSC455 | | QVQLVQSGPEVKKPGSSV | (SEQ ID |
| (anti-CD3) | | KVSCKASGYTFSRSTMHW | NO: 381) |
| TSC394 | | VRQAPGQGLEWIGYINPS | |
| F87Y scFv | | SAYTNYNQKFKDRVTITA | |
| | | DKSTSTAYMELSSLRSED | |
| | | TAVYYCARPQVHYDYNGF | |
| | | PYWGQGTLVTVSSGGGGS | |
| | | GGGGSGGGGSGGGGSDIQ | |
| | | MTQSPSTLSASVGDRVTM | |
| | | TCSASSSVSYMNWYQQKP | |
| | | GKAPKRWIYDSSKLASGV | |
| | | PSRFSGSGSGTEYTLTIS | |
| | | SLQPDDFATYYCQQWSRN | |
| | | PPTFGGGTKVEIKRSSS | |
| TSC456 | | QVQLVQSGPEVKKPGSSV | (SEQ ID |
| (anti-CD3) | | KVSCKASGYTFSRSTMHW | NO: 382) |
| TSC394 | | VRQAPGQGLEWIGYINPS | |
| E86D F87Y | | SAYTNYNQKFKDRVTITA | |
| scFv | | DKSTSTAYMELSSLRSED | |
| | | TAVYYCARPQVHYDYNGF | |
| | | PYWGQGTLVTVSSGGGGS | |
| | | GGGGSGGGGSGGGGSDIQ | |
| | | MTQSPSTLSASVGDRVTM | |
| | | TCSASSSVSYMNWYQQKP | |
| | | GKAPKRWIYDSSKLASGV | |
| | | PSRFSGSGSGTDYTLTIS | |
| | | SLQPDDFATYYCQQWSRN | |
| | | PPTFGGGTKVEIKRSSS | |
| TSC455 and | | QVQLVQSGPEVKKPGSSV | (SEQ ID |
| TSC456 | | KVSCKASGYTFSRSTMHW | NO: 383) |
| variable | | VRQAPGQGLEWIGYINPS | |
| heavy | | SAYTNYNQKFKDRVTITA | |
| domain | | DKSTSTAYMELSSLRSED | |
| | | TAVYYCARPQVHYDYNGF | |
| | | PYWGQGTLVTVSS | |
| TSC455 | | DIQMTQSPSTLSASVGDR | (SEQ ID |
| variable | | VTMTCSASSSVSYMNWYQ | NO: 384) |
| light | | QKPGKAPKRWIYDSSKLA | |
| domain | | SGVPSRFSGSGSGTEYTL | |
| | | TISSLQPDDFATYYCQQW | |
| | | SRNPPTFGGGTKVEIKRS | |
| TSC456 | | DIQMTQSPSTLSASVGDR | (SEQ ID |
| variable | | VTMTCSASSSVSYMNWYQ | NO: 385) |
| light | | QKPGKAPKRWIYDSSKLA | |
| domain | | SGVPSRFSGSGSGTDYTL | |
| | | TISSLQPDDFATYYCQQW | |
| | | SRNPPTFGGGTKVEIKRS | |
| DRA222 | | QVQLVESGGGVVQPGRSL | (SEQ ID |
| (anti-CD3) | | RLSCKASGYTFTRSTMHW | NO: 386) |
| scFv | | VRQAPGQGLEWIGYINPS | |
| | | SAYTNYNQKFKDRFTISA | |
| | | DKSKSTAFLQMDSLRPED | |
| | | TGVYFCARPQVHYDYNGF | |
| | | PYWGQGTPVTVSSGGGGS | |
| | | GGGGSGGGGSAQDIQMTQ | |
| | | SPSSLSASVGDRVTMTCS | |
| | | ASSSVSYMNWYQQKPGKA | |
| | | PKRWIYDSSKLASGVPAR | |
| | | FSGSGSGTDYTLTISSLQ | |
| | | PEDFATYYCQQWSRNPPT | |
| | | FGGGTKLQITSSS | |
| DRA222 | | QVQLVESGGGWVQPGRSL | (SEQ ID |
| variable | | RLSCKASGYTFTRSTMHW | NO: 387) |
| heavy | | VRQAPGQGLEWIGYINPS | |
| domain | | SAYTNYNQKFKDRFTISA | |
| | | DKSKSTAFLQMDSLRPED | |
| | | TGVYFCARPQVHYDYNGF | |
| | | PYWGQGTPVTVSS | |
| DRA222 | | DIQMTQSPSSLSASVGDR | (SEQ ID |
| variable | | VTMTCSASSSVSYMNWYQ | NO: 388) |
| light | | QKPGKAPKRWIYDSSKLA | |
| domain | | SGVPARFSGSGSGTDYTL | |
| | | TISSLQPEDFATYYCQQW | |
| | | SRNPPTFGGGTKLQITS | |

TABLE 5
Composition of Humanized Constructs

| Construct | scFv | Nucleotide | Amino acid |
|---|---|---|---|
| ID | Orientation | SEQ ID NO | SEQ ID NO |
| TRI129 | VHVL | 309 | 310 |
| TRI130 | VLVH | 311 | 312 |
| TRI123 | VHVL | 313 | 314 |
| TRI124 | VLVH | 315 | 316 |
| TRI139 | VHVL | 317 | 318 |
| TRI137 | VHVL | 319 | 320 |
| TRI125 | VHVL | 321 | 322 |
| TRI126 | VHVL | 323 | 324 |
| TRI127 | VLVH | 325 | 326 |
| TRI131 | VHVL | 327 | 328 |
| TRI132 | VHVL | 329 | 330 |
| TRI134 | VHVL | 331 | 332 |
| TRI128 | VHVL | 333 | 334 |
| TRI138 | VHVL | 335 | 336 |

TABLE 6
Amino acid sequences of exemplary binding protein constructs

| Construct | | SEQ |
|---|---|---|
| name | Sequence | ID NO |
| TRI130 | MEAPAQLLFLLLLWLPDTTGDIVMTQSPDSLAVSLGERATINCKS | 337 |
| (CD123 | SHSVLYSSNNKNYLAWYQQKPGQPPKLLIYWASTRESGVPDR | |
| binding | FSGSGSGTDFTLTISSLQAEDVAVYYCQQYYSTPPTTFGGGTKV | |
| domain in | EIKGGGGSGGGGSGGGGSGGGGSEVQLLESGGGLVQPGGSL | |
| bold, CD3 | RLSCAASGFTFSSYGMSWVRQAPGKGLEGVSAISGSGGSTYY | |
| binding | ADSVKGRFTISRDNSKNTLYLQMNSLRAEDTAVYYCAKEKLRY | |
| domain in | FDWLSDAFDIWGQGTMVTVSSSEPKSSDKTHTCPPCPAPEAAG | |
| italics) | APSVFLFPPKPKDTLMISRTPEVTCVVVDVSHEDPEVKFNWYVD | |
| (CDR | GVEVHNAKTKPREEQYNSTYRVVSVLTVLHQDWLNGKEYKCAV | |
| sequences | SNKALPAPIEKTISKAKGQPREPQVYTLPPSRDELTKNQVSLTCL | |
| are single- | VKGFYPSDIAVEWESNGQPENNYKTTPPVLDSDGSFFLYSKLTV | |
| underlined) | DKSRWQQGNVFSCSVMHEALHNHYTQKSLSLSPGSGGGGSGG | |
| | GGSGGGGSPSQVQLVQSGPEVKKPGSSVKVSCKASGYTFSRS | |
| | TMHWVRQAPGQGLEWIGYINPSSAYTNYNQKFKDRVTITADKST | |
| | STAYMELSSLRSEDTAVYYCARPQVHYDYNGFPYWGQGTLVTV | |
| | SSGGGGSGGGGSGGGGSGGGGSDIQMTQSPSTLSASVGDRV | |
| | TMTCSASSSVSYMNWYQQKPGKAPKRWIYDSSKLASGVPSRFS | |
| | GSGSGTDYTLTISSLQPDDFATYYCQQWSRNPPTFGGGTKVEIK | |
| | RS | |
| TRI185 | MEAPAQLLFLLLLWLPDTTGDIVMTQSPDSLAVSLGERATINCKS | 338 |
| (CD123 | SHSVLYSSNNKNYLAWYQQKPGQPPKLLIYWASTRESGVPDR | |
| binding | FSGSGSGTDFTLTISSLQAEDVAVYYCQQYYSTPPTTFGGGTKV | |
| domain in | EIKGGGGSGGGGSGGGGSGGGGSEVQLLESGGGLVQPGGSL | |
| bold, CD3 | RLSCAASGFTFSSYGMSWVRQAPGKGLEGVSAISGSGGSTYY | |
| binding | ADSVKGRFTISRDNSKNTLYLQMNSLRAEDTAVYYCAKEKLRY | |
| domain in | FDWLSDAFDIWGQGTMVTVSSSEPKSSDKTHTCPPCPAPEAAG | |
| italics) | APSVFLFPPKPKDTLMISRTPEVTCVVVDVSHEDPEVKFNWYVD | |
| (CDR | GVEVHNAKTKPREEQYNSTYRVVSVLTVLHQDWLNGKEYKCAV | |
| sequences | SNKALPAPIEKTISKAKGQPREPQVYTLPPSRDELTKNQVSLTCL | |
| are single- | VKGFYPSDIAVEWESNGQPENNYKTTPPVLDSDGSFFLYSKLTV | |
| underlined) | DKSRWQQGNVFSCSVMHEALHNHYTQKSLSLSPGSGGGGSGG | |
| | GGSGGGGSPSEVQLVESGGGLVQPGGSLRLSCAASGFTFSTYA | |
| | MNWVRQAPGKGLEWVGRIRSKYNNYATYYADSVKDRFTISRDD | |
| | SKNSLYLQMNSLKTEDTAVYYCVRHGNFGNSYVSWFAYWGQG | |
| | TLVTVSSGGGGSGGGGSGGGGSGGGGSQAVVTQEPSLTVSPG | |
| | GTVTLTCRSSTGAVTTSNYANWVQQKPGQAPRGLIGGTNKRAP | |
| | WTPARFSGSLLGGKAALTITGAQAEDEADYYCALWYSNLWVFG | |
| | GGTKLTVLRS | |
| TRI168-dual- | MEAPAQLLFLLLLWLPDTTGQAVVTQEPSLTVSPGGTVTLTCRS | 339, |
| affinity | STGAVTTSNYANWVQQKPGQAPRGLIGGTNKRAPWTPARFSG | 340 |
| re-targeting | SLLGGKAALTITGAQAEDEADYYCALWYSNLWVFGGGTKLTVLG | |
| molecule | GGGSGGGGEVQLVQSGAELKKPGASVKVSCKASGYTFTDYY | |
| contains 2 | MKWVRQAPGQGLEWIGDIIPSNGATFYNQKFKGRVTITVDKSTS | |
| chains | TAYMELSSLRSEDTAVYYCARSHLLRASWFAYWGQGTLVTVS | |
| (CD123 | SGGCGGGEVAALEKEVAALEKEVAALEKEVAALEK | |
| binding | DFVMTQSPDSLAVSLGERVTMSCKSSQSLLNSGNQKNYLTWY | |
| domain in | QQKPGQPPKLLIYWASTRESGVPDRFSGSGSGTDFTLTISSLQ | |
| bold, CD3 | AEDVAVYYCQNDYSYPYTFGQGTKLEIKGGGSGGGGEVQLVE | |
| binding | SGGGLVQPGGSLRLSCAASGFTFSTYAMNWVRQAPGKGLEWV | |
| domain in | GRIRSKYNNYATYYADSVKDRFTISRDDSKNSLYLQMNSLKTED | |
| italics) | TAVYYCVRHGNFGNSYVSWFAYWGQGTLVTVSSGGCGGGKVA | |
| (CDR | ALKEKVAALKEKVAALKEKVAALKESSSLNDIFEAQKIEWHEDYK | |
| sequences | DDDDKDYKDDDDKDYKDDDDKHHHHHHHHHH | |
| are single- | | |
| underlined) | | |

In certain embodiments, the CD123-binding domain comprises (i) an immunoglobulin light chain variable region (VL) comprising CDRs LCDR1, LCDR2, and LCDR3, and (ii) an immunoglobulin heavy chain variable region (VH) comprising CDRs HCDR1, HCDR2, and HCDR3 with HCDR1 comprising an amino acid sequence as set forth in SEQ ID NO:144, with HCDR2 comprising an amino acid sequence as set forth in SEQ ID NO:146 and with HCDR3 comprising an amino acid sequence as set forth in SEQ ID NO:148. In certain embodiments, the CD123-binding domain comprises (i) an immunoglobulin light chain variable region (VL) comprising CDRs LCDR1, LCDR2, and LCDR3, and (ii) an immunoglobulin heavy chain variable region (VH) comprising CDRs HCDR1, HCDR2, and HCDR3. In some such embodiments, (i) the LCDR1 has an amino acid sequence set forth in SEQ ID NO:138 or a sequence that differs from SEQ ID NO:138 by at least one amino acid substitution; (ii) the LCDR2 has an amino acid sequence set forth in SEQ ID NO:140 or a sequence that differs from SEQ ID NO:140 by at least one amino acid substitution; (iii) the LCDR3 has an amino acid sequence set forth in SEQ ID NO:142 or a sequence that differs from SEQ ID NO:142 by at least one amino acid substitution; (iv) the HCDR1 has an amino acid sequence set forth in SEQ ID NO:144 or a sequence that differs from SEQ ID NO:144 by at least one amino acid substitution; (v) the HCDR2 has an amino acid sequence set forth in SEQ ID NO:146 or a sequence that differs from SEQ ID NO:146 by at least one amino acid substitution; and (vi) the HCDR3 has an amino acid sequence set forth in SEQ ID NO:148 or a sequence that differs from SEQ ID NO:148 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody. For instance, the invention includes a recombinant polypeptide comprising (i) the LCDR1 has an amino acid sequence set forth in SEQ ID NO:138 or a sequence that differs from SEQ ID NO:138 by one or two amino acid substitutions; (ii) the LCDR2 has an amino acid sequence set forth in SEQ ID NO:140 or a sequence that differs from SEQ ID NO:140 by one or two amino acid substitutions; (iii) the LCDR3 has an amino acid sequence set forth in SEQ ID NO:142 or a sequence that differs from SEQ ID NO:142 by one or two amino acid substitutions; (iv) the HCDR1 has an amino acid sequence set forth in SEQ ID NO:144 or a sequence that differs from SEQ ID NO:144 by one or two amino acid substitutions; (v) the HCDR2 has an amino acid sequence set forth in SEQ ID NO:146 or a sequence that differs from SEQ ID NO:146 by one or two amino acid substitutions; and (vi) the HCDR3 has an amino acid sequence set forth in SEQ ID NO:148 or a sequence that differs from SEQ ID NO:148 by one or two amino acid substitutions. The amino acid substitution described above may be a conservative or a non-conservative amino acid substitution.

In related embodiments, a recombinant polypeptide of the invention comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:134) or to a heavy chain variable region (V_{H}) (e.g., SEQ ID NO:136), or both. In some embodiments, the CD123-binding domain of the recombinant polypeptide is an scfv comprising a variable heavy chain comprising SEQ ID NO:136 and a variable light chain comprising SEQ ID NO:134 in the VHVL orientation. In some embodiments, the CD123-binding domain of the recombinant polypeptide is an scFv comprising a variable light chain comprising SEQ ID NO:134 and a variable heavy chain comprising SEQ ID NO:136 in the VLVH orientation. For instance, in some embodiments, the polypeptide of the invention comprises an amino acid sequence of SEQ ID NO:337. The invention includes a recombinant polypeptide that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of SEQ ID NO:337.

In some embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:154 or a sequence that differs from SEQ ID NO:154 by at least one amino acid substitution; (ii) the LCDR2 has an amino acid sequence set forth in SEQ ID NO:156 or a sequence that differs from SEQ ID NO:156 by at least one amino acid substitution; (iii) the LCDR3 has an amino acid sequence set forth in SEQ ID NO:158 or a sequence that differs from SEQ ID NO:158 by at least one amino acid substitution; (iv) the HCDR1 has an amino acid sequence set forth in SEQ ID NO:160 or a sequence that differs from SEQ ID NO:160 by at least one amino acid substitution; (v) the HCDR2 has an amino acid sequence set forth in SEQ ID NO:162 or a sequence that differs from SEQ ID NO:162 by at least one amino acid substitution; and (vi) the HCDR3 has an amino acid sequence set forth in SEQ ID NO:164 or a sequence that differs from SEQ ID NO:164 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In some embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In some embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (VL) (e.g., SEQ ID NO:150) or to a heavy chain variable region (VH) (e.g., SEQ ID NO:152), or both.

In some embodiments, a CD123-binding domain comprises humanized immunoglobulin V_{L} and/or V_{H} regions. Techniques for humanizing immunoglobulin V_{L} and V_{H} regions are known in the art and are discussed, for example, in U.S. Patent Application Publication No. 2006/0153837. In some embodiments, a CD123-binding domain comprises human immunoglobulin V_{L} and/or V_{H} regions.

Essentially, humanization by CDR grafting involves recombining only the CDRs of a non-human antibody onto a human variable region framework and a human constant region. Theoretically, this should substantially reduce or eliminate immunogenicity (except if allotypic or idiotypic differences exist). However, it has been reported that some framework residues of the original antibody also may need to be preserved (Reichmann et al., Nature, 332:323 (1988); Queen et al., Proc. Natl. Acad. Sci. USA, 86:10, 029 (1989)).

The framework residues that need to be preserved are amenable to identification through computer modeling. Alternatively, critical framework residues can potentially be identified by comparing known antigen-binding site structures (Padlan, Molec. Immunol., 31(3):169-217 (1994), incorporated herein by reference).

The residues that potentially affect antigen binding fall into several groups. The first group comprises residues that are contiguous with the antigen site surface, which could therefore make direct contact with antigens. These residues include the amino-terminal residues and those adjacent to the CDRs. The second group includes residues that could alter the structure or relative alignment of the CDRs, either by contacting the CDRs or another peptide chain in the antibody. The third group comprises amino acids with buried side chains that could influence the structural integrity of the variable domains. The residues in these groups are usually found in the same positions (Padlan, 1994, supra) although their positions as identified may differ depending on the numbering system (see Kabat et al., “Sequences of proteins of immunological interest, 5th ed., Pub. No. 91-3242, U.S. Dept. Health & Human Services, NIH, Bethesda, Md., 1991).

Knowledge about humanized antibodies in the art is applicable to the polypeptides according to the disclosure, even if these polypeptides are not antibodies.

In some embodiments, the disclosure relates to CD123-binding domains wherein (i) the immunoglobulin light chain variable region comprises an amino acid sequence that is at least 88%, at least 90%, at least 92%, at least 95%, at least 97%, at least 98% or at least 99% identical to the amino acid sequence set forth in SEQ ID NO:134 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:136; (ii) the immunoglobulin light chain variable region comprises an amino acid sequence that is at least 88%, at least 90%, at least 92%, at least 95%, at least 97%, at least 98% or at least 99% identical to the amino acid sequence set forth in SEQ ID NO:150 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:152; (iii) the immunoglobulin light chain variable region comprises an amino acid sequence that is at least 88%, at least 90%, at least 92%, at least 95%, at least 97%, at least 98% or at least 99% identical to the amino acid sequence set forth in SEQ ID NO:150 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:168; (iv) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:150 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:184; (v) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:198 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:200; (vi) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:214 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:216; (vii) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:230 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:232; (viii) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:166 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:296; (ix) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:166 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:248; (x) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:166 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:264; or (xi) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:166 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:280.

In some embodiments, each CDR comprises no more than one, two, or three substitutions, insertions or deletions, as compared to that from a monoclonal antibody or fragment or derivative thereof that specifically binds to a target of interest (e.g., CD123).

In some embodiments, a CD123-binding domain does not inhibit IL-3 binding to CD123.

In some embodiments, a CD123-binding molecule or protein can comprise a T-cell binding domain for recruitment of T-cells to target cells expressing CD123. In some embodiments, a CD123-binding protein as described herein can comprise (i) a binding domain that specifically binds a TCR complex or a component thereof (e.g., TCRα, TCRβ, CD3γ, CD3δ, and CD3ε) and (ii) another binding domain that specifically binds to CD123. A CD123-binding protein can utilize essentially any binding domain that binds a T-cell, e.g., an antibody derived binding domain. Exemplary anti-CD3 antibodies from which the CD3 binding domain can be derived include the CRIS-7 monoclonal antibody (Reinherz, E. L. et al. (eds.), Leukocyte typing II., Springer Verlag, New York, (1986); VL and VH amino acid sequences respectively shown in SEQ ID NO:341 (QWLTQSPAIMSAFPGEKVTMTCSASSSVSYMNWYQQKSGTSPKRWIYDSSKLA SGVPARFSGSGSGTSYSLTISSMETEDAATYYCQQWSRNPPTFGGGTKLQITR) and SEQ ID NO:342 (QVQLQQSGAELARPGASVKMSCKASGYTFTRSTMHWVKQRPGQGLEWIGYINP SSAYTNYNQKFKDKATLTADKSSSTAYMQLSSLTSEDSAVYYCASPQVHYDYNGF PYWGQGTLVTVSA)); HuM291 (Chau et al. (2001) Transplantation 71:941-950; V_{L} and V_{H} amino acid sequences respectively shown in SEQ ID NO:343 (diqmtqspsslsasvgdrvtitcsasssysymnwyqqkpgkapkrliydtsklasgvpsrfsgsgsgtdftltisslqp edfatyycqqwssnpptfgggtkveik) and SEQ ID NO:344 (qvqlvqsgaevkkpgasvkvsckasgytfisytmhwvrqapgqglewmgyinprsgythynqklkdkatltadks astaymelsslrsedtavyycarsayydydgfaywgqgtivtvss)); BC3 monoclonal antibody (Anasetti et al. (1990) J. Exp. Med. 172:1691); OKT3 monoclonal antibody (Ortho multicenter Transplant Study Group (1985) N. Engl. J. Med. 313:337) and derivatives thereof such as OKT3 ala-ala (also referred to as OKT3 AA-FL or OKT3 FL), a humanized, Fc variant with alanine substitutions at positions 234 and 235 (Herold et al. (2003) J. Clin. Invest. 11:409); visilizumab (Carpenter et al. (2002) Blood 99:2712), G19-4 monoclonal antibody (Ledbetter et al., 1986, J. Immunol. 136:3945), 145-2C11 monoclonal antibody (Hirsch et al. (1988) J. Immunol. 140: 3766) and I2C monoclonal antibody (see, e.g., US 2011/0293619 and US20120244162). For example, a CD3 binding domain may comprise a CD3 binding domain disclosed in U.S. Patent Application Publication No. 2012/0244162, including a CD3 binding domain comprising a VL region selected from SEQ ID NO: 17, 21, 35, 39, 53, 57, 71, 75, 89, 83, 107, 111, 125, 129, 143, 147, 161, 165, 179 and 183 of US 2012/0244162 and/or a VH region selected from SEQ ID NO:15, 19, 33, 37, 51, 55, 69, 73, 87, 91. 105, 109, 123, 127, 141, 145, 159, 163, 177 and 181 of US 2012/0244162. In some embodiments, a CD3 binding domain comprises an amino acid sequence selected from SEQ ID NO: 23, 25, 41, 43, 59, 61, 77, 79, 95, 97, 113, 115, 131, 133, 149, 151, 167, 169, 185, and 187 of US 2012/0244162. In some embodiments, a CD3 binding domain is one described in WO2004/106380, WO2005/040220A1, US 2014/0099318 or derived from a CD3 binding domain thereof. An exemplary anti-TCR antibody is the BMA031 monoclonal antibody (Borst et al. (1990) Human Immunology 29:175-188). The CD3 binding domain may be derived from any of the antibodies or sequences described in WO 2013/158856 (incorporated herein by reference in its entirety).

In some embodiments, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs:348, 349 and 350, respectively, and the HCDR1, HCDR2, and HCDR3 has the amino acid sequences set forth in SEQ ID NOs: 345, 346 and 347, respectively; or (b) the LCDR1, LCDR2 and LCDR3 has the amino acid sequences set forth in SEQ ID NO:354, SEQ ID NO:355, and SEQ ID NO:356, respectively, and the HCDR1, HCDR2, and HCDR3 has the amino acid sequences set forth in SEQ ID NO: 351, SEQ ID NO:352, and SEQ ID NO:353, respectively. In some embodiments, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs: 351, 352 and 353, respectively, and the HCDR1, HCDR2, and HCDR3 has the amino acid sequences set forth in SEQ ID NOs: 357, 359 and 359, respectively; or (b) the LCDR1, LCDR2 and LCDR3 has the amino acid sequences set forth in SEQ ID NOs: 359, 367 and 368, respectively, and the HCDR1, HCDR2, and HCDR3 has the amino acid sequences set forth in SEQ ID NOs: 363, 364 and 365, respectively. In some embodiments, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs: 372, 373 and 374, respectively, and the HCDR1, HCDR2, and HCDR3 has the amino acid sequences set forth in SEQ ID NOs: 369, 370 and 371, respectively; or (b) the LCDR1, LCDR2 and LCDR3 has the amino acid sequences set forth in SEQ ID NOs: 378, 379 and 380, respectively, and the HCDR1, HCDR2, and HCDR3 has the amino acid sequences set forth in SEQ ID NOs: 375, 376 and 377, respectively. In some embodiments, the second binding domains comprising the CDR sequences recited in this paragraph are humanized.

In some embodiments of a CD123-binding protein comprising a second binding domain that specifically binds CD3c, the second binding domain competes for binding to CD3c with the CRIS-7, HuM291 or I2C monoclonal antibody. In some embodiments, the CD3-binding domain comprises an immunoglobulin light chain variable region (V_{L}) and an immunoglobulin heavy chain variable region (V_{H}) derived from the CRIS-7, HuM291 or I2C monoclonal antibody (e.g., the V_{L} and V_{H} of the second binding domain can be humanized variable regions comprising, respectively, the light chain CDRs and the heavy chain CDRs of the monoclonal antibody). A second binding domain may comprise the light chain variable region, the heavy chain variable region, or both, of the DRA222, TSC455, or TSC456 CD3-binding domains. The amino acid sequences of DRA222, TSC455, and TSC456 are provided in Table 4. The DRA222 binding domains are also described in WO 2013/158856. TSC455 may also be referred to as TSC394 F87Y. TSC455 may also be referred to as TSC394 E86D F87Y or TSC394 DY. In some embodiments, the second binding domain specifically binds CD3 and comprises an immunoglobulin light chain variable region and an immunoglobulin heavy chain variable region; wherein the immunoglobulin light chain variable region comprises an amino acid sequence that is at least about 93% identical, at least about 95% identical, at least about 97% identical, at least about 98% identical or at least about 99% identical to the amino acid sequence in SEQ ID NO:384; or at least about 94% identical, at least about 95% identical, at least about 97% identical, at least about 98% identical or at least about 99% identical to the amino acid sequence in SEQ ID NO:385; and wherein the immunoglobulin heavy chain variable region comprises an amino acid sequence that is at least about 82% identical, at least about 85% identical, at least about 87% identical, at least about 90% identical, at least about 92% identical, at least about 95% identical, at least about 97% identical, at least about 98% identical or at least about 99% identical to the amino acid sequence in SEQ ID NO:383. In some embodiments, a CD123-binding polypeptide or protein further comprising a CD3-binding domain may have a low level of high molecular weight aggregates produced during recombinant expression of the polypeptide or protein. A CD123-binding polypeptide or protein further comprising a CD3-binding domain may exhibit a relatively long stability in human serum, depending on the CD3-binding domain present in the polypeptide or protein.

In certain variations, the CD3-binding domain and comprises one or more of the CD3-binding sequences (e.g., CDRs or variable regions) disclosed in US 2013/0129730, US 2011/0293619, U.S. Pat. No. 7,635,472, WO 2010/037836, WO 2004/106381, or WO 2011/121110; each incorporated herein by reference in its entirety. In some embodiments, a CD3-binding domain comprises one or more of the sequences shown in Table 7.

TABLE 7
Exemplary CD3-binding domain light chain CDRs

| LCDR1 | LCDR2 | LCDR3 |
|---|---|---|
| GSSTGAVTSGYYPN | GTKFLAP | ALWYSNRWV |
| (SEQ ID NO: 110) | (SEQ ID NO: 113) | (SEQ ID NO: 116) |
| RSSTGAVTSGYYPN | ATDMRPS | ALWYSNRWV |
| (SEQ ID NO: 111) | (SEQ ID NO: 114) | (SEQ ID NO: 117) |
| GSSTGAVTSGNYPN | GTKFLAP | VLWYSNRWV |
| (SEQ ID NO: 112) | (SEQ ID NO: 115) | (SEQ ID NO: 118) |

In various embodiments, a CD3-binding domain comprises one or more of the sequences shown in Table 8.

TABLE 8
Exemplary CD3-binding domain heavy chain CDRs

| HCDR1 | HCDR2 | HCDR3 |
|---|---|---|
| IYAMN | RIRSKYNNYATYYADS | HGNFGNSYVSFFAY |
| (SEQ ID NO: 119) | VKS | (SEQ ID NO: 125) |
| | (SEQ ID NO: 122) | |
| KYAMN | RIRSKYNNYATYYADS | HGNFGNSYISYWAY |
| (SEQ ID NQ: 120) | VKD | (SEQ ID NO: 126) |
| | (SEQ ID NO: 123) | |
| SYAMN | RIRSKYNNYATYYADS | HGNFGNSYLSFWAY |
| (SEQ ID NO: 121) | VKG | (SEQ ID NO: 127) |
| | (SEQ ID NO: 124) | |

In some embodiments, a therapeutic protein comprises, in order from amino terminus to carboxyl terminus a first binding domain, a hinge region, an immunoglobulin constant region, and a second binding domain. In some embodiments, the immunoglobulin constant region comprises immunoglobulin CH2 and CH3 domains of IgG1, IgG2, IgG3, IgG4, IgA1, IgA2 or IgD. In some embodiments, the first binding domain comprises: an immunoglobulin heavy chain variable region (VH) comprising HCDR1, HCDR2, and HCDR3; and an immunoglobulin light chain variable region (VL) comprising LCDR1, LCDR2, and LCDR3. In some embodiments, the HCDR1 comprises SEQ ID NO: 10, the HCDR2 comprises SEQ ID NO: 11, and the HDCR3 comprises SEQ ID NO: 12. In some embodiments, the LCDR1 comprises SEQ ID NO: 13, the LCDR2 comprises SEQ ID NO: 14, and the LCDR3 comprises SEQ ID NO: 15. In some embodiments, the HCDR1 comprises SEQ ID NO: 10, the HCDR2 comprises SEQ ID NO: 11, and the HDCR3 comprises SEQ ID NO: 12; and the LCDR1 comprises SEQ ID NO: 13, the LCDR2 comprises SEQ ID NO: 14, and the LCDR3 comprises SEQ ID NO: 15. In some embodiments, the VH comprises a sequence of SEQ ID NO: 16, or a sequence at least 90% or at least 95% identical thereto. In some embodiments, the VL comprises a sequence of SEQ ID NO: 17, or a sequence at least 90% or at least 95% identical thereto. In some embodiments, the first binding domain comprises a sequence at least 95% identical to SEQ ID NO: 18. In some embodiments, the second binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3. In some embodiments, the HCDR1 comprises SEQ ID NO: 19, the HCDR2 comprises SEQ ID NO: 20, and the HDCR3 comprises SEQ ID NO: 21. In some embodiments, the LCDR1 comprises SEQ ID NO: 22, the LCDR2 comprises SEQ ID NO: 23, and the LCDR3 comprises SEQ ID NO: 24. In some embodiments, the HCDR1 comprises SEQ ID NO: 19, the HCDR2 comprises SEQ ID NO: 20, and the HDCR3 comprises SEQ ID NO: 21; and the LCDR1 comprises SEQ ID NO: 22, the LCDR2 comprises SEQ ID NO: 23, and the LCDR3 comprises SEQ ID NO: 24. In some embodiments, the VH comprises a sequence of SEQ ID NO: 25, or a sequence at least 90% or at least 95% identical thereto. In some embodiments, the VL comprises a sequence of SEQ ID NO: 26, or a sequence at least 90% or at least 95% identical thereto. In some embodiments, the second binding domain comprises a sequence at least 95% or 100% identical to SEQ ID NO: 27. In some embodiments, the therapeutic protein comprises the sequence of SEQ ID NO: 31.

### Drug Delivery Systems

The compositions for preventing protein adsorption described herein may be used with many different types of drug delivery systems known to those of skill in the art.

Drug delivery systems according to the present disclosure may include one or more components configured to hold a liquid, for example an IV bag. In some embodiments, the therapeutic protein is suspended in the liquid inside the IV bag. The component configured to hold a liquid may have a volume of about 50, about 100, about 150, about 200, about 250, about 350, about 450, or about 500 ml. The component may be made from, for example, polyvinyl chloride (PVC), ethylene vinyl acetate, polypropylene, or copolyester ether.

The drug delivery systems may additionally comprise one or more tubes. The tubes may be attached to the component configured to hold a liquid.

The drug delivery systems of the instant disclosure may additionally comprise a needle for insertion into the patient.

In some embodiments, a drug delivery system for delivering a therapeutic protein to a patient comprises at least one component adapted for delivery of the therapeutic protein, wherein the component is selected from the group consisting of a container configured to hold a liquid, a tube, and a needle; wherein an interior surface of the at least one component is contacted with a composition comprising about 1 to about 10 mM succinate, and about 0.001% to 0.01% (w/v) polysorbate 80 before it is contacted with the therapeutic protein.

In some embodiments, a drug delivery system for delivering a therapeutic protein to a patient comprises at least one container adapted to hold the therapeutic protein, wherein an interior surface of the at least one container is contacted with a composition comprising about 1 to about 10 mM succinate, and about 0.001% to 0.01% (w/v) polysorbate 80 before it is contacted with a composition comprising the therapeutic protein.

A container adapted for holding a therapeutic protein is also provided. In some embodiments, an interior surface of the container is first contacted with a composition of the disclosure before it is contacted with a composition comprising the therapeutic protein. In some embodiments, the container is substantially free of latex. In some embodiments, the container is substantially free of bis(2-ethylhexyl) phthalate (DEHP). In some embodiments, the container is selected from the group consisting of an IV bag, a syringe, and a tube.

In some embodiments, a method of preparing an intravenous drug delivery system for delivery of a therapeutic protein comprises providing at least one container adapted to hold the therapeutic protein, and before the therapeutic protein is added to the at least one container, contacting an interior surface of the at least one container with a composition comprising about 1 to about 10 mM succinate, and about 0.001% to 0.01% (w/v) polysorbate 80. In some embodiments, the composition coats the interior surface of the at least one container and prevents the therapeutic protein from binding to the interior surface of the container.

Methods of Treating

The disclosure also provides a method of treating a subject by intravenous administration (e.g., intravenous infusion) of a therapeutic protein. The subject may be, for example, a mammal. In some embodiments, the subject is a human, a rabbit, a dog, a cat, a guinea pig, a hamster, a rat, a mouse, a horse, or a cow. In some embodiments, the subject is a human.

In some embodiments, the method comprises providing at least one container adapted to hold the therapeutic protein, contacting an interior surface of the container with a composition comprising about 1 to about 10 mM succinate and about 0.001% to about 0.01% (w/v) polysorbate 80, contacting the interior surface of the container with a composition comprising the therapeutic protein, and intravenously administering the therapeutic protein to the patient. In some embodiments, the composition coats an interior surface of the at least one container and prevents the therapeutic protein from binding to the interior surface of the container.

### EXAMPLES

The invention is further described in detail by reference to the following examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Preparation of IVSS Solution

On day T=1, a 20×IVSS composition comprising 10 mM succinate and 0.08% polysorbate-80 at pH 6.0 was prepared. The composition was placed into a 10 mL clear glass vial with a nitrogen overlay, and sealed using a 20 mm stopper/flip off overseal. This composition is referred to throughout the Examples as “succinate formulation.”

A comparator 20×IVSS composition was prepared comprising 333 mM Histidine and 0.067% polysorbate-80 at pH 6.0. The composition was similarly placed into a 10 mL clear glass vial with a nitrogen overlay, and sealed using a 20 mm stopper/flip off overseal. This composition is referred to throughout the Examples as “histidine formulation.”

### Example 2: Stability of Polysorbate in the Succinate and Histidine Formulations

The stability of polysorbate-80 in the succinate formulation and in the histidine formulation was determined by quantification of polysorbate-80 by HPLC and/or by qualitative assessment of the UV spectral scan profile. For HPLC, polysorbate concentration of both formulations was determined on an Agilent® HPLC equipped with an ELSD detector. For UV spectral scan, the absorbance from 100 to 600 nm of both formulations was scanned using a spectrophotometer.

Stability data is shown in Table 9. Polysorbate-80 in the succinate formulation is stable at 40° C. for at least 270 days, whereas the polysorbate-80 in the histidine formulation is stable for less than two months at 25° C. This result demonstrates that the polysorbate-80 is more stable in the succinate formulation compared to the histidine formulation.

TABLE 9
Percent Polysorbate-80 (PS80) in Histidine and Succinate Buffers

| | |
|---|---|
| % PS80 at T = 2-months | % PS80 at T = 270 |
| at 25° C. | Days at 40° C. |

Histidine Formulation

| | | |
|---|---|---|
| PS80 Specification: | <0.04%* | Not Tested |
| 0.04%-0.08% | | |

Succinate Formulation

| | | |
|---|---|---|
| PS80 Specification: | Not Tested | 0.074** |
| 0.06%-0.1% | | |

*Assay conducted with a plate-based fluorescence assay
**Assay conducted using a HPLC with ELSD detector

Table 10 shows polysorbate 80 quantification in the succinate formulation by the HPLC method. The sample was held at 40° C. for 270 days. Polysorbate-80 in the succinate buffer was still within specification after 270 days, with polysorbate-80 quantified at 0.07%. (The polysorbate 80 specification is set at 0.06% to 0.1% polysorbate-80)

TABLE 10
Percent polysorbate-80 in succinate formulation
held at 40° C., quantified by the HPLC method.

| | % Polysorbate 80 | | | | |
|---|---|---|---|---|---|
| Temperature (° C.) | T = 13-Day | T = 77-Day | T = 144-Day | T = 214-Day | T = 270-Day |
| 40 | 0.08 | 0 08 | 0.08 | 0.07 | 0.07 |

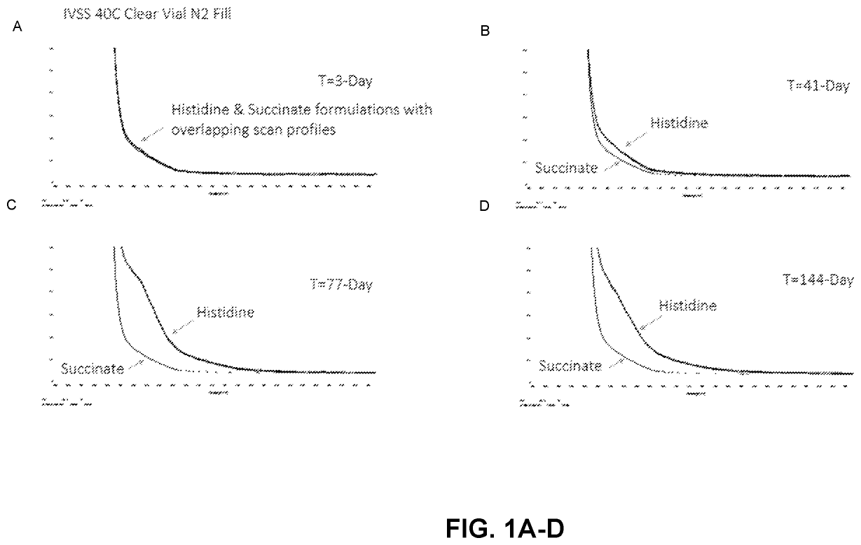

Stability was also assessed using a UV scan method. The UV scan data corroborated the data obtained by the HPLC method (FIG. 1A-D). At T=41 days (FIG. 1B), the histidine formulation showed a change in the UV spectral scan profile, indicative of the breakdown of polysorbate-80. At T=77 (FIG. 1C) and T=144 days (FIG. 1D), the histidine formulation showed further polysorbate-80 degradation as compared to T=41 days. There was no change in the spectral scan profile for the succinate formulation for the duration of this experiment.

Ongoing stability of the succinate formulation was also observed for 6-months in samples held at 2-8° C. and at 25° C. The succinate formulation was stable at all time points and temperatures tested. Table 11(a), below, shows appearance data, pH, osmolality, polysorbate 80 concentration, spectral scan, and Micro Flow Imaging (MFI) for initial, 1-month, 2-month, 3-month, and 6-month time points. Values are rounded to the nearest whole number (e.g., 1.2 is rounded to 1.0).

TABLE 11(a)
Stability data for succinate formulation

| | | | Time Point | | | | |
|---|---|---|---|---|---|---|---|
| Test | Specification | Condition | Initial | 1 Months | 2 Months | 3 Months | 6 Months |
| Appearance | Colorless | 2-8° C. | Clear, | CCL, NVP | CCL, NVP | CCL, NVP | CCL, NVP |
| | to slightly | Upright | colorless | | | | |
| | yellow | 2-8° C. | liquid | CCL, NVP | CCL, NVP | CCL, NVP | CCL, NVP |
| | | Inverted | w/no | | | | |
| | | 25° C. | visible | CCL, NVP | CCL, NVP | CCL, NVP | CCL, NVP |
| | | Upright | particles | | | | |
| | | 25° C. | (CCL, NVP) | CCL, NVP | CCL, NVP | CCL, NVP | CCL, NVP |
| | | Inverted | | | | | |
| pH | 5.7 to 6.3 | 2-8° C. | 6.0 | 6.0 | 6.0 | 6.0 | 6.0 |
| | | Upright | | | | | |
| | | 2-8° C. | | 6.0 | 6.0 | 6.1 | 6.0 |
| | | Inverted | | | | | |
| | | 25° C. | | 6.0 | 6.0 | 6.0 | 6.0 |
| | | Upright | | | | | |
| | | 25° C. | | 6.0 | 6.0 | 6.0 | 6.0 |
| | | Inverted | | | | | |
| Osmolality | 200 to 300 | 2-8° C. | 247 | 249 | 246 | 244 | 244 |
| | mOsm/kg | Upright | | | | | |
| | | 2-8° C. | | 248 | 246 | 247 | 245 |
| | | Inverted | | | | | |
| | | 25° C. | | 249 | 248 | 246 | 244 |
| | | Upright | | | | | |
| | | 25° C. | | 268 | 248 | 246 | 245 |
| | | Inverted | | | | | |
| PS80 | 0.06% to | 2-8° C. | 0.08% | Not | Not | Not | 0.08% |
| Concentration | 0.1% (w/v) | Upright | | Tested | Tested | Tested | |
| | | 2-8° C. | | Not | Not | Not | 0.08% |
| | | Inverted | | Tested | Tested | Tested | |
| | | 25° C. | | Not | Not | Not | 0.08% |
| | | Upright | | Tested | Tested | Tested | |
| | | 25° C. | | Not | Not | Not | 0.06% |
| | | Inverted | | Tested | Tested | Tested | |
| Spectral | Comparable | 2-8° C. | Other time | Spectral is | Spectral is | Spectral is | Spectral is |
| Scan | to Initial | Upright | points | comparable | comparable | comparable | comparable |
| | Scan | 2-8° C. | compared | to T = 0 | to T = 0 | to T = 0 | to T = 0 |
| | | Inverted | to | with no | with no | with no | with no |
| | | 25° C. | Initial | shift in | shift in | shift in | shift in |
| | | Upright | Scan | absorbance | absorbance | absorbance | absorbance |
| | | 25° C. | | between | between | between | between |
| | | Inverted | | 300 nm to | 300 nm to | 300 nm to | 300 nm to |
| | | | | 450 nm | 450 nm | 450 nm | 450 nm |
| MFI | Report | 2-8° C. | ≥2 μm = 28 | ≥2 μm = 102 | ≥2 μm = 503 | ≥2 μm = 67 | ≥2 μm = 124 |
| | number of | Upright | ≥5 μm = 7 | ≥5 μm = 24 | ≥5 μm = 83 | ≥5 μm = 19 | ≥5 μm = 38 |
| | particles/mL | | ≥10 μm = 4 | ≥10 μm = 11 | ≥10 μm = 18 | ≥10 μm = 8 | ≥10 μm = 15 |
| | of each | | ≥25 μm = 1 | ≥25 μm = 3 | ≥25 μm = 3 | ≥25 μm = 0 | ≥25 μm = 1 |
| | size | 2-8° C. | | ≥2 μm = 33 | ≥2 μm = 266 | ≥2 μm = 40 | ≥2 μm = 115 |
| | | Inverted | | ≥5 μm = 18 | ≥5 μm = 39 | ≥5 μm = 13 | ≥5 μm = 43 |
| | | | | ≥10 μm = 6 | ≥10 μm = 15 | ≥10 μm = 7 | ≥10 μm = 17 |
| | | | | ≥25 μm = 0 | ≥25 μm = 7 | ≥25 μm = 0 | ≥25 μm = 0 |
| | | 25° C. | | ≥2 μm = 43 | ≥2 μm = 542 | ≥2 μm = 38 | ≥2 μm = 298 |
| | | Upright | | ≥5 μm = 24 | ≥5 μm = 363 | ≥5 μm = 11 | ≥5 μm = 96 |
| | | | | ≥10 μm =4 | ≥10 μm = 35 | ≥10 μm = 3 | ≥10 μm = 24 |
| | | | | ≥25 μm = 1 | ≥25 μm = 6 | ≥25 μm = 0 | ≥25 μm = 0 |
| | | 25° C. | | ≥2 μm = 67 | ≥2 μm = 231 | ≥2 μm = 38 | ≥2 μm = 147 |
| | | Inverted | | ≥5 μm = 32 | ≥5 μm = 38 | ≥5 μm = 10 | ≥5 μm = 64 |
| | | | | ≥10 μm = 8 | ≥10 μm = 14 | ≥10 μm = 6 | ≥10 μm = 25 |
| | | | | ≥25 μm = 1 | ≥25 μm = 3 | ≥25 μm = 1 | ≥25 μm = 6 |

CCL = clear, colorless liquid;
NVP = non-visible particles

Table 11(b), below, shows appearance data, pH, osmolality, spectral scan information, polysorbate 80 concentration, spectral scan, and Micro Flow Imaging (MFI) data for initial (TO), 1-month (T1), 2-month (T2), 3-month (T3), 6-month (T6), 9-month (T9), and 12-month (T12) time points, at various temperatures (2-8° C., 25° C.) and conditions (inverted, upright). In Table 11(b), the data shows the number of particles per milliliter observed, wherein the particles had a diameter of ≥2 μm, ≥5 μm, ≥10 μm, or ≥25 μm. Values are rounded up to the next whole number (e.g., 1.2 is rounded to 2).

TABLE 11(b)
Additional Stability Data

| | | | | | | | MFI (particles/ml) | | | |
|---|---|---|---|---|---|---|---|---|---|---|
| Test | | | | Osmolal- | | | Report the number of particles | | | |
| Temp | Time | | | ity | | Polysor- | ≥2 | ≥5 | ≥10 | ≥25 |
| (° C.) | points | Appearance | pH | (mOsm/kg) | Spectral Scan (AU) | bate 80 | μm | μm | μm | μm |
| NA | T0 | Clear, Colorless, Liquid, | 6.0 | 247 | T = 0 | 0.08% | 28 | 7 | 5 | 2 |
| | | No visible particles | | | | | | | | |
| 25° C. | 1 | Clear, Colorless, Liquid, | 6.0 | 268 | Spectral is comparable to T = 0 | NT | 67 | 33 | 9 | 2 |
| Inverted | | No visible particles | | | with no shift in Absorbance between | | | | | |
| | | | | | 300 nm to 450 nm | | | | | |
| | 2 | Clear, Colorless, Liquid, | 6.0 | 248 | Spectral is comparable to T = 0 | NT | 231 | 38 | 14 | 3 |
| | | No visible particles | | | with no shift in Absorbance between | | | | | |
| | | | | | 300 nm to 450 nm | | | | | |
| | 3 | Clear, Colorless, Liquid, | 6.0 | 246 | Spectral is comparable to T = 0 | NT | 38 | 10 | 6 | 2 |
| | | No visible particles | | | with no shift in Absorbance between | | | | | |
| | | | | | 300 nm to 450 nm | | | | | |
| | 6 | Clear, Colorless, Liquid, | 6.0 | 245 | Spectral is comparable to T = 0 | 0.06% | 148 | 64 | 26 | 6 |
| | | No visible particles | | | with a slight shift in Absorbance between | | | | | |
| | | | | | 300 nm to 450 nm | | | | | |
| | 9 | Clear, Colorless, Liquid, | 6.0 | 244 | Spectral has a slight shift in Absorbance | 0.04% | 120 | 31 | 16 | 9 |
| | | No visible particles | | | between 300 nm to 450 nm | | | | | |
| | 12 | Clear, Colorless, Liquid, | 6.0 | 248 | Spectral has a slight shift in Absorbance | 0.04% | 109 | 23 | 12 | 0 |
| | | No visible particles | | | between 300 nm to 450 nm | | | | | |
| NA | T0 | Clear, Colorless, Liquid, | 6.0 | 247 | T = 0 | 0.08% | 28 | 7 | 5 | 2 |
| | | No visible particles | | | | | | | | |
| 25° C. | 1 | Clear, Colorless, Liquid, | 6.0 | 249 | Spectral is comparable to T = 0 | NT | 44 | 24 | 5 | 2 |
| Upright | | No visible particles | | | with no shift in Absorbance between | | | | | |
| | | | | | 300 nm to 450 nm | | | | | |
| | 2 | Clear, Colorless, Liquid, | 6.0 | 248 | Spectral is comparable to T = 0 | NT | 543 | 363 | 35 | 6 |
| | | No visible particles | | | with no shift in Absorbance between | | | | | |
| | | | | | 300 nm to 450 nm | | | | | |
| | 3 | Clear, Colorless, Liquid, | 6.0 | 246 | Spectral is comparable to T = 0 | NT | 38 | 12 | 3 | 0 |
| | | No visible particles | | | with no shift in Absorbance between | | | | | |
| | | | | | 300 nm to 450 nm | | | | | |
| | 6 | Clear, Colorless, Liquid, | 6.0 | 244 | Spectral is comparable to T = 0 | 0.08% | 298 | 96 | 24 | 0 |
| | | No visible particles | | | with no shift in Absorbance between | | | | | |
| | | | | | 300 nm to 450 nm | | | | | |
| | 9 | Clear, Colorless, Liquid, | 6.0 | 244 | Spectral has a slight shift in Absorbance | 0.05% | 325 | 73 | 30 | 9 |
| | | No visible particles | | | between 300 nm to 450 nm | | | | | |
| | 12 | Clear, Colorless, Liquid, | 6.0 | 246 | Spectral is comparable to T = 0 | 0.08% | 140 | 21 | 5 | 2 |
| | | No visible particles | | | with no shift in Absorbance between | | | | | |
| | | | | | 300 nm to 450 nm | | | | | |

| | | | | | | | MFI (particles/ml) | | | |
|---|---|---|---|---|---|---|---|---|---|---|
| | | | | | | | Report the number | | | |
| Test | | | | Osmolal- | | Polysor- | of particles | | | |
| Temp | Time | | | ity | | bate | ≥2 | ≥5 | ≥10 | ≥25 |
| (° C.) | points | Appearance | pH | (mOsm/kg) | Spectral Scan (AU) | 80 (%) | μm | μm | μm | μm |
| NA | T0 | Clear, Colorless, Liquid, | 6.0 | 247 | T = 0 | 0.078 | 28 | 7 | 5 | 2 |
| | | No visible particles | | | | | | | | |
| 2-8° C. | 1 | Clear, Colorless, Liquid, | 6.0 | 248 | Spectral is comparable to T = 0 | NT | 34 | 19 | 6 | 0 |
| Inverted | | No visible particles | | | with no shift in Absorbance between | | | | | |
| | | | | | 300 nm to 450 nm | | | | | |
| | 2 | Clear, Colorless, Liquid, | 6.0 | 246 | Spectral is comparable to T = 0 | NT | 266 | 39 | 16 | 7 |
| | | No visible particles | | | with no shift in Absorbance between | | | | | |
| | | | | | 300 nm to 450 nm | | | | | |
| | 3 | Clear, Colorless, Liquid, | 6.1 | 247 | Spectral is comparable to T = 0 | NT | 41 | 13 | 7 | 0 |
| | | No visible particles | | | with no shift in Absorbance between | | | | | |
| | | | | | 300 nm to 450 nm | | | | | |
| | 6 | Clear, Colorless, Liquid, | 6.0 | 245 | Spectral is comparable to T = 0 | 0.08 | 116 | 44 | 17 | 0 |
| | | No visible particles | | | with no shift in Absorbance between | | | | | |
| | | | | | 300 nm to 450 nm | | | | | |
| | 9 | Clear, Colorless, Liquid, | 6.0 | 243 | Spectral is comparable to T = 0 | 0.08 | 84 | 37 | 16 | 2 |
| | | No visible particles | | | with no shift in Absorbance between | | | | | |
| | | | | | 300 nm to 450 nm | | | | | |
| | 12 | Clear, Colorless, Liquid, | 6.0 | 244 | Spectral is comparable to T = 0 | 0.08 | 56 | 32 | 9 | 2 |
| | | No visible particles | | | with no shift in Absorbance between | | | | | |
| | | | | | 300 nm to 450 nm | | | | | |
| | 18 | Clear, Colorless, Liquid, | 6.0 | 244 | Spectral is comparable to T = 0 | 0.08 | 152 | 57 | 24 | 11 |
| | | No visible particles | | | with no shift in Absorbance between | | | | | |
| | | | | | 300 nm to 450 nm | | | | | |
| | 24 | Clear, Colorless, Liquid, | 6.0 | 244 | Spectral is comparable to T = 0 | 0.08 | 217 | 103 | 46 | 11 |
| | | One visible particle | | | with no shift in Absorbance between | | | | | |
| | | | | | 300 nm to 450 nm | | | | | |
| | 30 | Clear, Colorless, Liquid, | 6.0 | 243 | Spectral is comparable to T = 0 | 0.08 | 476 | 174 | 78 | 11 |
| | | No visible particles | | | with no shift in Absorbance between | | | | | |
| | | | | | 300 nm to 450 nm | | | | | |
| | 36 | Clear, Colorless, Liquid, | 6.0 | 244 | Spectral is comparable to T = 0 | 0.08 | 203 | 78 | 31 | 4 |
| | | No visible particles | | | with no shift in Absorbance between | | | | | |
| | | | | | 300 nm to 450 nm | | | | | |
| NA | T0 | Clear, Colorless, Liquid, | 6.0 | 247 | T = 0 | 0.08 | 28 | 7 | 5 | 2 |
| | | No visible particles | | | | | | | | |
| 2-8° C. | 1 | Clear, Colorless, Liquid, | 6.0 | 249 | Spectral is comparable to T = 0 | NT | 102 | 24 | 12 | 3 |
| Upright | | No visible particles | | | with no shift in Absorbance between | | | | | |
| | | | | | 300 nm to 450 nm | | | | | |
| | 2 | Clear, Colorless, Liquid, | 6.0 | 246 | Spectral is comparable to T = 0 | NT | 504 | 84 | 19 | 3 |
| | | No visible particles | | | with no shift in Absorbance between | | | | | |
| | | | | | 300 nm to 450 nm | | | | | |
| | 3 | Clear, Colorless, Liquid, | 6.0 | 244 | Spectral is comparable to T = 0 | NT | 67 | 20 | 9 | 0 |
| | | No visible particles | | | with no shift in Absorbance between | | | | | |
| | | | | | 300 nm to 450 nm | | | | | |
| | 6 | Clear, Colorless, Liquid, | 6.0 | 244 | Spectral is comparable to T = 0 | 0.08 | 124 | 38 | 16 | 2 |
| | | No visible particles | | | with no shift in Absorbance between | | | | | |
| | | | | | 300 nm to 450 nm | | | | | |
| | 9 | Clear, Colorless, Liquid, | 6.0 | 241 | Spectral is comparable to T = 0 | 0.08 | 162 | 28 | 9 | 2 |
| | | No visible particles | | | with no shift in Absorbance between | | | | | |
| | | | | | 300 nm to 450 nm | | | | | |
| | 12 | Clear, Colorless, Liquid, | 6.0 | 244 | Spectral is comparable to T = 0 | 0.08 | 234 | 24 | 5 | 0 |
| | | No visible particles | | | with no shift in Absorbance between | | | | | |
| | | | | | 300 nm to 450 nm | | | | | |
| | 18 | Clear, Colorless, Liquid, | 6.0 | 243 | Spectral is comparable to T = 0 | 0.08 | 367 | 57 | 15 | 4 |
| | | No visible particles | | | with no shift in Absorbance between | | | | | |
| | | | | | 300 nm to 450 nm | | | | | |
| | 24 | Clear, Colorless, Liquid, | 6.0 | 243 | Spectral is comparable to T = 0 | 0.08 | 317 | 103 | 24 | 3 |
| | | No visible particles | | | with no shift in Absorbance between | | | | | |
| | | | | | 300 nm to 450 nm | | | | | |
| | 30 | Clear, Colorless, Liquid, | 6.1 | 241 | Spectral is comparable to T = 0 | 0.08 | 1551 | 181 | 65 | 8 |
| | | No visible particles | | | with no shift in Absorbance between | | | | | |
| | | | | | 300 nm to 450 nm | | | | | |
| | 36 | Clear, Colorless, Liquid, | 6.0 | 244 | Spectral is comparable to T = 0 | 0.08 | 2532 | 492 | 144 | 38 |
| | | No visible particles | | | with no shift in Absorbance between | | | | | |
| | | | | | 300 nm to 450 nm | | | | | |

Taken together, this data suggests that polysorbate-80 is much more stable in a succinate-based formulation than in a histidine-based formulation.

### Example 3: Method of Using IVSS Solution with TRI130

IVSS is supplied with TRI130, a CD123× CD3 bispecific, in clinical trials.

IVSS is shipped refrigerated in single-use 10 mL vials to clinical trial sites. IVSS is stored in the pharmacy or a designated locked area at 2-8° C. until use.

Before administration of TRI130 to a patient, TRI130 is diluted to prepare a final dose. The TRI130 dilution is prepared in an empty IV bag; this is referred to as the drug dilution bag.

To prepare the drug dilution bag, a vial of TRI130 is swirled gently (not shaken) 5-6 times without inversion to make sure the product is adequately mixed for use in dose preparation. 190 mL of normal saline is added into an empty drug dilution bag. 9.7 mL of IVSS is added into the drug dilution bag, and the bag is gently mixed by inverting 5-6 times. 0.3 mL of TRI130 is added into the drug dilution bag, and the bag is gently mixed by inverting 5-6 times.

To prepare a syringe for administration to the patient, the following procedure is followed. A 50 or 60 mL syringe is labeled with the patient name, study number, drug name, dose, and date and time of preparation. This is referred to as the patient administration syringe. An amount (“A” mL) of Normal Saline is added into this labelled empty 60 mL syringe. See Table 13 for the amount of saline, “A” to be added, as it depends on the dose cohort.

An amount (“B” mL) of IVSS is then added from one vial of IVSS using a syringe and needle. See Table 13 for the amount of IVSS, “B” to be added, as it depends on the dose cohort. The needle is then removed, and that volume (“B” mL) is transferred, into the 60 mL patient administration syringe using a Baxter RAPIDFILL connector (luer lock-to-luer lock). The contents of the syringe containing IVSS are pushed into the 60 mL patient administration syringe. The patient administration syringe is then slightly loosened from the connector, and the plunger is pulled back an additional 1 mL to ensure that all of the IVSS is transferred from the syringe and connector. The syringe is then re-tightened to the connector and the contents of the patient administration syringe are mixed by gently inverting 5 or 6 times. The IVSS syringe and connector are then disconnected and discarded.

An amount (“C” mL) of TRI130 is withdrawn from the drug dilution bag (prepared as described above) using a syringe and needle. See Table 13 for the amount of drug from the drug dilution bag, “C” to be added, as it depends on the dose cohort. The needle is then removed and the TRI130 (volume “C”) is transferred into the patient administration syringe using a Baxter RAPIDFILL connector.

The contents of the syringe containing TRI130 (“C” mL) are pushed into the 60 mL patient administration syringe. The patient administration syringe is slightly loosened from the connector and the plunger is pulled back an additional 1 mL to ensure that all of the TRI130 is transferred from the syringe and connector. The syringe is then retightened to the connector and the contents of the patient administration syringe are mixed by gently inverting 5 or 6 times. The TRI130 syringe and connector are disconnected and discarded.

Subsequently, the IV extension line with filter is attached to the patient administration syringe, and the end cap is removed from the IV line. 1 mL of solution from the patient administration syringe is pushed through the IV extension line and filter to prime the line. The IV extension line and filter will use approximately 0.84 mL, so approximately 0.16 mL will exit the IV tubing and should be discarded appropriately. The end cap on the IV line is replaced. The patient administration syringe and IV line and filter are then sent to the hospital floor or the infusion center for patient administration.

Further details regarding the preparation of TRI130 for administration to patients in various cohorts is shown in Tables 12(a), 12(b), 13(a), and 13(b). Tables 12(a) and 12(b) provide volumes of normal saline, IVSS, and TRI130 drug product for preparation of the drug dilution bag. Tables 13(a) and 13(b) provide volumes of normal saline, IVSS, and TRI130 drug solution (from the drug dilution bag) for preparation of the patient administration syringe.

TABLE 12(a)
Component volumes for preparation of drug dilution bag

| | | 0.9% | | | | |
|---|---|---|---|---|---|---|
| | | Sodium | | TRI130 | | |
| | | Chloride | | (1 mL | Resulting | Resulting |
| | TRI130 | (normal | | vial of 2 | Mixture | Mixture |
| Cohort | Dose | Saline) | IVSS | mg/mL) | Volume | Concentration |
| 1 to 4 | 0.3 to 9 mcg | 900 mL | 45 mL | 0.5 mL | 945.5 mL | 1.058 mcg/mL |
| 5 to 10 | 12 to 100 mcg | 92 mL | 4.6 mL | 0.5 mL | 97.1 mL | 10.30 mcg/mL |

TABLE 12(b)
Component volumes for preparation of drug dilution bag

| | Normal | | | | |
|---|---|---|---|---|---|
| | Saline | | | | |
| | (0.9% | IVSS | TRI130 | Resulting | Resulting |
| TRI130 | Sodium | (10 mL | (1 mL vial | Mixture | TRI130 |
| Dose | Chloride) | vial) | of 2 mg/mL) | Volume | Concentration |
| 3 to 100 | 190 mL | 9.7 mL | 0.3 mL (600 | 200 mL | 3 mcg/mL |
| mcg | | | mcg) | | |

Table 13(a): Component volumes for preparation of patient administration syringe

| | | | | | | Volume used | | |
|---|---|---|---|---|---|---|---|---|
| | | | | Step 6 “C” | | to prime | | |
| | | | | TRI130 from | | IV line and | | |
| | | Step 4 “A” | | DRUG | Total | filter. | | |
| | | Normal | | DILUTION | Volume | Volume is | Total | |
| | | Saline into | Step 5 “B” | BAG INTO | prepared in | run through | Volume in | |
| | | PATIENT | IVSS into | PATIENT | PATIENT | line and any | PATIENT | Concen- |
| | | ADMINIS- | PATIENT | ADMINIS- | ADMINIS- | extra exiting | ADMINIS- | tration |
| | TRI130 | TRATION | ADMINISTRATON | TRATION | TRATION | end of IV line | TRATION | of TRI130 |
| Cohort | Dose | SYRINGE | SYRINGE | SYRINGE | SYRINGE | is discarded. | SYRINGE | in Syringe |

Use Table 12 to prepare drug solution in the
DRUG DILUTION BAG for Cohort 1 to 4

| | | | | | | | | | |
|---|---|---|---|---|---|---|---|---|---|
| 1 | 0.3 | mcg | 9 mL | 0.5 mL | 0.3 mL | 9.8 mL | 1 mL | 8.8 mL | 0.03 mcg/mL |
| 2 | 1 | mcg | 30 mL | 1.5 mL | 1.0 mL | 32.5 mL | 1 mL | 31.5 mL | 0.03 mcg/mL |
| 3 | 3 | mcg | 46 mL | 2.4 mL | 2.9 mL | 51.3 mL | 1 mL | 50.3 mL | 0.06 mcg/mL |
| 4 | 9 | mcg | 40 mL | 2.0 mL | 8.7 mL | 50.7 mL | 1 mL | 49.7 mL | 0.18 mcg/mL |

Use Table 12 to prepare drug solution in the
DRUG DILUTION BAG for Cohort 5 to 10

| | | | | | | | | | |
|---|---|---|---|---|---|---|---|---|---|
| 5 | 12 | mcg | 48 mL | 2.5 mL | 1.2 mL | 51.7 mL | 1 mL | 50.7 mL | 0.24 mcg/mL |
| 6 | 20 | mcg | 46 mL | 2.4 mL | 2.0 mL | 50.4 mL | 1 mL | 49.4 mL | 0.41 mcg/mL |
| 7 | 30 | mcg | 44 mL | 2.3 mL | 3.0 mL | 49.3 mL | 1 mL | 48.3 mL | 0.63 mcg/mL |
| 8 | 50 | mcg | 44 mL | 2.3 mL | 4.9 mL | 51.2 mL | 1 mL | 50.2 mL | 0.99 mcg/mL |
| 9 | 75 | mcg | 42 mL | 2.2 mL | 7.4 mL | 51.6 mL | 1 mL | 50.6 mL | 1.48 mcg/mL |
| 10 | 100 | mcg | 40 mL | 2.0 mL | 9.9 mL | 51.9 mL | 1 mL | 50.9 mL | 1.96 mcg/mL |

TABLE 13(b)
Component volumes for preparation of patient administration syringe

| | Step 2 “A” | Step 3 “B” | Step 5 “C” | | |
|---|---|---|---|---|---|
| | Normal Saline into | IVSS into | TRI130 from DRUG DILUTION | Total Volume prepared | |
| | PATIENT | PATIENT | BAG into PATIENT | for administration in | Concentration of |
| | ADMINISTRATION | ADMINISTRATION | ADMINISTRATION | PATIENT | TRI130 in |
| TRI130 Dose | SYRINGE | SYRINGE | SYRINGE | ADMINISTRATION | Syringe |
| (mcg) | (mL) | (mL) | (mL) | SYRINGE (mL) | (mcg/mL) |
| 3 | 46.5 | 2.5 | 1 | 50 | 0.06 |
| 6 | 45.6 | 2.4 | 2 | 50 | 0.12 |
| 9 | 44.7 | 2.3 | 3 | 50 | 0.18 |
| 12 | 43.7 | 2.3 | 4 | 50 | 0.24 |
| 15 | 42.8 | 2.2 | 5 | 50 | 0.3 |
| 18 | 41.8 | 2.2 | 6 | 50 | 0.36 |
| 24 | 39.8 | 2.2 | 8 | 50 | 0.48 |
| 36 | 36 | 2 | 12 | 50 | 0.72 |
| 48 | 32.3 | 1.7 | 16 | 50 | 0.96 |
| 60 | 28.5 | 1.5 | 20 | 50 | 1.2 |
| 100 | 15.8 | 0.9 | 33.3 | 50 | 2.0 |

### REFERENCES

1. Cleland, J., Powell M., et al; Crit Rev Ther Drug Carrier Syst 10(4):307-377; The development of stable protein formulations: a close look at protein aggregation, deamidation, and oxidation
2. Shire, S., et al; J Pharma Science 93(6):1390-1402 (2004); Challenges in the development of high protein concentration formulations
3. Bruce Kerwin; Journal of Pharm Sciences 97(8):2924-2935(2008); Polysorbate 20 and 80 used in the formulation of protein biotherapeutics: Structure and degradation pathways
4. Nema, S and Brendel R; Journal of Pharmaceutical Science and Technology Vol 65, No 3, May-June 2011; Excipients and their role in approved injectable products: current usage and future directions

1. A composition for reducing adsorption of a therapeutic protein to one or more components of an intravenous drug delivery system, the composition comprising succinate and polysorbate 80.

2. The composition of claim 1, wherein the composition comprises:
about 1 to about 10 mM succinate, and
about 0.001% (w/v) to about 0.01% (w/v) polysorbate 80.

3. The composition of claim 2, wherein the composition comprises about 4 mM to about 6 mM succinate.

4. The composition of claim 3, wherein the composition comprises about 5 mM succinate.

5. The composition of any one of claims 2-4, wherein the composition comprises about 0.002% (w/v) to about 0.008% (w/v) polysorbate 80.

6. The composition of claim 5, wherein the composition comprises about 0.004% (w/v) polysorbate 80.

7. The composition of any one of claims 2-6, wherein the pH of the composition is about 5.0 to about 7.0.

8. The composition of claim 7, wherein the pH of the composition is about 6.0.

9. The composition of any one of claims 1-8, wherein the composition comprises the therapeutic protein.

10. The composition of claim any one of claims 1-9, wherein the therapeutic protein comprises at least a first binding domain.

11. The composition of claim 10, wherein the first binding domain is a single chain variable fragment (scFv).

12. The composition of any one of claims 1-9, wherein the therapeutic protein comprises at least a first binding domain and a second binding domain.

13. The composition of claim 12, wherein the first binding domain is a single chain variable fragment (scFv) and the second binding domain is a scFv.

14. The composition of claim 12 or 13, wherein the first binding domain specifically binds to CD123.

15. The composition of any one of claims 12-14, wherein the second binding domain specifically binds CD3c.

16. The composition of any one of claims 12-15, wherein the therapeutic protein comprises, in order from amino terminus to carboxyl terminus:
(a) the first binding domain
(b) a hinge region;
(c) an immunoglobulin constant region; and
(d) the second binding domain.

17. The composition of claim 16, wherein the immunoglobulin constant region comprises immunoglobulin CH2 and CH3 domains of IgG1, IgG2, IgG3, IgG4, IgA1, IgA2 or IgD.

18. The composition of any one of claims 12-17, wherein the first binding domain comprises:
(i) an immunoglobulin heavy chain variable region (VH) comprising HCDR1, HCDR2, and HCDR3; and
(ii) an immunoglobulin light chain variable region (VL) comprising LCDR1, LCDR2, and LCDR3.

19. The composition of claim 18, wherein the HCDR1 comprises SEQ ID NO: 10, the HCDR2 comprises SEQ ID NO: 11, and the HDCR3 comprises SEQ ID NO: 12.

20. The composition of claim 18, wherein the LCDR1 comprises SEQ ID NO: 13, the LCDR2 comprises SEQ ID NO: 14, and the LCDR3 comprises SEQ ID NO: 15.

21. The composition of claim 18, wherein:
the HCDR1 comprises SEQ ID NO: 10, the HCDR2 comprises SEQ ID NO: 11, and the HDCR3 comprises SEQ ID NO: 12; and
the LCDR1 comprises SEQ ID NO: 13, the LCDR2 comprises SEQ ID NO: 14, and the LCDR3 comprises SEQ ID NO: 15.

22. The composition of any one of claims 12-21, wherein the first binding domain comprises a sequence at least 95% identical to SEQ ID NO: 18.

23. The composition of claim any one of claims 12-22, wherein the second binding domain comprises:
(i) an immunoglobulin heavy chain variable region (VH) comprising HCDR1, HCDR2, and HCDR3; and
(ii) an immunoglobulin light chain variable region (VL) comprising LCDR1, LCDR2, and LCDR3.

24. The composition of claim 23, wherein the HCDR1 comprises SEQ ID NO: 19, the HCDR2 comprises SEQ ID NO: 20, and the HDCR3 comprises SEQ ID NO: 21.

25. The composition of claim 23, wherein the LCDR1 comprises SEQ ID NO: 22, the LCDR2 comprises SEQ ID NO: 23, and the LCDR3 comprises SEQ ID NO: 24.

26. The composition of claim 23, wherein:
the HCDR1 comprises SEQ ID NO: 19, the HCDR2 comprises SEQ ID NO: 20, and the HDCR3 comprises SEQ ID NO: 21; and
the LCDR1 comprises SEQ ID NO: 22, the LCDR2 comprises SEQ ID NO: 23, and the LCDR3 comprises SEQ ID NO: 24.

27. The composition of any one of claims 12-26, wherein the second binding domain comprises a sequence at least 95% or 100% identical to SEQ ID NO: 27.

28. The composition of any one of claims 12-27, wherein the therapeutic protein comprises the sequence of SEQ ID NO: 31.

29. The composition of claim 12 or 13, wherein the therapeutic protein comprises, in order from amino terminus to carboxyl terminus a CD86 binding domain, an immunoglobulin hinge domain, an immunoglobulin Fc domain, and a monomeric IL-10 domain,
wherein the CD86 binding domain comprises a variable heavy chain and a variable light chain that specifically bind CD86,
wherein the immunoglobulin Fc domain is an IgG1 Fc domain that comprises two or more mutations that prevent or significantly reduce binding to Fc receptors FcγR, FcγRIIa, FcγRIIb, and FcγRIIIb,
wherein the monomeric IL-10 domain comprises two subunits of human IL-10 separated by a short linker, and
wherein the therapeutic protein is a homodimer.

30. The composition of claim 29, wherein the CD86 binding domain comprises (i) an immunoglobulin heavy chain variable region (VH) comprising HCDR1, HCDR2, and HCDR3; and (2) an immunoglobulin light chain variable region (VL) comprising LCDR1, LCDR2, and LCDR3.

31. The composition of claim 30, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6.

32. The composition of any one of claims 29-31, wherein the CD86 binding domain comprises a variable heavy chain with an amino acid sequence at least 95% identical to SEQ ID NO: 7 and a variable light chain with an amino acid sequence at least 95% identical to SEQ ID NO: 8.

33. The composition of any one of claims 29-32, wherein the CD86 binding domain comprises an amino acid sequence that is at least about 95% or 100% identical to SEQ ID NO: 9.

34. The composition of any one of claims 29-33, wherein the monomeric IL-10 domain comprises an amino acid sequence at least 95% or 100% identical to SEQ ID NO: 28.

35. The composition of any one of claims 29-34, wherein the therapeutic protein comprises SEQ ID NO: 30 or an amino acid sequence at least about 90%, at least about 95%, at least about 98%, or at least about 99% identical to SEQ ID NO: 30.

36. The composition of any one of claims 9-36, wherein the concentration of the therapeutic protein is about 0.01 μg/mL to about 2.0 μg/mL.

37. The composition of claim 36, wherein the concentration of the therapeutic protein is about 0.01, about 0.02, about 0.03, about 0.04, about 0.05, about 0.06, about 0.07, about 0.08, or about 0.09 μg/mL.

38. The composition of claim 36, wherein the concentration of the therapeutic protein is about 0.1, about 0.2, about 0.3, about 0.4, about 0.5, about 0.6, about 0.7, about 0.8, or about 0.9 μg/mL.

39. The composition of claim 36, wherein the concentration of the therapeutic protein is about 1.0, about 1.1, about 1.2, about 1.3, about 1.4, about 1.5, about 1.6, about 1.7, about 1.8, about 1.9, or about 2.0 μg/mL.

40. The composition of any one of claim 1-39, wherein the composition comprises about 5 mM succinate and about 0.0004% (w/v) polysorbate 80 in water, wherein the pH of the composition is about 6.0, and wherein the composition is formulated for injection.

41. The composition of claim 1, wherein the composition comprises about 25 to about 150 mM succinate, and about 0.01% to about 0.1% (w/v) polysorbate 80.

42. The composition of claim 41, wherein the composition is at a 10×-50× concentration.

43. The composition of claim 42, wherein the composition is at a 20× concentration.

44. The composition of any one of claims 41-43, wherein the composition comprises about 75 mM to about 125 mM succinate.

45. The composition of claim 44, wherein the composition comprises about 100 mM succinate.

46. The composition of any one of claims 41-45, wherein the composition comprises about 0.05% (w/v) to about 0.1% (w/v) polysorbate 80.

47. The composition of claim 46, wherein the composition comprises about 0.08% (w/v) polysorbate 80.

48. The composition of any one of claims 41-47, wherein the pH of the composition is about 5.0 to about 7.0.

49. The composition of claim 48, wherein the pH of the composition is about 6.0.

50. The composition of any one of claims 41-49, wherein the composition comprises about 100 mM succinate and about 0.08% (w/v) polysorbate 80 in water, wherein the pH of the composition is about 6.0, and wherein the composition is formulated for injection.

51. A composition for reducing protein adsorption to one or more components of an intravenous drug delivery system, the composition comprising:
about 100 mM succinate;
about 0.08% (w/v) polysorbate 80; and
about therapeutically effective amount of a therapeutic protein.

52. A composition for reducing protein adsorption to one or more components of an intravenous drug delivery system, the composition comprising:
about 1 to about 10 mM succinate;
about 0.001% (w/v) to about 0.01% (w/v) polysorbate 80; and
about 0.01 μg/mL to about 2.0 μg/mL of a therapeutic protein;
wherein the therapeutic protein comprises, in order from amino terminus to carboxyl terminus:
(a) a first binding domain that specifically binds to a first target;
(b) a hinge region;
(c) an immunoglobulin constant region; and
(d) a second binding domain that specifically binds to a second target.

53. The composition of claim 52, wherein the first target is CD86.

54. The composition of claim 52, wherein the first target is CD123.

55. The composition of claim 52, wherein the second target is a receptor of IL-10.

56. The composition of claim 52, wherein the second target is CD3c.

57. The composition of claim 52, wherein the first target is CD86 and the second target is a receptor of IL-10.

58. The composition of claim 52, wherein the first target is CD123 and the second target is CD3c.

59. A composition for reducing protein adsorption to one or more components of an intravenous drug delivery system, the composition comprising:
about 1 to about 10 mM succinate,
about 0.001% (w/v) to about 0.01% (w/v) polysorbate 80; and
about 0.01 μg/mL to about 2.0 μg/mL of a therapeutic protein;
wherein the therapeutic protein comprises, in order from amino terminus to carboxyl terminus:
(a) a first binding domain;
(b) a hinge region;
(c) an immunoglobulin constant region; and
(d) a second binding domain;
wherein the first binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3; wherein the HCDR1 comprises SEQ ID NO: 10, the HCDR2 comprises SEQ ID NO: 11, and the HDCR3 comprises SEQ ID NO: 12; and wherein the LCDR1 comprises SEQ ID NO: 13, the LCDR2 comprises SEQ ID NO: 14, and the LCDR3 comprises SEQ ID NO: 15;
wherein the second binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3; wherein the HCDR1 comprises SEQ ID NO: 19, the HCDR2 comprises SEQ ID NO: 20, and the HDCR3 comprises SEQ ID NO: 21; and wherein the LCDR1 comprises SEQ ID NO: 22, the LCDR2 comprises SEQ ID NO: 23, and the LCDR3 comprises SEQ ID NO: 24.

60. A composition for reducing protein adsorption to one or more components of an intravenous drug delivery system, the composition comprising:
about 1 to about 10 mM succinate;
about 0.001% (w/v) to about 0.01% (w/v) polysorbate 80; and
about 0.01 μg/mL to about 2.0 μg/mL of a therapeutic protein;
wherein the therapeutic protein comprises the sequence of SEQ ID NO: 31.

61. A composition for reducing protein adsorption to one or more components of an intravenous drug delivery system, the composition comprising:
about 1 to about 10 mM succinate;
about 0.001% (w/v) to about 0.01% (w/v) polysorbate 80; and
about 0.01 μg/mL to about 2.0 μg/mL of a therapeutic protein;
wherein the therapeutic protein comprises, in order from amino terminus to carboxyl terminus: a CD86 binding domain, an immunoglobulin hinge domain, an immunoglobulin Fc domain, and a monomeric IL-10 domain,
wherein the CD86 binding domain comprises a variable heavy chain and a variable light chain that specifically bind CD86,
wherein the immunoglobulin Fc domain is an IgG1 Fc domain that comprises two or more mutations that prevent or significantly reduce binding to Fc receptors FcγR, FcγRIIa, FcγRIIb, and FcγRIIIb,
wherein the monomeric IL-10 domain comprises two subunits of human IL-10 separated by a short linker, and
wherein the therapeutic protein is a homodimer.

62. A composition for reducing protein adsorption to one or more components of an intravenous drug delivery system, the composition comprising:
about 1 to about 10 mM succinate;
about 0.001% (w/v) to about 0.01% (w/v) polysorbate 80; and
about 0.01 μg/mL to about 2.0 μg/mL of a therapeutic protein;
wherein the therapeutic protein comprises, in order from amino terminus to carboxyl terminus: a CD86 binding domain, an immunoglobulin hinge domain, an immunoglobulin Fc domain, and a monomeric IL-10 domain;
wherein the CD86 binding domain comprises (i) an immunoglobulin heavy chain variable region (VH) comprising HCDR1, HCDR2, and HCDR3; and (2) an immunoglobulin light chain variable region (VL) comprising LCDR1, LCDR2, and LCDR3, wherein the amino acid sequence of HCDR1 is SEQ ID NO: 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6;
wherein the monomeric IL-10 domain has an amino acid sequence of SEQ ID NO:28.

63. A composition for reducing protein adsorption to one or more components of an intravenous drug delivery system, the composition comprising:
about 1 to about 10 mM succinate;
about 0.001% (w/v) to about 0.01% (w/v) polysorbate 80; and
about 0.01 μg/mL to about 2.0 μg/mL of a therapeutic protein;
wherein the therapeutic protein comprises, in order from amino terminus to carboxyl terminus: a CD86 binding domain, an immunoglobulin hinge domain, an immunoglobulin Fc domain, and a monomeric IL-10 domain;
wherein the CD86 binding domain comprises the amino acid sequence of SEQ ID NO: 9; and
wherein the monomeric IL-10 domain comprises the amino acid sequence of SEQ ID NO: 28.

64. A composition for reducing protein adsorption to one or more components of an intravenous drug delivery system, the composition comprising:
about 1 to about 10 mM succinate;
about 0.001% (w/v) to about 0.01% (w/v) polysorbate 80; and
about 0.01 μg/mL to about 2.0 μg/mL of a therapeutic protein;
wherein the therapeutic protein comprises the amino acid sequence of SEQ ID NO: 30.

65. A container adapted for holding a therapeutic protein, wherein an interior surface of the container is first contacted with the composition of any one of claims 1-64 before it is contacted with a composition comprising the therapeutic protein.

66. The container of claim 65, wherein the container is substantially free of latex.

67. The container of any one of claims 65-66, wherein the container is substantially free of bis(2-ethylhexyl) phthalate (DEHP).

68. The container of any one of claims 65-67, wherein the container is selected from the group consisting of an IV bag, a syringe, and a tube.

69. A method of preparing an intravenous drug delivery system for delivery of a therapeutic protein, the method comprising:
providing at least one container adapted to hold the therapeutic protein; and
before the therapeutic protein is added to the at least one container, contacting an interior surface of the at least one container with a composition comprising about 1 to about 10 mM succinate, and about 0.001% to 0.01% (w/v) polysorbate 80.

70. The method of claim 69, wherein the composition coats the interior surface of the at least one container and prevents the therapeutic protein from binding to the interior surface of the container.

71. The method of any one of claims 69-70, wherein the at least one container is substantially free of latex.

72. The method of any one of claims 69-71, wherein the at least one container is substantially free of bis(2-ethylhexyl) phthalate (DEHP).

73. The method of any one of claims 69-72, wherein the at least one container is selected from the group consisting of an IV bag, a syringe, and a tube.

74. A method of treating a subject by intravenous administration of a therapeutic protein, the method comprising:
providing at least one container adapted to hold the therapeutic protein;
contacting an interior surface of the container with a composition comprising about 1 to about 10 mM succinate and about 0.001% to about 0.01% (w/v) polysorbate 80;
contacting the interior surface of the container with a composition comprising the therapeutic protein; and
intravenously administering the therapeutic protein to the patient.

75. The method of claim 74, wherein the therapeutic protein comprises at least a first binding domain.

76. The method of claim 75, wherein the first binding domain is a single chain variable fragment (scFv).

77. The method of claim 74, wherein the therapeutic protein comprises at least a first binding domain and a second binding domain.

78. The method of claim 77, wherein the first binding domain is a single chain variable fragment (scFv) and the second binding domain is an scFv.

79. The method of any one of claim 77 or 78, wherein the first binding domain specifically binds to CD123.

80. The method of any one of claims 77 to 79, wherein the second binding domain specifically binds CD3c.

81. The method of any one of claims 77 to 80, wherein the therapeutic protein comprises, in order from amino terminus to carboxyl terminus:
(a) the first binding domain;
(b) a hinge region;
(c) an immunoglobulin constant region; and
(d) the second binding domain.

82. The method of claim 81, wherein the immunoglobulin constant region comprises immunoglobulin CH2 and CH3 domains of IgG1, IgG2, IgG3, IgG4, IgA1, IgA2 or IgD.

83. The method of any one of claims 77 to 82, wherein the first binding domain comprises:
(i) an immunoglobulin heavy chain variable region (VH) comprising HCDR1, HCDR2, and HCDR3; and
(ii) an immunoglobulin light chain variable region (VL) comprising LCDR1, LCDR2, and LCDR3.

84. The method of claim 83, wherein the HCDR1 comprises SEQ ID NO: 10, the HCDR2 comprises SEQ ID NO: 11, and the HDCR3 comprises SEQ ID NO: 12.

85. The method of claim 83, wherein the LCDR1 comprises SEQ ID NO: 13, the LCDR2 comprises SEQ ID NO: 14, and the LCDR3 comprises SEQ ID NO: 15.

86. The method of claim 83, wherein:
the HCDR1 comprises SEQ ID NO: 10, the HCDR2 comprises SEQ ID NO: 11, and the HDCR3 comprises SEQ ID NO: 12; and
the LCDR1 comprises SEQ ID NO: 13, the LCDR2 comprises SEQ ID NO: 14, and the LCDR3 comprises SEQ ID NO: 15.

87. The method of claim any one of claims 77 to 86, wherein the first binding domain comprises a sequence at least 95% or 100% identical to SEQ ID NO: 18.

88. The method of claim 77, wherein the second binding domain comprises:
(i) an immunoglobulin heavy chain variable region (VH) comprising HCDR1, HCDR2, and HCDR3; and
(ii) an immunoglobulin light chain variable region (VL) comprising LCDR1, LCDR2, and LCDR3.

89. The method of claim 88, wherein the HCDR1 comprises SEQ ID NO: 19, the HCDR2 comprises SEQ ID NO: 20, and the HDCR3 comprises SEQ ID NO: 21.

90. The method of claim 88, wherein the LCDR1 comprises SEQ ID NO: 22, the LCDR2 comprises SEQ ID NO: 23, and the LCDR3 comprises SEQ ID NO: 24.

91. The method of claim 88, wherein:
the HCDR1 comprises SEQ ID NO: 19, the HCDR2 comprises SEQ ID NO: 20, and the HDCR3 comprises SEQ ID NO: 21; and
the LCDR1 comprises SEQ ID NO: 22, the LCDR2 comprises SEQ ID NO: 23, and the LCDR3 comprises SEQ ID NO: 24.

92. The method of any one of claims 77-91, wherein the second binding domain comprises a sequence at least 95% identical to SEQ ID NO: 27.

93. The method of any one of claims 77-91, wherein the therapeutic protein comprises the sequence of SEQ ID NO: 31.

94. The method of any one of claims 77-80, wherein the therapeutic protein comprises, in order from amino terminus to carboxyl terminus a CD86 binding domain, an immunoglobulin hinge domain, an immunoglobulin Fc domain, and a monomeric IL-10 domain;
wherein the CD86 binding domain comprises a variable heavy chain and a variable light chain that specifically bind CD86;
wherein the immunoglobulin Fc domain is an IgG1 Fc domain that comprises two or more mutations that prevent or significantly reduce binding to Fc receptors FcγR, FcγRIIa, FcγRIIb, and FcγRIIIb;
wherein the monomeric IL-10 domain comprises two subunits of human IL-10 separated by a short linker; and
wherein the therapeutic protein is a homodimer.

95. The method of claim 94, wherein the CD86 binding domain comprises (i) an immunoglobulin heavy chain variable region (VH) comprising HCDR1, HCDR2, and HCDR3; and (2) an immunoglobulin light chain variable region (VL) comprising LCDR1, LCDR2, and LCDR3.

96. The method of claim 95, wherein the amino acid sequence of HCDR1 is SEQ ID NO 1, the amino acid sequence of HCDR2 is SEQ ID NO: 2, the amino acid sequence of HCDR3 is SEQ ID NO: 3, the amino acid sequence of LCDR1 is SEQ ID NO: 4, the amino acid sequence of LCDR2 is SEQ ID NO: 5, and the amino acid sequence of LCDR3 is SEQ ID NO: 6.

97. The method of any one of claims 94-96, wherein the CD86 binding domain comprises a variable heavy chain with an amino acid sequence at least 95% or 100% identical to SEQ ID NO: 7 and a variable light chain with an amino acid sequence at least 95% or 100% identical to SEQ ID NO: 8.

98. The method of any one of claims 94-97, wherein the CD86 binding domain comprises an amino acid sequence with at least about 95% or 100% identical to SEQ ID NO: 9.

99. The method of any one of claims 94-98, wherein the monomeric IL-10 domain comprises an amino acid sequence at least 95% or 100% identical to SEQ ID NO: 28.

100. The method of any one of claims 94-99, wherein the therapeutic protein comprises SEQ ID NO: 30 or an amino acid sequence at least about 90%, at least about 95%, at least about 98%, or at least about 99% identical to SEQ ID NO: 30.

101. The method of any one of claims 74-100 wherein the therapeutic protein is administered by intravenous infusion.

102. The method of any one of claims 74-101, wherein the composition coats an interior surface of the at least one container and prevents the therapeutic protein from binding to the interior surface of the container.

103. The method of any one of claims 74-102, wherein the subject is a mammal.

104. The method of claim 103, wherein the subject is a human.

105. A drug delivery system for delivering a therapeutic protein to a patient, the system comprising:
at least one container adapted to hold the therapeutic protein;
wherein an interior surface of the at least one container is contacted with a composition comprising about 1 to about 10 mM succinate, and about 0.001% to 0.01% (w/v) polysorbate 80 before it is contacted with a composition comprising the therapeutic protein.


SEQUENCE LISTING (389 sequences)

SEQ ID NO: 1 - protein, 5 aa, Artificial Sequence
  anti-CD86 HCDR1
DYNMN

SEQ ID NO: 2 - protein, 16 aa, Artificial Sequence
  anti-CD86 HCDR2
IDPYYGGTSYNQKFKG

SEQ ID NO: 3 - protein, 16 aa, Artificial Sequence
  anti-CD86 HCDR3
WDYRYDDGRAYYVMDF

SEQ ID NO: 4 - protein, 17 aa, Artificial Sequence
  anti-CD86 LCDR1
KSSQSVLYSSNQKNYLA

SEQ ID NO: 5 - protein, 7 aa, Artificial Sequence
  anti-CD86 LCDR2
WASTRES

SEQ ID NO: 6 - protein, 8 aa, Artificial Sequence
  anti-CD86 LCDR3
HQYLYSWT

SEQ ID NO: 7 - protein, 123 aa, Artificial Sequence
  humanized anti-CD86 VH
QVQLVQSGAEVKKPGASVKVSCKASGYTFTDYNMNWVRQAPGQGLEWMGNIDPYYGGTSYNQKFKGRVTMTRDTSISTAYMELSRLRSDDTAVYYCARWDYRYDDGRAYYVMDFWGQGTTVTV

SEQ ID NO: 8 - protein, 114 aa, Artificial Sequence
  humanized anti-CD86 VL
DIVMTQSPDSLAVSLGERATINCKSSQSVLYSSNQKNYLAWYQQKPGQPPKLLIYWASTRESGVPDRFSGSGSGTDFTLTISSLQAEDVAVYYCHQYLYSWTFGQGTKLEIKRL

SEQ ID NO: 9 - protein, 259 aa, Artificial Sequence
  humanized anti-CD86 scFv
QVQLVQSGAEVKKPGASVKVSCKASGYTFTDYNMNWVRQAPGQGLEWMGNIDPYYGGTSYNQKFKGRVTMTRDTSISTAYMELSRLRSDDTAVYYCARWDYRYDDGRAYYVMDFWGQGTTVTVSSGGGGSGGGGSGGGGSGGGGSDIVMTQSPDSLAVSLGERATINCKSSQSVLYSSNQKNYLAWYQQKPGQPPKLLIYWASTRESGVPDRFSGSGSGTDFTLTISSLQAEDVAVYYCHQYLYSWTFGQGTKLEIKRL

SEQ ID NO: 10 - protein, 8 aa, Artificial Sequence
  anti-CD123 HCDR1
GFTFSSYG

SEQ ID NO: 11 - protein, 8 aa, Artificial Sequence
  anti-CD123 HCDR2
ISGSGGST

SEQ ID NO: 12 - protein, 17 aa, Artificial Sequence
  anti-CD123 HCDR3
AKEKLRYFDWLSDAFDI

SEQ ID NO: 13 - protein, 12 aa, Artificial Sequence
  anti-CD123 LCDR1
HSVLYSSNNKNY

SEQ ID NO: 14 - protein, 3 aa, Artificial Sequence
  anti-CD123 LCDR2
WAS

SEQ ID NO: 15 - protein, 10 aa, Artificial Sequence
  anti-CD123 LCDR3
QQYYSTPPTT

SEQ ID NO: 16 - protein, 114 aa, Artificial Sequence
  ANTI-CD123 VH
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIK

SEQ ID NO: 17 - protein, 124 aa, Artificial Sequence
  ANTI-CD123 VL
EVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSS

SEQ ID NO: 18 - protein, 258 aa, Artificial Sequence
  anti-CD123 scFv
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSS

SEQ ID NO: 19 - protein, 8 aa, Artificial Sequence
  anti-CD3? HCDR1
GYTFSRST

SEQ ID NO: 20 - protein, 8 aa, Artificial Sequence
  anti-CD3? HCDR2
INPSSAYT

SEQ ID NO: 21 - protein, 14 aa, Artificial Sequence
  anti-CD3? HCDR3
ARPQVHYDYNGFPY

SEQ ID NO: 22 - protein, 7 aa, Artificial Sequence
  anti-CD3? LCDR1
ASSSVSY

SEQ ID NO: 23 - protein, 3 aa, Artificial Sequence
  anti-CD3? LCDR2
DSS

SEQ ID NO: 24 - protein, 9 aa, Artificial Sequence
  anti-CD3? LCDR3
QQWSRNPPT

SEQ ID NO: 25 - protein, 121 aa, Artificial Sequence
  anti-CD3? VH
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSS

SEQ ID NO: 26 - protein, 108 aa, Artificial Sequence
  anti-CD3? VL
DIQMTQSPSTLSASVGDRVTMTCSASSSVSYMNWYQQKPGKAPKRWIYDSSKLASGVPSRFSGSGSGTEYTLTISSLQPDDFATYYCQQWSRNPPTFGGGTKVEIKRS

SEQ ID NO: 27 - protein, 249 aa, Artificial Sequence
  anti-CD3? scFv
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 28 - protein, 166 aa, Artificial Sequence
  Monomeric IL-10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 29 - DNA, 2112 bp, Artificial Sequence
  anti-CD86 x monomeric IL-10 bispecific construct DNA sequence
aagcttgccgccatggaagcaccagcgcagcttctcttcctcctgctactctggctcccagataccaccggtcaggtgcagctggtgcagtctggggctgaggtgaagaagcctggggcctcagtgaaggtctcctgcaaggcttctggatacaccttcaccgactacaacatgaactgggtgcgacaggcccctggacaagggcttgagtggatgggaaatattgatccttactatggtggtactagttacaatcagaagttcaagggcagggtcaccatgaccagggacacgtccatcagcacagcctacatggagctgagcaggctgagatctgacgacacggccgtgtattactgtgcgagatgggactataggtacgacgacgggagggcttactatgttatggacttctggggccaagggaccacggtcaccgtctcctcaggtggaggcggttcaggcggaggtggatccggcggtggcggatcgggtggcggcggatctgacatcgtgatgacccagtctccagactccctggctgtgtctctgggcgagagggccaccatcaactgcaagtccagccagagtgttttatacagctccaaccagaagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcatcaatacctctactcgtggacgtttggccaggggaccaagctggagatcaaacggctcgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttcttccctgaatacaggaactcagccgaattctagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacggtggtggatccggcggtaagagcaaggccgtggagcaggtgaagaatgcctttaataagctccaagagaaaggcatctacaaagccatgagtgagtttgacatcttcatcaactacatagaagcctacatgacaatgaagatacgaaactaacgtacgttcgaacccgggcacgtgcggaccgaattc

SEQ ID NO: 30 - protein, 667 aa, Artificial Sequence
  anti-CD86 x monomeric IL-10 bispecific construct amino acid sequence
QVQLVQSGAEVKKPGASVKVSCKASGYTFTDYNMNWVRQAPGQGLEWMGNIDPYYGGTSYNQKFKGRVTMTRDTSISTAYMELSRLRSDDTAVYYCARWDYRYDDGRAYYVMDFWGQGTTVTVSSGGGGSGGGGSGGGGSGGGGSDIVMTQSPDSLAVSLGERATINCKSSQSVLYSSNQKNYLAWYQQKPGQPPKLLIYWASTRESGVPDRFSGSGSGTDFTLTISSLQAEDVAVYYCHQYLYSWTFGQGTKLEIKRLEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSSLNTGTQPNSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 31 - protein, 777 aa, Artificial Sequence
  anti-CD123 x anti-CD3? bispecific construct amino acid sequence
MEAPAQLLFLLLLWLPDTTGDIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 32 - DNA, 699 bp, Artificial Sequence
  modified immunoglobulin constant region
tcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 33 - protein, 233 aa, Artificial Sequence
  modified immunoglobulin constant region
SSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 693 bp, Artificial Sequence
  modified immunoglobulin constant region
gagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 35 - protein, 231 aa, Artificial Sequence
  modified immunoglobulin constant region
EPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 15 aa, Artificial Sequence
  immunoglobulin hinge region
EPKSSDKTHTSPPSS

SEQ ID NO: 37 - protein, 15 aa, Artificial Sequence
  immunoglobulin hinge region
EPKSCDKTHTSPPCS

SEQ ID NO: 38 - protein, 15 aa, Artificial Sequence
  immunoglobulin hinge region
EPKSSDKTHTSPPCS

SEQ ID NO: 39 - protein, 15 aa, Artificial Sequence
  immunoglobulin hinge region
EPKSSDKTHTCPPCS

SEQ ID NO: 40 - protein, 15 aa, Artificial Sequence
  immunoglobulin hinge region
EPKSCDKTHTSPPSS

SEQ ID NO: 41 - protein, 15 aa, Artificial Sequence
  immunoglobulin hinge region
EPKSSDKTHTCPPSS

SEQ ID NO: 42 - protein, 15 aa, Artificial Sequence
  immunoglobulin hinge region
EPKSCDKTHTSPPCS

SEQ ID NO: 43 - protein, 15 aa, Artificial Sequence
  immunoglobulin hinge region
EPKSCDKTHTSPPCP

SEQ ID NO: 44 - protein, 15 aa, Artificial Sequence
  immunoglobulin hinge region
EPKSSDKTHTSPPSP

SEQ ID NO: 45 - protein, 15 aa, Artificial Sequence
  immunoglobulin hinge region
EPKSCDKTHTSPPCP

SEQ ID NO: 46 - protein, 15 aa, Artificial Sequence
  immunoglobulin hinge region
EPKSSDKTHTSPPCP

SEQ ID NO: 47 - protein, 15 aa, Artificial Sequence
  immunoglobulin hinge region
EPKSSDKTHTCPPCP

SEQ ID NO: 48 - protein, 15 aa, Artificial Sequence
  immunoglobulin hinge region
EPKSCDKTHTSPPSP

SEQ ID NO: 49 - protein, 15 aa, Artificial Sequence
  immunoglobulin hinge region
EPKSSDKTHTCPPSP

SEQ ID NO: 50 - protein, 6 aa, Artificial Sequence
  Fc binding domain linker
SCPPCP

SEQ ID NO: 51 - protein, 20 aa, Artificial Sequence
  Fc binding domain linker
NYGGGGSGGGGSGGGGSGNS

SEQ ID NO: 52 - protein, 38 aa, Artificial Sequence
  Fc binding domain linker
NYGGGGSGGGGSGGGGSGNYGGGGSGGGGSGGGGSGNS

SEQ ID NO: 53 - protein, 8 aa, Artificial Sequence
  Fc binding domain linker
GGGGSGNS

SEQ ID NO: 54 - protein, 10 aa, Artificial Sequence
  Fc binding domain linker
NYGGGGSGNS

SEQ ID NO: 55 - protein, 13 aa, Artificial Sequence
  Fc binding domain linker
GGGGSGGGGSGNS

SEQ ID NO: 56 - protein, 15 aa, Artificial Sequence
  Fc binding domain linker
NYGGGGSGGGGSGNS

SEQ ID NO: 57 - protein, 18 aa, Artificial Sequence
  Fc binding domain linker
GGGGSGGGGSGGGGSGNS

SEQ ID NO: 58 - protein, 8 aa, Artificial Sequence
  Fc binding domain linker
GCPPCPNS

SEQ ID NO: 59 - protein, 15 aa, Artificial Sequence
  Fc binding domain linker
GGGGSGGGGSGGGGS

SEQ ID NO: 60 - protein, 16 aa, Artificial Sequence
  Fc binding domain linker
SGGGGSGGGGSGGGGS

SEQ ID NO: 61 - protein, 20 aa, Artificial Sequence
  Fc binding domain linker
GGGGSGGGGSGGGGSGGGGS

SEQ ID NO: 62 - protein, 25 aa, Artificial Sequence
  Fc binding domain linker
GGGGSGGGGSGGGGSGGGGSGGGGS

SEQ ID NO: 63 - protein, 21 aa, Artificial Sequence
  Fc binding domain linker
QRHNNSSLNTGTQMAGHSPNS

SEQ ID NO: 64 - protein, 16 aa, Artificial Sequence
  Fc binding domain linker
SSLNTGTQMAGHSPNS

SEQ ID NO: 65 - protein, 18 aa, Artificial Sequence
  Fc binding domain linker
QRHNNSSLNTGTQMAGHS

SEQ ID NO: 66 - protein, 14 aa, Artificial Sequence
  Fc binding domain linker
EVQIPLTESYSPNS

SEQ ID NO: 67 - protein, 20 aa, Artificial Sequence
  Fc binding domain linker
NSLANQEVQIPLTESYSPNS

SEQ ID NO: 68 - protein, 19 aa, Artificial Sequence
  Fc binding domain linker
SGGGGSGGGGSGGGGSPNS

SEQ ID NO: 69 - protein, 19 aa, Artificial Sequence
  Fc binding domain linker
SGGGGSGGGGSGGGGSPGS

SEQ ID NO: 70 - protein, 17 aa, Artificial Sequence
  Fc binding domain linker
GGGGSGGGGSGGGGSPS

SEQ ID NO: 71 - protein, 15 aa, Artificial Sequence
  Hinge or linker sequence
LSVKADFLTPSIGNS

SEQ ID NO: 72 - protein, 20 aa, Artificial Sequence
  Hinge or linker sequence
LSVKADFLTPSISCPPCPNS

SEQ ID NO: 73 - protein, 15 aa, Artificial Sequence
  Hinge or linker sequence
LSVLANFSQPEIGNS

SEQ ID NO: 74 - protein, 20 aa, Artificial Sequence
  Hinge or linker sequence
LSVLANFSQPEISCPPCPNS

SEQ ID NO: 75 - protein, 15 aa, Artificial Sequence
  Hinge or linker sequence
LKIQERVSKPKISNS

SEQ ID NO: 76 - protein, 20 aa, Artificial Sequence
  Hinge or linker sequence
LKIQERVSKPKISCPPCPNS

SEQ ID NO: 77 - protein, 15 aa, Artificial Sequence
  Hinge or linker sequence
LNVSERPFPPHIQNS

SEQ ID NO: 78 - protein, 21 aa, Artificial Sequence
  Hinge or linker sequence
LDVSERPFPPHIQSCPPCPNS

SEQ ID NO: 79 - protein, 15 aa, Artificial Sequence
  Hinge or linker sequence
REQLAEVTLSLKANS

SEQ ID NO: 80 - protein, 20 aa, Artificial Sequence
  Hinge or linker sequence
REQLAEVTLSLKACPPCPNS

SEQ ID NO: 81 - protein, 15 aa, Artificial Sequence
  Hinge or linker sequence
RIHQMNSELSVLANS

SEQ ID NO: 82 - protein, 20 aa, Artificial Sequence
  Hinge or linker sequence
RIHQMNSELSVLACPPCPNS

SEQ ID NO: 83 - protein, 15 aa, Artificial Sequence
  Hinge or linker sequence
DTKGKNVLEKIFSNS

SEQ ID NO: 84 - protein, 15 aa, Artificial Sequence
  Hinge or linker sequence
LPPETQESQEVTLNS

SEQ ID NO: 85 - protein, 15 aa, Artificial Sequence
  Hinge or linker sequence
RIHLNVSERPFPPNS

SEQ ID NO: 86 - protein, 20 aa, Artificial Sequence
  Hinge or linker sequence
RIHLNVSERPFPPCPPCPNS

SEQ ID NO: 87 - protein, 13 aa, Artificial Sequence
  Hinge or linker sequence
GCPPCPGGGGSNS

SEQ ID NO: 88 - protein, 9 aa, Artificial Sequence
  Hinge or linker sequence
GCPPCPANS

SEQ ID NO: 89 - protein, 9 aa, Artificial Sequence
  Hinge or linker sequence
GCPPCPANS

SEQ ID NO: 90 - protein, 8 aa, Artificial Sequence
  Hinge or linker sequence
GCPPCPNS

SEQ ID NO: 91 - protein, 13 aa, Artificial Sequence
  Hinge or linker sequence
GGGASCPPCPGNS

SEQ ID NO: 92 - protein, 13 aa, Artificial Sequence
  Hinge or linker sequence
GGGASCPPCAGNS

SEQ ID NO: 93 - protein, 12 aa, Artificial Sequence
  Hinge or linker sequence
GGGASCPPCANS

SEQ ID NO: 94 - protein, 15 aa, Artificial Sequence
  Hinge or linker sequence
LSVKADFLTPSIGNS

SEQ ID NO: 95 - protein, 11 aa, Artificial Sequence
  Hinge or linker sequence
ADFLTPSIGNS

SEQ ID NO: 96 - protein, 15 aa, Artificial Sequence
  Hinge or linker sequence
LSVLANFSQPEIGNS

SEQ ID NO: 97 - protein, 15 aa, Artificial Sequence
  Hinge or linker sequence
LSVLANFSQPEIGNS

SEQ ID NO: 98 - protein, 11 aa, Artificial Sequence
  Hinge or linker sequence
SQPEIVPISNS

SEQ ID NO: 99 - protein, 16 aa, Artificial Sequence
  Hinge or linker sequence
SQPEIVPISCPPCPNS

SEQ ID NO: 100 - protein, 19 aa, Artificial Sequence
  Hinge or linker sequence
SVLANFSQPEISCPPCPNS

SEQ ID NO: 101 - protein, 15 aa, Artificial Sequence
  Hinge or linker sequence
RIHQMNSELSVLANS

SEQ ID NO: 102 - protein, 12 aa, Artificial Sequence
  Hinge or linker sequence
QMNSELSVLANS

SEQ ID NO: 103 - protein, 10 aa, Artificial Sequence
  Hinge or linker sequence
VSERPFPPNS

SEQ ID NO: 104 - protein, 15 aa, Artificial Sequence
  Hinge or linker sequence
KPFFTCGSADTCPNS

SEQ ID NO: 105 - protein, 15 aa, Artificial Sequence
  Hinge or linker sequence
KPFFTCGSADTCPNS

SEQ ID NO: 106 - protein, 15 aa, Artificial Sequence
  Hinge or linker sequence
QYNCPGQYTFSMPNS

SEQ ID NO: 107 - protein, 21 aa, Artificial Sequence
  Hinge or linker sequence
EPAFTPGPNIELQKDSDCPNS

SEQ ID NO: 108 - protein, 21 aa, Artificial Sequence
  Hinge or linker sequence
QRHNNSSLNTRTQKARHCPNS

SEQ ID NO: 109 - protein, 20 aa, Artificial Sequence
  Hinge or linker sequence
NSLFNQEVQIPLTESYCPNS

SEQ ID NO: 110 - protein, 14 aa, Artificial Sequence
  anti-CD3 LCDR1
GSSTGAVTSGYYPN

SEQ ID NO: 111 - protein, 14 aa, Artificial Sequence
  anti-CD3 LCDR1
RSSTGAVTSGYYPN

SEQ ID NO: 112 - protein, 14 aa, Artificial Sequence
  anti-CD3 LCDR1
GSSTGAVTSGNYPN

SEQ ID NO: 113 - protein, 7 aa, Artificial Sequence
  anti-CD3 LCDR2
GTKFLAP

SEQ ID NO: 114 - protein, 7 aa, Artificial Sequence
  anti-CD3 LCDR2
ATDMRPS

SEQ ID NO: 115 - protein, 7 aa, Artificial Sequence
  anti-CD3 LCDR2
GTKFLAP

SEQ ID NO: 116 - protein, 9 aa, Artificial Sequence
  anti-CD3 LCDR3
ALWYSNRWV

SEQ ID NO: 117 - protein, 9 aa, Artificial Sequence
  anti-CD3 LCDR3
ALWYSNRWV

SEQ ID NO: 118 - protein, 9 aa, Artificial Sequence
  anti-CD3 LCDR3
VLWYSNRWV

SEQ ID NO: 119 - protein, 5 aa, Artificial Sequence
  anti-CD3 HCDR1
IYAMN

SEQ ID NO: 120 - protein, 5 aa, Artificial Sequence
  anti-CD3 HCDR1
KYAMN

SEQ ID NO: 121 - protein, 5 aa, Artificial Sequence
  anti-CD3 HCDR1
SYAMN

SEQ ID NO: 122 - protein, 19 aa, Artificial Sequence
  anti-CD3 HCDR2
RIRSKYNNYATYYADSVKS

SEQ ID NO: 123 - protein, 19 aa, Artificial Sequence
  anti-CD3 HCDR2
RIRSKYNNYATYYADSVKD

SEQ ID NO: 124 - protein, 19 aa, Artificial Sequence
  anti-CD3 HCDR2
RIRSKYNNYATYYADSVKG

SEQ ID NO: 125 - protein, 14 aa, Artificial Sequence
  anti-CD3 HCDR3
HGNFGNSYVSFFAY

SEQ ID NO: 126 - protein, 14 aa, Artificial Sequence
  anti-CD3 HCDR3
HGNFGNSYISYWAY

SEQ ID NO: 127 - protein, 14 aa, Artificial Sequence
  anti-CD3 HCDR3
HGNFGNSYLSFWAY

SEQ ID NO: 128 - protein, 5 aa, Artificial Sequence
  peptide linker
GGGGS

SEQ ID NO: 129 - protein, 25 aa, Artificial Sequence
  peptide linker
  SITE (6)..(10): may be present or absent
  SITE (11)..(15): may be present or absent
  SITE (16)..(20): may be present or absent
  SITE (21)..(25): may be present or absent
GGGGSGGGGSGGGGSGGGGSGGGGS

SEQ ID NO: 130 - protein, 6 aa, Artificial Sequence
  DE loop insertion peptide
GGGSGG

SEQ ID NO: 131 - protein, 216 aa, Artificial Sequence
  Modified immunoglobulin constant region
APEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 11 aa, Artificial Sequence
  Fc binding domain linker
SSLNTGTQPNS

SEQ ID NO: 133 - DNA, 342 bp, Artificial Sequence
  OMT1 variable light chain domain
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaa

SEQ ID NO: 134 - protein, 114 aa, Artificial Sequence; identical to SEQ ID NO: 16
  OMT1 variable light chain domain

SEQ ID NO: 135 - DNA, 372 bp, Artificial Sequence
  OMT1 variable heavy chain domain
gaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcttca

SEQ ID NO: 136 - protein, 124 aa, Artificial Sequence; identical to SEQ ID NO: 17
  OMT1 variable heavy chain domain

SEQ ID NO: 137 - DNA, 36 bp, Artificial Sequence
  OMT1 CDRL1
cacagtgttttatacagctccaacaataagaactac

SEQ ID NO: 138 - protein, 12 aa, Artificial Sequence
  OMT1 CDRL1
HSVLYSSNNKNY

SEQ ID NO: 139 - protein, 9 aa, Artificial Sequence
  OMT1 CDRL2
TGGGCATCT

SEQ ID NO: 140 - protein, 3 aa, Artificial Sequence
  OMT1 CDRL2
WAS

SEQ ID NO: 141 - DNA, 30 bp, Artificial Sequence
  OMT1 CDRL3
cagcaatattatagtactcctccgaccact

SEQ ID NO: 142 - protein, 10 aa, Artificial Sequence
  OMT1 CDRL3
QQYYSTPPTT

SEQ ID NO: 143 - DNA, 24 bp, Artificial Sequence
  OMT1 CDRH1
ggattcacctttagcagctatggc

SEQ ID NO: 144 - protein, 8 aa, Artificial Sequence
  OMT1 CDRH1
GFTFSSYG

SEQ ID NO: 145 - DNA, 24 bp, Artificial Sequence
  OMT1 CDRH2
attagtggtagtggtggtagcaca

SEQ ID NO: 146 - protein, 8 aa, Artificial Sequence
  OMT1 CDRH2
ISGSGGST

SEQ ID NO: 147 - DNA, 51 bp, Artificial Sequence
  OMT1 CDRH3
gcgaaagaaaagttacgatattttgactggttatccgatgcttttgatatc

SEQ ID NO: 148 - protein, 17 aa, Artificial Sequence
  OMT1 CDRH3
AKEKLRYFDWLSDAFDI

SEQ ID NO: 149 - DNA, 321 bp, Artificial Sequence
  DB8 variable light chain domain
gacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaa

SEQ ID NO: 150 - protein, 107 aa, Artificial Sequence [sequence omitted]

SEQ ID NO: 151 - DNA, 366 bp, Artificial Sequence
  DB8 variable heavychain domain
caggtgcagctggtgcagtctggggctgaggtgaagaagcctggggcctcagtgaaggtttcctgcaaggcatctggatacatcttcaccgactactatatgcactgggtgcgtcaggcccctggacaagggcttgagtggatgggatggatgagccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatgcggcggattacggtgactacgttgcttttgatatctggggccaagggacaatggtcaccgtctcttca

SEQ ID NO: 152 - protein, 122 aa, Artificial Sequence
  DB8 variable heavy chain domain
QVQLVQSGAEVKKPGASVKVSCKASGYIFTDYYMHWVRQAPGQGLEWMGWMSPNSGNTGYAQKFQGRVTMTRDTSTSTVYMELSSLRSEDTAVYYCARDAADYGDYVAFDIWGQGTMVTVSS

SEQ ID NO: 153 - DNA, 18 bp, Artificial Sequence
  DB8 CDR L1
cagagcattagcagctat

SEQ ID NO: 154 - protein, 6 aa, Artificial Sequence
  DB8 CDR L1
QSISSY

SEQ ID NO: 155 - DNA, 9 bp, Artificial Sequence
  DB8 CDR L2
gctgcatcc

SEQ ID NO: 156 - protein, 3 aa, Artificial Sequence
  DB8 CDR L2
AAS

SEQ ID NO: 157 - DNA, 27 bp, Artificial Sequence
  DB8 CDR L3
caacagagttacagtacccctctcact

SEQ ID NO: 158 - protein, 9 aa, Artificial Sequence
  DB8 CDR L3
QQSYSTPLT

SEQ ID NO: 159 - DNA, 24 bp, Artificial Sequence
  DB8 CDR H1
ggatacatcttcaccgactactat

SEQ ID NO: 160 - protein, 8 aa, Artificial Sequence
  DB8 CDR H1
GYIFTDYY

SEQ ID NO: 161 - DNA, 24 bp, Artificial Sequence
  DB8 CDR H2
atgagccctaacagtggtaacaca

SEQ ID NO: 162 - protein, 8 aa, Artificial Sequence
  DB8 CDR H2
MSPNSGNT

SEQ ID NO: 163 - DNA, 45 bp, Artificial Sequence
  DB8 CDR H3
gcgagagatgcggcggattacggtgactacgttgcttttgatatc

SEQ ID NO: 164 - protein, 15 aa, Artificial Sequence
  DB8 CDR H3
ARDAADYGDYVAFDI

SEQ ID NO: 165 - DNA, 321 bp, Artificial Sequence; identical to SEQ ID NO: 149
  DB60 variable light chain domain

SEQ ID NO: 166 - protein, 107 aa, Artificial Sequence [sequence omitted]

SEQ ID NO: 167 - DNA, 357 bp, Artificial Sequence
  DB60 variable heavy chain domain
caggtgcagctggtgcagtctggggctgaggtgaagaagcctggggcctcagtgaaggtttcctgcaaggcatctggatacaccttcaccagctactatatgcactgggtgcgtcaggcccctggacaagggcttgagtggatggggtggatcaaccctaacagtggtgacacaagctatgcacagaagttccagggccgtgtcaccatgacccgcgacacgtccacgagcacagtctacatggagctgagcagcctgcgttctgaggacacggccgtgtattactgtgcgcaggatagtagtggttccggggcttttgatatctggggccaagggacaatggtcaccgtctcttca

SEQ ID NO: 168 - protein, 119 aa, Artificial Sequence
  DB60 variable heavy chain domain
QVQLVQSGAEVKKPGASVKVSCKASGYTFTSYYMHWVRQAPGQGLEWMGWINPNSGDTSYAQKFQGRVTMTRDTSTSTVYMELSSLRSEDTAVYYCAQDSSGSGAFDIWGQGTMVTVSS

SEQ ID NO: 169 - DNA, 18 bp, Artificial Sequence
  DB60 CDR L1
cagagcattagcagctat

SEQ ID NO: 170 - protein, 6 aa, Artificial Sequence
  DB60 CDR L1
QSISSY

SEQ ID NO: 171 - DNA, 9 bp, Artificial Sequence
  DB60 CDR L2
gctgcatcc

SEQ ID NO: 172 - protein, 3 aa, Artificial Sequence
  DB60 CDR L2
AAS

SEQ ID NO: 173 - DNA, 27 bp, Artificial Sequence
  DB60 CDR L3
caacagagttacagtacccctctcact

SEQ ID NO: 174 - protein, 9 aa, Artificial Sequence
  DB60 CDR L3
QQSYSTPLT

SEQ ID NO: 175 - DNA, 24 bp, Artificial Sequence
  DB60 CDR H1
ggatacaccttcaccagctactat

SEQ ID NO: 176 - protein, 8 aa, Artificial Sequence
  DB60 CDR H1
GYTFTSYY

SEQ ID NO: 177 - DNA, 24 bp, Artificial Sequence
  DB60 CDR H2
atcaaccctaacagtggtgacaca

SEQ ID NO: 178 - protein, 8 aa, Artificial Sequence
  DB60 CDR H2
INPNSGDT

SEQ ID NO: 179 - DNA, 36 bp, Artificial Sequence
  DB60 CDR H3
gcgcaggatagtagtggttccggggcttttgatatc

SEQ ID NO: 180 - protein, 12 aa, Artificial Sequence
  DB60 CDR H3
AQDSSGSGAFDI

SEQ ID NO: 181 - DNA, 321 bp, Artificial Sequence; identical to SEQ ID NO: 149
  DB65 variable light chain domain

SEQ ID NO: 182 - protein, 107 aa, Artificial Sequence [sequence omitted]

SEQ ID NO: 183 - DNA, 369 bp, Artificial Sequence
  DB65 variable heavy chain domain
caggtgcagctggtgcagtctggggctgaggtgaagaagcctggggcctcagtgaaggtttcctgcaaggcatctggatacaccttcaccggctactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgaaagaggaaccgatttttggagtggttatggatgcttttgatatctggggccaagggacaatggtcaccgtctcctca

SEQ ID NO: 184 - protein, 123 aa, Artificial Sequence
  DB65 variable heavy chain domain
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCAKEEPIFGVVMDAFDIWGQGTMVTVSS

SEQ ID NO: 185 - DNA, 18 bp, Artificial Sequence
  DB65 CDR L1
cagagcattagcagctat

SEQ ID NO: 186 - protein, 6 aa, Artificial Sequence
  DB65 CDR L1
QSISSY

SEQ ID NO: 187 - DNA, 9 bp, Artificial Sequence
  DB65 CDR L2
gctgcatcc

SEQ ID NO: 188 - protein, 3 aa, Artificial Sequence
  DB65 CDR L2
AAS

SEQ ID NO: 189 - DNA, 27 bp, Artificial Sequence
  DB65 CDR L3
caacagagttacagtacccctctcact

SEQ ID NO: 190 - protein, 9 aa, Artificial Sequence
  DB65 CDR L3
QQSYSTPLT

SEQ ID NO: 191 - DNA, 24 bp, Artificial Sequence
  DB65 CDR H1
ggatacaccttcaccggctactat

SEQ ID NO: 192 - protein, 8 aa, Artificial Sequence
  DB65 CDR H1
GYTFTGYY

SEQ ID NO: 193 - DNA, 24 bp, Artificial Sequence
  DB65 CDR H2
atgaaccctaacagtggtaacaca

SEQ ID NO: 194 - protein, 8 aa, Artificial Sequence
  DB65 CDR H2
MNPNSGNT

SEQ ID NO: 195 - DNA, 48 bp, Artificial Sequence
  DB65 CDR H3
gcgaaagaggaaccgatttttggagtggttatggatgcttttgatatc

SEQ ID NO: 196 - protein, 16 aa, Artificial Sequence
  DB65 CDR H3
AKEEPIFGVVMDAFDI

SEQ ID NO: 197 - DNA, 321 bp, Artificial Sequence
  DB82 variable light chain domain
gacatccagatgacccagtctccatcctccctgtctgcatctgtaggagaccgcgtcaccatcacttgccgggcaagtcagaccataaacaactatttgaactggtatcagcagaaaccagggaaagcccctaagctcctgatctattctgcatctactttgcaaagtggggtcccatcacgtttcagtggcagtggatctgggacagatttcactctcaccatcagcagtctgcaacctgaagattttgcaacttactactgtcaccagagttacacttcacctctcactttcggcggaggtaccaaggtggagatcaaa

SEQ ID NO: 198 - protein, 107 aa, Artificial Sequence
  DB82 variable light chain domain
DIQMTQSPSSLSASVGDRVTITCRASQTINNYLNWYQQKPGKAPKLLIYSASTLQSGVPSRFSGSGSGTDFTLTISSLQPEDFATYYCHQSYTSPLTFGGGTKVEIK

SEQ ID NO: 199 - DNA, 363 bp, Artificial Sequence
  DB82 variable heavy chain domain
gaggtgcagctggtggagtctgggggaggcttggtacagcctggggggtccctgcgcctctcctgtgcagcctctggattcacctttagcagctatgccatgagctgggtccgccaggctccagggaaggggctggagtgggtctcagttattagtgccaatagtgctggtctaggccatgcggactctgtgaagggccggttcaccatctcccgcgacaattccaagaacacgctgtatctgcaaatgaacagcctgcgcgccgaggacacggccgtatattactgtgcgagagtgggctatagcagctcggctgatgcttttgatatctggggccaagggacaatggtcaccgtctcctcg

SEQ ID NO: 200 - protein, 121 aa, Artificial Sequence
  DB82 variable heavy chain domain
EVQLVESGGGLVQPGGSLRLSCAASGFTFSSYAMSWVRQAPGKGLEWVSVISANSAGLGHADSVKGRFTISRDNSKNTLYLQMNSLRAEDTAVYYCARVGYSSSADAFDIWGQGTMVTVSS

SEQ ID NO: 201 - DNA, 18 bp, Artificial Sequence
  DB82 CDR L1
cagaccataaacaactat

SEQ ID NO: 202 - protein, 6 aa, Artificial Sequence
  DB82 CDR L1
QTINNY

SEQ ID NO: 203 - DNA, 9 bp, Artificial Sequence
  DB82 CDR L2
tctgcatct

SEQ ID NO: 204 - protein, 3 aa, Artificial Sequence
  DB82 CDR L2
SAS

SEQ ID NO: 205 - DNA, 27 bp, Artificial Sequence
  DB82 CDR L3
caccagagttacacttcacctctcact

SEQ ID NO: 206 - protein, 9 aa, Artificial Sequence
  DB82 CDR L3
HQSYTSPLT

SEQ ID NO: 207 - DNA, 24 bp, Artificial Sequence
  DB82 CDR H1
ggattcacctttagcagctatgcc

SEQ ID NO: 208 - protein, 8 aa, Artificial Sequence
  DB82 CDR H1
GFTFSSYA

SEQ ID NO: 209 - DNA, 24 bp, Artificial Sequence
  DB82 CDR H2
attagtgccaatagtgctggtcta

SEQ ID NO: 210 - protein, 8 aa, Artificial Sequence
  DB82 CDR H2
ISANSAGL

SEQ ID NO: 211 - DNA, 42 bp, Artificial Sequence
  DB82 CDR H3
gcgagagtgggctatagcagctcggctgatgcttttgatatc

SEQ ID NO: 212 - protein, 14 aa, Artificial Sequence
  DB82 CDR H3
ARVGYSSSADAFDI

SEQ ID NO: 213 - DNA, 336 bp, Artificial Sequence
  DB83 variable light chain domain
gatgttgtgatgactcagtctccactctccctgcccgtcacccctggagagccggcctccatctcctgcaggtctagtcagagcctcctgcatagtaatggagacaactatttggattggtacctgcagaagccagggcagtctccacagctcctgatctatttgggttctaatcgggcctccggggtccctgaccgtttcagtggcagtggatcaggcacagattttacactgaaaatcagccgtgtggaggctgaggatgttggggtttattactgcatgcaagctacacactggccactcactttcggccctggtaccaaagtggatatcaaa

SEQ ID NO: 214 - protein, 112 aa, Artificial Sequence
  DB83 variable light chain domain
DVVMTQSPLSLPVTPGEPASISCRSSQSLLHSNGDNYLDWYLQKPGQSPQLLIYLGSNRASGVPDRFSGSGSGTDFTLKISRVEAEDVGVYYCMQATHWPLTFGPGTKVDIK

SEQ ID NO: 215 - DNA, 375 bp, Artificial Sequence
  DB83 variable heavy chain domain
caggtgcagctggtgcagtctggggctgaggtgaagaagcctggggcctcagtgaaggtttcctgcaaggcatctggatacaccttcactagctatgctatgcattgggtgcgtcaggcccctggacaagggcttgagtggatgggacttgttgatcctgaagatggtgaaacaatatatgcagagaagttccagggccgtgtcaccatgacccgcgacacgtccacgagcacagtctacatggagctgagcagcctgcgttctgaggacacggccgtgtattactgtgcgagacgaacgtattactatgatagtagtggttcccgttatgcttttgatatctggggccaagggaccacggtcaccgtctcttca

SEQ ID NO: 216 - protein, 125 aa, Artificial Sequence
  DB83 variable heavy chain domain
QVQLVQSGAEVKKPGASVKVSCKASGYTFTSYAMHWVRQAPGQGLEWMGLVDPEDGETIYAEKFQGRVTMTRDTSTSTVYMELSSLRSEDTAVYYCARRTYYYDSSGSRYAFDIWGQGTTVTVSS

SEQ ID NO: 217 - DNA, 33 bp, Artificial Sequence
  DB83 CDR L1
cagagcctcctgcatagtaatggagacaactat

SEQ ID NO: 218 - protein, 11 aa, Artificial Sequence
  DB83 CDR L1
QSLLHSNGDNY

SEQ ID NO: 219 - DNA, 9 bp, Artificial Sequence
  DB83 CDR L2
ttgggttct

SEQ ID NO: 220 - protein, 3 aa, Artificial Sequence
  DB83 CDR L2
LGS

SEQ ID NO: 221 - DNA, 27 bp, Artificial Sequence
  DB83 CDR L3
atgcaagctacacactggccactcact

SEQ ID NO: 222 - protein, 9 aa, Artificial Sequence
  DB83 CDR L3
MQATHWPLT

SEQ ID NO: 223 - DNA, 24 bp, Artificial Sequence
  DB83 CDR H1
ggatacaccttcactagctatgct

SEQ ID NO: 224 - protein, 8 aa, Artificial Sequence
  DB83 CDR H1
GYTFTSYA

SEQ ID NO: 225 - DNA, 24 bp, Artificial Sequence
  DB83 CDR H2
gttgatcctgaagatggtgaaaca

SEQ ID NO: 226 - protein, 8 aa, Artificial Sequence
  DB83 CDR H2
VDPEDGET

SEQ ID NO: 227 - DNA, 54 bp, Artificial Sequence
  DB83 CDR H3
gcgagacgaacgtattactatgatagtagtggttcccgttatgcttttgatatc

SEQ ID NO: 228 - protein, 18 aa, Artificial Sequence
  DB83 CDR H3
ARRTYYYDSSGSRYAFDI

SEQ ID NO: 229 - DNA, 321 bp, Artificial Sequence
  DB86 variable light chain domain
gacatccagatgacccagtctccatcctccctgtctgcatctgtaggagaccgcgtcaccatcacttgccgggcaagtcagggcatcagaaatgatttaggttggtatcagcagaaaccagggaaagcccctaagctcctgatctatgctgcatccactttgcaatcaggggtcccatcacgtttcagtggcagtggatctgggacagatttcactctcaccatcagcagtctgcaacctgaagattttgcaacttactactgtcaacagagttacggtgcccccctcactttcggcggaggtaccaaggtggagatcaaa

SEQ ID NO: 230 - protein, 107 aa, Artificial Sequence
  DB86 variable light chain domain
DIQMTQSPSSLSASVGDRVTITCRASQGIRNDLGWYQQKPGKAPKLLIYAASTLQSGVPSRFSGSGSGTDFTLTISSLQPEDFATYYCQQSYGAPLTFGGGTKVEIK

SEQ ID NO: 231 - DNA, 360 bp, Artificial Sequence
  DB86 variable heavy chain domain
caggtgcagctggtgcagtctggggctgaggtgaagaagcctggggcctcagtgaaggtttcctgcaaggcatctggatatatgttcagtggccattctgcacactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatagcagtggctggtacgatgtctttgactactggggccaggggaccctggtcaccgtctcctca

SEQ ID NO: 232 - protein, 120 aa, Artificial Sequence
  DB86 variable heavy chain domain
QVQLVQSGAEVKKPGASVKVSCKASGYMFSGHSAHWVRQAPGQGLEWMGWMNPNSGNTGYAQKFQGRVTMTRDTSTSTVYMELSSLRSEDTAVYYCARDSSGWYDVFDYWGQGTLVTVSS

SEQ ID NO: 233 - DNA, 18 bp, Artificial Sequence
  DB86 CDR L1
cagggcatcagaaatgat

SEQ ID NO: 234 - protein, 6 aa, Artificial Sequence
  DB86 CDR L1
QGIRND

SEQ ID NO: 235 - DNA, 9 bp, Artificial Sequence
  DB86 CDR L2
gctgcatcc

SEQ ID NO: 236 - protein, 3 aa, Artificial Sequence
  DB86 CDR L2
AAS

SEQ ID NO: 237 - DNA, 24 bp, Artificial Sequence
  DB86 CDR L3
caacagagttacggtgcccccctc

SEQ ID NO: 238 - protein, 9 aa, Artificial Sequence
  DB86 CDR L3
QQSYGAPLT

SEQ ID NO: 239 - DNA, 24 bp, Artificial Sequence
  DB86 CDR H1
ggatatatgttcagtggccattct

SEQ ID NO: 240 - protein, 8 aa, Artificial Sequence
  DB86 CDR H1
GYMFSGHS

SEQ ID NO: 241 - DNA, 24 bp, Artificial Sequence
  DB86 CDR H2
atgaaccctaacagtggtaacaca

SEQ ID NO: 242 - protein, 8 aa, Artificial Sequence
  DB86 CDR H2
MNPNSGNT

SEQ ID NO: 243 - DNA, 39 bp, Artificial Sequence
  DB86 CDR H3
gcgagagatagcagtggctggtacgatgtctttgactac

SEQ ID NO: 244 - protein, 13 aa, Artificial Sequence
  DB86 CDR H3
ARDSSGWYDVFDY

SEQ ID NO: 245 - DNA, 321 bp, Artificial Sequence; identical to SEQ ID NO: 149
  DB280 variable light chain domain

SEQ ID NO: 246 - protein, 107 aa, Artificial Sequence [sequence omitted]

SEQ ID NO: 247 - DNA, 378 bp, Artificial Sequence
  DB280 variable heavy chain domain
caggtgcagctggtgcagtctggggctgaggtgaagaagcctggggcctcagtgaaggtttcctgcaaggcatctggatacagcctcaacttatactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgagcctcgattgtagtggtggtagctgctactccgaatatgatgcttttgatatctggggccaagggaccacggtcaccgtctcctca

SEQ ID NO: 248 - protein, 126 aa, Artificial Sequence
  DB280 variable heavy chain domain
QVQLVQSGAEVKKPGASVKVSCKASGYSLNLYYMHWVRQAPGQGLEWMGWMNPNSGNTGYAQKFQGRVTMTRDTSTSTVYMELSSLRSEDTAVYYCASLDCSGGSCYSEYDAFDIWGQGTTVTVSS

SEQ ID NO: 249 - DNA, 18 bp, Artificial Sequence
  DB280 CDR L1
cagagcattagcagctat

SEQ ID NO: 250 - protein, 6 aa, Artificial Sequence
  DB280 CDR L1
QSISSY

SEQ ID NO: 251 - DNA, 9 bp, Artificial Sequence
  DB280 CDR L2
gctgcatcc

SEQ ID NO: 252 - protein, 3 aa, Artificial Sequence
  DB280 CDR L2
AAS

SEQ ID NO: 253 - DNA, 27 bp, Artificial Sequence
  DB280 CDR L3
caacagagttacagtacccctctcact

SEQ ID NO: 254 - protein, 9 aa, Artificial Sequence
  DB280 CDR L3
QQSYSTPLT

SEQ ID NO: 255 - DNA, 24 bp, Artificial Sequence
  DB280 CDR H1
ggatacagcctcaacttatactat

SEQ ID NO: 256 - protein, 8 aa, Artificial Sequence
  DB280 CDR H1
GYSLNLYY

SEQ ID NO: 257 - DNA, 24 bp, Artificial Sequence
  DB280 CDR H2
atgaaccctaacagtggtaacaca

SEQ ID NO: 258 - protein, 8 aa, Artificial Sequence
  DB280 CDR H2
MNPNSGNT

SEQ ID NO: 259 - DNA, 57 bp, Artificial Sequence
  DB280 CDR H3
gcgagcctcgattgtagtggtggtagctgctactccgaatatgatgcttttgatatc

SEQ ID NO: 260 - protein, 19 aa, Artificial Sequence
  DB280 CDR H3
ASLDCSGGSCYSEYDAFDI

SEQ ID NO: 261 - DNA, 321 bp, Artificial Sequence; identical to SEQ ID NO: 149
  DB331 variable light chain domain

SEQ ID NO: 262 - protein, 107 aa, Artificial Sequence [sequence omitted]

SEQ ID NO: 263 - DNA, 357 bp, Artificial Sequence
  DB331 variable heavy chain domain
caggtgcagctggtgcagtctggggctgaggtgaagaagcctggggcctcagtgaaggtttcctgcaaggcatctggatacaccttcaccagctactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcaacagatctcgcgggggaagccttgttcgacccctggggccagggcaccctggtcaccgtctcctca

SEQ ID NO: 264 - protein, 119 aa, Artificial Sequence
  DB331 variable heavy chain domain
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCATDLAGEALFDPWGQGTLVTVSS

SEQ ID NO: 265 - DNA, 18 bp, Artificial Sequence
  DB331 CDR L1
cagagcattagcagctat

SEQ ID NO: 266 - protein, 6 aa, Artificial Sequence
  DB331 CDR L1
QSISSY

SEQ ID NO: 267 - DNA, 9 bp, Artificial Sequence
  DB331 CDR L2
gctgcatcc

SEQ ID NO: 268 - protein, 3 aa, Artificial Sequence
  DB331 CDR L2
AAS

SEQ ID NO: 269 - DNA, 27 bp, Artificial Sequence
  DB331 CDR L3
caacagagttacagtacccctctcact

SEQ ID NO: 270 - protein, 9 aa, Artificial Sequence
  DB331 CDR L3
QQSYSTPLT

SEQ ID NO: 271 - DNA, 24 bp, Artificial Sequence
  DB331 CDR H1
ggatacaccttcaccagctactat

SEQ ID NO: 272 - protein, 8 aa, Artificial Sequence
  DB331 CDR H1
GYTFTSYY

SEQ ID NO: 273 - DNA, 24 bp, Artificial Sequence
  DB331 CDR H2
atgaaccctaacagtggtaacaca

SEQ ID NO: 274 - protein, 8 aa, Artificial Sequence
  DB331 CDR H2
MNPNSGNT

SEQ ID NO: 275 - DNA, 36 bp, Artificial Sequence
  DB331 CDR H3
gcaacagatctcgcgggggaagccttgttcgacccc

SEQ ID NO: 276 - protein, 12 aa, Artificial Sequence
  DB331 CDR H3
ATDLAGEALFDP

SEQ ID NO: 277 - DNA, 321 bp, Artificial Sequence; identical to SEQ ID NO: 149
  DB415 variable light chain domain

SEQ ID NO: 278 - protein, 107 aa, Artificial Sequence [sequence omitted]

SEQ ID NO: 279 - DNA, 351 bp, Artificial Sequence
  DB415 variable heavy chain domain
gaggtgcagctggtggagtctgggggaggcttggtacagcctggggggtccctgcgcctctcctgtgcagcctctggaatcaccttcagtagttatggcatgcattgggtccgccaggctccagggaaggggctggagtgggtctcaggtattagttggaatagtggtaacagagtctatgtggactctgtgaagggccggttcaccatctcccgcgacaattccaagaacacgctgtatctgcaaatgaacagcctgcgcgccgaggacacggccgtatattactgtgcgagagatactaatgatgcttttgatatctggggccaagggaccacggtcaccgtctcctca

SEQ ID NO: 280 - protein, 117 aa, Artificial Sequence
  DB415 variable heavy chain domain
EVQLVESGGGLVQPGGSLRLSCAASGITFSSYGMHWVRQAPGKGLEWVSGISWNSGNRVYVDSVKGRFTISRDNSKNTLYLQMNSLRAEDTAVYYCARDTNDAFDIWGQGTTVTVSS

SEQ ID NO: 281 - DNA, 18 bp, Artificial Sequence
  DB415 CDR L1
cagagcattagcagctat

SEQ ID NO: 282 - protein, 6 aa, Artificial Sequence
  DB415 CDR L1
QSISSY

SEQ ID NO: 283 - DNA, 9 bp, Artificial Sequence
  DB415 CDR L2
gctgcatcc

SEQ ID NO: 284 - protein, 3 aa, Artificial Sequence
  DB415 CDR L2
AAS

SEQ ID NO: 285 - DNA, 27 bp, Artificial Sequence
  DB415 CDR L3
caacagagttacagtacccctctcact

SEQ ID NO: 286 - protein, 6 aa, Artificial Sequence
  DB415 CDR L3
QSISSY

SEQ ID NO: 287 - DNA, 24 bp, Artificial Sequence
  DB415 CDR H1
ggaatcaccttcagtagttatggc

SEQ ID NO: 288 - protein, 8 aa, Artificial Sequence
  DB415 CDR H1
GITFSSYG

SEQ ID NO: 289 - DNA, 24 bp, Artificial Sequence
  DB415 CDR H2
attagttggaatagtggtaacaga

SEQ ID NO: 290 - protein, 8 aa, Artificial Sequence
  DB415 CDR H2
ISWNSGNR

SEQ ID NO: 291 - DNA, 30 bp, Artificial Sequence
  DB415 CDR H3
gcgagagatactaatgatgcttttgatatc

SEQ ID NO: 292 - protein, 10 aa, Artificial Sequence
  DB415 CDR H3
ARDTNDAFDI

SEQ ID NO: 293 - DNA, 321 bp, Artificial Sequence; identical to SEQ ID NO: 149
  DB435 variable light chain domain

SEQ ID NO: 294 - protein, 107 aa, Artificial Sequence [sequence omitted]

SEQ ID NO: 295 - DNA, 357 bp, Artificial Sequence
  DB435 variable heavy chain domain
caggtgcagctggtgcagtctggggctgaggtgaagaagcctggggcctcagtgaaggtttcctgcaaggcatctggaggcaccttcagcagctatgctatcagctgggtgcgtcaggcccctggacaagggcttgagtggatgggctggatcacccctcacaatggtaacataaagtatgcacgggagttccagggccgtgtcaccatgacccgcgacacgtccacgagcacagtctacatggagctgagcagcctgcgttctgaggacacggccgtgtattactgtgcgaaagatctgaactggaacgcagcctttgactactggggccaggggaccctggtcaccgtctcctca

SEQ ID NO: 296 - protein, 119 aa, Artificial Sequence
  DB435 variable heavy chain domain
QVQLVQSGAEVKKPGASVKVSCKASGGTFSSYAISWVRQAPGQGLEWMGWITPHNGNIKYAREFQGRVTMTRDTSTSTVYMELSSLRSEDTAVYYCAKDLNWNAAFDYWGQGTLVTVSS

SEQ ID NO: 297 - DNA, 18 bp, Artificial Sequence
  DB435 CDR L1
cagagcattagcagctat

SEQ ID NO: 298 - protein, 6 aa, Artificial Sequence
  DB435 CDR L1
QSISSY

SEQ ID NO: 299 - DNA, 9 bp, Artificial Sequence
  DB435 CDR L2
gctgcatcc

SEQ ID NO: 300 - protein, 3 aa, Artificial Sequence
  DB435 CDR L2
AAS

SEQ ID NO: 301 - DNA, 27 bp, Artificial Sequence
  DB435 CDR L3
caacagagttacagtacccctctcact

SEQ ID NO: 302 - protein, 9 aa, Artificial Sequence
  DB435 CDR L3
QQSYSTPLT

SEQ ID NO: 303 - DNA, 24 bp, Artificial Sequence
  DB435 CDR H1
ggaggcaccttcagcagctatgct

SEQ ID NO: 304 - protein, 8 aa, Artificial Sequence
  DB435 CDR H1
GGTFSSYA

SEQ ID NO: 305 - DNA, 24 bp, Artificial Sequence
  DB435 CDR H2
atcacccctcacaatggtaacata

SEQ ID NO: 306 - protein, 8 aa, Artificial Sequence
  DB435 CDR H2
ITPHNGNI

SEQ ID NO: 307 - DNA, 36 bp, Artificial Sequence
  DB435 CDR H3
gcgaaagatctgaactggaacgcagcctttgactac

SEQ ID NO: 308 - protein, 12 aa, Artificial Sequence
  DB435 CDR H3
AKDLNWNAAFDY

SEQ ID NO: 309 - DNA, 2340 bp, Artificial Sequence
  humanized bispecific construct TRI129
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcttcaggtggaggcggttcaggcggaggtggatccggcggtggcggctccggtggcggcggatctgacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 310 - protein, 759 aa, Artificial Sequence
  humanized bispecific construct TRI129
EVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSGGGGSGGGGSGGGGSGGGGSDIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 311 - DNA, 2334 bp, Artificial Sequence
  humanized bispecific construct TRI130
atggaagcaccagcgcagcttctcttcctcctgctactctggctcccagataccaccggtgacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 312 - protein, 757 aa, Artificial Sequence
  humanized bispecific construct TRI130
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 313 - DNA, 2313 bp, Artificial Sequence
  humanized bispecific construct TRI123
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacatcttcaccgactactatatgcactgggtgcgtcaggcccctggacaagggcttgagtggatgggatggatgagccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatgcggcggattacggtgactacgttgcttttgatatctggggccaagggacaatggtcaccgtctcttcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 314 - protein, 750 aa, Artificial Sequence
  humanized bispecific construct TRI123
QVQLVQSGAEVKKPGASVKVSCKASGYIFTDYYMHWVRQAPGQGLEWMGWMSPNSGNTGYAQKFQGRVTMTRDTSTSTVYMELSSLRSEDTAVYYCARDAADYGDYVAFDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 315 - DNA, 2307 bp, Artificial Sequence
  humanized bispecific construct TRI124
atggaagcaccagcgcagcttctcttcctcctgctactctggctcccagataccaccggtgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaaggcggcggcggcagcggcggcggcggcagcggcggcggaggctccggcggcggcggcagccaggtgcagctggtgcagtctggggctgaggtgaagaagcctggggcctcagtgaaggtttcctgcaaggcatctggatacatcttcaccgactactatatgcactgggtgcgtcaggcccctggacaagggcttgagtggatgggatggatgagccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatgcggcggattacggtgactacgttgcttttgatatctggggccaagggacaatggtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 316 - protein, 748 aa, Artificial Sequence
  humanized bispecific construct TRI124
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGASVKVSCKASGYIFTDYYMHWVRQAPGQGLEWMGWMSPNSGNTGYAQKFQGRVTMTRDTSTSTVYMELSSLRSEDTAVYYCARDAADYGDYVAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 317 - DNA, 2304 bp, Artificial Sequence
  humanized bispecific construct TRI139
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggaggcaccttcagcagctatgctatcagctgggtgcgtcaggcccctggacaagggcttgagtggatgggctggatcacccctcacaatggtaacataaagtatgcacgggagttccagggccgtgtcaccatgacccgcgacacgtccacgagcacagtctacatggagctgagcagcctgcgttctgaggacacggccgtgtattactgtgcgaaagatctgaactggaacgcagcctttgactactggggccaggggaccctggtcaccgtctcctcaggtggaggcggttcaggcggaggtggatccggcggtggcggctccggtggcggcggatctgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactggagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcatga

SEQ ID NO: 318 - protein, 747 aa, Artificial Sequence
  humanized bispecific construct TRI139
QVQLVQSGAEVKKPGASVKVSCKASGGTFSSYAISWVRQAPGQGLEWMGWITPHNGNIKYAREFQGRVTMTRDTSTSTVYMELSSLRSEDTAVYYCAKDLNWNAAFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 319 - DNA, 2313 bp, Artificial Sequence
  humanized bispecific construct TRI137
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacatcttcaccgactactatatgcactgggtgcgtcaggcccctggacaagggcttgagtggatgggatggatgagccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatgcggcggattacggtgactacgttgcttttgatatctggggccaagggacaatggtcaccgtctcttcaggtggaggcggttcaggcggaggtggatccggcggtggcggctccggtggcggcggatctgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcatga

SEQ ID NO: 320 - protein, 750 aa, Artificial Sequence; identical to SEQ ID NO: 314
  humanized bispecific construct TRI137

SEQ ID NO: 321 - DNA, 2304 bp, Artificial Sequence
  humanized bispecific construct TRI125
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacaccttcaccagctactatatgcactgggtgcgtcaggcccctggacaagggcttgagtggatggggtggatcaaccctaacagtggtgacacaagctatgcacagaagttccagggccgtgtcaccatgacccgcgacacgtccacgagcacagtctacatggagctgagcagcctgcgttctgaggacacggccgtgtattactgtgcgcaggatagtagtggttccggggcttttgatatctggggccaagggacaatggtcaccgtctcttcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 322 - protein, 747 aa, Artificial Sequence
  humanized bispecific construct TRI125
QVQLVQSGAEVKKPGASVKVSCKASGYTFTSYYMHWVRQAPGQGLEWMGWINPNSGDTSYAQKFQGRVTMTRDTSTSTVYMELSSLRSEDTAVYYCAQDSSGSGAFDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 323 - DNA, 2316 bp, Artificial Sequence
  humanized bispecific construct TRI126
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacaccttcaccggctactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgaaagaggaaccgatttttggagtggttatggatgcttttgatatctggggccaagggacaatggtcaccgtctcctcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 324 - protein, 751 aa, Artificial Sequence
  humanized bispecific construct TRI126
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCAKEEPIFGVVMDAFDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 325 - DNA, 2304 bp, Artificial Sequence
  humanized bispecific construct TRI127
atggaagcaccagcgcagcttctcttcctcctgctactctggctcccagataccaccggtgacatccagatgacccagtctccatcctccctgtctgcatctgtaggagaccgcgtcaccatcacttgccgggcaagtcagaccataaacaactatttgaactggtatcagcagaaaccagggaaagcccctaagctcctgatctattctgcatctactttgcaaagtggggtcccatcacgtttcagtggcagtggatctgggacagatttcactctcaccatcagcagtctgcaacctgaagattttgcaacttactactgtcaccagagttacacttcacctctcactttcggcggaggtaccaaggtggagatcaaaggcggcggcggcagcggcggcggcggcagcggcggcggaggctccggcggcggcggcagcgaggtgcagctggtggagtctgggggaggcttggtacagcctggggggtccctgcgcctctcctgtgcagcctctggattcacctttagcagctatgccatgagctgggtccgccaggctccagggaaggggctggagtgggtctcagttattagtgccaatagtgctggtctaggccatgcggactctgtgaagggccggttcaccatctcccgcgacaattccaagaacacgctgtatctgcaaatgaacagcctgcgcgccgaggacacggccgtatattactgtgcgagagtgggctatagcagctcggctgatgcttttgatatctggggccaagggacaatggtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 326 - protein, 747 aa, Artificial Sequence
  humanized bispecific construct TRI127
DIQMTQSPSSLSASVGDRVTITCRASQTINNYLNWYQQKPGKAPKLLIYSASTLQSGVPSRFSGSGSGTDFTLTISSLQPEDFATYYCHQSYTSPLTFGGGTKVEIKGGGGSGGGGSGGGGSGGGGSEVQLVESGGGLVQPGGSLRLSCAASGFTFSSYAMSWVRQAPGKGLEWVSVISANSAGLGHADSVKGRFTISRDNSKNTLYLQMNSLRAEDTAVYYCARVGYSSSADAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 327 - DNA, 2325 bp, Artificial Sequence
  humanized bispecific construct TRI131
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacagcctcaacttatactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgagcctcgattgtagtggtggtagctgctactccgaatatgatgcttttgatatctggggccaagggaccacggtcaccgtctcctcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 328 - protein, 754 aa, Artificial Sequence
  humanized bispecific construct TRI131
QVQLVQSGAEVKKPGASVKVSCKASGYSLNLYYMHWVRQAPGQGLEWMGWMNPNSGNTGYAQKFQGRVTMTRDTSTSTVYMELSSLRSEDTAVYYCASLDCSGGSCYSEYDAFDIWGQGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 329 - DNA, 2304 bp, Artificial Sequence
  humanized bispecific construct TRI132
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacaccttcaccagctactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcaacagatctcgcgggggaagccttgttcgacccctggggccagggcaccctggtcaccgtctcctcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 330 - protein, 747 aa, Artificial Sequence
  humanized bispecific construct TRI132
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCATDLAGEALFDPWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 331 - DNA, 2337 bp, Artificial Sequence
  humanized bispecific construct TRI134
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacaccttcactagctatgctatgcattgggtgcgtcaggcccctggacaagggcttgagtggatgggacttgttgatcctgaagatggtgaaacaatatatgcagagaagttccagggccgtgtcaccatgacccgcgacacgtccacgagcacagtctacatggagctgagcagcctgcgttctgaggacacggccgtgtattactgtgcgagacgaacgtattactatgatagtagtggttcccgttatgcttttgatatctggggccaagggaccacggtcaccgtctcttcaggcggcggcggcagcggcggcggcggcagcggcggcggaggctccggcggcggcggcagcgatgttgtgatgactcagtctccactctccctgcccgtcacccctggagagccggcctccatctcctgcaggtctagtcagagcctcctgcatagtaatggagacaactatttggattggtacctgcagaagccagggcagtctccacagctcctgatctatttgggttctaatcgggcctccggggtccctgaccgtttcagtggcagtggatcaggcacagattttacactgaaaatcagccgtgtggaggctgaggatgttggggtttattactgcatgcaagctacacactggccactcactttcggccctggtaccaaagtggatatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 332 - protein, 758 aa, Artificial Sequence
  humanized bispecific construct TRI134
QVQLVQSGAEVKKPGASVKVSCKASGYTFTSYAMHWVRQAPGQGLEWMGLVDPEDGETIYAEKFQGRVTMTRDTSTSTVYMELSSLRSEDTAVYYCARRTYYYDSSGSRYAFDIWGQGTTVTVSSGGGGSGGGGSGGGGSGGGGSDVVMTQSPLSLPVTPGEPASISCRSSQSLLHSNGDNYLDWYLQKPGQSPQLLIYLGSNRASGVPDRFSGSGSGTDFTLKISRVEAEDVGVYYCMQATHWPLTFGPGTKVDIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 333 - DNA, 2307 bp, Artificial Sequence
  humanized bispecific construct TRI128
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatatatgttcagtggccattctgcacactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatagcagtggctggtacgatgtctttgactactggggccaggggaccctggtcaccgtctcctcaggtggaggcggttcaggcggaggtggatccggcggtggcggctccggtggcggcggatctgacatccagatgacccagtctccatcctccctgtctgcatctgtaggagaccgcgtcaccatcacttgccgggcaagtcagggcatcagaaatgatttaggttggtatcagcagaaaccagggaaagcccctaagctcctgatctatgctgcatccactttgcaatcaggggtcccatcacgtttcagtggcagtggatctgggacagatttcactctcaccatcagcagtctgcaacctgaagattttgcaacttactactgtcaacagagttacggtgcccccctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 334 - protein, 748 aa, Artificial Sequence
  humanized bispecific construct TRI128
QVQLVQSGAEVKKPGASVKVSCKASGYMFSGHSAHWVRQAPGQGLEWMGWMNPNSGNTGYAQKFQGRVTMTRDTSTSTVYMELSSLRSEDTAVYYCARDSSGWYDVFDYWGQGTLVTVSSGGGGSGGGGSGGGGSGGGGSDIQMTQSPSSLSASVGDRVTITCRASQGIRNDLGWYQQKPGKAPKLLIYAASTLQSGVPSRFSGSGSGTDFTLTISSLQPEDFATYYCQQSYGAPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 335 - DNA, 2298 bp, Artificial Sequence
  humanized bispecific construct TRI138
atggaagcaccagcgcagcttctcttcctcctgctactctggctcccagataccaccggtgaggtgcagctggtggagtctgggggaggcttggtacagcctggggggtccctgcgcctctcctgtgcagcctctggaatcaccttcagtagttatggcatgcattgggtccgccaggctccagggaaggggctggagtgggtctcaggtattagttggaatagtggtaacagagtctatgtggactctgtgaagggccggttcaccatctcccgcgacaattccaagaacacgctgtatctgcaaatgaacagcctgcgcgccgaggacacggccgtatattactgtgcgagagatactaatgatgcttttgatatctggggccaagggaccacggtcaccgtctcctcaggtggaggcggttcaggcggaggtggatccggcggtggcggctccggtggcggcggatctgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactggagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcatga

SEQ ID NO: 336 - protein, 745 aa, Artificial Sequence
  humanized bispecific construct TRI138
EVQLVESGGGLVQPGGSLRLSCAASGITFSSYGMHWVRQAPGKGLEWVSGISWNSGNRVYVDSVKGRFTISRDNSKNTLYLQMNSLRAEDTAVYYCARDTNDAFDIWGQGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 337 - protein, 777 aa, Artificial Sequence; identical to SEQ ID NO: 31
  humanized bispecific construct TRI130

SEQ ID NO: 338 - protein, 784 aa, Artificial Sequence
  humanized bispecific construct TRI185
MEAPAQLLFLLLLWLPDTTGDIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLRS

SEQ ID NO: 339 - protein, 292 aa, Artificial Sequence
  TRI168 - dual-affinity re-targeting molecule chain 1
MEAPAQLLFLLLLWLPDTTGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGEVAALEKEVAALEKEVAALEKEVAALEK

SEQ ID NO: 340 - protein, 331 aa, Artificial Sequence
  TRI168 - dual-affinity re-targeting molecule chain 2
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGKVAALKEKVAALKEKVAALKEKVAALKESSSLNDIFEAQKIEWHEDYKDDDDKDYKDDDDKDYKDDDDKHHHHHHHHHH

SEQ ID NO: 341 - protein, 107 aa, Artificial Sequence
  Cris-7 variable light chain sequence
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITR

SEQ ID NO: 342 - protein, 121 aa, Artificial Sequence
  Cris-7 variable heavy chain sequence
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 343 - protein, 106 aa, Artificial Sequence [sequence omitted]

SEQ ID NO: 344 - protein, 120 aa, Artificial Sequence [sequence omitted]

SEQ ID NO: 345 - protein, 5 aa, Artificial Sequence
  Cris7 and DRA222 VH CDR1 (Kabat)
RSTMH

SEQ ID NO: 346 - protein, 16 aa, Artificial Sequence
  Cris7 and DRA222 VH CDR2 (Kabat)
YINPSSAYTNYNQKFK

SEQ ID NO: 347 - protein, 11 aa, Artificial Sequence
  Cris7 and DRA222 VH CDR3 (Kabat)
QVHYDYNGFPY

SEQ ID NO: 348 - protein, 10 aa, Artificial Sequence
  Cris7 and DRA222 VL CDR1 (Kabat)
SASSSVSYMN

SEQ ID NO: 349 - protein, 7 aa, Artificial Sequence
  Cris7 and DRA222 VL CDR2 (Kabat)
DSSKLAS

SEQ ID NO: 350 - protein, 9 aa, Artificial Sequence
  Cris7 and DRA222 VL CDR3 (Kabat)
QQWSRNPPT

SEQ ID NO: 351 - protein, 8 aa, Artificial Sequence
  Cris7 and DRA222 VH CDR1 (IMGT)
GYTFTRST

SEQ ID NO: 352 - protein, 8 aa, Artificial Sequence
  Cris7 and DRA222 VH CDR2 (IMGT)
INPSSAYT

SEQ ID NO: 353 - protein, 9 aa, Artificial Sequence
  Cris7 and DRA222 VH CDR3 (IMGT)
QQWSRNPPT

SEQ ID NO: 354 - protein, 7 aa, Artificial Sequence
  Cris7 and DRA222 VL CDR1 (IMGT)
ASSSVSY

SEQ ID NO: 355 - protein, 3 aa, Artificial Sequence
  Cris7 and DRA222 VL CDR2 (IMGT)
DSS

SEQ ID NO: 356 - protein, 9 aa, Artificial Sequence
  Cris7 and DRA222 VL CDR3 (IMGT)
QQWSRNPPT

SEQ ID NO: 357 - protein, 5 aa, Artificial Sequence
  I2C VH CDR1 (Kabat)
KYAMN

SEQ ID NO: 358 - protein, 19 aa, Artificial Sequence
  I2C VH CDR2 (Kabat)
RIRSKYNNYATYYADSVKD

SEQ ID NO: 359 - protein, 14 aa, Artificial Sequence
  I2C VH CDR3 (Kabat)
HGNFGNSYISYWAY

SEQ ID NO: 360 - protein, 14 aa, Artificial Sequence
  I2C VL CDR1 (Kabat)
GSSTGAVTSGNYPN

SEQ ID NO: 361 - protein, 7 aa, Artificial Sequence
  I2C VL CDR2 (Kabat)
GTKFLAP

SEQ ID NO: 362 - protein, 9 aa, Artificial Sequence
  I2C VL CDR3 (Kabat)
VLWYSNRWV

SEQ ID NO: 363 - protein, 8 aa, Artificial Sequence
  I2C VH CDR1 (IMGT)
GFTFNKYA

SEQ ID NO: 364 - protein, 10 aa, Artificial Sequence
  I2C VH CDR2 (IMGT)
IRSKYNNYAT

SEQ ID NO: 365 - protein, 16 aa, Artificial Sequence
  I2C VH CDR3 (IMGT)
VRHGNFGNSYISYWAY

SEQ ID NO: 366 - protein, 9 aa, Artificial Sequence
  I2C VL CDR1 (IMGT)
TGAVTSGNY

SEQ ID NO: 367 - protein, 3 aa, Artificial Sequence
  I2C VL CDR2 (IMGT)
GTK

SEQ ID NO: 368 - protein, 9 aa, Artificial Sequence
  I2C VL CDR3 (IMGT)
VLWYSNRWV

SEQ ID NO: 369 - protein, 5 aa, Artificial Sequence
  HuM291 VH CDR1 (Kabat)
SYTMH

SEQ ID NO: 370 - protein, 17 aa, Artificial Sequence
  HuM291 VH CDR2 (Kabat)
YINPRSGYTHYNQKLKD

SEQ ID NO: 371 - protein, 11 aa, Artificial Sequence
  HuM291 VH CDR3 (Kabat)
SAYYDYDGFAY

SEQ ID NO: 372 - protein, 10 aa, Artificial Sequence
  HuM291 VL CDR1 (Kabat)
SASSSVSYMN

SEQ ID NO: 373 - protein, 7 aa, Artificial Sequence
  HuM291 VL CDR2 (Kabat)
DTSKLAS

SEQ ID NO: 374 - protein, 9 aa, Artificial Sequence
  HuM291 VL CDR3 (Kabat)
QQWSSNPPT

SEQ ID NO: 375 - protein, 8 aa, Artificial Sequence
  HuM291 VH CDR1 (IMGT)
GYTFISYT

SEQ ID NO: 376 - protein, 8 aa, Artificial Sequence
  HuM291 VH CDR2 (IMGT)
INPRSGYT

SEQ ID NO: 377 - protein, 13 aa, Artificial Sequence
  HuM291 VH CDR3 (IMGT)
ARSAYYDYDGFAY

SEQ ID NO: 378 - protein, 7 aa, Artificial Sequence
  HuM291 VL CDR1 (IMGT)
ASSSVSY

SEQ ID NO: 379 - protein, 3 aa, Artificial Sequence
  HuM291 VL CDR2 (IMGT)
DTS

SEQ ID NO: 380 - protein, 9 aa, Artificial Sequence
  HuM291 VL CDR3 (IMGT)
QQWSSNPPT

SEQ ID NO: 381 - protein, 251 aa, Artificial Sequence
  TSC455 (anti-CD3) TSC394 F87Y scFv
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTEYTLTISSLQPDDFATYYCQQWSRNPPTFGGGTKVEIKRSSS

SEQ ID NO: 382 - protein, 251 aa, Artificial Sequence
  TSC456 (anti-CD3) TSC394 E86D F87Y scFv
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRSSS

SEQ ID NO: 383 - protein, 121 aa, Artificial Sequence; identical to SEQ ID NO: 25
  TSC455 and TSC456 variable heavy domain

SEQ ID NO: 384 - protein, 108 aa, Artificial Sequence; identical to SEQ ID NO: 26
  TSC455 variable light domain

SEQ ID NO: 385 - protein, 108 aa, Artificial Sequence
  TSC456 variable light domain
DIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 386 - protein, 247 aa, Artificial Sequence
  DRA222 (anti-CD3) scFv
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 387 - protein, 121 aa, Artificial Sequence
  DRA222 variable heavy domain
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS

SEQ ID NO: 388 - protein, 107 aa, Artificial Sequence
  DRA222 variable light domain
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITS

SEQ ID NO: 389 - protein, 9 aa, Artificial Sequence
  anti-CD3 LCDR3
VLWYSNRWV